(19) United States
(12) Patent Application Publication
(10) Pub. No.: US 2024/0358855 A1
(43) Pub. Date: Oct. 31, 2024

(54) MULTIGENE CONSTRUCTS FOR TREATMENT OF AGE-RELATED MACULAR DEGENERATION AND OTHER COMPLEMENT DYSREGULATION-RELATED CONDITIONS

(71) Applicant: University of Utah Research Foundation, Salt Lake City, UT (US)

(72) Inventors: Gregory S. Hageman, Bountiful, UT (US); Burt T. Richards, Salt Lake City, UT (US)

(21) Appl. No.: 18/684,547

(22) PCT Filed: Aug. 18, 2022
(86) PCT No.: PCT/US2022/040800

§ 371 Date: Feb. 16, 2024

Related U.S. Application Data
(60) Provisional application No. 63/234,644, filed on Aug. 18, 2021.

(51) Int. Cl. A61K 48/00; A61K 38/17; A61P 21/00; C07K 16/22; C12N 9/22; C12N 15/113; C12N 15/86

(52) U.S. Cl. CPC A61K 48/005; A61K 38/1725; A61P 21/00; C07K 16/22; C12N 9/22; C12N 15/113; C12N 15/86; C07K 2317/31; C07K 2317/622; C12N 2310/122; C12N 2310/14; C12N 2310/20; C12N 2750/14143; C12N 2830/50; C12N 2840/203

(57) ABSTRACT

Gene therapy methods, vectors and cargo described herein can reduce or prevent C3b amplification and/or MAC formation within the neural retina, retinal pigmented epithelium (RPE), choroid, choriocapillaris (CC), Bruch's membrane and other ocular cells and tissues to re-establish appropriate control of the complement system. Gene therapy methods disclosed will benefit patients with complement related diseases such as AMD. Some gene therapy methods use a viral or nonviral vector comprising a cargo encoding two, three, or more than three activities selected from: a) one or more of a complement proteins selected from CFHT, oCFHT, CFH, and CFI; b) one of more binding proteins that specifically binds CFB, CFD, CFP, CFHR-1, CFHR-2, CFHR-3, CFHR-4, CFHR-5, C3, C4A, C4B, C5, C6, C7, C8A, C8B, C8G, or C9; c) HTRA1 protein or a transcriptional activator protein that increases expression of HTRA1; d) a binding protein that specifically binds ApoE2 or VEGFA; e) an inhibitory RNA that targets CFB, CFD, CFP, FHR-1, CFHR-2, CFHR-3, CFHR-4, CFHR-5, C3, C4A, C4B, C5, C6, C7, C8A, C8B, C8G, or C9; and f) an inhibitory RNA that targets ApoE2 or VEGFA. Also disclosed are optimized forms of truncated Complement Factor H (oCFHT) useful for treatment of patients in gene therapy (delivered alone or in combination with other activities).

### CROSS-REFERENCE TO RELATED APPLICATIONS

This application claims benefit of U.S. provisional application No. 63/234,644, filed Aug. 18, 2021, which is herein incorporated by reference for all purposes.

### STRUCTURE AND PROPERTIES OF MULTIGENE THERAPY ELEMENTS BACKGROUND

The complement system is continuously activated at low levels and both membrane-bound and soluble intraocular complement regulatory proteins tightly regulate this spontaneous complement activation. However, dysregulation of the complement system is associated with numerous diseases and conditions, including autoimmune diseases, angioedema, ocular disease, and others. In particular, over-activation of the complement system can result in ocular inflammation, which contributes to vision loss in a number of ocular diseases such as, for example, age-related macular degeneration (AMD), and diabetic macular edema.

Three distinct pathways activate the complement cascade. The classical, lectin, and alternative pathways all converge on an amplification loop that, if over-activated, results in inflammation and cell lysis. A fourth pathway, termed the intrinsic pathway, has been suggested. The cleavage of C3 into C3b and its deposition onto surfaces, (e.g., cells or extracellular matrix) initiates the amplification loop. Complement factor B (FB) and factor D (FD) contribute to the formation of a C3-convertase (C3bBb), which drives forward the amplification loop by cleaving additional C3 into C3b. However, this cascade can be prevented through inactivation of C3b (forming iC3b) by the enzyme complement factor I (FI) and its cofactor, complement factor H (FH) or its truncated splice variant FHL-1. iC3b cannot generate C3-convertase. If these regulators fail to control the complement cascade, downstream consequences include the release of the anaphylatoxins C3a and C5a, and ultimately the production of the terminal complement complex (TCC). TCC is also commonly referred to as the membrane attack complex (MAC), which can integrate into cell membranes causing lysis and death. Thus, the deposition of TCC/MAC can be regarded as a marker of complement activation.

Complement dysregulation occurs when the activation and control mechanisms of complement that together play crucial roles in maintaining health and tissue homeostasis fail; when the extremely delicate balance between activation and control is disturbed, tissue damage and disease ensue (Ricklin et al. 2016, 2018; Zelek et al 2019; Harris et al. 2018; Morgan & Harris 2015; Thurman and Holers 2006; Barlow 2017; Gavriilaki and Brodsky 2020; Zipfel et al. 2006; Schmidt et al. 2016).

Complement-based therapies are being developed that will play a key role in the treatment of complement-mediated diseases (Zelek et al. 2019; Harris et al. 2018; Morgan and Harris 2015; Ricklin and Lambris 2007; Kassa et al 2019; Read et al. 2004; Ren et al. 2010; Dobo et al. 2018; Gavriilaki and Brodsky 2020; Huber-Lang et al. 2016). However, additional and more effective therapies are needed.

### BRIEF SUMMARY

The gene therapy methods described herein can reduce or prevent C3b amplification and exert downstream effects, including MAC formation within the neural retina, retinal pigmented epithelium (RPE), choroid, choriocapillaris (CC), Bruch's membrane, and/or other ocular cells and tissues to re-establish appropriate control of the complement system.

In an aspect, disclosed herein is a gene therapy vector for treatment of conditions associated with complement dysfunction comprising a genetic cargo encoding two, three, or more than three activities selected from: a) one or more a complement proteins selected from CFH (FH), CFHT (FHL-1), oCFHT (oFHL-1), and CFI (FI); b) one of more binding proteins that specifically binds CFB (FB), CFD (FD), CFP, CFHR-1(FHR-1), CFHR-2 (FHR-2), CFHR-3 (FHR-3), CFHR-4 (FHR-4), CFHR-5 (FHR-5), C3, C4A, C4B, C5, C6, C7, C8A, C8B, C8G, or C9; c) HTRA1 protein or a transcriptional activator protein that increases expression of HTRA1; d) a binding protein that specifically binds ApoE2 or VEGFA; e) an inhibitory RNA that targets CFB, CFD, CFP, CFHR-1, CFHR-2, CFHR-3, CFHR-4, CFHR-5, C3, C4A, C4B, C5, C6, C7, C8A, C8B, C8G, or C9; and f) an inhibitory RNA that targets ApoE2 or VEGFA.

### DESCRIPTION OF THE DRAWINGS

FIG. 1 depicts the important role of the CFH/CFHT SCR7 domain in recognizing cell surface-associated GAGs and other ligands (top) and designed modified versions showing the additions of 1, 2 and 3 additional SCR7 domains to protective CFHT (bottom).

FIG. 2 shows SCR7-encoding sequences optimized by four different methods. [SEQ ID NO: 2-5].

FIG. 3 illustrates the addition of a FHR-1 dimerization domain (SCR1-2) sequence [SEQ ID NO: 5] which will provide advantageous characteristics to protective CFHT and other modified CFHT proteins.

FIG. 4 shows C7 mRNA levels in the RPE-choroid and retina in Chr1 and Chr10 AMD risk donor samples and Chr1-I62/Del protection samples. Each data point is an individual donor extramacular RPE-choroid (left) or extramacular retina (right) sample.

FIG. 5 shows plasma C7 protein concentration stratified by phenotype in Chr1-Risk only Patients. Significance was determined using the Mann-Whitney t-test.

FIG. 6 shows C7 protein levels following transient co-transformation of a human C7 cDNA expression construct and plasmid expressing C7 shRNA in Cos-7 cells. Measurements are 48 h post-transfection.

FIG. 7 provides data from an illustrative experiment showing the amount of secreted C7 protein in SK-N-AS and SK-N-SH cell supernatants following transduction with increasing amounts of pCTM467, pCTM468 and pCTM469 rAAV2 viral particles. Supernatants were analyzed for C7 protein concentration (top panel) and percent C7 protein reduction (bottom panel) by ELISAs and were normalized to non-transduced supernatants (0).

FIG. 8 provides data from an illustrative experiment showing protective CFHT protein expression levels in SK-N-AS and SK-N-SH cell supernatants after transduction with pCTM467, pCTM468 and pCTM469 rAAV2 particles.

FIG. 9 provides data from an illustrative experiment showing MDA-LDL binding activities of Cos-7 supernatants following transduction with increasing amounts of pCTM297 (CFHT.0), pCTM452 (mCFHT.1) and pCTM455 (dCFHT.0) AAV2-based gene therapy constructs.

FIG. 10 provides data from an illustrative experiment showing activities of purified recombinant CFHT and optimized CFHT proteins in a decay acceleration activity (DAA) assay.

FIG. 11 provides a map of plasmid pCTM467.

### DETAILED DESCRIPTION

### 1. Terminology

A “gene therapy vector” is a viral or nonviral vector used to deliver a polynucleotide (“cargo”) to a target cell or tissue. In some cases a gene therapy vector is a viral vector such as a recombinant adeno-associated virus vector (rAAV or AAV), adeno virus (AV), anellovirus, or lentivirus vector.

As used herein, “cargo” refers to the entire nucleic acid delivered to a cell using a gene therapy vector. The cargo may be DNA or RNA, depending on the choice of vector. In the case of certain viral vectors the cargo includes DNA between and including terminal repeats (e.g., inverted terminal repeats in the case of AAV and other vectors, or long terminal repeats in the case of lentiviral and other vectors). The cargo can include sequences that encode transcribed RNA and/or protein coding sequences, as well as regulatory elements, spacer sequences, terminal sequences, and the like. A cargo may include coding sequences for gene product (RNA or protein) one or more than one (e.g., two, three or four) gene products. Vectors encoding multiple gene products are used for multigene therapy. As used herein, a cargo is delivered by a single vector. Generally, a cargo is a single polynucleotide (e.g., a single contiguous DNA sequence encoding multiple coding sequences.

As used herein, “gene product” refers to a polypeptide or RNA encoded in a vector cargo and produced in a cell transduced by a gene therapy vector. Without limitation a gene product may be an mRNA, a polypeptide encoded by an mRNA, or an inhibitory nucleic acid (e.g., an inhibitory RNA). One, two, three, or more than three gene products can be encoded in a single cargo that is delivered by a single vector.

As used herein, “multigene cargo” refers a cargo that encodes multiple (e.g., 2, 3 or 4) gene products.

As used herein, “multigene therapy” refers to gene therapy in which a single cargo encoding two or more (e.g., two, three or four) gene products is administered.

As used herein, “transgene” refers to a portion of a cargo that encodes one protein.

As used herein, the term “activity” refers to the biological effect that results from expression of a gene product encoded in a gene therapy cargo. See Section 2.1, below.

As used herein, “delivery” (e.g., delivery of a gene therapy vector, delivery of a gene therapy cargo, delivery of an activity, delivery of a gene product, delivery of a cargo, delivery of a polynucleotide, etc.) refers to associating a viral or nonviral vector with a target cell under conditions in which the vector cargo is introduced (e.g., transduced, in the case of a viral vector) into the cell allowing the gene product(s) encoded in the cargo to be expressed in the cell. A gene therapy vector can be delivered to a subject by injection, infusion, and other methods. Generally a gene therapy vector results in delivery (directly or indirectly) to the eye. In some approaches, delivery results in transduction of RPE cells, endothelial choroidal cells or melanocyte cells.

As used herein, a “target cell” refers to the cell(s) or cell type(s) in which a gene product delivered by a therapy cargo is expressed. Importantly, a gene product expressed in a target cell may have a biological effect in neighboring cells and surrounding tissues. For example, the gene product may be secreted from the cell in which it is expressed and have effects in other cells. For example, CFHT may be expressed in and secreted by RPE cells and have effects in surrounding choroid tissue.

As used herein, the term “complement protein” refers to any protein associated with the complement system. Complement proteins may be synthesized by the liver and other tissues, and circulate in the blood and extracellular compartments as inactive precursors. When stimulated by one of several triggers, proteases in the system cleave specific proteins to release cytokines and initiate an amplifying cascade of further cleavages. The end result of this complement activation or complement fixation cascade is stimulation of phagocytes to clear foreign and damaged material, inflammation to attract additional phagocytes, and activation of the cell-killing membrane attack complex. About 50 proteins and protein fragments make up the complement system, including serum proteins, and cell membrane receptors. Three biochemical pathways activate the complement system: the classical complement pathway, the alternative complement pathway, and the lectin pathway. The alternative pathway accounts for the majority of terminal pathway activation and so therapeutic efforts in disease have revolved around its inhibition. Some proteins of the alternative pathway can be classified as “early,” “intermediate,” and “late” based on where they act in the alternative complement pathway.

TABLE 1

| Stage | Illustrative Proteins |
|---|---|
| Early | CFB, CFD, C3 |
| Intermediate | CFH, CFHT, CFHR1-5 |
| Late | C3a, C5a, C5, C6, C7, C8, C9 |

As used herein, the term “complement regulatory protein” refers broadly to membrane-bound and soluble intraocular complement regulatory proteins including: CFH, CFHT, CFI, CFB, CFD, CFP, CFHR1-5, C3, C4, C5, C6, C7, C8, C9, CD46, CD55, and CD59. As used herein, “complement regulatory proteins” are a subcategory of “complement proteins.”

As used herein, “exogenous protein” refers to a protein encoded in a gene therapy cargo. In some cases, the exogenous protein or a variant thereof is normally expressed in cells of the subject receiving gene therapy. For example, 402Y 621 CFH produced from a cargo is exogenous, while CFH (e.g., 402Y 621; 402Y 62V or 402H 62V) expressed from genomic sequences of the subject is an endogenous protein. In some cases, an exogenous protein is a not found in nature (e.g., a specific nanobody or trap) or a variant of a complement protein found in nature. Examples of variants include an optimized protein such as described in Section 3.4, below.

As used herein, an “endogenous protein” refers to a protein expressed by a subject prior to or in the absence of, treatment with a gene therapy vector.

As used herein, a “disease modifying protein” is a protein other than a complement protein that is associated with risk of or development of an ocular disease such as AMD. Examples of disease modifiers include HTRA1, VEGF, and ApoE2.

As used herein, “protective CFH” and “protective CFHT” refer to the CFH/T variant in which residue 62 is isoleucine (621) and residue 402 is tyrosine (402Y). See Hageman et al., 2005, Proc. Natl Acad Sci 102:7227-32 and U.S. Pat. No. 7,745,389, which is incorporated by reference in its entirety.

“Reference sequence” refers to a nucleic acid or protein sequence for which a sequence, database accession number, or published patent or literature reference is provided in this disclosure (see, e.g., Table 19). Reference sequences are provided to identify proteins and sequence elements. When an amino acid or nucleic acid sequence is provided in this disclosure for a protein, gene product, promoter, regulatory element, or the like, it will be understood that artificially or naturally occurring variants with substantially the same functional properties may be used. For example, protein or nucleic acid variants with at least about 85%, at least about 90% or at least about about 95% sequence identity to the reference sequence (i.e., variants substantially identical to a reference sequence) can be used in the methods and compositions of the invention and any reference to a protein of nucleotide sequence should be understood to refer to functionally similar variants. In addition, it will be understood that, due to codon degeneracy, a specified amino acid sequence can be encoded by a number of different DNA or RNA sequences. Provision of a particular protein-encoding nucleic acid sequence is not intended to limit the invention to that single sequence but encompasses other nucleic acid sequences encoding the protein or a variant or analog thereof. Similarly, an amino acid sequence provided as a translation of a nucleic acid sequence does not limit the nucleic acid sequence that encodes the protein to the particular nucleic acid sequence from which the protein was translated. A number of other nucleic acid sequences can also encode the protein.

As used herein, “sequence identity” in reference to similarity of two proteins (a target protein and a reference protein) or two nucleic acids (a target nucleic acid and a reference nucleic acid) is a quantification of identity of amino acids or nucleobases when the reference and target sequence are optimally aligned. Sequence identity can be determined manually by inspection, especially when the target and reference have greater than 90% identity and are easy to align. Alternatively, for nucleotide sequences, percent identity to a reference nucleic acid sequence can be determined using a BLAST or BLAST 2.0 comparison program (described in Altschul et al. (1990) J. Mol. Biol. 215: 403-410 and Altschul et al. (1977) Nucleic Acids Res. 25: 3389-3402, respectively) with default parameters. BLASTP with default parameters can be used to determine percent to a reference polypeptide sequence. The BLASTN program uses as default parameters a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)). Software for BLAST analyses is publicly available through the National Center for Biotechnology Information (NCBI) web site.

As used herein, the term a “target,” in reference to a binding agent (such as an antibody) that targets a protein means the binding agent specifically binds to the protein. The term “target,” in reference to an inhibitory RNA that targets a polynucleotide means the binding is complementary to a portion of the polynucleotide and hybridizes to the polynucleotide.

### 2. Introduction

Dysregulation of complement activation is associated with development of age-related macular degeneration (AMD) and many other diseases. A variety of treatment approaches have been proposed or explored for modulating the complement system and restoring health, including administration of small molecule inhibitors, direct administration of therapeutic proteins, treatment with antibodies against complement activities, and gene therapy.

We have determined that coordinated modulation of multiple (e.g., two or three) proteins in target cells or tissues to intervene at multiple regulatory points within the alternative pathway amplification cascade can reestablish appropriate regulation of the complement system in Chromosome 1- (Chr1-) directed AMD. (See Hageman and Richards, WO2020019002A1, incorporated herein by reference, for a discussion of Chr1-directed AMD.) The mutigene therapy described herein is a superior intervention strategy for treating this multi-factoral and complex age-related disease.

In addition, patients suffering from AMD can manifest a range of pathology in the same eye. These can, for example, range from normal histology to the presence of drusen and other abnormal extracellular deposits to retinal, RPE and choroidal cell death to geographic atrophy to neovacularization.

Removal of proteins or mRNAs produced locally in the eye (e.g. RPE) or systemically (e.g. liver or endothelial cells) that target critical early alternative complement pathway steps (CFB, CFD, C3), in concert with perturbation of intermediate (e.g. CFH, CFHT, CFHR1-5) and later (e.g. C5, C6, C7, C8, C9) alternative complement pathway steps will allow for tighter control of dysfunctional complement and reduction of disease progression. Additionally, introduction of multigene constructs that contain cargo that can block disease modifying or accelerating steps (APOE, VEGFA, HTRA1) will provide added benefit. In contrast to augmentation or inhibition of a single complement protein, introduction of multigene cargo permits augmentation or diminution of complement elements active at two or more stages of activation of the alternative complement (AC) system. In one approach, administration of a multigene cargo results in augmentation or diminution of complement proteins in two, three, or four different classes as well as (optionally) with disease modifying proteins. This approach allows multiple steps during disease progression to be controlled.

Multigene therapy requires that several gene therapy elements work together, and involves challenges specific to use of a single cargo to deliver more than one gene product or more than one activity. Important factors in multigene therapy include (I) the combination of gene products or activities delivered using a gene therapy vector; (II) regulatory elements that ensure the delivered activities are expressed and biologically active at the location(s), level(s), and/or time(s) required for the desired therapeutic effect; (III) the organization of the gene therapy vector and cargo; and (IV) the selection of a delivery system such as a specific viral vector or class of viral vectors or class of viral or nonviral vectors.

### 2.1 Activities

Each gene product encoded on a multigene cargo and delivered to a cell or tissue is associated with an activity. As noted above, the term “activity” refers to the biological effect that results from expression of a gene product encoded in a multigene cargo. The combination of activities delivered by a multigene cargo provides therapeutic benefit. Delivery of a gene product of a multigene cargo results in a perturbation of a target or target function. In some cases, delivery of a gene product results in augmentation of a target function, e.g., introduction of an exogenous complement protein or variant thereof, or disease modifier protein or variant thereof, or introduction of an activator (e.g., CRISPRa) that increases transcription of an endogenous protein. In some cases, delivery of a gene product results in diminution of a target function (i.e., diminution of an activity characteristic of a target complement protein or a target disease modifier protein). For illustration, in one example the gene product is aflibercept and the activity is diminution (effect) of VEGFA (target function). In this case the perturbation is diminution and the target function is VEGFA activity. In a related activity, the gene product is an inhibitory anti-VEGFA nanobody, the perturbation is diminution, and the target function is VEGFA activity. Thus, two different gene products can have the same activity, and the same activity can result from administration of two multigene cargos encoding different gene products. As a third example, gene product is an shRNA that targets VEGFA RNA, the perturbation is diminution, and the target function is VEGFA activity. See Sections 5.21 (aflibercept), 5.3 (nanobodies), and 5.1.1 (shRNA).

TABLE 2 provides exemplary activities that, delivered in combination, provide therapeutic benefit. In some cases, each activity delivered by a multigene cargo relates to a different target. For example, a multigene cargo that carries two activities may deliver activities that perturb two different targets, e.g., CFHT+CFI or CFHT+C7. In some cases, a multigene cargo that carries two activities may deliver activities that perturb the same target (e.g., CFHT+CFHT). Likewise, a multigene cargo that carries three activities may deliver two activities that perturb the same target and one activity that perturbs a different target (e.g., CFHT+CFHT+HTRA1). A multigene cargo that delivers two activities that perturb the same target may deliver two copies or versions of the same gene product (e.g., two copies of a CFHT encoding sequence) or may deliver two different gene products that perturb the same target (e.g., a nanobody that targets C7 and an shRNA that targets C7).

As noted above, the perturbation resulting from administration of a gene product can be characterized as diminution or augmentation.

As used herein, “augmentation” can refer to increasing the amount, quantity or activity of a target, by, for example, introducing a cargo comprising a nucleic acid sequence encoding an exogenous protein. In an other approach, the cargo includes protein and RNA components of a CRISPR activation (CRISPRa) system to increase endogenous expression of the target (e.g., HTRA1; see Williams et al. WO2020210724A1, incorporated herein by reference). In some cases an activity delivered by the multigene cargo is an exogenous complement protein (e.g., the cargo comprises a nucleic acid encoding a complement protein), and delivery of the cargo results in expression of the exogenous protein. The exogenous protein may be a wild-type human complement protein, an optimized, variant or protective complement protein, a disease modifying protein, or the like. In this approach, augmentation of an activity is achieved by delivering a cargo to a cell, wherein a protein encoded by the cargo is expressed. Some expressed proteins are secreted from the cell in which they are synthesized. As noted in Section 1, the gene product may be secreted from the cell in which it is expressed and have effects in other cells.

As used herein, “diminution” refers broadly to reducing the quantity or activity of a target in the eye or other tissue. Diminution can refer to reducing expression of a target, reducing the amount of active target (e.g., by trapping), inactivating, altering or inhibiting the biological effect of a the target. In one approach diminution results from expression of cargo encoding an inhibitory RNA or inhibitory binding agent (e.g., trap, antibody, aptamer). In some cases an activity delivered by the single vector is a binding activity (e.g., nanobody, trap, aptamer) that, when delivered and expressed, reduces the amount or activity of an endogenous protein (e.g., complement protein) produced locally (e.g., RPE) or systemically (e.g., liver, endothelial cells) such as FHR1-5. In some cases an activity delivered by the single vector is an inhibitory RNA that, when delivered and expressed, reduces the amount or activity of an endogenous polypeptide locally in the transduced cell (e.g., C7).

TABLE 2
ACTIVITIES DELIVERED BY MULTIGENE CARGO

| | | | TARGET |
|---|---|---|---|
| | | | OR |
| | | | TARGET |
| ACTIVITY | GENE PRODUCT | PERTUBATION | FUNCTION |
| Enhance CFHT/CFH/CFI-mediated cleavage of | Transgene encoding protective or neutral complement factor H | Augmentation | CFH |
| C3b. Decorates self via GAGs, basement | (CFH) polypeptide | | |
| membrane (BM) and C3b. | Transgene encoding protective or neutral truncated | Augmentation | CFHT |
| | complement factor H (CFHT) polypeptide | | |
| CFHT with enhanced half-life, binding affinity | Transgene encoding optimized (modified) CFHT polypeptide | Augmentation | CFHT |
| and avidity to cell surface-associated GAGs, BM | Transgene encoding optimized (dimerized) CFHT polypeptide | Augmentation | CFHT |
| and C3b. | Transgene encoding optimized (modified and dimerized) CFHT | Augmentation | CFHT |
| | polypeptide mdCFHT | | |
| Augmentation of CFI co-factor activity with | Transgene encoding complement factor I (CFI) polypeptide | Augmentation | CFI |
| CFHT or CFH | | | |
| Enhance HTRA1 for concurrent treatment of | Transgene encoding HTRA1 polypeptide | Augmentation | HTRA1 |
| Ch10 disease-See WO2020210724 | HTRA1 expression activator | Augmentation | HTRA1 |
| Block CFB to decrease competition by CFB with | Binding agent for complement factor B (CFB) polypeptide | Diminution | CFB |
| CFHT for C3b binding, thus enhancing | | | |
| regulation. | Inhibitory RNA for complement factor B (CFB) polypeptide | Diminution | CFB |
| Prevent cleavage and activation of CFB and the | Binding agent for complement factor D (CFD) polypeptide | Diminution | CFD |
| ensuing formation of C3 convertase. | Inhibitory RNA for complement factor D (CFD) polypeptide | Diminution | CFD |
| Prevent stabilization of C3 convertase | Binding agent for complement factor Properdin (CFP) | Diminution | CFP |
| | polypeptide | | |
| Prevent stabilization of C3 convertase | Inhibitory RNA for complement factor Properdin (CFP) | Diminution | CFP |
| | polypeptide | | |
| Reduce function of central complement | Binding agent for complement C3 (C3) polypeptide | Diminution | C3 |
| pathway activator | | | |
| Reduce levels of central complement pathway | Inhibitory RNA for complement C3 (C3) polypeptide | Diminution | C3 |
| activator | | | |
| Reduce C3 and C5 convertase activity | Binding agent for complement C4A (C4A) polypeptide | Diminution | C4A |
| Reduce C3 and CS convertase activity | Inhibitory RNA for complement C4A (C4A) polypeptide | Diminution | C4A |
| Reduce C3 and C5 convertase activity | Binding agent for complement C4B (C4B) polypeptide | Diminution | C4B |
| Reduce C3 and C5 convertase activity | Inhibitory RNA for complement C4B (C4B) polypeptide | Diminution | C4B |
| Reduce C5 convertase activity and MAC | Binding agent for complement C5 (C5) polypeptide inhibitor | Diminution | C5 |
| formation | | | |
| Reduce C5 convertase activity, MAC formation | Inhibitory RNA for complement C5 (C5) polypeptide | Diminution | C5 |
| and MAC intercalation into cell membranes and | | | |
| prevent cell dysfunction and death | | | |
| Slow the rate of MAC formation and MAC | Binding agent for complement C6 (C6) polypeptide | Diminution | C6 |
| intercalation into cell membranes and prevent | Inhibitory RNA for complement C6 (C6) polypeptide | Diminution | C6 |
| cell dysfunction and death | Binding agent for complement C7 (C7) polypeptide | Diminution | C7 |
| | Inhibitory RNA for complement C7 (C7) polypeptide | Diminution | C7 |
| | Binding agent for complement C8A (C8A) polypeptide | Diminution | C8A |
| | Binding agent for complement C8B (C8B) polypeptide | Diminution | C8B |
| | Binding agent for complement C8G (C8G) polypeptide | Diminution | C8G |
| | Inhibitory RNA for complement C8G (C8G) polypeptide | Diminution | C8G |
| | Binding agent for complement C9 (C9) polypeptide | Diminution | C9 |
| | Inhibitory RNA for complement C9 (C9) polypeptide | Diminution | C9 |
| Decrease competition of endogenous | Binding agent for complement factor H related protein 1 | Diminution | CFHR1 |
| CFH/CFHT and exogenous delivered protective | (CFHR1) polypeptide | | |
| CFH/CFHT for endogenous ligands (including, | Inhibitory RNA for complement factor H related protein 1 | Diminution | CFHR1 |
| but not limited to, GAGs, Bruch′s membrane, | (CFHR1) polypeptide | | |
| C3b, lipid and protein modified adducts, | Binding agent for complement factor H related protein 2 | Diminution | CFHR2 |
| carbohydrates, adrenomedullin, CRP, SIBLING | (CFHR2) polypeptide | | |
| proteins, etc.) | Inhibitory RNA for complement factor H related protein 2 | Diminution | CFHR2 |
| | (CFHR2) polypeptide | | |
| | Binding agent for complement factor H related protein 3 | Diminution | CFHR3 |
| | (CFHR3) polypeptide | | |
| | Inhibitory RNA for complement factor H related protein 3 | Diminution | CFHR3 |
| | (CFHR3) polypeptide | | |
| | Binding agent for complement factor H related protein 4 | Diminution | CFHR4 |
| | (CFHR4) polypeptide | | |
| | Inhibitory RNA for complement factor H related protein 4 | Diminution | CFHR4 |
| | (CFHR4) polypeptide | | |
| | Binding agent for complement factor H related protein 5 | Diminution | CFHR5 |
| | (CFHR5) polypeptide | | |
| | Inhibitory RNA for complement factor H related protein 5 | Diminution | CFHR5 |
| | (CFHR5) polypeptide | | |
| Inhibit VEGF activity to reduce formation of | Binding agent, Aflibercept Vascular Endothellal Growth factor A | Diminution | VEGFA |
| neovascular membranes | (VEGFA) polypeptide | | |
| Inhibit VEGF activity to reduce formation of | Inhibitory RNA for Vascular Endothelial Growth factor A | Diminution | VEGFA |
| neovascular membranes | (VEGFA) polypeptide | | |
| Reduce accumulation of ApoE2 | Binding agent for ApoE2 polypeptide | Diminution | APOE |

TABLES 3 and 4 describe exemplary combinations of targets and categories of gene products that can be administered in a multigene cargo for coordinate expression. TABLE 3 illustrates delivery of two gene products. TABLE 4 illustrates delivery of three gene products. The labels “Activity 1,” “Activity 2,” and “Activity 3” do not indicate a particular order or 5′ to 3′ arrangement of genes in the cargo. “(P)” refers to expression of a complement protein (naturally occurring, modified, or optimized), disease modifier protein, or other protein to augment levels of the complement polypeptide in the target cell. “B” refers to a binding agent. Examples of binding activities include single chain antibodies (e.g., nanobodies), aptamers, and traps. “R” refers to an inhibitory RNA such as shRNA, siRNA, miRNA.

TABLE 3

| Activity 1 | Activity 2 |
|---|---|
| CFHT (P) | CFHT (P) |
| CFH (P) | CFHT (P), oCFHT (P) |
| CFH (P), CFHT (P), or | CFI (P) |
| oCFHT (P) | |
| CFH (P), CFHT (P), or | aflibercept (B) |
| oCFHT (P) | |
| CFHT (P) or oCFHT (P) | C5 (R), C6 (R), C7(R) VEGF (R, B), |
| | HtrA1 (P), APOε2 (R, B). |
| CFHT (P) or oCFHT (P) | CFB (B,R), CFHR1 (B,R), CFHR2 |
| | (B,R), CFHR3 (B,R), CFHR4 (B,R), |
| | CFHRS (B,R) |

TABLE 4

| ACTIVITY 1 | ACTIVITY 2 | ACTIVITY 3 |
|---|---|---|
| Aflibercept (B) | CFHT or oCFHT (P) | C7 (B,R) |
| CFHT (P) | CFHR1 (B) | C7 (B,R) |
| CFHT (P) | CFHR4 (B) | C7 (B,R) |
| CFHT (P) | CFI (P) | C7 (B,R) |
| CFH, CFHT, | CFI (P) | C7 (B,R) |
| oCFHT (P) | | |
| CFHT (P) | CFHR1 (B) | CS (B,R), C6 (B,R), |
| | | C7 (B,R) |
| CFHT (P) | Bispecific CFHR1- | C7 (B,R) |
| | CFHR4 (B) | |

In some approaches the cargo encodes two activities under control of one promoter. In some approaches the cargo encodes three activities under control of two or three promoters. In some approaches at least one activity is an inhibitory RNA (e.g., shRNA). In some approaches at least one activity is an exogenous complement protein (e.g., CFH, CFHT, CFI) and at least one activity is an inhibitory RNA (e.g., shRNA).

TABLE 5 is provided for illustration and not limitation and shows various schema for organizing transgenes according to the invention. “ITR” indicates vector is AAV; “LTR” indicates the vector is Lentivirus; [NV] indicates the vector is non-viral vector.

TABLE 5

| | |
|---|---|
| 1. | ITR-Promoter-Gene#1-Internal Ribosome Entry Site-Gene#2-PolyA-ITR |
| 2. | ITR-Promoter-Gene#1-CleavageSite-Gene#2-PolyA-ITR |
| 3. | ITR-Promoter-Gene#1-CleavageSite-Gene#2-PolyA-U6-hpRNA-ITR |
| 4. | ITR-PolyA#1-Gene#1-BidirectionalPromoter-Gene#2-PolyA#2-ITR |
| 5. | ITR-PolyA#1-Gene#1-BidirectionalPromoter-Gene#2-PolyA#2-U6-hpRNA-ITR |
| 6. | LTR-Promoter-Gene#1-InternalRibosomeEntrySite-Gene#2-PolyA-LTR |
| 7. | LTR-Promoter-Gene#1-InternalRibosomeEntrySite-Gene#2-PolyA-U6-hpRNA-LTR |
| 8. | LTR-Promoter-Gene#1-CleavageSite-Gene#2-PolyA-LTR |
| 9. | LTR-Promoter-Gene#1-CleavageSite-Gene#2-PolyA-U6-hpRNA-LTR |
| 10. | LTR-PolyA#1-Gene#1-BidirectionalPromoter-Gene#2-PolyA#2-LTR |
| 11. | LTR-PolyA#1-Gene#1-BidirectionalPromoter-Gene#2-PolyA#2-U6-hpRNA-LTR |
| 12. | LTR-U6-hpRNA#1-PolyA-eCFH/CFHT-Promoter-LTR |
| 13. | [NV]-Promoter-Gene#1-InternalRibosomeEntrySite-Gene#2-PolyA-U6-hpRNA |
| 14. | [NV]-Promoter#1-Gene#1-CleavageSite-Gene#2-PolyA#1-Promoter#2-Gene#3-PolyA#2 |
| 15. | [NV]-Promoter#1-Gene#1-CleavageSite-Gene#2-PolyA#1-Promoter#2-Gene#3-PolyA#2- |
| | U6-hpRNA |

### 3. Brief Descriptions of Complement Proteins and Disease Modifying Proteins (“Targets”)

### 3.1 Complement Factor H (CFH; FH-1; FH1)

Complement Factor H (CFH) is secreted into the bloodstream or locally by cells and tissues and acts as an inhibitor of complement activation in fluid phases (e.g. blood; extracellular compartments) and on self cell surfaces. CFH accelerates the decay of the complement alternative pathway (AP) C3 convertase C3bBb, thus preventing local formation of more C3b and, as a cofactor of the serine protease factor I, regulates proteolytic degradation of already-deposited C3b. As further explained in the following paragraph, multiple haplotypes associated with AMD risk or protection have been identified for the CFH/CFHT gene (Risk, Protective, Neutral and Protective-Deletion). For purposes of the compositions and methods described herein, CFH is preferably a protective form (I62, 402Y). Alternatively, a neutral form (62V, 402Y) may be employed. It will be recognized that the CFH protein exists in various forms in nature and that it is possible to modify the protein sequence without loss of biological activity. In some approaches, CFH variants with at least about 90% or about 95% sequence identity to a naturally occurring CFH sequence can be used.

### 3.2 Truncated CFH (CFHT, FLH-1; FHL1)

CFHT is encoded by a CFH gene splice variant. CFHT contains the first 7 SCRs of CFH and a unique four amino acid carboxy-terminal tail. It performs many of the same functions as CFHT. Multiple haplotypes associated with AMD risk or protection have been identified for the CFH/CFHT gene (Risk, Protective, Neutral and Protective-Deletion). For purposes of the compositions and methods described herein, CFHT is preferably a protective form (I62, 402Y). See, e.g., U.S. Pat. No. 7,745,389; WO2020019002; U.S. Pat. No. 7,867,727; and Hageman et al., Proc. Nat. Acad. Sci. USA, 202(20):7227-32, 2005, all incorporated by reference. An illustrative protective CFHT polypeptide sequence, lacking the signal peptide, is provided in SEQ ID NO:253. Alternatively, a neutral form of CFHT (62V, 402Y) may be used. It will be recognized that the CFHT protein exists in various forms in nature (see, e.g., Skerka et al., Br. J. Pharmacol 178:2823-2831, 2021) and that it is possible to modify the protein sequence without loss of biological activity. In some approaches, CFHT variants with at least about 90% or about 95% sequence identity to SEQ ID NO:253 can be used.

### 3.3 eCFH/T

eCFH/T is an artificial gene sequence that, when delivered to a cell, results in expression of CFH and a modified CFHT (comprising a carboxy-terminal sequence CIRVSKSFTL. eCFH/T is described in detail in WO2020019002, incorporated herein by reference.

### 3.4 Optimized Truncated CFH (oCFHT)

Optimized CFHT (oCFHT) proteins are designed to increase the affinity and/or avidity of CFHT binding to cell surfaces and CFHT ligands such as glycosaminoglycans (GAGs) associated with extracellular matrices, such as basement membrane associated GAGs. oCFHTs comprise a CFHT domain along with additional copies of short consensus repeat 7 (SCR7), SCR7 variants, and/or dimerization domains. As indicated above, CFHT is preferably a protective form, but alternatively may be a neutral form. The introduction of optimized CFHT complement pathway regulator proteins will be employed to reduce the degree of risk in AMD patients—by shifting the balance at the RPE/CC interface from risk toward protection—in patients with chromosome-1-directed risk AMD. This approach should reduce visual loss associated with early, late stage geographic atrophy and late-stage neovascular forms of AMD. In addition to AMD, these agents should be useful in reducing the complications of Chr1-directed pathologies in other diseases that exhibit alternative complement pathway dysfunction (e.g. membranoproliferative glomerulonephritis, dense deposit disease, IgA nephropathy, etc.). TABLE 6 lists three forms of oCFHT, which are also discussed below.

TABLE 6
Optimized CFHT (oCFTH)

| | |
|---|---|
| mCFHT | addition of 1, 2, 3 or more than 3 additional SCR7 domain(s) |
| dCFHT | addition of a SCR1-2 dimerization domain |
| mdCFHT | addition of a SCR1-2 dimerization domain and SCR7 |
| | domain(s) |

### 3.4.1 Modified CFHT (mCFHT)

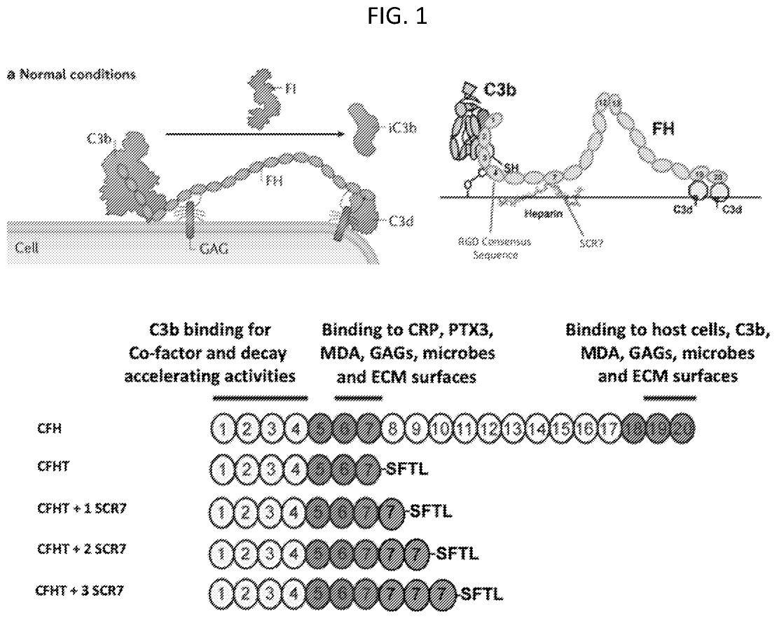

In one form of optimized CFHT, increased avidity of modified protective or non-protective CFHT binding is accomplished by the addition of 1, 2,3 or more additional single consensus repeat 7 (SCR7) domains. SCR7 domains contain important amino acid residues for binding to endogenous ligands (including, but not limited to, GAGs, C3b, lipid and protein malondialdehyde (MDA) and malondialdehyde-acetalaldehyde (MAA) modified adducts, carbohydrates, adrenomedullin, CRP, SIBLING proteins, etc.) (Sanchez-Corral et al., 2018, Front. Immunol. 9:1607-1625) in both CFH and CFHT. An exemplary amino acid sequence of CFH SCR7 is provided as SEQ ID NO:242. Exemplary DNA sequences encoding SCR7 are provided at SEQ ID NO:252 (wild-type) and SEQ ID NOs:2-5 (human codon-optimized). As illustrated herein, e.g., Section 10.5, the in vitro functions of mCFHT (cofactor, decay accelerating activity and ligand binding activity) will better regulate complement activation than non-modified CFHT protein, as has been demonstrated for other engineered mini-FH/mini-CFH proteins (Schmidt, C. Q., et al., 2013, J Immunol 190:5712-5721). FIG. 1 (top) illustrates the important role of the CFH/CFHT SCR7 domain in recognizing cell surface-associated GAGs. FIG. 1 (bottom) illustrates modified CFHT versions with 1, 2 and 3 additional SCR7 domains. An increased density of modified CFHT proteins on cell surfaces and extracellular surfaces, including basement membrane surfaces, will reduce or prevent uncontrolled activation of complement, and will stabilize binding of CFHT to C3b, thus diminishing C3b binding with complement factor B (CFB) and eventual MAC accumulation.

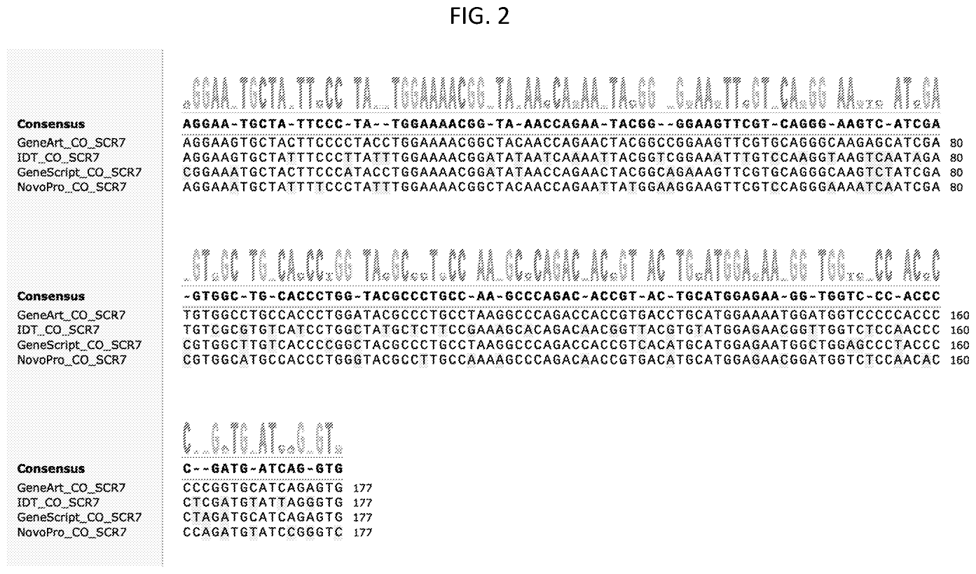

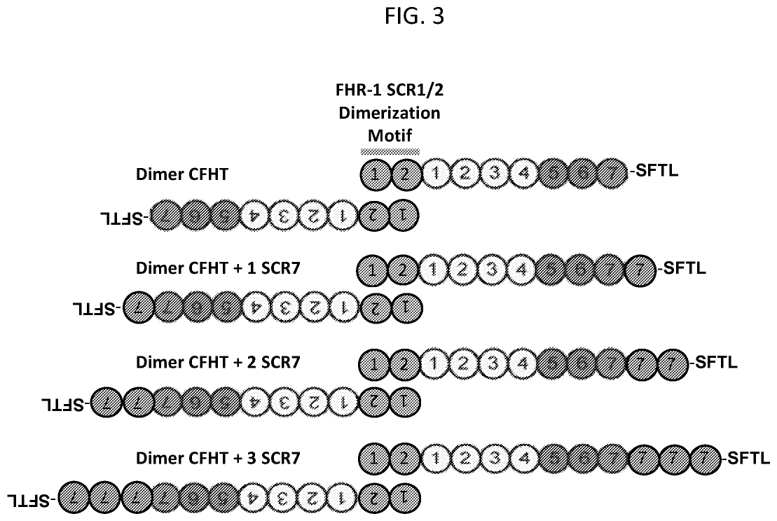

FIG. 2 demonstrates the nucleotide diversity between four human codon-optimized SCR7 sequences (SEQ ID NOS:2-5) to avoid plasmid DNA instability due to highly homologous nucleotide sequences. In one approach, in a cargo encoding mCFHT each SCR7-encoding module comprises a different nucleotide sequence.

In addition to the human SCR-7 sequence (e.g., SEQ ID NO:242), polypeptide variants with at least 85% sequence identity, preferably at least about 90% sequence identity, sometimes at least about 95% sequence identity may be used provided the SCR-7 sequence confers improved binding to C3b, MDA-LDL and CRP when incorporated into CFHT as described herein (e.g., to produce mCFHT.1).

### 3.4.2 Dimer CFHT (dCFHT)

In a second form of optimized CFHT (herein referred to dCFHT), a dimerization domain (FHR-1 SCR1-2, also referred to as “D”) sequence is included at the N- and/or C-terminal regions of CFHT to provide increased avidity for cell surfaces and to increase protein half-life and avidity (see Yang et al, 2018, “An Engineered Complement Factor H Construct For Treatment Of C3 Glomerulopathy” J Am Soc Nephrol. 29(6):1649-1661). In a preferred embodiment the dimerization domain is derived from SCRs 1 and 2 from Complement Factor H-Related 1 (CFHR-1, FHR-1). Exemplary nucleotide sequences encoding the FHR-1 SCR1 and SCR2 domains are provided at SEQ ID Nos: 52 to 55. In other embodiments dimerization sequences (SCRs 1-2) from CFHR2 or CFHR5 are used. SCRs 1-2 from CFHR1, 2 and 5 are conserved and share about 85% amino acid identity. See, e.g., Goicoechea de Jorge, 2013, Dimerization of complement factor H-related proteins modulates complement activation in vivo, PNAS, 110 (12) 4685-4690. In other approaches, dimerization domains from other proteins may be used. Exemplary amino acid sequences encoding the FHR-1, FHR-2 and FHR-5 SCR1 and SCR2 domains are available in the scientific literature and databases, and are provided at SEQ ID Nos: 1, 228 and 233. Exemplary nucleotide sequences encoding the FHR-2 and FHR-5 SCR1 and SCR2 domains are provided at SEQ ID Nos: 229-232 and 234-237. Dimerization domains for use in the invention include sequences of SCR1-2 from FHR-1, FHR-2 and FHR-5; as well as variants with at least 85% sequence identity, preferably at least about 90% sequence identity, sometimes at least about 95% sequence identity which retain the ability to homodimerize.

Modified-Dimer CFHT (mdCFHT)

A third form of enhanced CFHT protein contains both additional SCR7 domains (as found in mCFHT) and additional dimerization domain(s) (as found in dCFHT). mdCFHT will reduce complement activation and provide several potential therapeutic benefits. Without limiting the invention to specific embodiments, the following exemplary constructs are contemplated (numbers 1-7 refer to short consensus repeats (SCR) in CFHT:
1-2-3-4-5-6-7-SFTL-D
1-2-3-4-5-6-7-7-SFTL-D
1-2-3-4-5-6-7-7-7-SFTL-D
1-2-3-4-5-6-7-7-7-7-SFTL-D
D-1-2-3-4-5-6-7-SFTL
D-1-2-3-4-5-6-7-SFTL
D-1-2-3-4-5-6-7-7-SFTL
D-1-2-3-4-5-6-7-7-7-SFTL-D
D-1-2-3-4-5-6-7-7-7-7-SFTL-D

As a result of these modifications, the half-life of CFHT protein on cell and extracellular surfaces will be increased, as has been demonstrated with other engineered mini-FH/mini-CFH proteins (Yang et al., J Am Soc Nephrol 29:1649-1661, 2018). CFHT is a ‘negative’ regulator of the AP system and expression of the modified CFHT protein, containing the CFHR-1 dimerization domain, will bind to monomeric CFHR1 (or CHFR2 or CFHR-5) protein in the eye, thereby preventing it from competing with CFH/CFHT for C3b, MDA, PTX3, adrenomedullin and/or CRP binding and thus reducing activation of C3b or other inflammatory molecules. Lastly, when the region of the CFHT protein dimer containing extra SCR7 domain(s) binds to self, it will bind nearby C3b protein(s), leaving the non-SCR-containing portion of the dimer to bind additional C3b protein(s) or GAG(s) on cell surfaces or extracellular matrices (e.g., BM; choroidal stroma, etc.) or other endogenous ligands (including, but not limited to, GAGs, lipid and protein modified adducts, carbohydrates, adrenomedullin, CRP, PTX, SIBLING proteins, etc.). Binding to as many C3b and GAG molecules as possible will reduce CFB binding to C3b and block generation of the C3 convertase complex that amplifies AP complement activation. Inhibition of C3 convertase generation will significantly reduce both the inflammatory anaphylatoxins C3a and C5a and membrane attack complex (MAC) that drive all stages of AMD progression.

### 3.5 Complement Factor B (CFB)

CFB circulates in the blood as a single chain polypeptide. Upon activation of the alternative pathway, it is cleaved by complement factor D, yielding the noncatalytic chain Ba and the catalytic subunit Bb. The active subunit Bb is a serine protease which associates with C3b to form the alternative pathway C3 convertase. Polymorphisms in this gene are associated with a reduced risk of age-related macular degeneration. Human CFB cDNA and amino acid sequences are provided at Genbank accession nos. NM_001710 (reference sequences). It will be recognized that the CFB protein exists in various forms in nature and that it is possible to modify the protein sequence without loss of biological activity. In some approaches, CFB variants with at least about 90% or about 95% sequence identity to the reference sequence can be used.

### 3.6 Complement Factor I

CFI regulates complement activation by cleaving C3b and C4b. Complement Factor I is produced by furin cleavage of a proprotein to generate a glycoprotein heterodimer consisting of a disulfide linked heavy chain and light chain. Human CFI cDNA and amino acid sequences are provided at Genbank accession nos. NM_000204 (reference sequences). It will be recognized that the CFI protein exists in various forms in nature and that it is possible to modify the protein sequence without loss of biological activity. In some approaches, CFI variants with at least about 90% or about 95% sequence identity to the reference sequence can be used.

### 3.7 Complement Factor P

CFP (properdin) is a positive and sometimes negative regulator of the complement alternate pathway depending on disease context. See Chen et al., 2018, “Properdin: A Multifaceted Molecule Involved In Inflammation And Diseases” Mol Immunol. 102:58-72. It binds to and stabilizes C3- and C5-convertase enzyme complexes and inhibits CFI-CFH mediated degradation of Complement C3 beta chain (C3b). Properdin promotes the association of C3b with Factor B and provides a focal point for the assembly of C3bBb on a surface. It binds to preformed alternative pathway C3-convertases and inhibits the Factor H—mediated cleavage of C3b by Factor I.

### 3.8 Complement Component C3

Complement Component C3 plays a central role in the activation of the complement system. Its activation is required for both classical and alternative complement activation pathways. One form of C3-convertase, also known as C4b2a, is formed by a heterodimer of activated forms of C4 and C2. It catalyzes the proteolytic cleavage of C3 into C3a and C3b, generated during activation through the classical pathway as well as the lectin pathway. C3a is an anaphylatoxin and the precursor of some cytokines such as ASP, and C3b serves as an opsonizing agent. Factor I can cleave C3b into C3c and C3d, the latter of which plays a role in enhancing B cell responses. In the alternative complement pathway, C3 is cleaved by C3bBb, another form of C3-convertase composed of activated forms of C3 (C3b) and factor B (Bb). Once C3 is activated to C3b, it exposes a reactive thioester that allows the peptide to covalently attach to any surface that can provide a nucleophile such as a primary amine or a hydroxyl group. Activated C3 can then interact with factor B. Factor B is then activated by factor D, to form Bb. The resultant complex, C3bBb, is called the alternative pathway (AP) C3 convertase. C3bBb is deactivated in steps. First, the proteolytic component of the convertase, Bb, is removed by complement regulatory proteins having decay-accelerating factor (DAF) activity. Next, C3b is broken down progressively to first iC3b, then C3c+C3dg, and then finally C3d. Factor I is the protease that cleaves C3b but requires a cofactor (e.g. Factor H, CR1, MCP or C4BP) for activity.

### 3.9 Complement Component C4 (C4A, C4B)

Complement Component C4 serves a number of critical functions in immunity, tolerance, and autoimmunity with the other numerous components. Furthermore, it is a crucial factor in connecting the recognition pathways of the overall complement system instigated by antibody-antigen (Ab-Ag) complexes to the other effector proteins of the innate immune response. The C4 protein derives from the C4A-C4B genes, which allows for an abundant variation in the levels of their respective proteins within a population. Inhibition of C4A reduces C3 and C5 convertase activity.

### 3.10 Complement Component C5

The Complement Component C5 gene encodes a preproprotein that is proteolytically processed to generate multiple protein products, including the C5 alpha chain, C5 beta chain, C5a anaphylatoxin and C5b. The C5 protein is composed of the C5 alpha and beta chains, which are linked by a disulfide bridge.

### 3.11 Complement Component C6 (C6)

The Complement Component C6 gene encodes a component of the complement cascade. The encoded protein is part of the membrane attack complex that can be incorporated into the cell membrane and cause cell lysis. Mutations in this gene are associated with complement component-6 deficiency. Transcript variants encoding the same protein have been described.

### 3.12 Complement Component C7 (C7)

The Complement Component C7 gene encodes a serum glycoprotein that forms a membrane attack complex together with complement components C5b, C6, C8, and C9 as part of the terminal complement pathway of the innate immune system. Elevated Levels of C7 are associated with Chr1 Risk AMD. Exemplary C7 gene and cDNA sequences are provided at Genbank accession nos. NG-11692 and NM_000587 (reference sequences).

### 3.13 Complement Component C8 (C8A, C8B, C8G)

The Complement Component C8A, C8B and C8G genes encode protein components of the complement system that contains three polypeptides, alpha, beta and gamma. C8 proteins participate in the formation of the membrane attack complex.

### 3.14 Complement Component C9 (C9)

The Complement Component C9 gene encodes the final component of the complement system. It participates in the formation of the membrane attack complex (MAC). The MAC assembles on membranes to form a pore, permitting disruption and cell death. Mutations in this gene cause C9 deficiency. Exemplary C9 gene and cDNA sequences are provided at Genbank accession nos. NG_009894 and NM_001737 (reference sequences).

### 3.15 Complement Factor H Related 1(CFHR1; FHR1; HFL1; CFHL1)

The Complement Complement Factor H Related 1 gene encodes a secreted protein involved in complement regulation belonging to the complement factor H protein family. It binds to Pseudomonas aeruginosa elongation factor Tuf together with plasminogen, which is proteolytically activated. It is proposed that Tuf acts as a virulence factor by acquiring host proteins to the pathogen surface, controlling complement, and facilitating tissue invasion. Dimerized forms of CFHR1 protein have avidity for tissue-bound complement fragments and efficiently compete with the physiological complement inhibitor CFH. It can also associate with lipoproteins and may play a role in lipid metabolism.

### 3.16 Complement Factor H Related 2 (CFHR2; FHR2; HFL2; CFHL2)

The Complement Factor H Related 2 gene encodes a protein involved in regulation of complement. Mutations in CFHR genes have been associated with dense deposit disease and atypical haemolytic-uremic syndrome. Alternatively spliced transcript variants have been found for this gene. Its dimerized forms have avidity for tissue-bound complement fragments and efficiently compete with the physiological complement inhibitor CFH. It can also associate with lipoproteins and may play a role in lipid metabolism.

### 3.17 Complement Factor H Related 3 (CFHR3; FHR3; CFHL3)

The Complement Factor H Related 3 gene encodes a secreted protein which binds to heparin, and is likely involved in complement regulation. Mutations in this gene are associated with decreased risk of age-related macular degeneration, and with an increased risk of atypical hemolytic-uremic syndrome. Alternatively spliced transcript variants encoding different isoforms have been described.

### 3.18 Complement Factor H Related 4 (CFHR4; FHR4; CFHL4)

The Complement Factor H Related 4 gene encodes a protein that enhances the cofactor activity of CFH, and is involved in complement regulation. This protein can associate with lipoproteins and may play a role in lipid metabolism. Alternatively spliced transcript variants encoding different isoforms (varying in the number of SCRs) have been described.

### 3.19 Complement Factor H Related 5 (CFHR5; FHR5; CFHL5)

The Complement Factor H Related 5 gene encodes a protein having nine SCRs with the first two repeats having heparin binding properties, a region within repeats 5-7 having heparin binding and C reactive protein binding properties, and the C-terminal repeats being similar to a complement component 3 b (C3b) binding domain. This protein co-localizes with C3, binds C3b in a dose-dependent manner, and is recruited to tissues damaged by C-reactive protein. Allelic variations in this gene have been associated, but not causally linked, with two different forms of kidney disease: membranoproliferative glomerulonephritis type II (MPGNII) and hemolytic uraemic syndrome (HUS).

### 3.20 VEGF-A

VEGF-A is a member of the platelet-derived growth factor/vascular endothelial growth factor (PDGF/VEGF) growth factor family. It is a heparin-binding protein, which exists as a disulfide-linked homodimer. This growth factor induces proliferation and migration of vascular endothelial cells and is essential for both physiological and pathological angiogenesis.

### 3.21 VEGF-B

VEGF-B seems to play a role only in the maintenance of newly formed blood vessels during pathological conditions. VEGF-B plays also an important protective role on several types of neurons, including the retina.

### 3.22 VEGF-C

VEGF-C is a member of the PDGF/VEGF family that mainly functions to promote the growth of lymphatic vessels (lymphangiogenesis). It acts on lymphatic endothelial cells (LECs), mainly via its primary receptor VEGFR-3, promoting survival, growth and migration. It is a ligand for the orphan receptor VEGFR-3. It can also promote the growth of blood vessels and regulate their permeability. The effect on blood vessels can be mediated via VEGFR-3 or its secondary receptor VEGFR-2.

### 3.23 Aflibercept (VEGF-TRAP)

VEGF expression in the retinal pigment epithelium (RPE) is associated with neovascular AMD. Aflibercept is a recombinant fusion protein that binds VEGF-A that is used in treatment of AMD. See Holash et al., 2002, “VEGF-Trap: a VEGF blocker with potent antitumor effects” Proc Natl Acad Sci USA 99:11393-8. Sequences of aflibercept with various signal peptides are provided (see SEQ ID NOs.: 6-8).

### 3.24 High-Temperature Requirement a Serine Peptidase 1(HTRA1; HTRA Serine Peptidase 1)

HTRA1 is a Serine Peptidase 1(SEQ ID NO:240). This protein is a secreted enzyme that is proposed to regulate the availability of insulin-like growth factors (IGFs) by cleaving IGF-binding proteins. HtrA levels are reduced in subjects at risk of developing AMD. Methods for increasing ocular HTRA1 levels are described in WO2020210724. These include delivery of a cargo encoding the HTRA1 protein under control of an operably linked promoter and delivery of a CRISPR activation system (including CAS and sgRNA) as described in WO2020210724.

### 3.25 APOE2

APOE2 is an allelic isoform of APOE apolipoprotein. APOE functions in lipoprotein-mediated lipid transport between organs via the plasma and interstitial fluids. Serum APOE regulates the expression of inflammatory cytokines and vascular endothelial growth factor (VEGF) family of cytokines in retinal pigment epithelial (RPE) cells (Qureshi, et al., 2017, “Serum APOE, leptin, CFH and HTRA1 levels in Pakistani age related macular degeneration patients” JPMA J. Pakistan Med Assoc., 67:52-857). APOE2 is associated with enhanced risk of developing AMD. See Toops et al., 2016, “Apolipoprotein E Isoforms and AMD,” Adv. Exp. Med. Biol. 854: 3-9; Levy et al., 2015, “APOE Isoforms Control Pathogenic Subretinal Inflammation in Age-Related Macular Degeneration Neurosci 35(40):13568-76.

### 3.26 APOE4

APOE4 is an allelic isoform of APOE. In the circulation, APOE is present as part of several classes of lipoprotein particles, including chylomicron remnants, VLDL, IDL, and some HDL. APOE interacts significantly with the low-density lipoprotein receptor (LDLR), which is essential for the normal processing (catabolism) of triglyceride-rich lipoproteins. In peripheral tissues, APOE is primarily produced by the liver and macrophages, and mediates cholesterol metabolism. In the central nervous system, APOE is mainly produced by astrocytes and transports cholesterol to neurons via APOE receptors, which are members of the low density lipoprotein receptor gene family. APOE is the principal cholesterol carrier in the brain. APOE is required for cholesterol transportation from astrocytes to neurons. APOE qualifies as a checkpoint inhibitor of the classical complement pathway by complex formation with activated C1q.

### 4. Augmentation by Expression of Exogenous Proteins

Expression of exogenous CFH, CFHT, and CFI is carried out using art known techniques. For example, a cDNA sequence, which is optionally codon-optimized, is linked to a promoter and other regulatory elements and delivered to a target cell as described herein. A heterologous signal peptide can be linked to a protein. Exemplary transgenes encoding CFH, CFHT, eCFH/T, oCFHT, CFI, and HTRA1 are described herein.

### 5. Diminution of Complement and Other Proteins

Diminution of complement proteins is achieved using specific binding agents or inhibitory RNA as described below. As used herein, ‘binding agent’ refers to an agent (antibody, aptamer, trap or other) that binds to and sequesters and/or reduces the quantity or activity of the protein bound. For target molecules that are synthesized locally in occular tissue (e.g., by the RPE, retina and/or choroid) such as C3, C4, C5, C6, C7, C8G, C9, CFB, CFD, an inhibitory RNA (e.g., hpRNA) strategy may be preferred over binding agents. A “binding agent” that is a polypeptide or is a complex of linked or associated polypeptides can be referred to as a “binding protein.”

As noted in TABLE 2, diminution (e.g., inhibition of activity or reduction in levels) of certain complement components and other biomolecules will provide therapeutic benefit to subjects with, or at risk of developing, AMD. There are primarily two broad categories of inhibitors: Inhibitory RNAs (described below in § 5.1) and binding agents. Binding agents include traps such as aflibercept or variants thereof (described below in § 5.2), and single chain antibodies such as nanobodies (described below in § 5.3).

### 5.1 Diminution of Expression Using Inhibitory RNA

Reduction of expression of targets (diminution of target) can be achieved using inhibitory RNA molecules or binding agents as described below. For target molecules that are synthesized locally by the RPE, retina and/or choroid (e.g., C3, C4, C5, C6, C7, C8G, C9, CFB, CFD) an inhibitory RNA (e.g., hpRNA) may be used to reduce target expression. Alternatively or additionally, a binding agent may be used to reduce the target biological activity.

Inhibitory RNA techniques use engineered RNA molecules to inhibit gene expression through various biological mechanisms (e.g., transcript cleavage, sequestration, inhibition of protein translation or RNA aptamer that inhibit protein activity). Suitable inhibitory RNA molecules include small interfering RNA (siRNA), short hairpin RNA (shRNA), micro RNA (miRNA), and anti-sense RNA that target an endogenous RNA transcript. For example, administration of a vector encoding and expressing an inhibitory RNA directed against complement componant C7 will reduce endogenous C7 mRNA and/or C7 polypeptide. The reduction in C7 results in a therapeutically beneficial reduction in MAC formation.

As used herein, an “antisense strand” refers to the strand of a double stranded region of an RNAi agent (e.g., siRNA, shRNA, miRNA) that includes a region complementary or substantially complementary to a target RNA sequence or corresponding DNA (e.g., a human C7 mRNA including a 5′ UTR, exons of an open reading frame (ORF), or a 3′ UTR). The region of “complementarity” or “substantial complementarity” need not be fully complementary to the target sequence and may have sequence % identity or % similarity of least 70%, 75%, 80%, 85%, 90%, 95%, or 100%.

A “sense strand,” as used herein, refers to the strand of a RNAi agent (siRNA, shRNa, miRNA) that includes a region that is complementary or substantially complementary to a region of the antisense strand.

shRNA, siRNA, and miRNA can be designed using well-defined principles using publicly and commercially available software. Illustrative companies that provide shRNA design algorithms and services include Thermo Fisher, InvivoGen, Biosettia, Hairpin Technologies, Horizon Discovery, Eurofins Genomics and others. For example, shRNA can be identified using the Thermo Fisher website rnaidesigner.thermofisher.com/rnaiexpress/help/shrna_enter_sequence_parameters. Publicly available software Includes software available through The Broad Institute. See, also Fakhr et al, Cancer Gene Therapy 23:73-82, 2016, which describes various parameters from different algorithms for designing functional small inhibitory RNAs. Design considerations are additionally described by Ros XB-D, Gu S. Methods 103: 157-166, 2016.

The specificity or knockdown level of a shRNA, miRNA, or siRNA can be confirmed using real-time PCR (RT-PCR) analysis for mRNA level or ELISA assay for the protein level. Experimental controls may be run in parallel to assess knockdown. Some examples of experimental controls that may be used, include but are not limited to, a mock-infected or mock-transfected sample, an empty vector, an shRNA encoding a scrambled target or seed region, an shRNA targeting another gene entirely such as, housekeeping genes GAPDH or Actin, or a GFP positive control. In some embodiments, an shRNA or siRNA results in expression levels that are reduced by at least about 25%, 50%, 75%, 80%, 85%, 90%, 95%, 96%, 97%, 98%, 99%, or 100% compared to a control, such as a mock-infected or empty vector control.

### 5.1.1 Properties of Inhibitory RNAs-shRNA

In some embodiments, the inhibitor RNA is an shRNA that induces RNA interference in a cell. The shRNA comprises a hairpin structure such that the RNA molecule comprises a double stranded region of paired antisense and sense strand connected by a loop of unpaired nucleotides. shRNAs are generally designed to bypass Drosha/DGCR8 cleavage. After export from the nucleus by exportin 5, shRNAs are cleaved by Dicer to form a duplex that can be loaded into Argonaute-RISC complexes.

In typical embodiments, the double-stranded region is about 19-25 base pairs in length. The loop region is typically from 4-11 nucleotides in length. In some embodiments, the duplex region is 19, 20, 21, or 22 base pairs in length.

In some embodiments, an shRNA targets the 5′ or 3′ untranslated region of the target RNA sequence. In other embodiments, an shRNA targets a protein-coding region.

In some embodiments, an anti-sense strand of the shRNA is 100% complementary to a target sequence. In some embodiments, the antisense stranded of the shRNA has at least 80%, at least 85%, at least 90%, or at least 99% complementarity with the target sequence.

TABLE 7 shows examples of shRNA targeting sequences (antisense) useful in multigene therapy.

TABLE 7
Exemplary hairpin (HP)-RNA.

| SEQ | Exemplary HP-RNA | |
|---|---|---|
| ID NO | targeting C3 (Antisense 5′-3′) | |
| 62 | C3 Hairpin A | AATTGTTGGAGTTGCCCAC |
| 63 | C3 Hairpin B | ATCTTTAGCCTCCTGCAGC |
| 64 | C3 Hairpin C | TTCGAACAACAGAGTAGGG |
| 65 | C3 Hairpin D | TTATGGTGACCTTGAGGTC |
| 66 | C3 Hairpin E | AGATGGTCTTGTCTGTCTG |
| 67 | C3 Hairpin F | TTCCTCCAGGTTGTAATAGGC |
| 68 | C3 Hairpin G | TTTCTTGGTGTATCACGGGCG |
| 69 | C3 Hairpin H | ATGATGAGGGTGTTCCTATCG |
| 70 | C3 Hairpin I | TTCATTCTGATTCCTTCCGGC |
| 71 | C3 Hairpin J | TTGTTCTTCTTATTCAGCACG |
| SEQ | Exemplary HP-RNA | |
| ID NO | targeting C4A (Antisense 5′-3′) | |
| 72 | C4A Hairpin A | ATTCTTTCCCAGGTTCCAAGC |
| 73 | C4A Hairpin B | TTACGAGATGGGTTTCTCAGG |
| 74 | C4A Hairpin C | TTGACATCCTTGGTGAAGTGC |
| 75 | C4A Hairpin D | TAAGGTGAAGAAGCTGGATGC |
| 76 | C4A Hairpin E | TTACCATCCTCATCTAGGAGC |
| SEQ | Exemplary HP-RNA | |
| ID NO | targeting C4B (Antisense 5′-3′) | |
| 77 | C4B Hairpin A | TTATCCAGGTAGTTATAGAGG |
| 78 | C4B Hairpin B | TTTGCTTCCATCGTGTACTCG |
| 79 | C4B Hairpin C | AAGTGTCCAATGGATCTGAGG |
| 80 | C4B Hairpin D | TTCACCTCAAAGTTGGGAAGG |
| 81 | C4B Hairpin E | TTTCTGGATAAGGTCCACTGG |
| SEQ | Exemplary HP-RNA | |
| ID NO | targeting C5 (Antisense 5′-3′) | |
| 82 | C5 Hairpin A | TATTAACGCAGGCTCCATC |
| 83 | C5 Hairpin B | TTATGAGAGATATTAGCAC |
| 84 | C5 Hairpin C | AACACAGAACTGCATCCCA |
| 85 | C5 Hairpin D | ATTCCAATCACAGTAAAGG |
| 86 | C5 Hairpin E | AATTAAGTACTGTCTTCCT |
| 87 | C5 Hairpin F | ATTAGCTAAAAATGCTTGA |
| 88 | C5 Hairpin G | TAAGTGCAAACTGTATGCAGC |
| 89 | C5 Hairpin H | TTCAAAGAGTTCAAATATCCG |
| 90 | C5 Hairpin I | AACCAAATTCAGTTTGTAGGG |
| 91 | C5 Hairpin J | TTATGAGAGATATTAGCACGG |
| 92 | C5 Hairpin K | ATATAAGCTGTTCTCTCGGGC |
| SEQ | Exemplary HP-RNA | |
| ID NO | targeting C6 (Antisense 5′-3′) | |
| 93 | C6 Hairpin A | TTATTTAAACATTTCTCAG |
| 94 | C6 Hairpin B | TTGTGAGAGGCTTGAATGG |
| 95 | C6 Hairpin C | ATAGTTGCTTTTCATTCCG |
| 96 | C6 Hairpin D | AGTCTTGTCCTTTCAAGAC |
| 97 | C6 Hairpin E | TCTCTGACAGCTTGTTGGT |
| 98 | C6 Hairpin F | ACTCTTGCAAAGCTTTCCT |
| 99 | C6 Hairpin G | TATCAAGGACTTCTCCTCTGG |
| 100 | C6 Hairpin H | TAGAGGCAGATGGTTAAGTGC |
| 101 | C6 Hairpin I | TTTCAAAGCCAGTAAGGCATG |
| 102 | C6 Hairpin J | ATTGCATTCTAACTTTCTGGC |
| 103 | C6 Hairpin K | ATAGGCAGACACATTTGGAAG |
| SEQ | Exemplary HP-RNA | |
| ID NO | targeting C7 (Antisense 5′-3′) | |
| 58 | C7 Hairpin A | GAGCCACAGAGAAATGCTGAAGGACCTTC |
| 59 | C7 Hairpin B | CTGAGTCTTGGTACAGCCATTGCATTCTG |
| 60 | C7 Hairpin C | AACCAATGATTTGCTGATGCACTGACCTG |
| 61 | C7 Hairpin D | ATCTTTCAAGGCAGGAGGTGATGGACAGA |
| 104 | C7 Hairpin E | ACTGGTCGATTAATCTTCG |
| 105 | C7 Hairpin F | TCACTTCCACACAAATGCT |
| 106 | C7 Hairpin G | AAATCCATCTTTCAAGGCAGG |
| 107 | C7 Hairpin H | AAACCAATGATTTGCTGATGC |
| 108 | C7 Hairpin I | TACTGGTCGATTAATCTTCGG |
| 109 | C7 Hairpin J | TTTCTGTCGTTTCTCCAACGC |
| 110 | C7 Hairpin K | TTTGCTTCTCTCATCTTGAGC |
| SEQ | Exemplary HP-RNA | |
| ID NO | targeting C8A (Antisense 5′-3′) | |
| 111 | C8A Hairpin A | TACCCTACCAATTAATAAT |
| 112 | C8A Hairpin B | AGTTAACAGTCAACATCCA |
| 113 | C8A Hairpin C | TAACAGTCAACATCCAGTT |
| 114 | C8A Hairpin D | TTGCTTTGTCAATCACCAG |
| 115 | C8A Hairpin E | TACACAAGTTGTAGAACTG |
| 116 | C8A Hairpin F | ATGAAGTGTCTTGTGAGTGGG |
| 117 | C8A Hairpin G | TTACACAAGTTGTAGAACTGG |
| 118 | C8A Hairpin H | AATGGTCTCCTGATAAACCTC |
| 119 | C8A Hairpin I | TTTGCTTTGTCAATCACCAGG |
| 120 | C8A Hairpin J | ATACATGCCATAATTGTACTG |
| SEQ | Exemplary HP-RNA | |
| ID NO | targeting C8B (Antisense 5′-3′) | |
| 121 | C8B Hairpin A | TAGCTTTCCACATTGTAGG |
| 122 | C8B Hairpin B | TAAATCCTTCTACAGTTTG |
| 123 | C8B Hairpin C | TTCTCTGTGACATTGCGTT |
| 124 | C8B Hairpin D | TACACACCTTCCTGTCTGT |
| 125 | C8B Hairpin E | TTGTCTTACGTCTTCCAGAGC |
| 126 | C8B Hairpin F | TAGTATGAGAGAATCGTTTGG |
| 127 | C8B Hairpin G | ATTTCATTCAGAATACCTCTG |
| 128 | C8B Hairpin H | TAGACAGCTCACAATCAATGG |
| 129 | C8B Hairpin I | TTTCTCTGTGACATTGCGTTC |
| SEQ | Exemplary HP-RNA | |
| ID NO | targeting C8G (Antisense 5′-3′) | |
| 130 | C8G Hairpin A | CGGAAGGTACTGACAGCCA |
| 131 | C8G Hairpin B | TGGGGAAGTAGAAGATCTG |
| 132 | C8G Hairpin C | AAACTCTGGTAGTCGGTCTCA |
| 133 | C8G Hairpin D | ATCCCATCCAGCTTTCGGAAG |
| 134 | C8G Hairpin E | AAACCCACTCAGGACCGAGTC |
| 135 | C8G Hairpin F | TACAGGACAGCGAAACTCTGG |
| 136 | C8G Hairpin G | TTCGTCCAGGACGTGGAACTG |
| SEQ | Exemplary HP-RNA | |
| ID NO | targeting C9 (Antisense 5′-3′) | |
| 137 | C9 Hairpin A | ATGTTGATCCCATAGCCTG |
| 138 | C9 Hairpin B | TGTTCTTCGTAATGTTCGG |
| 139 | C9 Hairpin C | TATGAGTGAAACAACATCA |
| 140 | C9 Hairpin D | GAAACAACATCATCTATGA |
| 141 | C9 Hairpin E | AGTTCAACACCTTTCCGCT |
| 142 | C9 Hairpin F | AAAATTTGATGTCTTCTCT |
| 143 | C9 Hairpin G | TGAACGAAACATTTGTCTG |
| 144 | C9 Hairpin H | ATTATAGACCTAAGAGAGAAG |
| 145 | C9 Hairpin I | TTGTTTACTGATTTCACAGGC |
| 146 | C9 Hairpin J | AATGTTGATCCCATAGCCTGC |
| 147 | C9 Hairpin K | TTTGTTCTTCGTAATGTTCGG |
| 148 | C9 Hairpin L | TTAGCTCTGGGTCATAACTGG |
| SEQ | Exemplary HP-RNA | |
| ID NO | argeting CFB (Antisense 5′-3′) | |
| 149 | CFB Hairpin A | TATTCTTGAGCTTGATCAG |
| 150 | CFB Hairpin B | TGGTCTTGAGTCTTCAGGG |
| 151 | CFB Hairpin C | TGATGTAGACCTCCTTCCG |
| 152 | CFB Hairpin D | TGAATGAAACGACTTCTCT |
| 153 | CFB Hairpin E | ATTCTTGAGCTTGATCAGG |
| 154 | CFB Hairpin F | TGAGCTGCTTCGTGACCCA |
| 155 | CFB Hairpin G | AGGACTACTTCTATCTCCA |
| 156 | CFB Hairpin H | TTTGGCTCCTGTGAAGTTGCT |
| 157 | CFB Hairpin I | TACGCTGACCTTGATTGAGTG |
| 158 | CFB Hairpin J | TTGAAGGGCGAATGACTGAGA |
| 159 | CFB Hairpin K | TTGATGTGAAAGTCTCGGGCG |
| 160 | CFB Hairpin L | ATATCCTTGACTTTGAACACA |
| SEQ | Exemplary HP-RNA | |
| ID NO | targeting CFD (Antisense 5′-3′) | |
| 161 | CFD Hairpin A | TGCTCGGGACTTTGTTGCT |
| 162 | CFD Hairpin B | AGATCCCGGGCTTCTTGCG |
| 163 | CFD Hairpin C | TTGTTGCTTGGGTGACCCT |
| 164 | CFD Hairpin D | CACCCTTGCAGCTGTCCCG |
| 165 | CFD Hairpin E | ATTTGTAGAGATGGAGTCGCG |
| 166 | CFD Hairpin F | TTCTTGCGGTTGCCGCAAACG |
| 167 | CFD Hairpin G | AACCTGCACCTTCCCGTCGGC |
| 168 | CFD Hairpin H | TCCGCGCACATCAAGCGCTCG |
| SEQ | Exemplary HP-RNA | |
| ID NO | argeting VEGFA (Antisense 5′-3′) | |
| 169 | VEGFA Hairpin A | TATACCGGGATTTCTTGCG |
| 170 | VEGFA Hairpin B | TTCTGTATCGATCGTTCTG |
| 171 | VEGFA Hairpin C | TCTGTATCGATCGTTCTGT |
| 172 | VEGFA Hairpin D | ACTTCGTGATGATTCTGCCCT |
| 173 | VEGFA Hairpin E | TTATACCGGGATTTCTTGCGC |
| 174 | VEGFA Hairpin F | TTGGTGAGGTTTGATCCGCAT |
| 175 | VEGFA Hairpin G | TCACATCTGCAAGTACGTTCG |
| 176 | VEGFA Hairpin H | TTGCAGGAACATTTACACGTC |
| SEQ | Exemplary HP-RNA | |
| ID NO | targeting CFP (Antisense 5′-3′) | |
| 177 | CFP Hairpin A | AGGATTAGGTCCACAGGGG |
| 178 | CFP Hairpin B | AGACATGGTCGTTTCTCCT |
| 179 | CFP Hairpin C | TCGTGTCTCCTTAGGTTCG |
| 180 | CFP Hairpin D | TACGTTTCTGGTAGGCAAAGG |
| 181 | CFP Hairpin E | ATGGACTTCATGTTCCGTCGG |
| 182 | CFP Hairpin F | TTGCCGGAGGATTCTTCATAC |
| 183 | CFP Hairpin G | ATCGATGTCCGTCAAACTTGC |
| 184 | CFP Hairpin H | TTCGTGTCTCCTTAGGTTCGT |

### 5.1.2 Properties of Inhibitory RNAs—miRNA

A microRNA (miRNA) is a small nan-coding RNA molecule, that functions in RNA silencing and post-transcriptional regulation of gene expression. miRNAs base pair with complementary sequences within the mRNA transcript. As a result, the mRNA transcript may be silenced by one or more of the mechanisms such as cleavage of the mRNA strand, destabilization of the mRNA through shortening of its poly(A) tail, and decrease translation efficiency of the mRNA transcript into proteins by ribosomes. The term “miRNA” as used herein includes both naturally occurring and artificial miRNAs, e.g., a cellular miRNA in which the stem is modified to be partially complementary to an mRNA of interest.

Artificial miRNAs are typically designed to undergo cleavage both by Drosha/DGCR8 and Dicer. They can be transcribed from their own promoters, embedded in an intron, or located in the 3′-UTR of a protein-coding gene. Thus, in some embodiments, miRNAs resemble the siRNAs of the shRNA pathway, except that miRNAs derive from regions of RNA transcripts that fold back on themselves to form short hairpins (pri-miRNA). Once transcribed as pre-miRNA, the hairpins are cleaved out of the primary transcript in the nucleus by Drosha. The hairpins, or pre-miRNA, are then exported from the nucleus into the cytosol where the loop of the hairpin is cleaved by Dicer. The resulting product is a double strand RNA with overhangs at the 3′ end, which is then incorporated into RISC. Once in the RISC, the second strand is discarded and the miRNA that is now in the RISC is a mature miRNA, which binds to mRNAs that have complementary sequences.

In one aspect, a difference between miRNAs and siRNAs from the shRNA pathway is that base pairing with miRNAs comes from the 5′ end of the miRNA, which is also referred to as the seed sequence. Since the seed sequence is short, each miRNA may target many more mRNA transcripts.

In one embodiment, short hairpin RNA constructs are designed to be expressed as human miRNA (e.g., miR-30 or miR-21) primary transcripts. This design can add a Drosha processing site to the hairpin construct. The hairpin stem duplex of miRNA is typically 22 bp. In some embodiments, the antisense has perfect complementarity to desired target. In some embodiments, the duplex region is 22 bp and the loop region comprises a 15-19-nt loop from a human miR. Adding the miR loop and miR30 flanking sequences on either or both sides of the hairpin results in greater than 10-fold increase in Drosha and Dicer processing of the expressed hairpins when compared with conventional shRNA designs without microRNA. Increased Drosha and Dicer processing translates into greater siRNA/miRNA production and greater potency for expressed hairpins.

### 5.1.3 Properties of Inhibitory RNAs—siRNA

In some embodiments, an RNA inhibitor molecule that targets expression of a complement modulating gene, is a double stranded siRNA. In general, the guide strand of a given siRNA recognizes and binds to a target sequence that is complementary to the guide strand sequence. Stringent complementarity between guide strand and target sequence is, however, not required, as it is known in the art that a guide strand can still efficiently recognize and bind to a target sequence. Accordingly, in some embodiments, the guide strand sequence is 100% complementary to the target sequence. In some embodiments, the guide strand has as at least 80%, at least 85%, at least 90%, or at least 99% complementarity with the target sequence.

In certain embodiments, an siRNA comprises a first strand and a second strand that have the same number of nucleosides; however, the first and second strands are offset such that the two terminal nucleosides on the first and second strands are not paired with a residue on the complimentary strand. In certain instances, the two nucleosides that are not paired are thymidine resides. The siRNA should include a region of sufficient homology to the target gene, and be of sufficient length in terms of nucleotides, such that the siRNA, or a fragment thereof, can mediate down regulation of the target gene. Thus, as noted above, an siRNA includes a region which is at least partially complementary to the target RNA. It is not necessary that there be perfect complementarity between the siRNA and the target, but the correspondence must be sufficient to enable the siRNA, or a cleavage product thereof, to direct sequence specific silencing, such as by RNAi cleavage of the target RNA.

Although perfect complementarity, particularly in the antisense strand, is often desired, in some embodiments an siRNA has one or more, but preferably 10, 8, 6, 5, 4, 3, 2, or fewer mismatches with respect to the target RNA. The mismatches are typically most tolerated in the terminal regions. Thus, mismatches may be present in the terminal region or regions, e.g., within 6, 5, 4, or 3 nucleotides of the 5′ and/or 3′ terminus.

In some embodiments, the ds region of the siRNA can be about 14, 15, 16, 17, 18, 19, 20, 21, 22, 23, 24, 25, 26, 27, 28, 29, or 30 nucleotides in length. In some embodiments siRNAs have a duplex region of 7, 18, 19, 29, 21, 22, 23, 24, or 25 nucleotide pairs. In some embodiments, siRNAs have a duplex resin of 17, 18, 19, 29, 21, 22, 23, 24, or 25 nucleotide pairs, and one or more overhangs of 2-3 nucleotides, preferably one or two 3′ overhangs, of 2-3 nucleotides.

### 5.1.4 Properties of Inhibitory RNAs—Anti-Sense RNA

An antisense oligonucleotide (ASO) is a single-stranded nucleic acid molecule, typically a deoxyribonucleotide molecule, that is complementary to the mRNA target. Not to be bound by theory, down regulation of a target polypeptide is usually achieved by induction of RNase H endonuclease activity that cleaves the RNA-DNA heteroduplex leading to a reduction in translation of the target gene. Other ASO-driven mechanisms include inhibition of 5′ cap formation, alteration of splicing process (splice-switching), and steric hindrance of ribosomal activity.

Design considerations include ensuring that an ASO does partially binds to a nontarget mRNA, as 6-7 base pairs between the ASO and nontarget mRNA can be sufficient to induce RNase activity, leading to cleavage of the wrong target. Software that analyzes RNA secondary and tertiary structure can be used to select target sequences that do not have marked secondary structure. In some embodiments, the length of an ASO is about 20 nucleotides and can, for example, be selected to target either the methionine initiation codon or splice sites (to block splicing). ASOs typically include modifications to enhance stability, e.g., are phosphorothioated, which does not enhance RNase H activity or influence solubility. In some embodiments, ASO may include ribose modifications, e.g., substitution of the hydrogen at the 2-position by an O-alkyl group and locked nucleic acid technology (LNA)] that reduce conformational plasticity.

### 5.1.5 Promoters and Expression of Inhibitory RNA

Small inhibitory RNAs are transcribed endogenously with RNA Polymerase II (Pol III). Thus, in some embodiments, the promoter is a U6 promoter, H1 promoter, or 7SK promoter. In some embodiments the promoter is a variant of a naturally occurring human U6, H1, or 75K promoter, e.g., as described by Gao et al, Transcription 8:275-287, 2017. An exemplary U6 promoter is shown at SEQ ID NO:15.

In some embodiments, a multigene cargo as described herein comprises an RNA polymerase II promoter, i.e., a promoter sequence to initiate transcription from RNA polymerase II, such as a promoter for transcription of a transgene encoding a polypeptide such as CFHT; and a U6 or H1 promoter to drive expression of an inhibitory RNA molecule. In some embodiments, the second promoter is a U6 or H1 promoter.

In some embodiments, a single polymerase III promoter, e.g., a human H1 promoter, can be used to drive expression of both an inhibitory RNA and a protein-encoding transgene. (see, for example, Gao et al, Molecular Therapy: Nucleic Acids 14:32-40, 2019).

### 5.2 Diminution Using Binding Agents

As discussed above, agents that bind and complement proteins and disease modifier proteins are delivered. These binding agents are encoded in multigene cargo and delivered to and expressed in cells. Binding agents are particularly useful for diminution of proteins produced in liver or other non-occular tissues. Exemplary binding agents are aflibercept (see Section 3.23 above) and single chain antibodies, nanobodies, bivalent nanobodies and the like.

### 5.2.1 Aflibercept

Aflibercept is a recombinant fusion protein that binds VEGF-A that is used in treatment of AMD. VEGF expression in the retinal pigment epithelium (RPE) is associated with neovascular AMD.

Aflibercept is a recombinant inhibitory receptor (or “trap”) that binds VEGF and is used as a treatment for AMD. Exemplary DNA sequences encoding aflibercept are provided at SEQ ID NOs:6-8. Aflibercept-based gene therapy has been described (see, e.g., Kiss et al., 2020, “Analysis of Aflibercept Expression in NHPs following Intravitreal Administration of ADVM-022, a Potential Gene Therapy for nAMD” Mol Ther Methods Clin Dev. 18:345-353; Holash et al., 2002, “VEGF-Trap: a VEGF blocker with potent antitumor effects” Proc Natl Acad Sci USA 99:11393-8.). According to the present invention, a transgene encoding aflibercept, or a variant thereof, is encoded in multigene cargo and administered to the eye.

### 5.3 Antibodies

In one approach an antibody, e.g., an anti-complement protein antibody, is encoded in the multigene cargo and administered to a subject. Preferred antibodies are small, such as single chain antibodies (ScFv), single domain antibodies (nanobodies), dimerized nanobodies, bispecific nanobodies or bivalent nanobodies. In one approach, one or more nanobodies are encoded in multigene cargo. Nanobodies are single domain antibodies derived from heavy chain only antibodies found in the camelid family such as llamas. See, e.g., Jovlevska, 2020, The “Therapeutic Potential of Nanobodies” BioDrugs. 34:11-26; Zarantonello et el., 2021, “Nanobodies Provide Insight into the Molecular Mechanisms of the Complement Cascade and Offer New Therapeutic Strategies” Biomolecules 11:298; Pardon et al., 2014, “A general protocol for the generation of Nanobodies for structural biology”. Nat. Protoc. 9:674-693. Their relatively small size relative to human IgG make nanobodies suitable for DNA based delivery. In some embodiments humanized nanobodies are used. See Kazemi-Lomedasht et al., 2018, Design of a humanized anti vascular endothelial growth factor nanobody and evaluation of its in vitro function, Iran J Basic Med Sci. 21(3):260-266.

In some cases a bivalent nanobody is encoded in multigene cargo. Bivalent nanobodies are known therapeutic agents. For example, a single chain VHH bivalent nanobody targeting von Willebrand factor (vWF) has recently been approved for thrombotic thrombocytopenic purpura (aTTP) and several other antibodies are in clinical development (Morrison C., Nat Rev Drug Dis 18: 485-487, 2019). Examples include bispecific nanobodies targeting CFP and EGFR to kill tumor cells (Pedersen D. V., et al., Mol Immunol 124:200-210, 2020) or bispecific nanobodies targeting SARS-CoV-2 spike protein and albumin to block viral entry and to increase nanobody stability (Tijink, et al., Mol Cancer Ther 8:2288-2297, 2008).

In one approach, nanobodies or scFv antibodies are selected to be cross-reactive for (and thereby target) two or more related proteins. Of particular interest are nanobodies or scFv antibodies that bind two or more CFH related proteins (e.g., CFHR1/2, CFHR1/2/3/4/5, CFHR1/4). Methods for selection of cross-reactive antibodies are known. CFH related proteins are highly homologous and cross-reactive antibodies can be selected by raising or selecting antibodies against a highly conserved sequences or epitopes. The same strategy can be used to select anti-CFHR antibodies specific for specific CFHR(s) or that do not bind CFH or CFHT. In one approach two VHH nanobodies are linked via a peptide linker, to generate bispecific inhibitors with improved properties to uniquely inhibit multiple FHR family members. In some embodiments, nanobodies will be screened for cross-reactivity to homologous family members (e.g. FHR-1 nanobodies with affinity for FHR-2 or FHR-4 nanobodies with affinity for FHR-3), but such antibodies lack any appreciable affinity for CFH or CFHT proteins. In some embodiments, nanobodies will be constructed as a monotherapy to decrease the amount of endogenous complement pathway competition induced by FHR-1, FHR-2, FHR-3 and FHR-4 proteins or combined in a multigene therapy vector with CFHT or CFH and/or CFI and hpRNAs targeting C7 to provide therapeutic benefit in AMD patients. Nanobodies developed to target FHR-1 and/or FHR-4 can be used to block complement activation as single VHH domains, as bivalent VHH domains, or as bispecific VHH domains fused by a flexible linker region.

Nanobodies that specifically bind FHR-1 were produced as described in EXAMPLE 1 (See Section 10, below). Anti-FHR-1 nanobodies with cross-reactivity to CFH (FHR-1-CFH), CFHT (FHR-1-CFHT), and FHR-2 (FHR-1-FHR-2) were identified. Anti-FHR-1 nanobodies with cross-reactivity to FHR-2, FHR-3, FHR-4 and/or FHR-5, without cross-reactivity to CFH and/or CFHT are preferred. In one approach an anti-FHR-1 nanobody with cross-reactivity to FHR-2 is encoded in multigene cargo for delivery to cells. In one approach an anti-FHR-1 nanobody with cross-reactivity to FHR-3 is encoded in multigene cargo for delivery to cells. In one approach an anti-FHR-1 nanobody with cross-reactivity to FHR-2 is encoded in multigene cargo for delivery to cells. In one approach an anti-FHR-1 nanobody with cross-reactivity to FHR-5 is encoded in multigene cargo for delivery to cells. In one approach an anti-FHR-1 nanobody with cross-reactivity to two or more FHRs (FHR-2, FHR-3, FHR-4 and/or FHR-5) is encoded in multigene cargo for delivery to cells.

### 6. Monotherapies

In certain aspects monotherapies are also contemplated by the present invention. As used herein, “monotherapy” refers to gene therapy in which a viral or nonviral vector delivers a single activity to a cell (i.e., carries cargo encoding a single hpRNA, nanobody or optimized CFHT.

An exemplary vector for hpRNA monotherapy comprises a cargo expressing an hpRNA described in TABLE 7. An exemplary vector for administering a nanobody can deliver a nanobody described in Example 1. An exemplary vector for monotherapy encodes an optimized CFHT as described in Section 3.4 above. In one approach an oCFHT is expressed from a cargo that does not include other activities.

In one approach, a cargo encoding an anti-FHR-1 nanobody with cross-reactivity to FHR-2 is encoded in cargo for delivery to cells. In one approach an anti-FHR-1 nanobody without cross-reactivity to CFH or CFHT is encoded in cargo for delivery to cells. In one approach a single bispecific anti-FHR-1/anti-FHR-4 nanobody with cross-reactivity to FHR-2 and FHR-3 is encoded in cargo for delivery to cells.

### 7. Delivery and Coordinated Expression of Multiple Gene Products Genes in a Single Vector

This section describes methods for coordinated expression of multiple gene products encoded in a single cargo delivered to the target ocular tissue.

### 7.1 Vectors and Delivery Systems

Viral vectors are reviewed generally in Bulcha et al., 2021, “Viral vector platforms within the gene therapy landscape” Sig Transduct Target Ther 6, 53.

### 7.1.1 AAV: Adeno-Associated Virus Vectors

In some embodiments, a gene therapy vector is an adeno-associate virus vector (AAV). AAV is a primarily non-integrative virus with desirable safety characterises. However, AAV's limited packaging capacity means there are notable challenges to using this vector to deliver multiple (e.g., 2 or 3) activities. The total packaging capacity of AAV is about 4.7 Kb, including ITRs, and the vector can accommodate about 4.4 Kb of additional sequence including protein and/or inhibitory RNA encoding sequences and regulatory elements.

Multiple AAV serotypes are suitable for use in vectors of the present invention, including AAV1, AAV2, AAV3b, AAV4, AAV5, AAV6, AAV7, AAV8, AAV9, AAV10, AAV11, AAV12, AAV113, AAVrh.74, AAV2.7m8, AAV.ANC80, Anc80L65m, AAVDJ, pseudotyped AAV, and derivatives thereof. In one approach the serotype is selected from AAV5, AAV4, AAV2, AAV8, AAV9, and AAVrh8r. In some embodiments, the vector is a derivative of an AAV2 serotype, e.g., AAV2.7m8. In one approach AAV2 is used.

Most of the AAV wildtype genome is deleted from AAV vectors, but the vector retains functional flanking ITR sequences. The Right ITR is the identical reverse complement of the Left ITR (so that a single 5′-3′ nucleotide sequence can define both ITRs). A certain degree of mismatch between the left and right ITRs is tolerated. AAV can comprise ITRs that are of a heterologous serotype in comparison with the capsid serotype (e.g., AAV2 ITRs with AAV5, AAV6, or AAV8 capsids). Various ITRs are known and are suitable for use with AAV2-type vector. In some embodiments, the AAV2 vector comprises a 128-bp ITR (SEQ ID NO:48). AAV vectors are well described in the scientific literature. See, e.g., Wang et al., “Adeno-associated virus vector as a platform for gene therapy delivery.” Nat Rev Drug Discov 18: 358-378 (2019); Li, et al., “Engineering adeno-associated virus vectors for gene therapy.” Nat Rev Genet 21: 255-272 (2020); Zolotukin et al., 2002, Production And Purification Of Serotype 1, 2, And 5 Recombinant Adeno-Associated Viral Vectors” Methods 28:158-167; Aponte-Ubillus et al., 2018, “Molecular design for recombinant adeno-associated virus (rAAV) vector production” Applied microbiology and biotechnology 102.3:1045-1054; Naso et al., 2017, “Adeno-Associated Virus (AAV) as a Vector for Gene Therapy” BioDrugs 31:317; and Penaud-Budloo et al., 2018., “Pharmacology of Recombinant Adeno-associated Virus Production” Molecular Therapy: Methods & Clinical Development 8:166-180.

### 7.1.2 Lentiviral Vectors

In some embodiments, the vector for introducing a construct encoding multiple activities as described herein is a lentiviral vector. The basic structure of a lentiviral vector for gene therapy includes long terminal repeats (LTRs) that flank a nucleic acid sequence to be expressed by a cell. The LTRs can be divided into three elements: U3, R and U5. In one example, the viral construct comprises an inactivated or self-inactivating 3′ LTR from a lentivirus. The 3′ LTR may be made self-inactivating by any method known in the art. For example, the U3 element of the 3′ LTR can contain a deletion of its enhancer sequence, e.g., the TATA box, Sp1 and NF-kappa B sites. As a result of the self-inactivating 3′ LTR, the provirus that is integrated into the host genome will comprise an inactivated 5′ LTR. In some embodiments, the 5′ LTR of the lentiviral vector is truncated, relative to the native LTR. In some embodiments, the 5′ LTR comprises SEQ ID NO:49. In some embodiments, the 3′ LTR of the lentiviral vector is a Delta-U3 that comprises the polynucleotide sequence SEQ ID NO:50. Lentiviral vectors are well described in the medical literature. See, e.g., Milone, M. C., O'Doherty, U. Clinical use of lentiviral vectors. Leukemia 3:1529-1541 (2018); Esmaeili et al., 2020, Concise review on optimized methods in production and transduction of lentiviral vectors in order to facilitate immunotherapy and gene therapy, Biomedicine & Pharmacotherapy, 17:59-68.

### 7.1.3 Other Viral Vectors

Other viral vectors (including retroviruse, recombinant adenovirupox virus, alphavirus, retrovirus, arenavirus, measles, rabies, and herpes viruse based vectors may be used to deliver the cargo to the target tissue. Each vector system is well-understood by those of skill in the art. For general reviews see, e.g., Keeler et al., 2017, “Gene Therapy 2017: Progress And Future Directions” Clin Transl Sci (2017) 10, 242-248, incorporated by reference; Moore et al., 2017, “Gene Therapy For Age-Related Macular Degeneration” Expert Opinion on Biological Therapy 17:10: 1235-1244; Ochakovski et al., 2017, “Retinal Gene Therapy: Surgical Vector Delivery In The Translation To Clinical Trials” Frontiers in Neuroscience 11; Schon et al., 2015, “Retinal Gene Delivery By Adeno-Associated Virus (Aav) Vectors: Strategies And Applications” European Journal of Pharmaceutics and Biopharmaceutics 95:343-352; Naso et al., 2017, “Adeno-Associated Virus (AAV) As A Vector For Gene Therapy” BioDrugs 31:317; Dunbar et al., 2018, “Gene therapy comes of age,” Science 359: 6372, all incorporated herein by reference.

### 7.1.4 Non-Viral Vectors

In some embodiments, delivery vehicles such as liposomes, nanocapsules, nanoparticles, microspheres, and the like, may be used to facilitate administration of the vectors and/or to deliver other nucleic acids, including shRNa, miRNA, or siRNA to target cells. See, e.g., Itaka and Kataoka, 2009, “Recent development of nonviral gene delivery systems with virus-like structures and mechanisms,” Eur J Pharma and Biopharma 71:475-483; Thomas et al., 2020, “Biodegradable Polymers for Gene Delivery” Molecules 24(20): 3744doi: 10.3390/molecules24203744; Wooff et al., 2020, Small-Medium Extracellular Vesicles and Their miRNA Cargo in Retinal Health and Degeneration: Mediators of Homeostasis, and Vehicles for Targeted Gene Therapy, Frontiers in Cellular Neuroscience 14:1662-5102; Hong et al., “Functional Nanostructures for Effective Delivery of Small Interfering RNA Therapeutics.” Theranostics 4.12 (2014): 1211-1232. PMC. Web. 13 Sep. 2018, each of which is hereby incorporated by reference in its entirety for all purposes. Expression strategies

Various molecular strategies can be used to express two or more gene products from a single gene therapy vector, including use of multiple promoters, use of bidirectional promoters, use of IRES elements, use of ribosome skipping and cleavage factors. In some embodiments, a Pol 11 promoter drives expression of a sequence encoding an inhibitory RNA and an RNA polymerase II promoter to drive expression of a polypeptide gene product.

### 7.1.5 Multicistronic Vectors

In some embodiments, two or more gene products are expression in a single cistron. (see, e.g., Shaimardanova et al., “Production and Application of Multicistronic Constructs for Various Human Disease Therapies” Pharmaceutics 11:580, 2019). Thus, for example, in some embodiments, the multigene construct can comprise one promoter that drives expression of one or more gene products. Accordingly, such constructs can comprise various elements including IRES sequences and/or sequences encoding peptides that are cleaved.

### 7.1.5.1 Internal Ribosome Entry Site (IRES)

Incorporation of an internal ribosome entry site (IRES) can be employed to efficiently co-express multiple gene products from the same promoter. In some embodiments, a construct to express two or more different genes from a gene therapy vector can comprise an internal ribosome entry site (IRES). IRES elements can bypass the ribosome scanning model of 5′-methylated Cap dependent translation and begin translation at internal sites. IRES elements from members of the picornavirus family (e.g., polio and encephalomyocarditis) have been described, as well an IRES from a mammalian mRNA. In some embodiments, multiple open reading frames can be transcribed together, each separated by an IRES, creating polycistronic messages. By virtue of the IRES element, each open reading frame is accessible to ribosomes for efficient translation. In some embodiments, multiple genes are expressed using a single promoter/enhancer to transcribe a single message.

### 7.1.5.2 Ribosome Skipping Element (RSE)

In some embodiments, the gene products encoded by the vector are expressed bicistronically or multicistronically (i.e., two or more polypeptides, or inhibitory RNAs, are expressed from a single mRNA) by including one or more ribosomal skipping elements that encode a ribosome skipping polypeptide (also referred to herein as a “self-cleaving” peptide”) between a nucleic acid encoding a first polypeptide and a nucleic acid encoding a second polypeptide or inhibitory RNA. Similarly, three polypeptides can be expressed from one mRNA by including two ribosomal skipping elements. See, for example, Chang et al., 2015, “Cleavage efficient 2A peptides for high level monoclonal antibody expression in CHO cells,” MAbs 7(2): 403-412.

A “ribosomal skipping element” refers to a nucleotide sequence that encodes a short peptide sequence that generates two peptide chains from translation of one mRNA molecule. In some embodiments, the ribosomal skipping element encodes a peptide comprising a consensus motif of DX₁EX₂NPG wherein X₁ and X₂ are independently selected from any amino acid. In some embodiments, the ribosomal skipping element encodes a peptide comprising a consensus motif of DX₁EX₂NPG wherein X₁ is V or I and X₂ is any amino acid. In some embodiments, the ribosomal skipping element encodes a Thosea asigna virus 2A peptide (T2A), a porcine teschovirus-12A peptide (P2A), a foot-and-mouth disease virus 2A peptide (F2A), equine rhinitis A virus 2A peptide (E2A), a cytoplasmic polyhedrosis virus 2A peptide (BmCPV 2A), or a flacherie virus of B. mori 2A peptide (BmIFV 2A). Such peptides are typically from 18-22 amino acids in length.

While not wishing to be bound by theory, it is hypothesized that ribosomal skipping elements function by terminating translation of the first peptide chain and re-initiating translation of the second peptide chain; or by cleavage of a peptide bond in the peptide sequence encoded by the ribosomal skipping element by an intrinsic protease activity of the encoded peptide, or by another protease in the environment (e.g., cytosol). Exemplary self-cleaving peptide sequences are shown as SEQ ID Nos: 31, 33, 35 and 37, and examples of nucleic acid sequences that may be used to introduce the sequences is shown as SEQ ID Nos: 30, 32, 24, and 36 respectively.

In some approaches a glycine- and/or serine-containing linker sequence is introduced upstream from the self-cleaving peptide sequence to enhance cleavage. In some embodiments, a Gly-Ser spacer, e.g., a sequence Gly-Ser-Gly or Ser-Gly-Ser-Gly, is incorporated at the N terminus of the 2A sequence. Exemplary linker sequences include GlyGlyGlySer (SEQ ID NO:56), which can be repeated, e.g., the sequence can be present “n” times, e.g., where n=1-10 or 1-20; and GlyGlyGlyGlySer (SEQ ID NO:57), which can be present in the linker “n” times, e.g., where n=1-10 or 1-20. A furin cleavage site may be added upstream of the self-cleaving peptide sequence to eliminate or minimize amino acids appended to the amino terminus. Added amino acids at the amino terminus of the protein in position 2 can be removed with the removal of the signal peptide, for example, to generate a mature protein.

### 7.1.6 Furin Cleavage Site

In some embodiments, a nucleic acid sequence encoding a furin cleavage site may be incorporated into a multigene construct. In some embodiments, a furin cleavage site is upstream of the ribosomal skipping peptide, either with or without a Gly-Ser spacer. In some embodiments, a minimal furin cleavage site is Arg-X-X Arg (e.g., SEQ ID NO:39). In some embodiments, the cleavage site is Arg-X-(Lys/Arg)-Arg. In some embodiments, a furin cleavage site comprises GGRGRR (SEQ ID NO:39). In some embodiments, a furin cleavage site comprises GGRGRRGG (SEQ ID NO:40). In some embodiments, the furin cleavage site replaces an IRES and/or a ribosomal skipping peptide.

### 7.2 Regulatory Elements

### 7.2.1 Promoters

The relative levels and locations of polypeptide expression can be controlled by various promoters, including constitutive and cell-specific (e.g. RPE and choroidal endothelial cells, melanocytes, fibroblasts) promoters, e.g., promoters such as cytomegalovirus enhancer-chicken beta actin, CBA or CAG; small cytomegalovirus enhancer-chicken beta actin (smCBA); vitelliform macular dystrophy 2, VMD2; retinal pigment epithelium 65, RPE65; fms-related tyrosine kinase 1, FLT; von Willebrand factor, VWF and Endoglin, ENG) promoters; or promoters such as synthetic cytomegalovirus enhancer-vitelliform macular dystrophy 2, BEST1-v3-EP454 promoter enhancer; synthetic cytomegalovirus enhancer-retinal pigment epithelium 65, RPE65-F2/R20-EP415; and synthetic cytomegalovirus enhancer-retinal pigment epithelium 65, RPE65-F6/R26-EP419) promoters. In some embodiments the promotor is a large CMV enhancer and chicken beta actin promoter (CBA) promoter, BEST1-EP-454 promoter enhancer or spleen focus forming virus (SFFV) promoter (Hoffmann, D., et al., Gene Ther 24:298-307, 2017). Exemplary promoter sequences are provided at SEQ ID NOS:10-14, 16, 51, 185-191, 238, and 239.

Additional non-limiting examples of suitable eukaryotic promoters (i.e., promoters functional in eukaryotic cells) include a herpes simplex virus thymidine kinase gene promoter, early and late SV40 promoters, long terminal repeats from retrovirus, a human elongation factor-1 promoter, a bovine growth hormone promoter, a murine stem cell virus promoter, a phosphoglycerate kinase-1 locus promoter, and a ubiquitin gene promoter.

It will be understood by those of skill in the art that regulatory (promoter/enhancer) sequences can tolerate a certain degree of variation retaining the regulatory transcription regulatory function. In certain embodiments described herein in which a promoter/enhancer is designated, a substantially identical sequence (e.g., a sequence with at least about 90% identity, preferably at least about 91%, 92%, 93%, 94%, 95%, 96%, 97%, 98%, 99% nucleotide identity over the entire promoter/enhancer sequence) is contemplated as a suitable substitute for the exemplified sequence. As is well known in the art, variation is tolerated in the relationship (e.g., distance and orientation) between enhancers and promoters. Promoter variants and alternative promoters can be generated using well known techniques including truncation analysis, identification of transcription factor binding sites, motif analysis, and the like (see, e.g., Boeva, Frontiers in Genetics Vol. 7, Article 24, 2016); see Hageman and Richards WO2020019002A1, describing promoter analysis) to identify active promoters. Identification of active promoters can also comprise analysis of promoter activity as measured by art known means.

### 7.3.1.1 CBA/CAG

In some embodiments, a CBA (chicken beta-actin) promoter is used to drive expression of a gene product encoded by a cargo. In one embodiment, the CBA promoter includes a CMV enhancer sequence, the beta actin promoter, a spacer, a chicken b-actin intron, an intron acceptor b-globin, and a beta globin exon 3. An exemplary CBA promoter has a sequence of SEQ NO:12, or is a variant thereof with at least about 90% or 95% sequence identity to SEQ ID NO:12.

### 7.3.1.2 smCBA

In some embodiments, a smCBA (small modified chicken beta-actin) promoter is used to drive expression of a protein encoded by a transgene. See U.S. Pat. No. 8,298,818. In one embodiment, the smCBA promoter includes a CMV enhancer sequence, the beta actin promoter, a spacer, a chicken b-actin intron, an intron acceptor b-globin, and a beta globin exon 3. An exemplary sequence is provided at SEQ ID NOs: 10 and 11. It will be recognized that the sequence can be modified without loss of biological activity, and variants of the sequence with at least about 90% or about 95% sequence identity can be used.

### 7.3.1.3 sctmCBA

In some embodiments, a sctmCBA promoter is used to drive transcription of a gene product encoded by a cargo. In one embodiment, the sctmCBA promoter includes a CMV enhancer sequence, the beta actin promoter, a spacer, and a truncated chicken b-actin intron. An exemplary smtmCBA promoter is set forth as SEQ ID NO.:18. It will be recognized that the sequence can be modified without loss of biological activity, and variants of the sequence with at least about 90% or about 95% sequence identity can be used.

### 7.3.1.4 BEST1

In some embodiments, a BEST1-EP-454 promoter is used. An exemplary BEST1-EP-454 enhancer promoter is provided at SEQ ID NO:238. An exemplary BEST1-V3 promoter is provided at SEQ ID NO:239. It will be recognized that this sequence can be modified without loss of biological activity, and variants of the sequence with at least about 90% or about 95% sequence identity can be used.

### 7.3.1.5 VMD2

In one embodiment, a form of VMD2 promoter is used (see, e.g., SEQ NO:13). VMD2 has 680 bases from BEST1-743 and a 97 base 3′ enhancer sequence from SV40 intron. An illustrative 624 base promoter sequence is provided at SEQ ID NO:51. It will be recognized that this sequence can be modified without loss of biological activity, and variants of the sequence with at least about 90% or about 95% sequence identity can be used.

### 7.3.1.6 RPE65

In one embodiment, a truncated RPE65 promoter is used. An exemplary sequence is provided at SEQ ID NO:14. It will be recognized that this sequence can be modified without loss of biological activity, and variants of the sequence with at least about 90% or about 95% sequence identity can be used.

### 7.3.1.7 Other Promoters

Other useful promoters include (i) TYR Promoter (SEQ ID NO:185); (ii) TYRP1(SEQ ID NO:186); Choroid-enriched highly active promoters FLT1 (SEQ ID NO:187); VWF (SEQ ID NO:188); MGP (SEQ ID NO:189); RGS5 (SEQ ID NO:190); and SFFV (SEQ ID NO:191).

### 7.2.1.1 Bidirectional Promoters

Bidirectional promoters drive the expression of two adjacent genes coded on opposite DNA strands. Bidirectional promoters include naturally occurring and synthetic promoters.

In some embodiments, a bidirectional promoter comprises phosphoglycerate kinase 1(PGK1) and elongation factor 1 alpha EF1a in a back-to-back configuration (Goding & Mann, Gene Terh. 2011, August:18(8):817-26). In some embodiments, a bidirectional promoter comprises a chicken beta-actin promoter duplication in opposing directions with a CMV enhancer in the middle.

In some embodiments, the promoter is a bidirectional mouse CMV promoter, which comprises the mouse CMV immediate early 1 promoter in one direction and the mouse CMV immediate early 2 promoter in the opposite directions. Such a promoter may further comprises enhancers, such as the natural mouse CMV enhancer, that comprises a major immediate early 1 enhancer and a major immediate early 2 enhancer. Such bidirectional promoters are described, e.g., in European Pat. No. EP1601776 and U.S. Pat. No. 10,570,417.

In some embodiments, the bidirectional promoter comprises a promoter that drives expression of protein in one direction and an RNA in the other direction. For example, in some embodiments, such a promoter comprises a Pol 11 promoter that contains 3 external control elements: a distal sequence element (DSE), a proximal sequence element (PSE), and a TATA box; and (2) a second basic Pol III promoter that includes a PSE and a TATA box fused to the 5′ terminus of the DSE in reverse orientation. For example, in the H1 promoter, the DSE is adjacent to the PSE and the TATA box, and the promoter can be rendered bidirectional by creating a hybrid promoter in which transcription in the reverse direction is controlled by appending a PSE and TATA box derived from the U6 promoter. See, e.g., U.S. Pat. No. 9,907,863 (describing use of the H1 promoter to express CRISPR gRNA with the H1 promoter sequence as a bidirectional promoter to express Cas9 nuclease).

### 7.2.1.2 Other Cis Regulatory Elements

### WPRE

In some embodiments, the viral vector further comprises a posttranscriptional regulatory element, such as a Woodchuck Hepatitis Virus Posttranscriptional Regulatory Element (WPRE) or shortened versions of WPRE) to enhance expression of transgenes delivered using the viral vector (e.g., SEQ ID NO:25 and 26).

### Polyadenylation Sequences

Constructs for expression complement regulatory sequences as described herein include polyadenylation sequences. Exemplary polyadenylation sequences include sequences derived from the bovine Growth Hormone bGH polyadenylation signal (e.g., SEQ ID NO:29); sequences derived from the HSV Thymidine Kinase polyadenylation signal (e.g., SEQ ID NO:28); and sequences derived from the SV40 polyadenylation signal (e.g., SEQ ID NO:27).

### “Spacer” Sequences

Sequences encoding regulatory elements, protein or RNA encoding elements or other elements may be contiguous or may be separated by spacer sequences. For example and not limitation spacer sequences are generally 10-100 bp in length. For example and not limitation exemplary spacers are provided as SEQ ID Nos: 41 and 43.

### 8. Exemplary Constructs

TABLES 8-10 describe exemplary constructs.

TABLE 8

| A | B | C | F | E | F | G* |
|---|---|---|---|---|---|---|
| Name | Length* | Promoter | Activity 1 | Element | Activity 2 | AAV |
| 2-1 | 4819 | smCBA | CFHT | IRES | Aflib | ✓ |
| 2-2 | 4685 | smCBA | CFHT | mIRES | Aflib | ✓ |
| 2-3 | 4711 | smCBA | Aflib | mIRES | CFHT** | ✓ |
| 2-4 | 4723 | smCBA | Aflib | mIRES | CFHT | ✓ |
| 2-5 | 4714 | smCBA | Aflib | mIRES | CFHT | ✓ |
| 2-6 | 4714 | smCBA | Aflib | mIRES | CFHT | ✓ |
| 2-7 | 4726 | smCBA | Aflib | mIRES | CFHT | ✓ |
| 2-8 | 4726 | smCBA | Aflib | mIRES | CFHT | ✓ |
| 2-9 | 4735 | smCBA | Aflib | mIRES | CFHT | ✓ |
| 2-10 | 4738 | smCBA | Aflib | mIRES | CFHT | ✓ |
| 2-11 | 4738 | smCBA | Aflib | mIRES | CFHT | ✓ |
| 2-12 | 4224 | smCBA | Aflib | FCP | CFHT | ✓ |
| 2-14 | 4226 | smCBA | CFHT | FCP | Aflib | ✓ |
| 2-14 | 5216 | smCBA | CFHT | IRES | CFI | ✓ |
| 2-15 | 4609 | SFFV | CFHT | mIRES | CFI | ✓ |
| 2-16 | 7524 | CBA | CFHT | IRES | CFI | |
| 2-17 | 8960 | VMD2 | CFH | IRES | CFI | |
| 2-18 | 3166 | smCBA | CFHT | T2A | Anti-CFHR1 | ✓ |
| 2-19 | 3931 | CBA | CFHT | T2A | Anti-CFHR1 | ✓ |
| 2-20 | 3166 | smCBA | CFHT | T2A | Anti-CFHR4 | ✓ |
| 2-21 | 3931 | CBA | CFHT | T2A | Anti-CFHR4 | ✓ |
| 2-22 | 3980 | smCBA | CFHT | SFFV | Anti-CFHR1- | ✓ |
| | | | | | Linker-Anti- | |
| | | | | | CFHR4 | |
| 2-23 | 4364 | VMD2 | CFHT | smCBA | Anti-CFHR1- | ✓ |
| | | | | | Linker-Anti- | |
| | | | | | CFHR4 | |

*In column G, the absence of a checkmark indicates a lentiviral or non-viral based construct.

TABLE 9

| A | B | C | F | E | F | G | H | |
|---|---|---|---|---|---|---|---|---|
| Name | Length* | Promoter | Activity 1 | Element | Activity 2 | Promoter | Activity 3 | AAV |
| 3-1 | 4515 | SFFV | CFHT | FCP | CFI | U6 | hpC7 | ✓ |
| 3-2 | 4515 | SFFV | CFHT | T2A | CFI | U6 | hpC7 | ✓ |
| 3-3 | 7094 | smCBA | CFHT | IRES | CFI | U6 | hpC7 | |
| 3-4 | 9369 | smCBA | CFH | IRES | CFI | U6 | hpC7 | |
| 3-5 | 3534 | smCBA | CFHT | T2A | Anti- | U6 | hpC7 | ✓ |
| | | | | | CFHR1 | | | |
| 3-6 | 4299 | CBA | CFHT | T2A | Anti- | U6 | hpC7 | ✓ |
| | | | | | CFHR1 | | | |
| 3-7 | 4050 | smCBA | CFHT | T2A | Anti- | SFFV | Anti- | ✓ |
| | | | | | CFHR1 | | CFHR4 | |
| 3-8 | 3966 | VMD2 | CFHT | T2A | Anti- | smCBA | Anti- | ✓ |
| | | | | | CFHR1 | | CFHR4 | |

In the last column, the absence of a checkmark indicates a lentiviral or non-viral based construct.
*Including ITRs/LTRs, signal peptides, poly(A), Kozak, spacer(s)
**Optional spacer between F and E (Aflib stop codon and IRES start)

TABLE 10

| A | B | C | F | E | F | G | H | I | J | |
|---|---|---|---|---|---|---|---|---|---|---|
| Name | Length* | Promoter | Activity 1 | Element | Activity 2 | Promoter | Activity 3 | Promoter | Activity 4 | K* |
| 4-1 | 4444 | smCBA | CFHT | T2A | Anti- | SFFV | Anti- | U6 | hpC7 | ✓ |
| | | | | | CFHR1 | | CFHR4 | | | |
| 4-2 | 4360 | VMD2 | CFHT | T2A | Anti- | smCBA | Anti- | U6 | hpC7 | ✓ |
| | | | | | CFHR1 | | CFHR4 | | | |

*In column K, the absence of a checkmark indicates a lentiviral or non-viral based construct.

### 9. Administration and Conditions Treated

The administration and dose of the gene therapy vectors of the invention will depend on factors such as the complement dysregulation disease being treated. For example, the gene therapy vector may be administered by systemic administration (e.g., intravenous injection or infusion), local injection or infusion (e.g., subretinal, suprachoroidal, intravitreal, transscleral, trans corneal or other ocular), by use of an osmotic pump, by electroporation, by application (e.g., eye drops) and by other means. It is contemplated that transgenes of the invention may be introduced into, and expressed in, a variety of cell types including neural retinal cell types (such as rod photoreceptor cells, cone photoreceptor cells, ganglion cells), RPE, ciliary epithelial, scleral, iris, choroidal (such as choroidal endothelial cells, melanocytes, fibroblasts) and other ocular cells. See Xue et al., “Technique Of Retinal Gene Therapy: Delivery Of Viral Vector Into The Subretinal Space” Eye 31:1308-1316, 2017; Moore et al., 2017, “Gene Therapy For Age-Related Macular Degeneration” Expert Opinion on Biological Therapy 17:10: 1235-1244; Ochakovski et al., 2017, “Retinal Gene Therapy: Surgical Vector Delivery In The Translation To Clinical Trials” Frontiers in Neuroscience 11; Schon et al., 2015, “Retinal Gene Delivery By Adeno-Associated Virus (Aav) Vectors: Strategies And Applications” European Journal of Pharmaceutics and Biopharmaceutics 95:343-352.

In one approach, the vector is administered via a suprachoroidal injection, thereby allowing the agent access to choroidal, scleral and the RPE cells. See Ding et al., “AAV8-vectored suprachoroidal gene transfer produces widespread ocular transgene expression”, J Clin Invest 129(11):4901-4911, 2019. Also see Emami- and Yiu, Medical and Surgical Applications for the Suprachoroidal Space, Int Ophthalmol Clin 59(1):195-207, 2019. In one approach, the agent is administered via subretinal injection. See Xue et al., “Technique Of Retinal Gene Therapy: Delivery Of Viral Vector Into The Subretinal Space” Eye 31:1308-1316, 2017. In another approach, the agent can be injected into the vitreous. Kansar et al., “Suprachoroidal Delivery of Viral and Nonviral Gene Therapy for Retinal Disease”, J Ocular Pharmacol Ther 36:610.1. The amount of agent administered will be an “effective amount” or a “therapeutically effective amount,” i.e., an amount that is effective, at dosages and for periods of time necessary, to achieve a desired result. A desired result would include an improvement in a symptom associated with AMD progression. In another approach, the vector (viral or nonviral) is delivered to the eye by electroporation (Lebreton et al US patent application 20210128911; Behar-Cohen U.S. patent application Ser. No. 17/144,341; Bejjani et al, 2007, Survey Ophthalmol 52:196-208; Bloquel et al, 2006, Adv Drug Delivery Rev 58:1224-42).

As noted previously, complement dysregulation is an underlying element in many conditions and it is contemplated that these conditions can be treated using the methods described herein above. Treatable conditions include anti-neutrophil cytoplasmic antibody (ANCA)-associated vasculitis, Alzheimer's disease, atypical hemolytic uremic syndrome, acute kidney injury, age-related macular degeneration, antibody-mediated rejection, antiphospholipid syndrome, acute respiratory distress syndrome, Berger's disease, bullous pemphigoid, C3 glomerulopathy, C3 glomerulonephritis, cold agglutinin disease, chronic obstructive pulmonary disease, cardiopulmonary bypass, central serous chorioretinopathy, dense deposit disease, delayed graft function, early onset macular drusen, glaucoma, Guillain-Barré syndrome, generalized myasthenia gravis, granulomatosis with polyangiitis, graft versus host disease, hereditary angioedema, hidradenitis suppurativa, hematopoietic stem cell transplant-related thrombotic microangiopathy, IgA nephropathy, ischemia/reperfusion, immune complex-mediated membranoproliferative glomerulonephritis, immune-mediated necrotizing myopathy, idiopathic polypoidal choroidal vasculopathy, kidney transplant, lupus nephritis, membranous nephropathy, microscopic polyangiitis, multiple sclerosis, neuromyelitis optica (spectrum disorder), pyoderma Gangrenosum, paroxysmal nocturnal hemoglobinuria, proliferative diabetic retinopathyrheumatoid arthritis/osteoarthritis, systemic inflammatory response syndrome, systemic lupus erythematosus, Stargardt Disease, severe sepsis, septic shock, thrombotic microangiopathy, warm-type autoimmune hemolytic anemia and many others (Ricklin et al. 2016, 2018; Zelek et al 2019; Harris et al. 2018; Morgan & Harris 2015; Thurman and Holers 2006; Barlow 2017; Gavriilaki and Brodsky 2020; Zipfel et al. 2006; Schmidt et al. 2016; Mullins et al. 2017; Mohlin et al. 2017; Jha et al. 2007; Perkins et al. 2020; Taylor et al. 2019; Clark and Bishop 2018; Mandava et al 2020; de Jong et al. 2021; Bosco et al. 2018; Kaye 2020; Silverman and Wong 2019; Sohn et al. 2007; Xu and Chen 2016; Veszeli et al. 2014).

### 10. Examples

### 10.1 Example 1: Anti-CFHR-1 Nanobodies

Nanobodies were produced at Yurogen Biosystems (Worcester, MA) by camelid immunization with FHR-1A protein (Yurogen). PBMC were isolated from selected camelids and a VHH-contained cDNA library was prepared. Antigen-specific VHH were identified using a plate-based method and a VHH-specific detection antibody.

A total of 1536 individual B-cells were isolated 70 days after FHR-1 protein immunization and supernatant screened for FHR-1 direct binding. VHH positive clones were detected using a rabbit anti-camelid VHH antibody. A binding signal cut-off of 0.5 RLU (relative light units) in the direct FHR-1 ELISA identified 246 clones and 0.9 RLU signal cut-off resulted in 45 clones (2.93% of total). See TABLE 11.

TABLE 11
Nanobody Clones with Robust CFH-1 Binding Signal.

| | CFH-1 ELISA |
|---|---|
| Clone# | Signal (RLU) |
| 1C3 | 1.421 |
| 1E10 | 1.128 |
| 1G2 | 1.08 |
| 1G7 | 1.331 |
| 2C1 | 1.081 |
| 3A3 | 1.099 |
| 3B8 | 1.269 |
| 3C12 | 1.444 |
| 3G8 | 0.954 |
| 4B1 | 1.438 |
| 4C8 | 1.292 |
| 4D10 | 1.208 |
| 5B10 | 1.542 |
| 5C3 | 1.211 |
| 5C12 | 1.707 |
| 5H8 | 1.298 |
| 5H10 | 1.649 |
| 6A2 | 1.485 |
| 6D12 | 1.465 |
| 6E1 | 1.71 |
| 7C10 | 1.526 |
| 8A3 | 1.459 |
| 8E4 | 1.309 |
| 9A5 | 1.594 |
| 9H11 | 1.623 |
| 10B3 | 1.648 |
| 10C8 | 1.487 |
| 10D9 | 1.703 |
| 10G7 | 1.932 |
| 11C5 | 1.527 |
| 11C8 | 1.582 |
| 11D8 | 1.566 |
| 1.1E10 | 1.671 |
| 11H9 | 1.58 |
| 12C5 | 1.63 |
| 12D4 | 1.183 |
| 12D11 | 2.011 |
| 12F8 | 1.985 |
| 12H4 | 1.661 |
| 12H6 | 1.753 |
| 14A1 | 1.113 |
| 15C4 | 1.14 |
| 15F10 | 1.673 |
| 15G11 | 1.151 |
| 16B2 | 1.332 |

The top 45 FHR-1 positive VHH nanobody clones were screened against recombinant his-tagged CFH, CFHT, FHR-2 and FHR-4 proteins (0.5 mg/mL identical to the original FHR-1 binding assay). Approximately 71% of the FHR-1 nanobody clones exhibit cross-reactivity to CFH, 2.2% to CFHT, 38% to FHR-2 and 0% to FHR-4 protein (RLU cut-off of ≥0.1). See TABLE 12.

TABLE 12
VHH Antibody Clones Cross-reactivity Profile with Recombinant CFHT, CFH,
FHR-4 and FHR-2 Proteins.

| | Nanobody Primary | | | | |
|---|---|---|---|---|---|
| | Screen | Nanobody Counter-screen for Cross-reactivity | | | |
| Positive | CFH-1 ELISA Signal | CFHT ELISA | CFH ELISA | CFH-4 ELISA | CFH-2 ELISA |
| Clone # | (RLU) | Signal (RLU) | Signal (RLU) | Signal (RLU) | Signal (RLU) |
| 12D11 | 2.011 | 0.014 | 0.011 | 0.01 | 0.332 |
| 12F8 | 1.985 | 0.006 | 0.025 | 0.089 | 0.644 |
| 10G7 | 1.932 | 0.004 | 0.012 | 0.006 | 0.532 |
| 12H6 | 1.753 | 0.049 | 1.566 | 0.028 | 0.008 |
| 6E1 | 1.71 | 0.007 | 1.555 | 0.02 | 0.136 |
| 5C12 | 1.707 | 0.006 | 1.359 | 0.007 | 0.112 |
| 10D9 | 1.703 | 0.003 | 0.01 | 0.011 | 0.616 |
| 15F10 | 1.673 | 0.015 | 1.391 | 0.077 | 0.009 |
| 11E9 | 1.671 | 0.012 | 0.008 | 0.005 | 0.578 |
| 12H4 | 1.661 | 0.003 | 1.081 | 0.089 | 0.01 |
| 5H10 | 1.649 | 0.008 | 0.01 | 0.014 | 0.416 |
| 10B3 | 1.648 | 0.004 | 1.365 | 0.01 | 0.008 |
| 12C5 | 1.63 | 0.022 | 0.934 | 0.007 | 0.009 |
| 9H11 | 1.623 | 0.008 | 0.756 | 0.008 | 0.273 |
| 9A5 | 1.594 | 0.088 | 1.393 | 0.03 | 0.005 |
| 11C8 | 1.582 | 0.004 | 0.015 | 0.008 | 0.364 |
| 11H9 | 1.58 | 0.025 | 1.121 | 0.014 | 0.001 |
| 11D8 | 1.566 | 0.008 | 1.123 | 0.008 | 0.005 |
| 5B10 | 1.542 | 0.003 | 1.244 | 0.01 | 0.022 |
| 11C5 | 1.527 | 0.004 | 1.071 | 0.01 | 0.01 |
| 7C10 | 1.526 | 0.01 | 1.289 | 0.018 | 0.004 |
| 1008 | 1.487 | -0.001 | 0.015 | 0.044 | 0.314 |
| 6A2 | 1.485 | 0.006 | 1.233 | 0.015 | 0.039 |
| 6D12 | 1.465 | 0.007 | 0.009 | 0.015 | 0.472 |
| 8A3 | 1.459 | 0.028 | 0.611 | 0.017 | 0.006 |
| 3C12 | 1.444 | 0.025 | 1.315 | 0.022 | 0.106 |
| 4B1 | 1.438 | 0.009 | 1.185 | 0.074 | 0.018 |
| 1C3 | 1.421 | 0.041 | 1.144 | 0.005 | 0.054 |
| 16B2 | 1.332 | 0.014 | 0.358 | 0.005 | 0.008 |
| 1G7 | 1.331 | 0.019 | 1.505 | 0.012 | 0.027 |
| 8E4 | 1.309 | 0.015 | 0.006 | 0.016 | 0.313 |
| 5H8 | 1.298 | 0.005 | 0.007 | 0.007 | 0.262 |
| 4C8 | 1.292 | 0.013 | 0.987 | 0.019 | 0.019 |
| 3B8 | 1.269 | 0.022 | 1.338 | 0.007 | 0.016 |
| 5C3 | 1.211 | 0.009 | 0.016 | 0.005 | 0.11 |
| 4D10 | 1.208 | 0.101 | 0.756 | 0.044 | 0.019 |
| 12D4 | 1.183 | 0.017 | 0.007 | 0.026 | 0.146 |
| 15G11 | 1.151 | 0.013 | 1.187 | 0.027 | 0.006 |
| 15C4 | 1.14 | 0.013 | 1.082 | 0.02 | 0.008 |
| 1E10 | 1.128 | 0.022 | 1.05 | 0.006 | 0.042 |
| 14A1 | 1.113 | 0.012 | 1.31 | 0.007 | 0.004 |
| 3A3 | 1.099 | 0.021 | 0.753 | 0.007 | 0.072 |
| 2C1 | 1.081 | 0.01 | 0.84 | 0.013 | 0.007 |
| 1G2 | 1.08 | 0.042 | 0.814 | 0.003 | 0.015 |
| 3G8 | 0.954 | 0.014 | 0.78 | 0.004 | 0.052 |

### 10.2 Example 2: Exemplary Constructs

TABLE 13

| SeqID | Name | | |
|---|---|---|---|
| 219 | pCTM398 | 4819 | smCBA-CFHT-Spacer (18n)-IRES-TTR- |
| | | | aflibercept-Kozak-pA |
| 220 | pCTM418 | 4714 | smCBA-aflibercept-IRES-Spacer (49n)-IGF- |
| | | | 1CFHT-Kozak-pA |
| 221 | pCTM419 | 4714 | smCBA-aflibercept-IRES-Spacer (49n)-IGF- |
| | | | 1CFHT-Kozak-pA |
| 222 | pCTM428 | 4224 | smCBA-aflibercept-FCP-CFHT |
| 223 | pCTM429 | 4226 | smCBA-CFHT-FCP-aflibercept |
| 226 | pCTM417 | | Aflib-CFHT construct |
| 227 | pCTM369 | 5216 | smCBA-CFHT-Spacer (18n)-IRES-Spacer |
| | | | (45n)-CFI--Kozak-bGHpA |

### 10.3 Example 2: Exemplary Activity Assays

As noted above, where an exogenous protein, promotor, or regulatory element is described it is contemplated that variants could be used in place of the exemplified sequences, including naturally occurring variants of proteins and nucleic acids, and nonnaturally occurring sequences that are substantially identical to a reference sequence that is, or that encodes a protein that is, functionally similar to the reference sequence. Functional similarity can be determined using art-known assays for protein or nucleic acid function. Typically a variant that has a level of activity that is 90% or greater of that measured for the reference protein is functionally similar to the reference protein. This section describes certain assays for activity and/or quantity of various polynucleotides or encoded proteins. However, it will be understood that assay conditions can be adjusted. Further, it is understood that the scientific literature is repeat with assays for complement protein activity, promoter activity and the like and that the skilled PR actioner can easily identify alternative or additional assays. It is further understood that assay conditions can be adjusted.

### 10.3.1 C3b, CRP and MDA-LDL Assays to Monitor CFH, CFHT, and oCFHT Protein Binding

The C3b binding assay uses C3b protein (Complement Technology, Cat. #A114) diluted with 50 mM carbonate coating buffer (pH 9.6) at a final concentration of 50 μM. A total of 100 μl of C3b/carbonate solution was added to each well of a black MaxiSorp 96-wellmicroplate, covered and incubated overnight at 4° C. Blank control wells were incubated with carbonate coating buffer containing no C3b protein. Wells were washed 3 times with 300 μl PBST then blocked for 1 hour with reagent dilution buffer (1% BSA in PBS). Wells were washed again 3 times with 300 μl PBST. Dilutions of recombinant protective CFH, CFHT, oCFHT or supernatant from transiently transfected or transduced Cos-7 cells were prepared in reagent dilution buffer, added to wells in triplicate and incubated for 1 hour at room temperature. The plate was washed as above then incubated for 1 hour with anti-CFH/CFHT biotinylated aCTM87b antibody (OX-24, AbCam, Cat. #ab112197) at 1:1000 dilution. The plates were washed as above, then incubated for 1 hour with high sensitivity Streptavidin-HRP (Pierce Cat. #21130) at 1:10,000, washed as above then incubated for 5 minutes with SuperSignal ELISA Pico Chemiluminescent Substrate (Thermo Scientific, Cat. #37069) before detection using the BioTek Synergy4 plate reader.

To measure binding of CFH, CFHT, and oCFHT proteins to CRP (R&D Systems, Cat. #1707-CR/CF) or MDA-LDL ligands (Cell Biolabs, Cat. #STA-212), microtiter plates(Maxisorp, Nunc) were coated overnight at 4° C. at a concentration of 2.5 μg/ml in PBS. Wells treated with only PBS or non-modified LDL (Cell Biolabs, Cat. #STA-241) served as negative ligand binding controls. After three washes in PBST, plates were blocked with SuperBlock T20 (PBS) Blocking Buffer (Thermo Scientific, Cat. #37516) for 1 hour at room temperature. Various dilutions of recombinant protective CFH, CFHT, or oCFHT or supernatant from transiently transfected or transduced Cos-7 cells were added in blocking buffer and incubated at room temperature for 1 hour. The relative binding of CFH, CFHT, or oCFHT proteins was assessed using biotinylated aCTM87b followed by high sensitivity Streptavidin-HRP and signal was detected as described above.

### 10.3.2 LPS-driven Alternative Pathway (AP) Assay

LPS-dependent Alternative Pathway (AP) Assay to Monitor CFH, CFHT, oCFHT and/or CFI Protein Activity AP activation was determined using an established ELISA-based assay (Harder M J et al. (2015) J. Immunol. 196:866-876) with minor modifications. In brief, 50 μl LPS solution (50 g/ml) from Salmonella typhimurium (Sigma-Aldrich, Cat. #L7261) was coated onto 96-well plates (Maxisorp; Nunc) in PBS overnight at 4 C, followed by washing three times with PBS+Tween 20. Plates were then blocked with 1% BSA/PBS for 1 hour at room temperature. Various dilutions of recombinant protective CFH, CFHT, oCFHT, CFI or supernatant from transiently transfected or transduced Cos-7 cells were mixed with 30 μl 25% normal human serum containing 10 mM MgEGTA. The mixture of analytes in serum was added to LPS-coated wells and incubated for 1.5 hours at 37° C. prior to washing and subsequent exposure to HRP conjugated goat anti-human C3 (MP Biomedicals, Cat. #855237) at 1:10,000 dilution in 1% BSA/PBS for 1 hour at room temperature. After washing three times with PBST, C3b deposition on the plate was indirectly detected using SuperSignal ELISA Pico Chemiluminescent Substrate and the BioTek Synergy 4 plate reader. PBS and EDTA (final concentration 5 mM) were used as positive and negative controls, respectively, and cell culture supernatant provided tissue media control. All responses were normalized to the activity achieved when only PBS was added in the absence of a CFH, CFHT, oCFHT and/or CFI protein regulators. All data were plotted using a nonlinear regression log (inhibitor) versus response (three parameters) model in Prism 9.0.

### 10.3.3 RBC Lysis Assay to Monitor CFH, CFHT, oCFHT, and/or CFI Protein Activities

For fluid-phase alternative pathway activity, various dilutions of recombinant protective CFH, CFHT, CFI or supernatant from transiently transfected or transduced Cos-7 cells were mixed with 15 μl of CFH-depleted normal human serum (Complement Technology, Cat. #A337), 5 μl of Mg EGTA (0.1 M) and 25 μl of rabbit erythrocytes (Complement Technology, Cat. #B300) (5×108/ml in GVB) in a final volume of 100 μl of GVB. The reaction mixture was incubated at 37′C for 30 min and stopped by adding 1 ml of GVBE(GVB containing 10 mM EDTA). After centrifugation at 1000 g for 3 min, the absorbance of 100 μl of the supernatant from each sample was determined at 412 nm using the BioTek Synergy 4 plate reader. The percentage of lysis was normalized by setting 100% lysis to be equal to the degree of lysis occurring in the presence of normal human serum (Complement Technology, Cat. #NHS). CFI-Dependent Cofactor Activity Assay to Monitor CFH, CFHT, oCFHT, and/or CFI Protein Activity.

Co-Factor Activity Assay to Monitor CFH, CFHT, oCFHT and/or CFI Protein Activities

A fluid-phase assay was used to measure cofactor activity. Various dilutions of recombinant protective CFH, CFHT, oCFHt, CFI or supernatant from transiently transfected or transduced Cos-7 cells were incubated with C3b (Complement Technologies, Cat. #A114) and CFI (Complement Technologies, Cat. #A138) in a total volume of 20 μl PBS for 20 min at 37′C. For each reaction, 526 nM of C3b and 22.7 nM of CFI were used with varying concentrations of CFH, CFHT, or oCFHT proteins or Cos-7 supernatant. In some examples, CFI protein was eliminated from the experiment to monitor CFI activity in Cos-7 supernatant. The assay was stopped with the addition of 3 μl 10× NuPAGE sample reducing agent (Life Technologies, cat. #NP0009) and heated for 7 min at 95° C. Samples were electrophoresed on a 4-12% NuPAGE Bis-Tris gel at 200 V for 45 min to maximize separation of C3b breakdown products. Visualization of C3b and iC3b bands was accomplished by Coomassie blue staining (Life Technologies, Cat. #LC6065). The band intensity of C3b α-chain, C3b β-chain, and the 68-kDa and 43-kDa iC3b products were measured using Multi Gauge software (version 3.0) on the LAS4000 image analyzer and plotted using Prism GraphPad 9.0 software.

### 10.3.4 Assays to Monitor Aflibercept Protein Level and Activity

Aflibercept levels in Cos-7 cell culture supernatant after transfection or transduction with plasmid DNA were measured using an ELISA based assay (Kiss et al, Mol Ther Methods Clin Dev. 2020, 18: 345-353). Briefly, MaxiSorp plates were coated with 100 μl/well recombinant human VEGFA (rhVEGFA; R&D Systems Cat. #293-VE-010/CF) at a concentration of 1 μg/ml in coating buffer (R&D Systems, Cat. #DY006 Systems) and incubated overnight at 4′C. After being washed with wash buffer (KPL, VWR Cat. #95059-132), the plate was blocked with 300 μl/well Pierce protein-free blocking buffer (ThermoFisher Scientific, Cat. #37572) for 1.5 hr at room temperature. Afterward, the plate was washed then 1:25 diluted Cos-7 cell culture supernatant in blocking buffer and 100 μl added/well and incubated for 2 hr at room temperature. The plate was washed again and 100 μl/well anti-human Fcγ-specific antibody conjugated to horseradish peroxidase (HRP) (Jackson Immuno Research, Cat. #109-035-008) at 500 ng/ml in bovine serum albumin (1% in PBS) was added to the wells. After a washing step, 100 μl/well SuperSignal ELISA Pico Chemiluminescent Substrate (Thermo Fisher Scientific, Cat. #37069) was added to the wells and luminescence signal was measured using the BioTek Synergy 4 plate reader. EYLEA (Regeneron Pharmaceuticals) purchased from the Moran Eye Center Pharmacy was used to generate the Aflibercept ELISA protein standard curve and determine concentrations from cell culture supernatant samples.

The VEGF inhibitory activity of Aflibercept in Cos-7 cell culture supernatant was measured using a VEGF Bioassay kit from Promega (Cat. #GA2001) following the supplied protocol. Methods are described briefly below. Control (ELYEA) and cell culture supernatant samples were 2-fold serially diluted in kit supplied assay buffer (DMEM medium with 10% FBS). The assay procedure is as follows—1. Prepare cell culture supernatant by serial dilution in assay buffer. In our assay, ELYEA was used as positive control with a starting concentration of 500 ng/ml (3×=1.5 μg/ml). 2. Prepare 3×VEGF at EC80 concentration in assay buffer. The expected EC80 of VEGF is approximately 20 ng/ml (3×=60 ng/ml). 3. Transfer 0.4 ml of KDR/NFAT-RE HEK293 cells to a 15 ml conical tube containing 4.6 ml of assay buffer. Mix well by gently inverting 1-2 times. Dispense 25 μl of the cell suspension to each well of 96-well assay plate. 4. Dispense 25 μl of the 3×ELYEA and cell culture supernatant serial dilutions to the 25 μl of pre-plated cells. 5. Dispense 25 μl of the 3×EC80 VEGF mixture prepared to wells. The final assay volume is 75 μl. 6. Cover assay plate with a lid and incubate in a 37° C., 5% C02 humidified incubator for 6 hours. 7. Remove the assay plate from the incubator and equilibrate to room temperature for 10-15 minutes. 8. Add 75 μl of Bio-Glo Reagent to the wells, taking care not to create bubbles. 9. Incubate at room temperature for 5-10 minutes. 10. Measure luminescence using BioTek Synergy 4 plate reader and plot data using Prism GraphPad 9.0 software.

### 10.3.5 C7 ELISA to Monitor C7 hpRNA Activity

The C7 capture antibody (Complement Technology #A224, 1 mg/ml) was diluted 1:32000 in 50 mM carbonate coating buffer (pH 9.6); a total of 100 μl of antibody/carbonate solution was added to each well of a black MaxiSorp 96-well microplate, covered and incubated overnight at 4° C. Plates were washed using the BIOTEK EL404 Microplate washer consisting of 3 cycles of 350 μl PBST with 5 seconds of soak time and no shaking followed by aspiration then blocked for 1.5 hours with reagent dilution buffer (1% BSA in 1×PBS). Human C7 protein standards (Complement Technology #A124, 1 mg/ml) were diluted in RDB just prior to assay. Standards and Cos-7 supernatant samples (100 μl) were added to each well in duplicate and incubated for 1.5 hours at room temperature. Normal human serum standard (Quidel Cat. No. A100) and C7-depleted serum (Complement Technology Cat. #A324, 1 mg/ml) were added to each assay plate as positive and negative controls, respectively. The plate was washed as above then incubated for 1.5 hours with sandwich antibody at 1:5000 dilution (ThermoFisher Cat. #MA5-30114, 1 mg/ml). The plate was washed as above and then incubated for 1 hour with anti-rabbit-HRP (Jackson ImmunoResearch, Cat. #111-035-045) at 1:10,000 dilution. The plate was washed as above, then incubated for 5 minutes with SuperSignal ELISA pico chemiluminescent substrate (ThermoFisher Scientific, Cat. #37069) before detection using the BioTek Synergy4 plate reader.

### 10.4 Example 3: C7 Gene Expression and Protein Levels are Upregulated in AMD and can be Reduced Using Inhibitory HPRNA

This example shows that C7 gene expression is upregulated in RPE/choroid in AMD, and that C7 serum levels are elevated are elevated in AMD. We further demonstrate that hpRNA can be used to decrease C7 levels in vitro.

### 10.4.1.1 C7 mRNA is Elevated in RPE-Choroid but not Retina from Donors with AMD Risk-Associated Genotypes

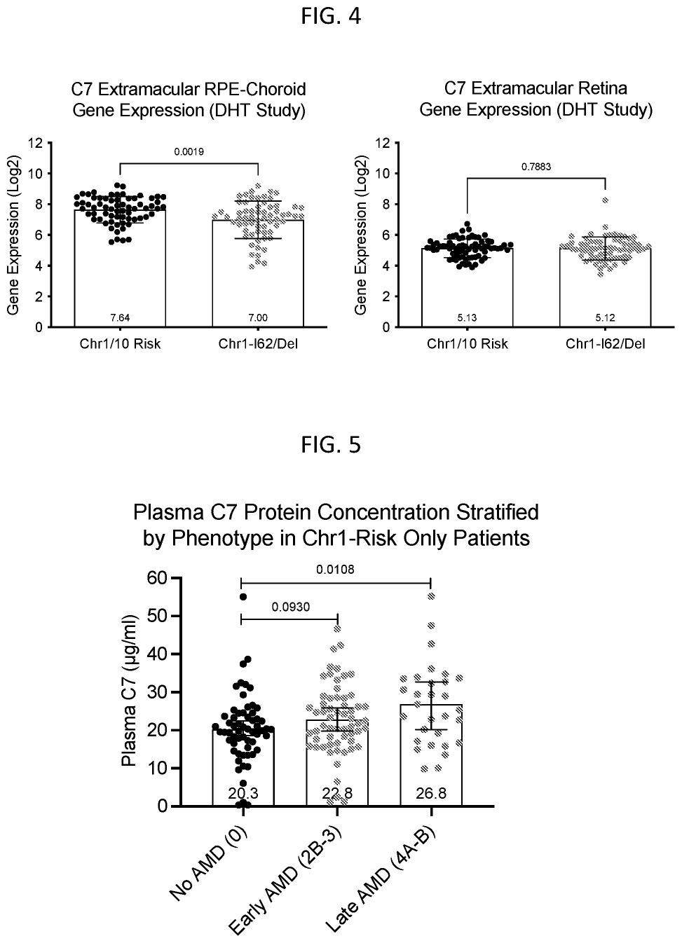

We measured C7 mRNA levels in the RPE-choroid and retina of donors with AMD risk-associated genotypes, e.g., homozygous Chr1-Risk (G1), Chr1-I62 (G8), Chr1-Del (G10) and homozygous Chr10-Risk (G25, G26, G27, G28, G29, G30) genotypes. See FIG. 4. AMD risk-associated genotypes are described in Table 14, below (also see WO2020019002, Table 15). C7 mRNA levels are elevated in the RPE-choroid (but not the retina) of Chr1 (G1) and Chr10 (G25, G26, G27, G28, G29, G30) AMD risk donor samples, but not in the RPE-choroid of Chr1-I62/Del protection (G8, G10) samples. Each data point is an individual donor extramacular RPE-choroid (left) or extramacular retina (right) sample.

TABLE 14
GENOTYPE GROUPS (BASED ON 4 SNPS) AND ASSOCIATED
AMD ODDS RATIOS

| | | | | AMD |
|---|---|---|---|---|
| | | | | Odds |
| Group | Chr 1 | Chr 10 | Protein Variants | Ratio |
| G1 | Risk/Risk | No Risk | VV62, HH402, EE936 | 8.3 |
| G8 | I62/I62 | No Risk | II62, YY402, EE936 | 1.2 |
| G10 | 3,1 del/3,1 del | No Risk | VV62, YY402, EE936 | 1 |
| G25 | Neut/Neut | Homo Risk | VV62, YY402, DD936 | 28.8 |
| G26 | Neut/I62 | Homo Risk | IV62, YY402, ED936 | 17.2 |
| G27 | Neut/3,1 del | Homo Risk | VV62, YY402, ED936 | 46.0 |
| G28 | I62/I62 | Homo Risk | II62, YY402, EE936 | 5.0 |
| G29 | I62/3,1 del | Homo Risk | IV62, YY402, EE936 | 9.3 |
| G30 | 3,1 del/3,1 del | Homo Risk | VV62, YY402, EE936 | 1.6 |

### 10.4.1.2 Plasma C7 Protein Levels are Elevated in Individuals with Homozygous Chr1 Risk Genotypes in Late Stage AMD, as Compared to Control No AMD Samples

Plasma levels of C7 protein were measured in individuals with homozygous Chr. 1 risk genotypes using a Human C7 ELISA as described in Example 3, above. In this population, a 32% increase in plasma C7 protein level was observed in individuals with late stage AMD compared to individuals with no clinical AMD. See FIG. 5. Plasma samples were from individuals homozygous for Chr1-Risk (G1) with no AMD, early AMD and late AMD ocular phenotypes.

### 10.4.1.3 C7 mRNA is Reduced in a Cell-Based Model Using shRNA Plasmid DNA

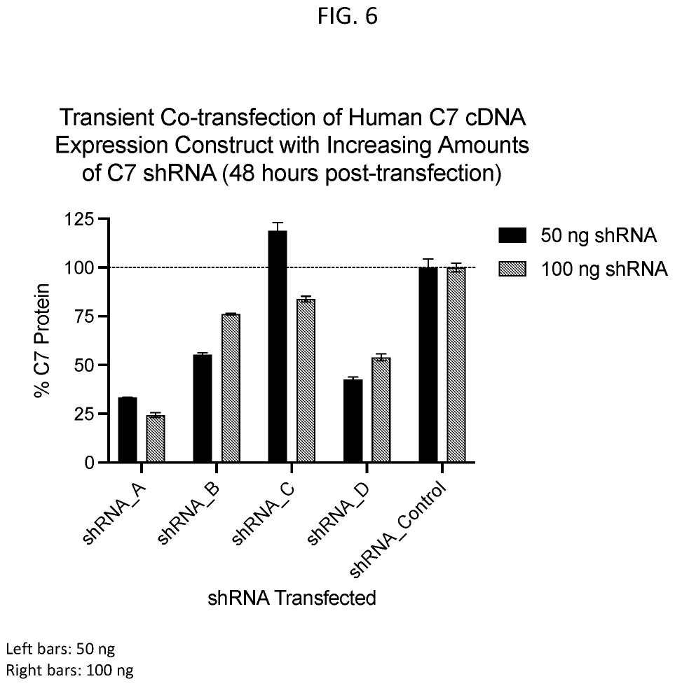

Cos-7 cells were transiently transfected with 50 ng human C7 cDNA expression construct and 50 or 100 ng of control (5′ GCACTACCAGAGCTAACTCAGATAGTACT 3′, SEQ ID NO:241) or a C7-specific shRNA plasmid DNA (see Table 7). C7 protein in supernatant was detected using a Human C7 ELISA as described in Example 3, above. Three independent shRNA plasmid DNAs (A, B and D) targeting C7 mRNA reduced C7 protein. See FIG. 6.

### 10.4.1.4 C7 Protein is Reduced in Neuroblastoma Cell Lines Transduced with Recombinant AAV2 Expressing shRNA and CFHT

Recombinant AAV2 viral particles containing expression constructs in which a U6 promoter is employed to express shRNA_A, shRNA_B, or shRNA_D were produced to assess reduction of endogenously expressed C7 in two neuroblastoma cell lines (SK-N-AS and SK-N-SH). The hpRNA sequences were each cloned into a pCTM297 KanR vector using standard restriction enzyme techniques. The constructs for shRNA_B and shRNA_D employ the same backbone vector. Plasmid pCTM297 also contains a cDNA sequence encoding CFHT (SEQ ID NO:9). Plasmid construction is described in greater detail in Section 10.5.

The sequences (sense-hairpin-antisense) for shRNA_A, shRNA_B and shRNA_D are as follows:

5′ to 3′ sequence for shRNA_A:
(SEQ ID NO: 243)
GAAGGTCCTTCAGCATTTCTCTGTGGCTCCAAGAGAGCCACAGAGAAATG
CTGAAGGACCTTC;
5′ to 3′ sequence for shRNA_B:
(SEQ ID NO: 244)
CAGAATGCAATGGCTGTACCAAGACTCAGCAAGACTGAGTCTTGGTACAG
CCATTGCATTCTG;
5′ to 3′ sequence for shRNA_D:
(SEQ ID NO: 245)
TCTGTCCATCACCTCCTGCCTTGAAAGATCAAGAATCTTTCAAGGCAGGA
GGTGATGGACAGA.

SK-N-AS (ATCC, Cat. #CRL-2137) and SK-N-SH (ATCC, Cat. #HTB-11) cells were maintained in Dulbecco's Modified Eagle's Medium with 10% FBS. Cells were plated at 10,000 cells per well in a 96-well plate format one day prior to rAAV2 transduction. The following day, rAAV2 particles were added to wells with 75 μl of DMEM. Viral particles at various multiplicity of infection (10E6, 10E5, 10E4, 10E3, 10E2, 10E1 and 10E0 vg/cell) were added to wells and 24 hours post-transduction, replaced with fresh media. After an additional 72 hours, supernatant was collected and tested for C7 level using a C7 ELISA.

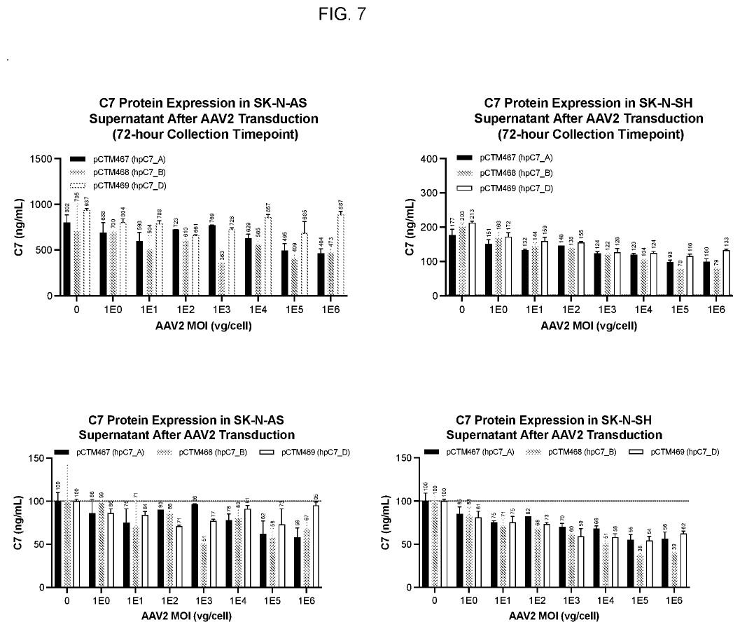

As shown in FIG. 7 (top panel), untreated wells lacking any viral particles (0) showed an average of 814 and 198 ng/mL of endogenous C7 protein in SK-N-AS and SK-N-SH cells, respectively. A dose-dependent reduction of C7 expression was evident with increasing amounts of viral particles in SK-N-AS for both shRNA_A- and shRNA_B-treated cells.

Percent C7 protein reduction is shown in the lower panel of FIG. 7. In the SK-N-AS neuroblastoma cell line, 1E+5 and 1E+6 viral particles of the shRNA_A and shRNA_B constructs resulted in ^{˜}40% reduction in endogenous C7 protein. In the lower C7 expressing SK-N-SH neuroblastoma cell line, all three hpRNA sequences are active with a 45% reduction detected with shRNA_A, ^{˜}60% for shRNA_B and ^{˜}45% reduction for shRNA_D.

Overall, these results indicate that C7 shRNAs are effective in down-regulating endogenous C7 protein expression in neuroblastoma cells lines, with shRNA_A and shRNA_B observed as the most effective in this experiment. Because C7 protein levels are much lower in the RPE-choroid region of human eyes, the results provide evidence that shRNA sequences will reduce C7 protein to levels that will significantly alter the amount of membrane attack complex (MAC).

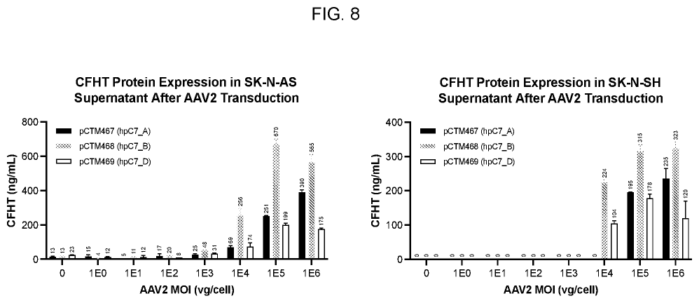

To confirm that AAV2 based multigene therapy vectors are capable of expressing another transgene, we also determined protective CFHT protein levels expressed in the same rAAV2 viral vectors. Supernatant used to assess C7 levels after hpRNA knockdown were similarly tested at each MOI in both cell lines for protective CFHT protein. As shown in FIG. 8, CFHT protein exhibited a dose-dependent increase in expression with increasing MOI. These results demonstrate that the multigene therapy vectors can produce both exogenous CFHT protein and reduce endogenous C7 protein using C7-targeting shRNA.

### 10.4.1.5 Summary

Reduction of C7 protein via multigene constructs that include expression of C7 inhibitory RNA (e.g., C7 shRNA_A, shRNA_B or shRNA_D) will reduce the amount of membrane attack complex (MAC) and cell death in the RPE-choroid region. Expression of optimized CFHT proteins by multigene constructs will additionally improve negative regulation of the alternative complement pathway.

### 10.5 Example 4: Optimized CFHT (oCFHT) Proteins for Improved Complement Control

Activities of oCFHT proteins containing additional SCR7 domains at the C-terminus and/or an FHR-1 SCR1/2 coding sequence at the N-terminus were evaluated. Human codon-optimized polynucleotides encoding one, two, or three CFHT SCR7 domains were cloned in-frame with the 3′ end of a protective CFHT cDNA to generate a total of two, three, or four SCR7 domains. Human-codon-optimized FHR-1 SCR1/2 coding sequences were also cloned in frame with the 5′end of protective CFHT cDNAs with one, two, three, and four SCR7 domains. The protective CFHT and oCFHT proteins are designated as follows: CFHT.0 (unmodified CFHT), mCFHT.1 (modified CFHT having one additional SCR7 domain at the C-terminus); mCFHT.2 (modified CFHT having two additional SCR7 domains at the C-terminus); mCFHT.3 (modified CFHT having three additional SCR7 domains at the C-terminus). CFHT proteins having an SCR1/2 coding sequence at the 5′ end are designated by reference to “dimer” or inclusion of a “d” in the CFHT protein designation, e.g., dCFHT.0 refers to CFHT modified to have a dimer at the N-terminus. mCFHT.1, mCFHT.2, and dCFHT cDNA and protein sequences are provided in SEQ ID NOS:246-251.

### Plasmid Construction

The plasmid constructs expressing optimized and unmodified CFHT proteins were based on plasmid pCTM297. pCTM297 was constructed from plasmid pCTM261 by replacing the AmpR selectable marker with KanR. vCTM261 (the AAV2 genome carried by the plasmid corresponding to pCTM261) is described in PCT publication WO 2020/019002A1. vCTM261 contains a codon-optimized sequence encoding a truncated complement factor H (CFHT) polypeptide, a CBA promoter, a bovine Growth Factor (bGH) polyadenylation sequence. ITR sequences corresponding to SEQ ID NO:18 of WO 2020/019002A1 (and its reverse complement) were used in vCTM261.

In constructs expressing oCFHT, the CFHT-encoding sequence was modified to add a downstream (3′ end) sequence encoding one, two, or three additional C-terminal SCR7 cDNA domains and/or modified upstream (5′ end) to add an in-frame FHR-1 SCR1/2 homodimerization domain.

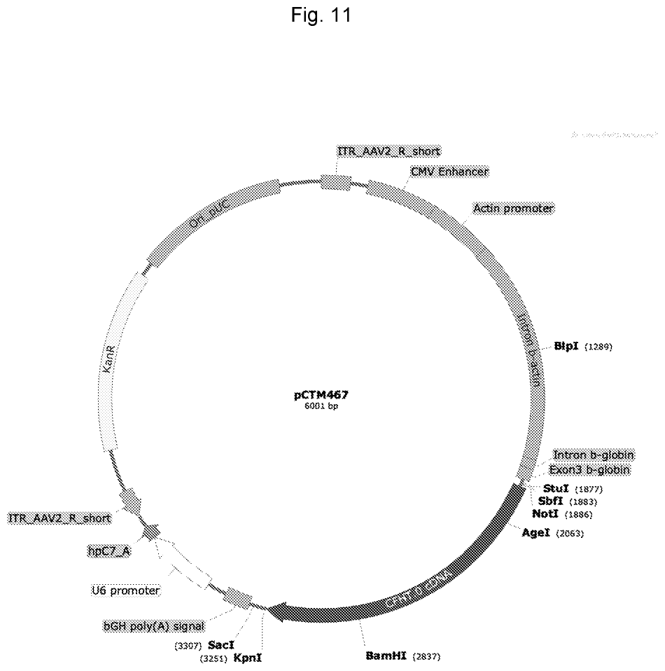

In some implementations, the plasmid was further modified to add a second gene sequence. For example, plasmids pCTM467-pCTM472 each include a U6 promoter-C7 shRNA hairpin sequence (see, Table 18). FIG. 11 provides a representative map, pCTM467, to illustrate the configuration of the shRNA-expressing sequence (in this instance shRNA_A) and CFHT/oCFHT cDNA (in this instance CFHT.0) in this series of plasmids.

In detail, the 6× His-tag cDNA was excised from Leader 6× His-TEV (GeneArt) and inserted into pCTM297 using StuI/AgeI cloning to produce pCTM447. The modified CFHT constructs containing one, two, or three additional C-terminal SCR7 cDNA domains were constructed by removal of GeneArt inserts with BamHI/SacI for subcloning into pCTM447 to generate pCTM459, pCTM460, and pCTM461. The dimerization domain of CFHR1, encoded by SCR1/2 was also synthesized by GeneArt and subcloned by StuI/AgeI digestion into pCTM459-461 to generate pCTM462-pCTM465 constructs. Table 15 provides a summary of 6×-His tagged CFHT and oCFHT expression constructs.

TABLE 15

| | | # Extra SCR7 | Transgene |
|---|---|---|---|
| CFHT | pCTM # | Domains | Size (bp) |
| CFHT.0 | pCTM297 | 0 | 1350 |
| His-CFHT.0 | pCTM447 | 0 | 1437 |
| His-mCFHT.1 | pCTM459 | 1 | 1614 |
| His-mCFHT.2 | pCTM460 | 2 | 1791 |
| His-mCFHT.3 | pCTM461 | 3 | 1968 |
| His-dCFHT.0 | pCTM462 | 0 | 1839 |
| His-mdCFHT.1 | pCTM463 | 1 | 2016 |
| His-mdCFHT.2 | pCTM464 | 2 | 2193 |
| His-mdCFHT.3 | pCTM465 | 3 | 2370 |
| (−) Control | pCTM395 | 0 | 0 |

CFHT expression for the plasmids listed in Table 15 was evaluated by transient expression in HEK293 cells. Cell culture supernatant containing secreted protein was assessed 48 hours post-transfection for CFHT protein levels using ELISA, and relative activity assessed using plate-based ligand binding and activity assays. Expression of CFHT.0, His-CFHT.0, His-mCFHT.1, and His-mCFHT.2 was observed, with CFHT.0 exhibiting the highest expression level. Expression of His-mCFHT.3 was not detected in this experiment. When the SCR1/2 dimerization domain of human FHR-1 protein is added to the N-terminal region of CFHT, we detect robust expression of dCFHT.0, but no detectable expression of modified dimer CFHT proteins with one, two, or three extra SCR7 domains (pCTM463-pCTM465). The pCTM395 construct served as a negative plasmid DNA control for all studies and does not have any detectable CFHT protein signal as expected. Western blots were also performed and depicted the increased size of CFHT proteins for pCTM447 (His-CFHT.0), pCTM459 (His-mCFHT.1), pCTM460 (His-mCFHT.2) and a broad band for pCTM462 (His-dCFHT.0), which was possibly due to N-linked glycosylation of the FHR-1 SCR2 domain.

Activities of the modified CFHT proteins was also compared to unmodified CFHT.0 and His-CFHT.0 proteins using the exemplary C3b, CRP and MDA-LDL assay protocols at the end of this section.

For C3b assays, 25 ng/mL of CFHT protein-containing supernatant was added to plates coated with C3b. A 43-, 99- and 22-fold increase in ligand binding was observed with mCFHT.1, mCFHT.2, and dCFHT.0, respectively, relative to control (see, Table 16). CFHT.0 (pCTM297) exhibited only a 1.5-fold increase in C3b binding relative to control supernatant at 25 ng/mL. Comparable results of significantly increased binding activity for mCFHT.1, mCFHT.2 and dCFHT.0 proteins were also observed for MDA-LDL- and CRP-coated plates (also summarized in Table 16). All three of the modified proteins performed significantly better than CFHT.0 expressed in supernatant (pCTM297 and pCTM447) or purified recombinant CFHT-I62 protein.

We then evaluated supernatants in an LPS-driven AP assay, summarized in Section 10.3.2. The mCFHT.1, mCFHT.2 and dCFHT.0 proteins again exhibited much better negative control of C3b deposition than CFHT.0 and rCFHT proteins (see, Table 16). The activities of recombinant CFHT-I62, CFHT.0- and His-CFHT.0-containing supernatants were negligible in the LPS-driven assay when tested at 25 ng/mL and lower.

TABLE 16
Summary of supernatant CFHT and oCFHT protein activities in
indicated assays.

| | | Fold Activity at Highest Dose | | | |
|---|---|---|---|---|---|
| | | (Relative to Control)* | | | |
| | | | | | LPS- |
| | | C3b | MDA-LDL | CRP | driven |
| Name | pCTM # | Binding | Binding | Binding | Assay |
| rCFHT-I62 | NA | 3.8 | 52 | 2.8 | 1 |
| Protein | | | | | |
| CFHT.0 | pCTM297 | 1.5 | 1.5 | 0.8 | 1.3 |
| His-CFHT.0 | pCTM447 | 1.5 | 1.4 | 2.7 | 1.2 |
| His-mCFHT.1 | pCTM459 | 43 | 102 | 6.3 | 0.8 |
| His-mCFHT.2 | pCTM460 | 99 | 78 | 49 | 0.2 |
| His-dCFHT.0 | pCTM462 | 22 | 88 | 6.4 | 0.7 |
| (−) Control | pCTM395 | 1 | 1 | 1 | 1 |

As noted above, a summary of all optimized CFHT and control CFHT protein activities assessed in this experiment is shown in Table 16. These studies demonstrate that modified CFHT proteins that contain extra SCR7 domains or a dimerization domain are functionally more potent than native protective CFHT protein.

We next tested the activity of purified recombinant mCFHT.1 and dCFHT.0 proteins purified from transfected HEK293 cells in comparison to unmodified wild-type CFHT.0 protein in C3b, MDA-LDL and CRP binding assays. In agreement with the activities observed above, activity of purified recombinant mCFHT.1 and dCFHT proteins were significantly improved, relative to CFHT.0, in C3b, MDA-LDL and CRP binding assays. Binding results are summarized in Table 17.

TABLE 17
Summary of purified recombinant CFHT protein activities in plate-based
binding assays.

| | | | Fold Activity | | |
|---|---|---|---|---|---|
| | Plasmid | | (Compared to CFHT-I62) | | |
| Recombinant | DNA | C3b | MDA-LDL | CRP | C3d |
| Protein Name | Number | Binding | Binding | Binding | Binding |
| CFHT.0 | pCTM134 | 1 | 1 | 1 | 1 |
| mCFHT.1 | pCTM459 | 40.1 | 6.8 | 12.2 | 3.8 |
| dCFHT.0 | pCTM462 | 55.3 | 10.9 | 2.2 | 2.1 |

Binding of the three recombinant proteins to C3d ligand was also evaluated. A four-fold increase in mCFHT.1 binding) and two-fold increase in dCFHT.0 binding were observed (Table 17).

Overall, the ability of the modified CFHT proteins to bind to key ligands indicates that modified CFHT proteins will control multiple events in the alternative complement pathway at much lower doses than CFHT.0.

We prepared AAV2 viral particles to transduce Cos-7 cell and express mCFHT.1, mCFHT.2, dCFHT.0 and mdCFHT.1 in cell culture supernatants. Cos-7 (ATCC, Cat. #CRL-1651) cells were maintained in Dulbecco's Modified Eagle's Medium with 10% FBS. Cells were plated at 10,000 cells per well in a 96-well plate format one day prior to rAAV2 transduction. The following day, rAAV2 particles were added to wells with 75 μl of DMEM containing 0.2% FBS at 1E+0 to 1E+5 in 10-fold increments in duplicate. The next day, cultures were replaced with fresh media and 3 days post-transduction supernatants were collected. The AAV2 constructs are shown in Table 18.

TABLE 18
AAV2 viral constucts expressing CFHT proteins with and without C7
hpRNA sequences

| | | C7 hpRNA Included | | |
|---|---|---|---|---|
| pCTM # | Protein Name | (Clone #) | Titer | Volume |
| pCTM452 | mCFHT.1 | No | 2.10E+11 | 100 ul |
| pCTM455 | dCFHT.0 | No | 2.90E+11 | 100 ul |
| pCTM456 | mdCFHT.1 | No | 3.18E+11 | 100 ul |
| pCTM467 | CFHT.0 | Yes (hpC7_A) | 3.89E+11 | 100 ul |
| pCTM468 | CFHT.0 | Yes (hpC7_B) | 4.65E+11 | 100 ul |
| pCTM469 | CFHT.0 | Yes (hpC7_D) | 3.48E+11 | 100 ul |
| pCTM470 | mCFHT.2 | Yes (hpC7_A) | 5.19E+11 | 100 ul |
| pCTM471 | mCFHT.2 | Yes (hpC7_B) | 1.11E+11 | 100 ul |
| pCTM472 | mCFHT.2 | Yes (hpC7_D) | 2.26E+11 | 100 ul |

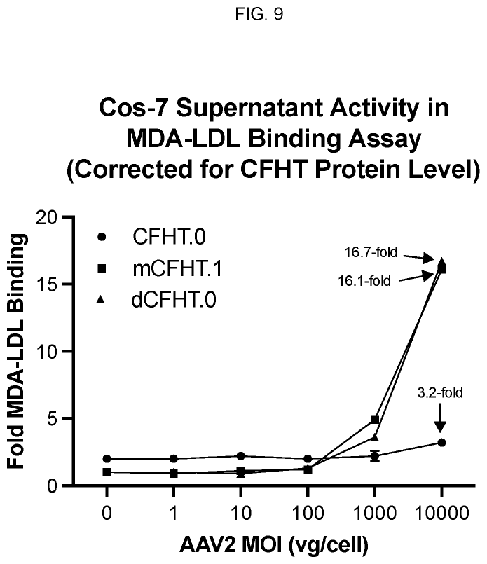

Supernatants were assayed in an MDA-LDL assay using the basic protocol described in Section 10.3.1. When supernatants for each MOI were added to MDA-LDL coated plates, a robust signal was detected for both mCFHT.1 and dCFHT.0 at the two highest MOIs tested (FIG. 9). Binding of mCFHT.1 and dCFHT.0 proteins was approximately 5-fold better than unmodified CFHT protein (CFHT.0).

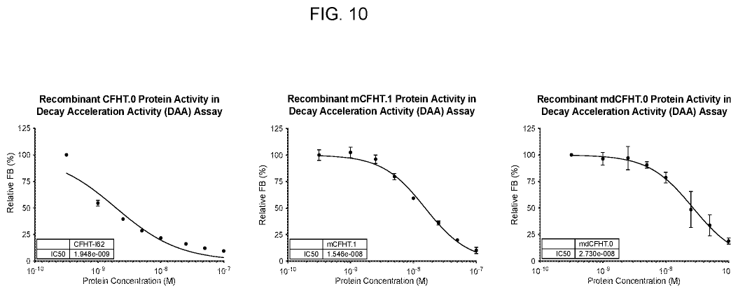

To confirm that mCFHT proteins have retained critical alternative complement pathway activity, we tested recombinant proteins in a decay acceleration activity (DAA) assay (described in Exemplary Assays below) that monitors the formation of the C3 convertase, C3bBb. Wells treated with increasing amounts of recombinant CFHT.0, mCFHT.1 and dCFHT.0, exhibited a potent reduction in the amount of C3 convertase in a dose-dependent manner. The unmodified CFHT recombinant protein exhibited an IC₅₀ of 1.95 nM while mCFHT.1 is 8-fold less active (IC₅₀=15.5 nM) and mdCFHT.1 is ^{˜}14-fold less active (IC₅₀=15.5 nM) when tested under these conditions (FIG. 10).

In summary, AAV2-produced optimized CFHT proteins mCFHT.1 and dCFHT.0 from Cos-7 supernatant and purified recombinant proteins potently bind to MDA ligand, inhibit LPS-driven AP activity and influence decay acceleration of C3 convertase (C3bBb).

### Exemplary Assays—Section 10.5

### CFHT ELISA

Cell culture media samples were diluted 1:100 with ELISA assay reagent diluent buffer (RDB, 1×PBS+1% BSA) for CFHT protein quantitation. Black MaxiSorp plates were coated with capture antibody anti-CFHT (aCTM113) at 1:600 in Maxisorp coating buffer (50 mM carbonate, pH 9.6) and placed overnight at 4° C. After washing with PBST (PBS with 0.05% Tween-20), plates were blocked with RDB for 90 minutes at room temperature. Plates were then washed again and 100 μl of diluted cell culture supernatant samples were added to each well and incubated for 90 minutes at room temperature. Plates were washed as above followed by CFHT detection with biotin conjugated aCTM87b (OX24 antibody, Thermo Fisher Scientific Cat. #MA5-17735) at 1:800, washing and incubation for 20 minutes with StreptAvidin-HRP (ThermoFisher Scientific, Cat. #21130) at 1:10,000 dilution followed by incubation with SuperSignal ELISA Pico Chemiluminescent Substrate (ThermoFisher Scientific, Cat. #37069) before detection using the BioTek Synergy 4 plate reader. A CFHT (in-house developed) protein standard curve was generated to determine concentration for each sample. CFHT protein concentration in cell culture supernatant was plotted in Prism 9 software with average and standard deviation plotted for duplicate transductions.

### MDA-LDL Ligand Binding Assay

To measure binding of recombinant CFHT protein to malondialdehyde modified LDL (MDA-LDL, Cell Biolabs Cat. #STA-212), a black MaxiSorp 96-well microplates were coated with 100 μl of 2.5 μg/ml MDA-LDL in PBS overnight at 4′C. After 3 washes in PBST, plates were blocked with SuperBlock T20 (PBS) Blocking Buffer (ThermoFisher Scientific, Cat. #37516) for 90 minutes at room temperature. 30 μl of cell culture supernatant was combined with 70 μl of SuperBlock T20 Blocking Buffer and then added to each well and incubated for 90 minutes at room temperature. Plates were then incubated for 1 hour with biotin conjugated aCTM87b (OX24 antibody, Thermo Fisher Scientific Cat. #MA5-17735) at 1:800 to detect CFHT protein followed by washing in PBST. Plates were next incubated for 20 minutes with StreptAvidin-HRP (ThermoFisher Scientific, Cat. #21130) conjugated secondary antibody at 1:10,000 dilution. Finally, plates were incubated for 5 minutes with SuperSignal ELISA Pico Chemiluminescent Substrate (ThermoFisher Scientific, Cat. #37069) followed by detection using a BioTek Synergy 4 plate reader. MDA-LDL ligand binding for Cos-7 supernatant was plotted in Prism 9 software with average and standard deviation plotted for duplicate transductions.

### LPS-driven Alternative Pathway (AP) Activation Assay

Alternative pathway (AP) activation was determined using an established direct ELISA (Harder M J et al. (2015) J. Immunol. 196:866-876) with minor modifications. In brief, 50 μl of LPS solution (50 μg/ml, from Salmonella enterica serotype typhimurium, Sigma-Aldrich Cat. #L7261) was coated on black 96-well Maxisorp plate in PBS overnight at 4° C. After washing with PBST, plates were blocked with RDB for 90 minutes at room temperature. To evaluate the impact of CFHT protein on AP activation, 30 μl of cell culture supernatant sample was mixed with 30 μl of 25% pooled normal human serum (Complement Tech. Cat. #NHS) containing 10 mM MgEGTA. As controls, 30 μl of recombinant rCFHT (I62-Y402), mCFHT.1 and dCFHT.0 proteins in PBS were serially diluted (100-0.14 nM final concentration) and mixed with 30 μl of 25% NHS containing 10 mM MgEGTA. The 60 μl mixture of supernatant or recombinant CFHT and serum was then added to LPS-coated wells and incubated for 90 minutes at 37′C prior to washing and subsequent exposure to HRP conjugated goat anti-human C3 at 1:10000 (MP Biomedicals, Cat. #855237) for 1 hour at room temperature. After 3 washes with PBST, C3b deposition in each well was indirectly detected using SuperSignal ELISA Pico Chemiluminescent Substrate and the BioTek Synergy 4 plate reader. PBS and EDTA (5 mM final concentration) were used as positive and negative controls, respectively. All dose responses were normalized to activity achieved when only PBS was added to the well. The percent change in C3b deposition relative to PBS was plotted in Prism 9 software with average and standard deviation plotted for duplicate transductions.

### Decoy Acceleration Activity (DAA) Assay

The DAA in fluid phase was performed by an ELISA-based method (Michelfelder S et al. 2015, J Am Soc Nephrol 28: 1462-1474). Briefly, 250 ng C3b in PBS was immobilized on Maxisorp plates overnight at 4° C. In order to generate the C3 convertases (C3bBb), 400 ng Factor B (CompTech Cat. #A135) and 25 ng Factor D (CompTech Cat #. A136) were incubated with immobilized C3b in phosphate buffer containing 2 mM nickel chloride, 25 mM sodium chloride and 4% BSA for 2 h at 37° C. After washing three times in PBST (0.05% Tween-20 in PBS), increasing concentrations (0.137 nM-100 nM) of recombinant CFH_I62 (SCTM produced) or mCFHT protein (Yurogen) in buffer containing 25 mM sodium chloride and 4% BSA were added to the preformed C3 convertase complex and incubated for 40 min at 37′C. The Bb fragments that remain bound to C3b were detected by an anti-factor B polyclonal antibody (CompTech Cat. #A235) at 1:10,000 dilution in reagent diluent buffer (RDB; 1% BSA in PBS). Finally, the plates were incubated with HRP-conjugated mouse anti-goat (Jackson Immuno research Cat. #205-035-108) at 1:10,000 dilution in RDB followed by SuperSignal ELISA Pico Chemiluminescent Substrate (ThermoFisher Scientific, Cat. #37069) to indirectly detect protein. The preformed C3 convertase without regulators was included as a positive control and the C3 proconvertase (C3bB) formation (FB without adding FD) was included as a negative control. All protein variant dose responses were normalized to activity achieved when only buffer was added to the well and results were plotted in Prism 9 with average and standard deviation plotted for duplicate wells.

It is understood that the examples and embodiments described herein are for illustrative purposes only and that various modifications or changes in light thereof will be suggested to persons skilled in the art and are to be included within the spirit and purview of this application and scope of the appended claims.

All publications, patents, patent applications, and accession numbers cited herein are hereby incorporated by reference with respect to the material for which they are expressly cited.

### 11. Reference Sequences and Sequence Listing

TABLE 19

| Gene | | | | Genbank | | |
|---|---|---|---|---|---|---|
| ID | Symbol | Gene name | Ensembl Gene ID | Accession # | UniProt ID | RefSeq Protein |
| 348 | APOE | apolipoprotein E | ENSG00000130203 | NM_000041 | P02649 | NP_000032.1 |
| 718 | C3 | complement C3 | ENSG00000125730 | NM_000064 | P01024 | NP_000055.2 |
| 720 | C4A | complement C4A (Rodgers | ENSG00000244731 | NM_007293 | P0C0L4 | NP_001002029.3 |
| | | bld grp) | | | | |
| 721 | C4B | complement C4B (Chido | ENSG00000224389 | NM_001002029 | P0C0L5 | NP_001002029.3 |
| | | bld grp) | | | | |
| 727 | C5 | complement C5 | ENSG00000106804 | NM_001735 | P01031 | NP_001304092.1 |
| 729 | C6 | complement C6 | ENSG00000039537 | NM_000065 | P13671 | NP_000056.2 |
| 730 | C7 | complement C7 | ENSG00000112936 | NM_000587 | P10643 | NP_000578.2 |
| 731 | C8A | complement C8 alpha chain | ENSG00000157131 | NM_000562 | P07357 | NP_000553.1 |
| 732 | C8B | complement C8 beta chain | ENSG00000021852 | NM_000066 | P07358 | NP_000057.2 |
| 733 | C8G | complement C8 gamma | ENSG00000176919 | NM_000606 | P07360 | NP_000597.2 |
| | | chain | | | | |
| 735 | C9 | complement C9 | ENSG00000113600 | NM_001737 | P02748 | NP_001728.1 |
| 629 | CFB | complement factor B | ENSG00000243649 | NM_001710 | P00751 | NP_001701.2 |
| 1675 | CFD | complement factor D | ENSG00000197766 | NM_001928 | P00746 | NP_001304264.1 |
| 3078 | CFHR1 | complement factor H related 1 | ENSG00000244414 | NM_002113 | Q03591 | NP_002104.2 |
| 3080 | CFHR2 | complement factor H related 2 | ENSG00000080910 | NM_005666 | P36980 | NP_001299601.1 |
| 10878 | CFHR3 | complement factor H related 3 | ENSG00000116785 | NM_021023 | Q02985 | NP_001160096.1 |
| 10877 | CFHR4 | complement factor H related 4 | ENSG00000134365 | NM_006684 | Q92496 | NP_001188479.1 |
| 81494 | CFHR5 | complement factor H related 5 | ENSG00000134389 | NM_030787 | Q9BXR6 | NP_110414.1 |
| 3426 | CFI | complement factor I | ENSG00000205403 | NM_000204 | P05156 | NP_000195.2 |
| 5199 | CFP | complement factor properdin | ENSG00000126759 | NM_002621 | P27918 | NP_001138724.1 |
| 5654 | HTRA1 | HtrA serine peptidase 1 | ENSG00000166033 | NM_002775 | Q92743 | NP_002766.1 |
| 7422 | VEGFA | Vascular endothelial growth | ENSG00000112715 | NM_003376 | P15692 | NP_001020537.2 |
| | | factor A | | | | |
| 7424 | VEGFC | vascular endothelial growth | ENSG00000150630 | NM_005429 | P49767 | NP_005420.1 |
| | | factor C | | | | |
| 2277 | VEGFD | vascular endothelial growth | ENSG00000165197 | NM_004469 | O43915 | NP_004460.1 |
| | | factor D | | | | |

TABLE 20

| SEQ | | |
|---|---|---|
| ID NO | | Description |
| 1 | SCR | FHR-1 dimerization domain (SCR1-2) amino |
| | | acid sequence |
| 2 | SCR7 | GeneArt - Codon - optimized SCR7 |
| 3 | SCR7.1 | DT - Codon - optimized SCR7 |
| 4 | SCR7.2 | Genescript - Codon optimized SCR7 |
| 5 | SCR7.3 | NovoPro - Codon optimized SCR7 |
| 6 | Aflibercept | Aflibercept 1353-bp with TTR ( transthyretin) |
| | | Signal Peptide |
| 7 | Aflibercept | Aflibercept 1359-bp with IGF1 Signal Peptide |
| 8 | Aflibercept | Aflibercept 1359-bp with IgK Signal Peptide |
| 9 | CFHT | GeneArt - Codon optimized CFHT cDNA |
| 10 | Promoter | smCBA - 881 |
| 11 | Promoter | smCBA - 886 |
| 12 | Promoter | CBA/CAG |
| 13 | Promoter | VMD2 + SV40 splice d/a |
| 14 | Promoter | RPE65 |
| 15 | Promoter | U6 |
| 16 | Promoter | SFFV |
| 17 | CFHT | CFHT cDNA 1350-BP |
| 18 | Promoter | sctmCBA Enhancer Promoter - 719 bp |
| 19 | Sig peptide | IGF-1 signal peptide |
| 20 | Sig peptide | lgK signal peptide |
| 21 | Sig peptide | TTR signal peptide |
| 22 | IRES | IRES |
| 23 | IRES | mIRES (minimal) |
| 24 | IRES + Spacers | 18-bp spacer - IRES - 45-bp spacer |
| 25 | WPRE | WPRE 589-bp |
| 26 | WPRE | WPRE3 247-bp |
| 27 | Poly A | SV40 Poly A UTRs - 122-bp |
| 28 | Poly A | HSV TK Poly A |
| 29 | Poly A | bGH pA |
| 30 | SCPS | Encodes SCPS T2A peptide 54-bp Adds T′ to |
| | | protein in position 1 and P to protein in |
| | | position 2 |
| 31 | SCPS | EGRGSLLTCGDVEENPG |
| 32 | SCPS | Encodes SCPS E2A 60-bp Add U′ to protein in |
| | | position 1 and P to protein in position 2 |
| 33 | SCPS | QCTNYALLKLAGDVESNPG |
| 34 | SCPS | Encodes SCPS P2A peptide (57-bp) |
| 35 | SCPS | ATNFSLLKQAGDVEENPG |
| 36 | SCPS | Encodes F2A peptide (66-bp) |
| 37 | SCPS | VKQTLNFDLLKLAGDVESNPG |
| 38 | SCPS | Encodes SCPS Furin 2A Cleavage Peptide |
| | | (38-bp) |
| 39 | SCPS | GGRGRR (Furin cleavage site) |
| 40 | SCPS | GGRGRRGG (Furin cleavage site) |
| 41 | Spacer | 12b spacer in pCTM420 |
| 41 | Spacer | 12b spacer in pCTM421 |
| 42 | Spacer | 12b spacer in pCTM422 |
| 43 | Spacer | 24b spacer in pCTM423, pCTM424, pCTM425 |
| 47 | IRES | mIRES and flanking |
| 48 | ITR | TR_AAV2_R_short 128-bp |
| 49 | LTR | Lentiviral 5′ LTR (Truncated) 181-bp |
| 50 | LTR | SEQ ID NO: 50 Lentiviral 3′ LTR (Delta-U3) |
| | | 234-bp |
| 51 | Promoter | VMD2 |
| 52 | SCR1 | FHR-1 SCR1 - NonCodon optimized |
| 53 | SCR1 | FHR-1 SCR1 - GeneArt codon-optimized |
| 54 | SCR2 | FHR-1 SCR2 - Non-codon-optimized |
| 55 | SCR2 | FHR-1 SCR2 - GeneArt codon-optimized |
| 56 | Linker | Linker amino acid sequence |
| 57 | Linker | Linker amino acid sequence |
| 185 | Promoter | TYR Promoter |
| 186 | Promoter | TYRP1 Promoter |
| 187 | Promoter | FLT1 Promoter |
| 188 | Promoter | VWF Promoter |
| 189 | Promoter | MGP Promoter |
| 190 | Promoter | RGS5 Promoter |
| 191 | Promoter | SFFV Promoter |
| 218 | Cargo | CFHT - 18-bp spacer - IRES (551-bp) - 45-bp |
| | | spacer - Aflib |
| 219 | pCTM398 | Aflib-CFHT construct |
| 220 | pCTM418 | Aflib-CFHT construct |
| 221 | pCTM419 | Aflib-CFHT construct |
| 222 | pCTM428 | Aflib-CFHT construct |
| 223 | pCTM429 | CFHT-Aflib construct |
| 226 | pCTM417 | Aflib-CFHT construct |
| 227 | pCTM369 | CFHT-CFI contruct |
| 228 | SCR | FHR-2 dimerization domain (SCR1-2) amino |
| | | acid sequence) |
| 229 | SCR | FHR-2 SCR1 - Non-codon Optimized |
| 230 | SCR | FHR-2 SCR1 - GeneArt Codon Optimized |
| 231 | SCR | FHR-2 SCR2 - Non-codon Optimized |
| 232 | SCR | FHR-2 SCR2 - GeneArt Codon Optimized |
| 233 | SCR | FHR-5 dimerization domain (SCR1-2) |
| 234 | SCR | FHR-5 SCR1 - Non-codon Optimized |
| 235 | SCR | FHR-5 SCR1 - GeneArt Codon Optimized |
| 236 | SCR | FHR-5 SCR2 - Non-codon Optimized |
| 237 | SCR | FHR-5 SCR2 - GeneArt Codon Optimized |
| 238 | Promoter | BEST1-EP-454 Enhancer Promoter |
| 239 | Promoter | BEST1-V3 Promoter - 144 b |
| 240 | HTRA1 | HTRA1 protein |
| 241 | shRNA | Control |
| 242 | SCR7 | sCR7 protein sequence |
| 243 | shRNA | C7 targeting |
| 244 | shRNA | C7 taqrgeting |
| 245 | shRNA | C7 targeting |
| 246 | mCFHT.1 | cDNA |
| 247 | mCFHT.1 | Protein |
| 248 | mCFHT.2 | cDNA |
| 249 | mCFHT.2 | Protein |
| 250 | dCFHT.0 | CDNA |
| 251 | dCFHT.0 | Protein |
| 252 | SCR7 | Wild-type cDNA |
| 253 | CFHT | protective CFHT protein sequence lacking the |
| | | signal polypeptide |

### 12. References

### Complement & AMD

Toomey et al. Complement Factor H in AMD: Bridging Genetic Associations and Pathobiology. Prog Retin Eye Res. 2018. 62: 38-57. doi:10.1016/j.preteyeres.2017.09.001.
Clark et al. Bruch's Membrane Compartmentalizes Complement Regulation in the Eye With Implications for Therapeutic Design in Age-related Macular Degeneration. Front. Immunol. 8:1778. doi: 10.3389/fimmu.2017.01778
Clark & Bishop. Role of Factor H and Related Proteins in Regulating Complement Activation in the Macula, and Relevance to Age-Related Macular Degeneration. J. Clin. Med. 2015, 4, 18-31; doi:10.3390/jcm4010018.
Bora et al. CD59, a Complement Regulatory Protein, Controls Choroidal Neovascularization in a Mouse Model of Wet-Type Age-Related Macular Degeneration. J Immunol 2007; 178:1783-1790. doi: 10.4049/jimmunol.178.3.1783.
Borras et al. Mechanisms of FH Protection Against Neovascular AMD. Front. Immunol. 11:443.doi: 10.3389/fimmu.2020.00443.
Lyzogubov et al. Role of Ocular Complement Factor H in a Murine Model of Choroidal Neovascularization. American Journal of Pathology, Vol. 177, No. 4, October 2010.
Tzoumas et al. Revisiting the role of factor H in age-related macular degeneration: insights from complement-mediated renal disease and rare genetic variants. Survey of Ophthalmology (2020), doi: https://doi.org/10.1016/j.survophthal.2020.10.008.

### Complement & Ocular Disease

Park et al. The Challenges and Promise of Complement Therapeutics for Ocular Diseases. 2019. Front. Immunol. 10:1007. doi: 10.3389/fimmu.2019.01007.
Mullins et al. From compliment to insult: genetics of the complement system in physiology and disease in the human retina. Human Molecular Genetics, 2017, Vol. 26, No. Ri R51-R57. doi: 10.1093/hmg/ddx181.
Mohlin et al. The link between morphology and complement in ocular disease. Molecular Immunology, 2017, 89 (2017) 84-99.
Rosa L. Schellevis, Role of the Complement System in Chronic Central Serous Chorioretinopathy: A Genome-Wide Association Study. JAMA Ophthalmol. 2018; 136(10):1128-1136. doi:10.1001/jamaophthalmol.2018.3190.
Jha et al. The Complement System and Ocular Diseases. Mol Immunol. 2007 September; 44(16): 3901-3908.
Perkins et al. Genetic and Protein Structural Evaluation of Atypical Hemolytic Uremic Syndrome and C3 Glomerulopathy. Adv Chronic Kidney Dis. 2020; 27(2):120-127.
Taylor et al. Loss-of-Function Mutations in the CFH Gene Affecting Alternatively Encoded Factor H-like 1 Protein Cause Dominant Early-Onset Macular Drusen. Ophthalmology 2019; 126:1410-1421.
Clark & Bishop. The eye as a complement dysregulation hotspot. Semin Immunopathol (2018) 40:65-74. DOI 10.1007/s00281-017-0649-6.
Mandava et al. Complement activation in the vitreous of patients with proliferative diabetic retinopathy. Invest Ophthalmol Vis Sci. 2020; 61(11):39. https://doi.org/10.1167/iovs.61.11.39.
Shahulhameed S et al. A Systematic Investigation on Complement Pathway Activation in Diabetic Retinopathy. Front. Immunol. 11:154. doi: 10.3389/fimmu.2020.00154.
de Jong et al. Implications of genetic variation in the complement system in age-related macular degeneration. PII: S1350-9462(21)00013-6. DOI: https://doi.org/10.1016/j.preteyeres. 2021.100952.
Bosco et al. Complement C3-Targeted Gene Therapy Restricts Onset and Progression of Neurodegeneration in Chronic Mouse Glaucoma. Molecular Therapy Vol. 26 No 10 Oct. 2018. Molecular Therapy Vol. 26 No 10 Oct. 2018.
Hubens et al. Increased ratios of complement factors C3a to C3 in aqueous humor and serum mark glaucoma progression. Experimental Eye Research (2021), doi: https://doi.org/10.1016/j.exer.2021.108460.
Kassa et al. Complement inhibition as a therapeutic strategy in retinal disorders. Expert Opin Biol Ther. 2019 April; 19(4):335-342. doi: 10.1080/14712598.2019.1575358. Epub 2019 Feb. 11.
Kaye. Central serous chorioretinopathy: An update on risk factors, pathophysiology and imaging modalities. Prog Retin Eye Res. 2020 November; 79:100865. doi: 10.1016/j.preteyeres.2020.100865. Epub 2020 May 11.
Kim et al. Prolonged Intraocular Residence of a Fourth Generation Compstatin Complement C3 Inhibitor Supports Its Clinical Development for Geographic Atrophy. ARVO?
Read et al. Clinical mini-review: systemic lupus erythematosus and the eye. Ocul Immunol Inflamm. 2004 June; 12(2):87-99. doi: 10.1080/09273940490895308.
Ren et al. A role for complement in glaucoma? Adv Exp Med Biol. 2010; 703:95-104. doi: 10.1007/978-1-4419-5635-4.
Silverman & Wong. Adaptive and Maladaptive Complement Activation in the Retina. Adv Exp Med Biol. 2019; 1185:33-37. doi: 10.1007/978-3-030-27378-1_6.
Sohn et al. Complement, innate immunity and ocular disease. Chem Immunol Allergy. 2007; 92:105-114. doi: 10.1159/000099261.
Xu & Chen. Diabetic retinopathy and dysregulated innate immunity.Vision Res. 2017 October; 139:39-46. doi: 10.1016/j.visres.2017.04.013. Epub 2017 Jun. 2.
Xu & Chen. Targeting the complement system for the management of retinal inflammatory and degenerative diseases. European Journal of Pharmacology 787:94-104, 2016.
Veszeli et al. A systematic analysis of the complement pathways in patients with neuromyelitis optica indicates alteration but no activation during remission. Molecular Immunology 57 (2014) 200-209.

### Complement & Diseases

Zelek et al. Compendium of current complement therapeutics. Molecular Immunology 114 (2019) 341-352.
Harris et al. Developments in anti-complement therapy; from disease to clinical trial. Molecular Immunology 102 (2018) 89-119. https://doi.org/10.1016/j.molimm.2018.06.008.
Ricklin et al. The renaissance of complement therapeutics. Nat Rev Nephrol. 2018 14 (1):
26-47. doi:10.108/nrneph.2017.156.
Morgan & Harris. Complement, a target for therapy in inflammatory and degenerative diseases. Nature Reviews 2015 14: 857-877.
Ricklin et al. Complement in disease: a defense system turning offensive. Nat Rev Nephrol. 2016; 12(7): 383-401. doi:10.1038/nrneph.2016.70.
Ricklin & Lambris. Complement-targeted therapeutics. Nat Biotechnol. 2007 November; 25(11): 1265-1275. doi:10.1038/nbt1342.
Thurman & Holers. The Central Role of the Alternative Complement Pathway in Human Disease. J Immunol 2006; 176:1305-1310; doi: 10.4049/jimmunol.176.3.1305.
Barlow. Complement and disease: better no factor H than bad factor H. Transl Cancer Res 2017; 6(Suppl 3):5606-S609
Immune Deficiency Foundation. Complement Deficiencies. https://primaryimmune.org/about-primary-immunodeficiencies/spec . . .
Dobo et al. Be on Target: Strategies of Targeting Alternative and Lectin Pathway Components in Complement-Mediated Diseases. Front. Immunol. 9:1851. doi: 10.3389/fimmu.2018.01851.
Gavriilaki & Brodsky. Complementopathies and precision medicine. J Clin Invest. 2020; 130(5):2152-2163. https://doi.org/10.1172/JCI136094.
Zipfel et al. Complement and diseases: Defective alternative pathway control results in kidney and eye diseases. Molecular Immunology 43 (2006) 97-106.
Schmidt et al. Protection of host cells by complement regulators. Immunol rev 2016 274(1): 152-171.
Huber-Lang et al. Complement therapeutic strategies in trauma, hemorrhagic shock and systemic inflammation—closing Pandora's box? Seminars Immunology 28 (2016): 278-284. http://dx.doi.org/10.1016/j.smim.2016.04.005.

### CFH & CFHT (Misc)

Makou et al. Functional Anatomy of Complement Factor H. Biochemistry 2013, 52, 3949-3962. dx.doi.org/10.1021/bi4003452.
Choudhury et al. FHL-1 interacts with human RPE cells through the 1 a5b1 integrin and confers protection against oxidative stress. bioRxiv preprint doi: https://doi.org/10.1101/2020.09.28.317263.
Dopier et al. Self versus Nonself Discrimination by the Soluble Complement Regulators Factor H and FHL-1. J Immunol 2019; 202:2082-2094; Prepublished online 11. doi: 10.4049/jimmunol.1801545.
Mannes et al. Tuning the Functionality by Splicing: Factor H and Its Alternative Splice Variant FHL-1 Share a Gene but Not All Functions. Front. Immunol. 11:596415. doi: 10.3389/fimmu.2020.596415.
Swinkles et al. C-reactive protein and pentraxin-3 binding of factor H-like protein 1 differs from complement factor H: implications for retinal inflammation. Scientific Reports (2018) 8:1643. DOI:10.1038/s41598-017-18395-7 1.
Haque et al. Characterization of Binding Properties of Individual Functional Sites of Human Complement Factor H. Front. Immunol. 11:1728. doi: 10.3389/fimmu.2020.01728.

### CFHRs (Misc)

Cipriani et al. Increased circulating levels of Factor H-Related Protein 4 are strongly associated with age-related macular degeneration. Nature Communications (2020) 11:778|https://doi.org/10.1038/s41467-020-14499-3.
Hakobyan et al. Identification of Factor H-like Protein 1 as the Predominant Complement Regulator in Bruch's Membrane: Implications for Age-related Macular Degeneration. J Immunol 2014; 193:4962-4970; Prepublished online 10. doi: 10.4049/jimmunol.1401613
Cserhalmi et al. Regulation of regulators: Role of the complement factor H-related proteins. Seminars in Immunology 45 (2019) 101341.

MULTIGENE CONSTRUCTS FOR TREATMENT OF AGE-RELATED MACULAR DEGENERATION
AND OTHER COMPLEMENT DYSREGULATION-RELATED CONDITIONS
SEQUENCES

SEQ ID NO: 1 FHR-1 dimerization domain (SCR1-2)
EATFCDFPKINHGILYDEEKYKPFSQVPTGEVFYYSCEYNFVSPSKSEWTRITCTEEGWSPTPK
CLRLCFFPFVENGHSESSGQTHLEGDTVQIICNTGYRLQNNENNISCVERGWSTPPKCRS
SEQ ID NO: 2 GeneArt Codon Optimized CFHT SCR7.1 cDNA
AGGAAGTGCTACTTCCCCTACCTGGAAAACGGCTACAACCAGAACTACGGCCGGAAGTTCGTGC
AGGGCAAGAGCATCGATGTGGCCTGCCACCCTGGATACGCCCTGCCTAAGGCCCAGACCACCGT
GACCTGCATGGAAAATGGATGGTCCCCCACCCCCCGGTGCATCAGAGTG
SEQ ID NO: 3 IDT Codon Optimized CFHT SCR7.1 cDNA
AGGAAGTGCTATTTCCCTTATTTGGAAAACGGATATAATCAAAATTACGGTCGGAAATTTGTCC
AAGGTAAGTCAATAGATGTCGCGTGTCATCCTGGCTATGCTCTTCCGAAAGCACAGACAACGGT
TACGTGTATGGAGAACGGTTGGTCTCCAACCCCTCGATGTATTAGGGTG
SEQ ID NO: 4 GeneScript Codon Optimized CFHT SCR7.2 cDNA
AGGAAGTGCTACTTCCCCTACCTGGAAAACGGCTACAACCAGAACTACGGCCGGAAGTTCGTGC
AGGGCAAGAGCATCGATGTGGCCTGCCACCCTGGATACGCCCTGCCTAAGGCCCAGACCACCGT
GACCTGCATGGAAAATGGATGGTCCCCCACCCCCCGGTGCATCAGAGTG
SEQ ID NO: 5 NovoPro Codon Optimized CFHT SCR7.3 cDNA
AGGAAATGCTATTITCCCTATTTGGAAAACGGCTACAACCAGAATTATGGAAGGAAGTTCGTCC
AGGGAAAATCAATCGACGTGGCATGCCACCCTGGGTACGCCTTGCCAAAAGCCCAGACAACCGT
GACATGCATGGAGAACGGATGGTCTCCAACACCCAGATGTATCCGGGTC
SEQ ID NO: 6 Aflibercept-TTR signal peptide
ATGGCCTCTCATAGACTGCTGCTGCTGTGTCTGGCCGGCCTGGTGTTTGTGTCTGAGGCCTCTG
ATACCGGCAGACCCTTCGTGGAAATGTACAGCGAGATCCCCGAGATCATCCACATGACCGAGGG
CAGAGAGCTGGTCATCCCCTGCAGAGTGACAAGCCCCAACATCACCGTGACTCTGAAGAAGTTC
CCTCTGGACACACTGATCCCCGACGGCAAGAGAATCATCTGGGACAGCCGGAAGGGCTTCATCA
TCAGCAACGCCACCTACAAAGAGATCGGCCTGCTGACCTGTGAAGCCACCGTGAATGGCCACCT
GTACAAGACCAACTACCTGACACACAGACAGACCAACACCATCATCGACGTGGTGCTGAGCCCT
AGCCACGGCATTGAACTGTCTGTGGGCGAGAAGCTGGTGCTGAACTGTACCGCCAGAACCGAGC
TGAACGTGGGCATCGACTTCAACTGGGAGTACCCCAGCAGCAAGCACCAGCACAAGAAACTGGT
CAACCGGGACCTGAAAACCCAGAGCGGCAGCGAGATGAAGAAATTCCTGAGCACCCTGACCATC
GACGGCGTGACCAGATCTGACCAGGGCCTGTACACATGTGCCGCCAGCTCTGGCCTGATGACCA
AGAAAAACAGCACCTTCGTGCGGGTGCACGAGAAGGACAAGACCCACACCTGTCCTCCATGTCC
TGCTCCAGAACTGCTCGGCGGACCTTCCGTGTTCCTGTTTCCTCCAAAGCCTAAGGACACCCTG
ATGATCAGCAGAACCCCTGAAGTGACCTGCGTGGTGGTGGATGTGTCCCACGAGGATCCCGAAG
TGAAGTTCAATTGGTACGTGGACGGCGTGGAAGTGCACAACGCCAAGACCAAGCCTAGAGAGGA
ACAGTACAATAGCACCTACAGAGTGGTGTCCGTGCTGACCGTGCTGCACCAGGATTGGCTGAAC
GGCAAAGAGTACAAGTGCAAGGTGTCCAACAAGGCCCTGCCTGCTCCTATCGAGAAAACCATCT
CCAAGGCCAAGGGCCAGCCTAGGGAACCCCAGGTTTACACACTGCCTCCAAGCAGGGACGAGCT
GACAAAGAACCAGGTGTCCCTGACCTGCCTGGTCAAGGGCTTCTACCCTTCCGATATCGCCGTG
GAATGGGAGAGCAATGGCCAGCCTGAGAACAACTACAAGACAACCCCTCCTGTGCTGGACAGCG
ACGGCTCATTCTTCCTGTACAGCAAGCTGACAGTGGACAAGAGCAGATGGCAGCAGGGCAACGT
GTTCAGCTGCAGCGTGATGCACGAGGCCCTGCACAACCACTACACCCAGAAGTCCCTGAGCCTG
TCTCCTGGC
SEQ ID NO: 7 Aflibercept 1359-bp with IGF1 Signal Peptide
ATGGGCAAGATTTCTAGCCTGCCTACACAGCTGTTCAAGTGCTGCTTCTGCGACTTCCTGAAGT
CCGATACCGGCAGACCCTTCGTGGAAATGTACAGCGAGATCCCCGAGATCATCCACATGACCGA
GGGCAGAGAGCTGGTCATCCCCTGCAGAGTGACAAGCCCCAACATCACCGTGACTCTGAAGAAG
TTCCCTCTGGACACACTGATCCCCGACGGCAAGAGAATCATCTGGGACAGCCGGAAGGGCTTCA
TCATCAGCAACGCCACCTACAAAGAGATCGGCCTGCTGACCTGTGAAGCCACCGTGAATGGCCA
CCTGTACAAGACCAACTACCTGACACACAGACAGACCAACACCATCATCGACGTGGTGCTGAGC
CCTAGCCACGGCATTGAACTGTCTGTGGGCGAGAAGCTGGTGCTGAACTGTACCGCCAGAACCG
AGCTGAACGTGGGCATCGACTTCAACTGGGAGTACCCCAGCAGCAAGCACCAGCACAAGAAACT
GGTCAACCGGGACCTGAAAACCCAGAGCGGCAGCGAGATGAAGAAATTCCTGAGCACCCTGACC
ATCGACGGCGTGACCAGATCTGACCAGGGCCTGTACACATGTGCCGCCAGCTCTGGCCTGATGA
CCAAGAAAAACAGCACCTTCGTGCGGGTGCACGAGAAGGACAAGACCCACACCTGTCCTCCATG
TCCTGCTCCAGAACTGCTCGGCGGACCTTCCGTGTTCCTGTTTCCTCCAAAGCCTAAGGACACC
CTGATGATCAGCAGAACCCCTGAAGTGACCTGCGTGGTGGTGGATGTGTCCCACGAGGATCCCG
AAGTGAAGTTCAATTGGTACGTGGACGGCGTGGAAGTGCACAACGCCAAGACCAAGCCTAGAGA
GGAACAGTACAATAGCACCTACAGAGTGGTGTCCGTGCTGACCGTGCTGCACCAGGATTGGCTG
AACGGCAAAGAGTACAAGTGCAAGGTGTCCAACAAGGCCCTGCCTGCTCCTATCGAGAAAACCA
TCTCCAAGGCCAAGGGCCAGCCTAGGGAACCCCAGGTTTACACACTGCCTCCAAGCAGGGACGA
GCTGACAAAGAACCAGGTGTCCCTGACCTGCCTGGTCAAGGGCTTCTACCCTTCCGATATCGCC
GTGGAATGGGAGAGCAATGGCCAGCCTGAGAACAACTACAAGACAACCCCTCCTGTGCTGGACA
GCGACGGCTCATTCTTCCTGTACAGCAAGCTGACAGTGGACAAGAGCAGATGGCAGCAGGGCAA
CGTGTTCAGCTGCAGCGTGATGCACGAGGCCCTGCACAACCACTACACCCAGAAGTCCCTGAGC
CTGTCTCCTGGCTAA
SEQ ID NO: 8 Aflibercept 1359-bp with IgK Signal Peptide
ATGGAAACAGATACACTGCTGCTGTGGGTGCTGCTCTTGTGGGTGCCAGGATCTACA
GGCGATTCTGATACCGGCAGACCCTTCGTGGAAATGTACAGCGAGATCCCCGAGATCATCCACA
TGACCGAGGGCAGAGAGCTGGTCATCCCCTGCAGAGTGACAAGCCCCAACATCACCGTGACTCT
GAAGAAGTTCCCTCTGGACACACTGATCCCCGACGGCAAGAGAATCATCTGGGACAGCCGGAAG
GGCTTCATCATCAGCAACGCCACCTACAAAGAGATCGGCCTGCTGACCTGTGAAGCCACCGTGA
ATGGCCACCTGTACAAGACCAACTACCTGACACACAGACAGACCAACACCATCATCGACGTGGT
GCTGAGCCCTAGCCACGGCATTGAACTGTCTGTGGGCGAGAAGCTGGTGCTGAACTGTACCGCC
AGAACCGAGCTGAACGTGGGCATCGACTTCAACTGGGAGTACCCCAGCAGCAAGCACCAGCACA
AGAAACTGGTCAACCGGGACCTGAAAACCCAGAGCGGCAGCGAGATGAAGAAATTCCTGAGCAC
CCTGACCATCGACGGCGTGACCAGATCTGACCAGGGCCTGTACACATGTGCCGCCAGCTCTGGC
CTGATGACCAAGAAAAACAGCACCTTCGTGCGGGTGCACGAGAAGGACAAGACCCACACCTGTC
CTCCATGTCCTGCTCCAGAACTGCTCGGCGGACCTTCCGTGTTCCTGTTTCCTCCAAAGCCTAA
GGACACCCTGATGATCAGCAGAACCCCTGAAGTGACCTGCGTGGTGGTGGATGTGTCCCACGAG
GATCCCGAAGTGAAGTTCAATTGGTACGTGGACGGCGTGGAAGTGCACAACGCCAAGACCAAGC
CTAGAGAGGAACAGTACAATAGCACCTACAGAGTGGTGTCCGTGCTGACCGTGCTGCACCAGGA
TTGGCTGAACGGCAAAGAGTACAAGTGCAAGGTGTCCAACAAGGCCCTGCCTGCTCCTATCGAG
AAAACCATCTCCAAGGCCAAGGGCCAGCCTAGGGAACCCCAGGTTTACACACTGCCTCCAAGCA
GGGACGAGCTGACAAAGAACCAGGTGTCCCTGACCTGCCTGGTCAAGGGCTTCTACCCTTCCGA
TATCGCCGTGGAATGGGAGAGCAATGGCCAGCCTGAGAACAACTACAAGACAACCCCTCCTGTG
CTGGACAGCGACGGCTCATTCTTCCTGTACAGCAAGCTGACAGTGGACAAGAGCAGATGGCAGC
AGGGCAACGTGTTCAGCTGCAGCGTGATGCACGAGGCCCTGCACAACCACTACACCCAGAAGTC
CCTGAGCCTGTCTCCTGGCTAA
SEQ ID NO: 9 CFHT cDNA
GeneArt Codon Optimized CFHT DNA (SCR7 Domain in Bold Underline)
ATGAGACTGCTGGCCAAGATCATCTGCCTGATGCTGTGGGCCATCTGCGTGGCCGAGGACTGCA
ACGAGCTGCCCCCCAGAAGAAACACCGAGATCCTGACCGGCTCTTGGAGCGACCAGACCTACCC
TGAGGGCACCCAGGCCATCTACAAGTGCAGACCCGGCTACCGGTCCCTGGGCAACATCATCATG
GTGTGCAGAAAGGGCGAGTGGGTGGCCCTGAACCCCCTGAGAAAGTGCCAGAAGAGGCCCTGCG
GACACCCCGGCGATACCCCTTTTGGCACCTTCACACTGACCGGCGGCAACGTGTTCGAGTACGG
CGTGAAGGCCGTGTACACCTGTAACGAGGGCTACCAGCTGCTGGGCGAGATCAACTACAGAGAG
TGCGACACCGACGGCTGGACCAACGATATCCCCATCTGCGAGGTCGTGAAGTGCCTGCCTGTGA
CCGCCCCAGAGAACGGCAAGATCGTGTCCAGCGCCATGGAACCCGACAGAGAGTACCACTTCGG
CCAGGCCGTCAGATTCGTGTGCAACAGCGGCTACAAGATCGAGGGCGACGAGGAAATGCACTGC
AGCGACGACGGCTTCTGGTCCAAAGAAAAGCCTAAGTGCGTGGAAATCAGCTGCAAGAGCCCCG
ACGTGATCAACGGCAGCCCCATCAGCCAGAAGATCATCTACAAAGAGAACGAGCGGTTCCAGTA
CAAGTGTAACATGGGCTACGAGTACAGCGAGCGGGGCGACGCCGTGTGTACAGAATCTGGATGG
CGGCCTCTGCCCAGCTGCGAGGAAAAGAGCTGCGACAACCCCTACATCCCCAACGGCGACTACA
GCCCCCTGCGGATCAAGCACAGAACCGGCGACGAGATCACCTACCAGTGCCGGAACGGCTTCTA
CCCCGCCACCAGAGGCAATACCGCCAAGTGTACCAGCACCGGCTGGATCCCTGCCCCCAGATGT
ACCCTGAAGCCCTGCGACTACCCTGACATCAAGCACGGCGGCCTGTACCACGAGAACATGCGGA
GGCCCTACTTCCCTGTGGCCGTGGGCAAGTACTACAGCTACTACTGCGACGAGCACTTCGAGAC
ACCCAGCGGCAGCTACTGGGACCACATCCACTGTACCCAGGACGGCTGGTCCCCTGCCGTGCCC
TGCCTGAGGAAGTGCTACTTCCCCTACCTGGAAAACGGCTACAACCAGAACTACGGCCGGAAGT
TCGTGCAGGGCAAGAGCATCGATGTGGCCTGCCACCCTGGATACGCCCTGCCTAAGGCCCAGAC
CACCGTGACCTGCATGGAAAATGGATGGTCCCCCACCCCCCGGTGCATCAGAGTGTCCTTCACC
CTGTGA
SEQ ID NO: 10 smCBA promoter 881-bp
CCGCGTTACATAACTTACGGTAAATGGCCCGCCTGGCTGACCGCCCAACGACCCCCGCCCATTG
ACGTCAATAATGACGTATGTTCCCATAGTAACGCCAATAGGGACTTTCCATTGACGTCAATGGG
TGGAGTATTTACGGTAAACTGCCCACTTGGCAGTACATCAAGTGTATCATATGCCAAGTACGCC
CCCTATTGACGTCAATGACGGTAAATGGCCCGCCTGGCATTATGCCCAGTACATGACCTTATGG
GACTTTCCTACTTGGCAGTACATCTACGTATTAGTCATCGCTATTACCATGGTCGAGGTGAGCC
CCACGTTCTGCTTCACTCTCCCCATCTCCCCCCCCTCCCCACCCCCAATTTTGTATTTATTTAT
TTTTTAATTATTTTGTGCAGCGATGGGGGGGGGGGGGGGGGGGGGGGGCGCGCCAGGCGGGGCG
GGGGGGGGCGAGGGGCGGGGCGGGGCGAGGCGGAGAGGTGCGGCGGCAGCCAATCAGAGCGGCG
CGCTCCGAAAGTTTCCTTTTATGGCGAGGCGGCGGCGGCGGCGGCCCTATAAAAAGCGAAGCGC
GCGGCGGGCGGGAGTCGCTGCGCGCTGCCTTCGCCCCGTGCCCCGCTCCGCCGCCGCCTCGCGC
CGCCCGCCCCGGCTCTGACTGACCGCGTTACTCCCACAGGTGAGCGGGGGGACGGCCCTTCTC
CTCCGGGCTGTAATTAGCGCTTGGTTTAATGACGGCTTGTTTCTTTTCTGTGGCTGCGTGAAAG
CCTTGAGGGGCTCCGGGAGCTAGAGCCTCTGCTAACCATGTTCATGCCTTCTTCTTTTTCCTAC
AGCTCCTGGGCAACGTGCTGGTTATTGTGCTGTCTCATCATTTTGGCA
SEQ ID NO: 11 smCBA promoter 886-bp
CGTTACATAACTTACGGTAAATGGCCCGCCTGGCTGACCGCCCAACGACCCCCGCCCATTGACG
TCAATAATGACGTATGTTCCCATAGTAACGCCAATAGGGACTTTCCATTGACGTCAATGGGTGG
ACTATTTACGGTAAACTGCCCACTTGGCAGTACATCAAGTGTATCATATGCCAAGTACGCCCCC
TATTGACGTCAATGACGGTAAATGGCCCGCCTGGCATTATGCCCAGTACATGACCTTATGGGAC
TTTCCTACTTGGCAGTACATCTACGTATTAGTCATCGCTATTACCATGGTCGAGGTGAGCCCCA
CGTTCTGCTTCACTCTCCCCATCTCCCCCCCCTCCCCACCCCCAATTTTGTATTTATTTATTTT
TTAATTATTTTGTGCAGCGATGGGGGGGGGGGGGGGGGGGGGGGGCGCGCCAGGCGGGGGGGG
CGGGGCGAGGGGGGGGGGGGGGCGAGGCGGAGAGGTGCGGCGGCAGCCAATCAGAGCGGCGCGC
TCCGAAAGTTTCCTTTTATGGCGAGGCGGCGGCGGCGGCGGCCCTATAAAAAGCGAAGCGCGCG
GCGGGCGGGAGTCGCTGCGACGCTGCCTTCGCCCCGTGCCCCGCTCCGCCGCCGCCTCGCGCCG
CCCGCCCCGGCTCTGACTGACCGCGTTACTCCCACAGGTGAGCGGGGGGACGGCCCTTCTCCT
CCGGGCTGTAATTAGCGCTTGGTTTAATGACGGCTTGTTTCTTTTCTGTGGCTGCGTGAAAGCC
TTGAGGGGCTCCGGGAGCTAGAGCCTCTGCTAACCATGTTCATGCCTTCTTCTTTTTCCTACAG
CTCCTGGGCAACGTGCTGGTTATTGTGCTGTCTCATCATTTTGGCAAAGAATTC
SEQ ID NO: 12 CBA/CAG promoter 1654-bp
CGTTACATAACTTACGGTAAATGGCCCGCCTGGCTGACCGCCCAACGACCCCCGCCCATTGACG
TCAATAATGACGTATGTTCCCATAGTAACGCCAATAGGGACTTTCCATTGACGTCAATGGGTGG
AGTATTTACGGTAAACTGCCCACTTGGCAGTACATCAAGTGTATCATATGCCAAGTACGCCCCC
TATTGACGTCAATGACGGTAAATGGCCCGCCTGGCATTATGCCCAGTACATGACCTTATGGGAC
TTTCCTACTTGGCAGTACATCTACGTATTAGTCATCGCTATTACCATGGTCGAGGTGAGCCCCA
CGTTCTGCTTCACTCTCCCCATCTCCCCCCCCTCCCCACCCCCAATTTTGTATTTATTTATTTT
TTAATTATTTTGTGCAGCGATGGGGGGGGGGGGGGGGGGGGGGGGCGCGCCAGGCGGGGCGGGG
CGGGGCGAGGGGGGGGGGGGGGCGAGGCGGAGAGGTGCGGCGGCAGCCAATCAGAGCGGCGCGC
TCCGAAAGTTTCCTTTTATGGCGAGGCGGCGGCGGCGGCGGCCCTATAAAAAGCGAAGCGCGCG
GCGGGCGGGAGTCGCTGCGCGCTGCCTTCGCCCCGTGCCCCGCTCCGCCGCCGCCTCGCGCCGC
CCGCCCCGGCTCTGACTGACCGCGTTACTCCCACAGGTGAGCGGGCGGGACGGCCCTTCTCCTC
CGGGCTGTAATTAGCGCTTGGTTTAATGACGGCTTGTTTCTTTTCTGTGGCTGCGTGAAAGCCT
TGAGGGGCTCCGGGAGGGCCCTTTGTGCGGGGGGAGCGGCTCGGGGGGTGCGTGCGTGTGTGTG
TGCGTGGGGAGCGCCGCGTGCGGCTCCGCGCTGCCCGGCGGCTGTGAGCGCTGCGGGCGCGGCG
CGGGGCTTTGTGCGCTCCGCAGTGTGCGCGAGGGGAGCGCGGCCGGGGGCGGTGCCCCGCGGTG
CGGGGGGGGCTGCGAGGGGAACAAAGGCTGCGTGCGGGGTGTGTGCGTGGGGGGGTGAGCAGGG
GGTGTGGGCGCGTCGGTCGGGCTGCAACCCCCCCTGCACCCCCCTCCCCGAGTTGCTGAGCACG
GCCCGGCTTCGGGTGCGGGGCTCCGTACGGGGCGTGGCGCGGGGCTCGCCGTGCCGGGGGGGGG
GTGGCGGCAGGTGGGGGTGCCGGGGGGGGCGGGGCCGCCTCGGGCCGGGGAGGGCTCGGGGGAG
GGGCGCGGCGGCCCCCGGAGCGCCGGCGGCTGTCGAGGCGCGGCGAGCCGCAGCCATTGCCTTT
TATGGTAATCGTGCGAGAGGGCGCAGGGACTTCCTTTGTCCCAAATCTGTGCGGAGCCGAAATC
TGGGAGGCGCCGCCGCACCCCCTCTAGCGGGCGCGGGGCGAAGCGGTGCGGCGCCGGCAGGAAG
GAAATGGGGGGGGAGGGCCTTCGTGCGTCGCCGCGCCGCCGTCCCCTTCTCCCTCTCCAGCCTC
GGGGCTGTCCGCGGGGGGACGGCTGCCTTCGGGGGGGACGGGGCAGGGCGGGGTTCGGCTTCTG
GCGTGTGACCGGCGGCTCTAGAGCCTCTGCTAACCATGTTCATGCCTTCTTCTTTTTCCTACAG
CTCCTGGGCAACGTGCTGGTTATTGTGCTGTCTCATCATTTTGGCAAAGAATTC
SEQ ID NO: 13 VMD2-796-bp (n. 1-624) + SV40 splice
donor/acceptor (n. 625-796)
CAATTCTGTCATTTTACTAGGGTGATGAAATTCCCAAGCAACACCATCCTTTTCAGATAAGGGC
ACTGAGGCTGAGAGAGGAGCTGAAACCTACCCGGCGTCACCACACACAGGTGGCAAGGCTGGGA
CCAGAAACCAGGACTGTTGACTGCAGCCCGGTATTCATTCTTTCCATAGCCCACAGGGCTGTCA
AAGACCCCAGGGCCTAGTCAGAGGCTCCTCCTTCCTGGAGAGTTCCTGGCACAGAAGTTGAAGC
TCAGCACAGCCCCCTAACCCCCAACTCTCTCTGCAAGGCCTCAGGGGTCAGAACACTGGTGGAG
CAGATCCTTTAGCCTCTGGATTTTAGGGCCATGGTAGAGGGGGTGTTGCCCTAAATTCCAGCCC
TGGTCTCAGCCCAACACCCTCCAAGAAGAAATTAGAGGGGCCATGGCCAGGCTGTGCTAGCCGT
TGCTTCTGAGCAGATTACAAGAAGGGACCAAGACAAGGACTCCTTTGTGGAGGTCCTGGCTTAG
GGAGTCAAGTGACGGCGGCTCAGCACTCACGTGGGCAGTGCCAGCCTCTAAGAGTGGGCAGGGG
CACTGGCCACAGAGTCCCAGGGAGTCCCACCAGCCTAGTCGCCAGACCGGGGATCCTCTAGAGG
ATCCGGTACTCGAGGAACTGAAAAACCAGAAAGTTAACTGGTAAGTTTAGTCTTTTTGTCTTTT
ATTTCAGGTCCCGGATCCGGTGGTGGTGCAAATCAAAGAACTGCTCCTCAGTGGATGTTGCCTT
TACTTCTAGGCCTGTACGGAAGTGTTAC
SEQ ID NO: 14 RPE65 promoter 750-bp
ATACTCTCAGAGTGCCAAACATATACCAATGGACAAGAAGGTGAGGCAGAGAGCAGACAGGCAT
TAGTGACAAGCAAAGATATGCAGAATTTCATTCTCAGCAAATCAAAAGTCCTCAACCTGGTTGG
AAGAATATTGGCACTGAATGGTATCAATAAGGTTGCTAGAGAGGGTTAGAGGTGCACAATGTGC
TTCCATAACATTTTATACTTCTCCAATCTTAGCACTAATCAAACATGGTTGAATACTTTGTTTA
CTATAACTCTTACAGAGTTATAAGATCTGTGAAGACAGGGACAGGGACAATACCCATCTCTGTC
TGGTTCATAGGTGGTATGTAATAGATATTTTTAAAAATAAGTGAGTTAATGAATGAGGGTGAGA
ATGAAGGCACAGAGGTATTAGGGGGAGGTGGGCCCCAGAGAATGGTGCCAAGGTCCAGTGGGGT
GACTGGGATCAGCTCAGGCCTGACGCTGGCCACTCCCACCTAGCTCCTTTCTTTCTAATCTGTT
CTCATTCTCCTTGGGAAGGATTGAGGTCTCTGGAAAACAGCCAAACAACTGTTATGGGAACAGC
AAGCCCAAATAAAGCCAAGCATCAGGGGGATCTGAGAGCTGAAAGCAACTTCTGTTCCCCCTCC
CTCAGCTGAAGGGGGGGGAAGGGCTCCCAAAG
SEQ ID NO: 15 U6 promoter 313-bp
TATCCGACGCCGCCATCTCTAGGCCCGCGCCGGCCCCCTCGCACAGACTTGTGGGAGAAGCTCG
GCTACTCCCCTGCCCCGGTTAATTTGCATATAATATTTCCTAGTAACTATAGAGGCTTAATGTG
CGATAAAAGACAGATAATCTGTTCTTTTTAATACTAGCTACATTTTACATGATAGGCTTGGATT
TCTATAAGAGATACAAATACTAAATTATTATTTTAAAAAACAGCACAAAAGGAAACTCACCCTA
ACTGTAAAGTAATTGTGTGTTTTGAGACTATAAATATCCCTTGGAGAAAAGCCTTGTT
SEQ ID NO: 16 SFFV promoter 443-bp
TGAAAGACCCCACCTGTAGGTTTGGCAAGaTAGCTGCAGTAACGCCATTTTGCAAGGCATGGAA
AAATACCAAACCAAGAATAGAGAAGTTCAGATCAAGGGCGGGTACATGAAAATAGCTAACGTTG
GGCCAAACAGGATATCTGCGGTGAGCAGTTTCGGCCCCGGCCCGGGGCCAAGAACAGATGGTCA
CCGCAGTTTCGGCCCCGGCCCGAGGCCAAGAACAGATGGTCCCCAGATATGGCCCAACCCTCAG
CAGTTTCTTAAGACCCATCAGATGTTTCCAGGCTCCCCCAAGGACCTGAAATGACCCTGCGCCT
TATTTGAATTAACCAATCAGCCTGCTTCTCGCTTCTGTTCGCGCGCTTCTGCTTCCCGAGCTCT
ATAAAAGAGCTCACAACCCCTCACTCGGCGCGCCAGTCCTCCGATTGACTGAGTCGCCC
SEQ ID NO: 17 CFHT 1350-bp
ATGAGACTGCTGGCCAAGATCATCTGCCTGATGCTGTGGGCCATCTGCGTGGCCGAGGACTGCA
ACGAGCTGCCCCCCAGAAGAAACACCGAGATCCTGACCGGCTCTTGGAGCGACCAGACCTACCC
TGAGGGCACCCAGGCCATCTACAAGTGCAGACCCGGCTACCGGTCCCTGGGCAACATCATCATG
GTGTGCAGAAAGGGCGAGTGGGTGGCCCTGAACCCCCTGAGAAAGTGCCAGAAGAGGCCCTGCG
GACACCCCGGCGATACCCCTTTTGGCACCTTCACACTGACCGGCGGCAACGTGTTCGAGTACGG
CGTGAAGGCCGTGTACACCTGTAACGAGGGCTACCAGCTGCTGGGCGAGATCAACTACAGAGAG
TGCGACACCGACGGCTGGACCAACGATATCCCCATCTGCGAGGTCGTGAAGTGCCTGCCTGTGA
CCGCCCCAGAGAACGGCAAGATCGTGTCCAGCGCCATGGAACCCGACAGAGAGTACCACTTCGG
CCAGGCCGTCAGATTCGTGTGCAACAGCGGCTACAAGATCGAGGGCGACGAGGAAATGCACTGC
AGCGACGACGGCTTCTGGTCCAAAGAAAAGCCTAAGTGCGTGGAAATCAGCTGCAAGAGCCCCG
ACGTGATCAACGGCAGCCCCATCAGCCAGAAGATCATCTACAAAGAGAACGAGCGGTTCCAGTA
CAAGTGTAACATGGGCTACGAGTACAGCGAGCGGGGCGACGCCGTGTGTACAGAATCTGGATGG
CGGCCTCTGCCCAGCTGCGAGGAAAAGAGCTGCGACAACCCCTACATCCCCAACGGCGACTACA
GCCCCCTGCGGATCAAGCACAGAACCGGCGACGAGATCACCTACCAGTGCCGGAACGGCTTCTA
CCCCGCCACCAGAGGCAATACCGCCAAGTGTACCAGCACCGGCTGGATCCCTGCCCCCAGATGT
ACCCTGAAGCCCTGCGACTACCCTGACATCAAGCACGGCGGCCTGTACCACGAGAACATGCGGA
GGCCCTACTTCCCTGTGGCCGTGGGCAAGTACTACAGCTACTACTGCGACGAGCACTTCGAGAC
ACCCAGCGGCAGCTACTGGGACCACATCCACTGTACCCAGGACGGCTGGTCCCCTGCCGTGCCC
TGCCTGAGGAAGTGCTACTTCCCCTACCTGGAAAACGGCTACAACCAGAACTACGGCCGGAAGT
TCGTGCAGGGCAAGAGCATCGATGTGGCCTGCCACCCTGGATACGCCCTGCCTAAGGCCCAGAC
CACCGTGACCTGCATGGAAAATGGATGGTCCCCCACCCCCCGGTGCATCAGAGTGTCCTTCACC
CTGTGA
SEQ ID NO: 18 sctmCBA Enhancer Promoter-719 bp
CGTTACATAACTTACGGTAAATGGCCCGCCTGGCTGACCGCCCAACGACCCCCGCCCATTGACG
TCAATAATGACGTATGTTCCCATAGTAACGCCAATAGGGACTTTCCATTGACGTCAATGGGTGG
AGTATTTACGGTAAACTGCCCACTTGGCAGTACATCAAGTGTATCATATGCCAAGTACGCCCCC
TATTGACGTCAATGACGGTAAATGGCCCGCCTGGCATTATGCCCAGTACATGACCTTATGGGAC
TTTCCTACTTGGCAGTACATCTACGTATTAGTCATCGCTATTACCATGGTCGAGGTGAGCCCCA
CGTTCTGCTTCACTCTCCCCATCTCCCCCCCCTCCCCACCCCCAATTTTGTATTTATTTATTTT
TTAATTATTTTGTGCAGCGATGGGGGGGGGGGGGGGGGGGGGGGGCGCGCCAGGCGGGGCGGGG
CGGGGCGAGGGGGGGGCGGGGCGAGGCGGAGAGGTGCGGCGGCAGCCAATCAGAGCGGCGCGC
TCCGAAAGTTTCCTTTTATGGCGAGGCGGCGGCGGCGGCGGCCCTATAAAAAGCGAAGCGCGCG
GCGGGCGGGAGTCGCTGCGCGCTGCCTTCGCCCCGTGCCCCGCTCCGCCGCCGCCTCGCGCCGC
CCGCCCCGGCTCTGACTGACCGCGTTACTCCCACAGGTGAGCGGGCGGGACGGCCCTTCTCCTC
CGGGCTGTAATTAGC
SEQ ID NO: 19 IGF-1 signal peptide
ATGGGCAAGATTTCTAGCCTGCCTACACAGCTGTTCAAGTGCTGCTTCTGCGACTTCCTGAAG
SEQ ID NO: 20 IgK signal peptide
ATGGAAACAGATACACTGCTGCTGTGGGTGCTGCTCTTGTGGGTGCCAGGATCTACAGGCGAT
SEQ ID NO: 21 TTR Signal peptide
ATGGCCTCTCATAGACTGCTGCTGCTGTGTCTGGCCGGCCTGGTGTTTGTGTCTGAGGCC
SEQ ID NO: 22 IRES 549-bp
CGTTACTGGCCGAAGCCGCTTGGAATAAGGCCGGTGTGCGTTTGTCTATATGTTATTTTCCACC
ATATTGCCGTCTTTTGGCAATGTGAGGGCCCGGAAACCTGGCCCTGTCTTCTTGACGAGCATTC
CTAGGGGTCTTTCCCCTCTCGCCAAAGGAATGCAAGGTCTGTTGAATGTCGTGAAGGAAGCAGT
TCCTCTGGAAGCTTCTTGAAGACAAACAACGTCTGTAGCGACCCTTTGCAGGCAGCGGAACCCC
CCACCTGGCGACAGGTGCCTCTGCGGCCAAAAGCCACGTGTATAAGATACACCTGCAAAGGCGG
CACAACCCCAGTGCCACGTTGTGAGTTGGATAGTTGTGGAAAGAGTCAAATGGCTCCCCTCAAG
CGTATTCAACAAGGGGCTGAAGGATGCCCAGAAGGTACCCCATTGTATGGGATCTGATCTGGGG
CCTCGGTGCACATGCTTTTCATGTGTTTAGTCGAGGTTAAAAAACGTCTAGGCCCCCCGAACCA
CGGGGACGTGGTTTTCCTTTGAAAAACACGATGATAA
SEQ ID NO: 23 Minimal IRES 462-bp
GAGGGCCCGGAAACCTGGCCCTGTCTTCTTGACGAGCATTCCTAGGGGTCTTTCCCCTCTCGCC
AAAGGAATGCAAGGTCTGTTGAATGTCGTGAAGGAAGCAGTTCCTCTGGAAGCTTCTTGAAGAC
AAACAACGTCTGTAGCGACCCTTTGCAGGCAGCGGAACCCCCCACCTGGCGACAGGTGCCTCTG
CGGCCAAAAGCCACGTGTATAAGATACACCTGCAAAGGCGGCACAACCCCAGTGCCACGTTGTG
AGTTGGATAGTTGTGGAAAGAGTCAAATGGCTCCCCTCAAGCGTATTCAACAAGGGGCTGAAGG
ATGCCCAGAAGGTACCCCATTGTATGGGATCTGATCTGGGGCCTCGGTGCACATGCTTTTCATG
TGTTTAGTCGAGGTTAAAAAACGTCTAGGCCCCCCGAACCACGGGGACGTGGTTTTCCTTTGAA
AAACACGATGATAA
SEQ ID NO: 24 18-bp spacer-IRES (551-bp)-45-bp spacer-
(used in CFHT/aflibercept contruct 398 and other constructs)
GTACCACTGTGCATATGCGTTACTGGCCGAAGCCGCTTGGAATAAGGCCGGTGTGCGTTTGTCT
ATATGTTATTTTCCACCATATTGCCGTCTTTTGGCAATGTGAGGGCCAGGAAACCTGGCCCTGT
CTTCTTGACGAGCATTCCTAGGGGTCTTTCCCCTCTCGCCAAAGGAATGCAAGGTCTGTTGAAT
GTCGTGAAGGAAGCAGTTCCTCTGGAAGCTTCTTGAAGACAAACAACGTCTGTAGCGACCCTTT
GCAGGCAGCGGAACCCCCCACCTGGCGACAGGTGCCTCTGCGGCCAAAAGCCACGTGTATAAGA
TACACCTGCAAAGGCGGCACAACCCCAGTGCCACGTTGTGAGTTGGATAGTTGTGGAAAGAGTC
AAATGGCTCCCCTCAAGCGTATTCAACAAGGGGCTGAAGGATGCCCAGAAGGTACCCCATTGTA
TGGGATCTGATCTGGGGCCTCGGTGCACATGCTTTTCATGTGTTTAGTCGAGGTTAAAAAACGT
CTAGGCCCCCCGAACCACGGGGACGTGGTTTTCCTTTGAAAAACACGATGATAATAGAATTCGA
TATCAAGCTTATCGATACCGTCGACTAGAGCTCCACC
SEQ ID NO: 25 WPRE 589-bp
AATCAACCTCTGGATTACAAAATTTGTGAAAGATTGACTGGTATTCTTAACTATGTTGCTCCTT
TTACGCTATGTGGATACGCTGCTTTAATGCCTTTGTATCATGCTATTGCTTCCCGTATGGCTTT
CATTTTCTCCTCCTTGTATAAATCCTGGTTGCTGTCTCTTTATGAGGAGTTGTGGCCCGTTGTC
AGGCAACGTGGCGTGGTGTGCACTGTGTTTGCTGACGCAACCCCCACTGGTTGGGGCATTGCCA
CCACCTGTCAGCTCCTTTCCGGGACTTTCGCTTTCCCCCTCCCTATTGCCACGGCGGAACTCAT
CGCCGCCTGCCTTGCCCGCTGCTGGACAGGGGCTCGGCTGTTGGGCACTGACAATTCCGTGGTG
TTGTCGGGGAAGCTGACGTCCTTTCCATGGCTGCTCGCCTGTGTTGCCACCTGGATTCTGCGCG
GGACGTCCTTCTGCTACGTCCCTTCGGCCCTCAATCCAGCGGACCTTCCTTCCCGCGGCCTGCT
GCCGGCTCTGCGGCCTCTTCCGCGTCTTCGCCTTCGCCCTCAGACGAGTCGGATCTCCCTTTGG
GCCGCCTCCCCGC
SEQ ID NO: 26 WPRE3 247-bp
ATAATCAACCTCTGGATTACAAAATTTGTGAAAGATTGACTGGTATTCTTAACTATGTTGCTCC
TTTTACGCTATGTGGATACGCTGCTTTAATGCCTTTGTATCATGCTATTGCTTCCCGTATGGCT
TTCATTTTCTCCTCCTTGTATAAATCCTGGTTAGTTCTTGCCACGGCGGAACTCATCGCCGCCT
GCCTTGCCCGCTGCTGGACAGGGGCTCGGCTGTTGGGCACTGACAATTCCGTGGT
SEQ ID NO: 27 SV40 Poly A 122-bp
TAAGATACATTGATGAGTTTGGACAAACCACAACTAGAATGCAGTGAAAAAAATGCTTTATTTG
TGAAATTTGTGATGCTATTGCTTTATTTGTAACCATTATAAGCTGCAATAAACAAGTT
SEQ ID NO: 28 HSV TK 49-bp
CGGCAATAAAAAGACAGAATAAAACGCACGGGTGTTGGGTCGTTTGTTC
SEQ ID NO: 29 bGH 185-bp
TTGCCAGCCATCTGTTGTTTGCCCCTCCCCCGTGCCTTCCTTGACCCTGGAAGGTGCCACTCCC
ACTGTCCTTTCCTAATAAAATGAGGAAATTGCATCGCATTGTCTGAGTAGGTGTCATTCTATTC
TGGGGGGTGGGGTGGGGCAGGACAGCAAGGGGGAGGATTGGGAAGACAATAGCAGGC
SEQ ID NO: 30 Encodes SCPS T2A peptide 54-bp
(Adds amino acid sequence SEQ ID NO: 31 to protein in position 1
and P to protein in position 2)
GAGGGCAGGGGAAGTCTTCTAACATGCGGGGACGTGGAGGAAAATCCCGGCCCC
SEQ ID NO: 31 SCPS T2A
EGRGSLLTCGDVEENPG
SEQ ID NO: 32 Encodes SCPS E2A 60-bp (Adds amino acid SEQ ID
sequence SEQ ID NO: 33 to protein in position 1 and P to protein
in position 2
CAGTGTACTAATTATGCTCTCTTGAAATTGGCTGGAGATGTTGAGAGCAACCCTGGACCT
SEQ ID NO: 33 SCPS E2A
QCTNYALLKLAGDVESNPG
SEQ ID NO: 34 Encodes SCPS P2A peptide 57-bp
(Adds amino acid SEQ ID sequence SEQ ID NO: 35 to protein in
position 1 and P to protein in position 2)
GCCACCAACTTCAGCCTGCTGAAGCAGGCCGGCGACGTGGAGGAGAACCCCGGCCCC
SEQ ID NO: 35 SCPS P2A
ATNFSLLKQAGDVEENPG
SEQ ID NO: 36 Encodes SCPS F2A peptide 66-bp
(Adds amino acid SEQ ID sequence SEQ ID NO: 37 to protein in
position 1 and P to protein in position 2)
GTGAAACAGACTTTGAATTTTGACCTTCTCAAGTTGGCGGGAGACGTGGAGTCCAACCCTGGAC
CT
SEQ ID NO: 37 SCPS F2A
VKQTLNFDLLKLAGDVESNPG
SEQ ID NO: 38 Encodes SCPS Furin 2A Cleavage Peptide 24-bp
Adds amino acid sequence SEQ ID NO: 39 to protein in position 1
and GG to protein in position 2)
GGCGGCAGGGGTCGACGCGGCGGC
SEQ ID NO: 39 SCPS Furin
GGRGRR
SEQ ID NO: 40 SCPS Furin
GGRGRRGG
SEQ ID NO: 41 SPACER
TAAGTGACCTAG
SEQ ID NO: 43 SPACER
TAAGTGACCTAGAAATTCAGGCAC
SEQ ID NO: 47 mIRES and flanking
GAGGGCCCGGAAACCTGGCCCTGTCTTCTTGACGAGCATTCCTAGGGGTCTTTCCCCTCTCGCC
AAAGGAATGCAAGGTCTGTTGAATGTCGTGAAGGAAGCAGTTCCTCTGGAAGCTTCTTGAAGAC
AAACAACGTCTGTAGCGACCCTTTGCAGGCAGCGGAACCCCCCACCTGGCGACAGGTGCCTCTG
CGGCCAAAAGCCACGTGTATAAGATACACCTGCAAAGGCGGCACAACCCCAGTGCCACGTTGTG
AGTTGGATAGTTGTGGAAAGAGTCAAATGGCTCCCCTCAAGCGTATTCAACAAGGGGCTGAAGG
ATGCCCAGAAGGTACCCCATTGTATGGGATCTGATCTGGGGCCTCGGTGCACATGCTTTTCATG
TGTTTAGTCGAGGTTAAAAAACGTCTAGGCCCCCCGAACCACGGGGACGTGGTTTTCCTTTGAA
AAACACGATGATAATAGAATTCGATATCAAGCTTATCGATACCGTCGACTAGAGCTCGCCACC
SEQ ID NO: 48 ITR_AAV2_R_short 128-bp
GCGCGCTCGCTCGCTCACTGAGGCCGCCCGGGCAAAGCCCGGGCGTCGGGCGACCTTTGGTCGC
CCGGCCTCAGTGAGCGAGCGAGCGCGCAGAGAGGGAGTGGCCAACTCCATCACTAGGGGTTCCT
SEQ ID NO: 49 Lentiviral 5′ LTR (Truncated) 181-bp
GGGTCTCTCTGGTTAGACCAGATCTGAGCCTGGGAGCTCTCTGGCTAACTAGGGAACCCACTGC
TTAAGCCTCAATAAAGCTTGCCTTGAGTGCTTCAAGTAGTGTGTGCCCGTCTGTTGTGTGACTC
TGGTAACTAGAGATCCCTCAGACCCTTTTAGTCAGTGTGGAAAATCTCTAGCA
SEQ ID NO: 50 Lentiviral 3/LTR (Delta-U3) 234-bp
TGGAAGGGCTAATTCACTCCCAACGAAGACAAGATCTGCTTTTTGCTTGTACTGGGTCTCTCTG
GTTAGACCAGATCTGAGCCTGGGAGCTCTCTGGCTAACTAGGGAACCCACTGCTTAAGCCTCAA
TAAAGCTTGCCTTGAGTGCTTCAAGTAGTGTGTGCCCGTCTGTTGTGTGACTCTGGTAACTAGA
GATCCCTCAGACCCTTTTAGTCAGTGTGGAAAATCTCTAGCA
SEQ ID NO: 51 VMD2 promoter
CAATTCTGTCATTTTACTAGGGTGATGAAATTCCCAAGCAACACCATCCTTTTCAGATAAGGGC
ACTGAGGCTGAGAGAGGAGCTGAAACCTACCCGGCGTCACCACACACAGGTGGCAAGGCTGGGA
CCAGAAACCAGGACTGTTGACTGCAGCCCGGTATTCATTCTTTCCATAGCCCACAGGGCTGTCA
AAGACCCCAGGGCCTAGTCAGAGGCTCCTCCTTCCTGGAGAGTTCCTGGCACAGAAGTTGAAGC
TCAGCACAGCCCCCTAACCCCCAACTCTCTCTGCAAGGCCTCAGGGGTCAGAACACTGGTGGAG
CAGATCCTTTAGCCTCTGGATTTTAGGGCCATGGTAGAGGGGGTGTTGCCCTAAATTCCAGCCC
TGGTCTCAGCCCAACACCCTCCAAGAAGAAATTAGAGGGGCCATGGCCAGGCTGTGCTAGCCGT
TGCTTCTGAGCAGATTACAAGAAGGGACCAAGACAAGGACTCCTTTGTGGAGGTCCTGGCTTAG
GGAGTCAAGTGACGGCGGCTCAGCACTCACGTGGGCAGTGCCAGCCTCTAAGAGTGGGCAGGGG
CACTGGCCACAGAGTCCCAGGGAGTCCCACCAGCCTAGTCGCCAGACC
SEQ ID NO: 52 FHR-1 SCR1-Non-codon Optimized
GAAGCAACATTTTGTGATTTTCCAAAAATAAACCATGGAATTCTATATGATGAAGAAAAATATA
AGCCATTTTCCCAGGTTCCTACAGGGGAAGTTTTCTATTACTCCTGTGAATATAATTTTGTGTC
TCCTTCAAAATCATTTTGGACTCGCATAACATGCACAGAAGAAGGATGGTCACCAACACCAAAG
TGTCTC
SEQ ID NO: 53 FHR-1 SCR1-GeneArt Codon Optimized
GAGGCCACCTTCTGCGACTTCCCCAAGATCAACCACGGCATCCTGTACGACGAGGAAAAGTACA
AGCCATTCAGCCAGGTGCCAACCGGCGAGGTGTTCTACTACAGCTGCGAGTACAACTTCGTGTC
CCCTAGCAAGAGCTTCTGGACCCGGATCACCTGTACCGAGGAAGGCTGGTCCCCTACACCTAAG
TGCCTG
SEQ ID NO: 54 FHR-1 SCR2-Non-codon Optimized
AGACTGTGTTTCTTTCCTTTTGTGGAAAATGGTCATTCTGAATCTTCAGGACAAACACATCTGG
AAGGTGATACTGTGCAAATTATTTGCAACACAGGATACAGACTTCAAAACAATGAGAACAACAT
TTCATGTGTAGAACGGGGCTGGTCCACCCCTCCCAAATGCAGGTCC
SEQ ID NO: 55 FHR-1 SCR2-GeneArt Codon Optimized
CGGCTGTGCTTCTTCCCCTTCGTGGAAAATGGCCACAGCGAGAGCAGCGGCCAGACACATCTTG
AGGGCGATACCGTGCAGATCATCTGTAACACCGGCTACCGGCTGCAGAACAACGAGAACAACAT
CAGCTGCGTGGAACGCGGCTGGTCTACCCCTCCTAAGTGCAGAAGC
SEQ ID NO: 56 Gly Ser Linker amino acid sequence
GlyGlyGlySer
SEQ ID NO: 57 Gly Ser Linker amino acid sequence
GlyGlyGlyGlySer
SEQ ID NO: 185 (TYR Promoter Region)
CACTAAATACACAAAACTTTAGAAGTCTTCACTACAGTTAAAAATATATCATGTTTAGAGGTTG
TTAGGTAGCCACATTATTATTCTGAAAATTGAAAAATGAAGGACAAGAATCAAACATTTACCCC
GCTTTTCTTGTACAGGGTAACTAAATAGTTCATGAAGAAAGTTATTCTTTACAAAAAAAAGGGG
ATAACCATTCTTGCAACCTTTAATAAAATACCAAATGTAGGATATACTTATCAGTGAAAATAAA
ACCATATAAAAGTTTGCTAGAGAATTATGTAATTGAAAGGACAGTCTGACATCACTTCAAGCTG
CTGATAAATTTTAAAAACAAATACTGAGAAAACCAGATAATATGGTCCTCTAGTATATCAAAAT
AAGTATTACTTAACAATGCCTGTGAAGTAATTGTTAAGAAAAGCAATAACAAAATTCAACTTGT
ATATAATAAAATCTCTAGATCTAACTCCCTGATTGTAGAAAATGCAAGATATTATTATTGAAAA
TGTATAAAATGATAGAAAATACAGGATAATGAGCATGTTAAATTCATAGAGATAAAAGTTGTTA
ACCCCGAAATGTGAGAAATCCTTAGGGCAAAAACTCAGTTTCCTCTAAAAATAAATTGCATTTT
TTTTTTTTTTTTTTTTTTTTTTGAGACGGAGTCTCGCTCTGTCGTCCAGGCTGGAGTGCAGTGG
CGGGATCTCGGCTCACTGCAAGCTCCGCCTCCCGGGTTCACGCCATTCTCCTGCCTCAGCCTCC
CAAGTAGCTGGGACTACAGGCGCCCGCCACTACGCCCGGCTAATTTTTTGTATTTTTAGTAGAG
ACGGGGTTTCACCGTTTTAGCCGGGATGGTCTCGATCTCCTGACCTCGTGATCCGCCCGCCTCG
GCCTCCCAAAGTGCTGGGATTACAGGCGTGAGCCACCGCGCCCGGCCGCATTTTTTTTTAAAAA
AGGAAACTTATAACTTAATAGAAATTGATAAAATAGATCAACCAAAATGCAGTGTATATACCTG
TTTGAATCTTGACAAAAATACTTTTGAGATAATTAAAGTAAATTAACCATAAATTTGATACCTG
ATATTAAAGAATTATTGTTCTTTTTGTTTGAGATGTGTTTGTGTAAGATACGTGGTTTTGTTTT
TATGTTCTTAATTGTTAGCAATATATACCGAAATTAGAGGTAGAATATGCTATCTGAGTTTTGT
TTTAAAACATTCCAGAATTTTTAAACTAGTAGAAACAAAACAGAAAAATGCTGGATGTTGAAAC
TTGGAGGTAAGTAAATGCTAGTTTATTATATTATTTTTTACTGATTTGAGCATATTTCTTTCCA
CAGTAAATTTTTTTCTTTAAATGGTAGCTACTGATACTGAGATAATTATATTAGTTGTATTTAA.
TAAATGGTAGCTACCATTATAATTGAAAGGAGGGCTGAGAAACAAAGACAGAAAATGGATTCAA
ACTACTACCAAATAAAAGAGCAAATACCAGTGATCTGAGTGAGGAAAATGAGGAAAATTGACCA
GGTTAACACAAGTGAGTGGCTGAATAATCTATACAAATACCAGAAGTAGAGGACCAGTGATTGT
GGCTAGTTGATACTCAAACTCAACTTATCTAAAATTGAGCCTATATTCAATTCCCATAAACATT
TATCCTCTATTGTTTCTTATTTTAGTTAATGGCACAAGCCAAAAACCTAGCACATAGCCCTAAA
TCTTCTTTCCCTTCACTTCTTACTGCTCTTGAATTTATCACCTATGTATCTCTCAAATTCATTA
ACTTATGCCCATCTCAGAGATCATTACTCTAGTCTAAGCAATGTCATGTTTGCCTGGTTTACAT
CAATAATATCCTAACAGTAATCTCCACATTTAATCTAGCTCCCACCCCACATCTGTTCTCACCT
CAGTAGCTTGAGTGAAAAGTTTTAAATTCATGTATCGTTACCCAAAATTTTGCTACAAAGCCTT
TATGGACTATGAGCTGGTCCATACACACACCTTCATTCTTATATCACGCCTCTACCATATTGGG
GGTTGCAGCCACCTTAACTGTCCTTAAATCCCTTGAATATACCAACCTACTTCCACTTTGGAGA
CTTTGCACATGCTATTCTCTCTGCCTGGAAAGCTCACACTCCTACTGTTCTCTCTGTCATCAAA
CAGACTTCTAGTGATCCCTCAAGTGTCACTTGAAATGTCACTTCTCCCATAAAATTATCCCTGA
CTATCCCTTCTCTTGTGAGACTAGAAAGGCCCTTCTGTCATGTTCCCTAATTTCTCTCTTTAAT
TTTCTCTTATAGCAACCTAAAAAGTAATTTAATCCAGAAAAAAAGTTATTTAAAGTAATGTAGT
TAAAATTATTATTTAAAATCACTAAAATTAAAATAGTATTTTAAGAGGCAATATCCTTCCTGAT
GATAGAGATTTTTGTTGTGCTTACCATCACATTCCCATCTCTCCCCATGTCTGCAAGTAAATAC
TTGCAGAGTATTTACTGAACCCCTAATTCTACTTTATAGAGCATTGCAGTAATTTCATGTCAGG
TCATACAATAATCTCCCAAAATAGGATAGGACCCTTTCAAATCCCACAACTGAAGATACCTAAA
GAATGTACATCTAGAGTCAGGAAGAAAGCACTCTAAATGGGATTTAATATTTAGGAACCCAAGA
CTGGCAATAAGACTGAAGACTACAACACGTGTAGGCCAGAGGAGACAGTGGCCTATACTGGGGA
CAAATAAAGAGGTCTGTCCTATTTAAGAAAATCAACCCTGTAAAGGAAATTAATAGGACTAAGT
ACATTTTAGTAATTCCTCTAAGCAGGCTCTAAAGATTATGAAAAATAGACGGGACAGCAGACAC
AAAAGCCCTTAAAGAGCATGATGAAGACTTTCTAAGTTATTTCACTGGAAGCCTGATAGTGGGG
CAAGTGTAAGGCAAAATTCTTAATTAAATTGAACATGATAAGTTGAATTCTGTCTTCGAGAACA
TAGAAAAGAATTATGAAATGCCACATGTGGTTACAAGTAATGCAGACCCAAGGCTCCCCAGGGA
CAAGAAGTCTTGTGTTAATCTCTTTGTGGCTCTGAAAGAAAGAGAGAGAGAAAAGATTAAGCCT
CCTTGTGGAGATCATGTGATGACTTCCTGATTCCAGCCAGAGGCAGCATTTCCATGGAAACTTC
TCTTCCTCTTCACCCACACACTGCTCCATGTACCTGCAAAGCCTGTTCTGTCTCAAAAAAGTTG
TTTGGATGAGCCGTGACTTTTTTTTTTCTTAAATAATGAGACAAACTCCAGAAAAAGAGAAAAA
AGCAGAGCAGTCTGACATTCCGGCATCATCGAAATAGTGATGGCTTTTCCTAGAATGCTTCAGC
TAAGGACCCAAATACTAATGATCTCCTCAAAGCTTTCACTTTCTTTTACTTTTTCATTAATTTC
AGTGGACCCCCAAACTTTAAGTATGGAAGAGGACAAAGAAGGAAGCTTCAGAGGGGCAACTTTG
ATTTGACTACTCTTTTTGTCACTCTTCAGCTCACAAAAGAGCTCACTTTAGTTCAAAACACAAA
GTCTTTAAGCCCCTCCATAGATTGGTCCCAGGTTTAATTTTCTATGATGTGTGGAGGCCTCAGT
TTAATGCTCCAACTTGATAGATGAAACACAGTTCCCACCTCTACACATTTCCCCTGTCTCAGGA
GTTGTATATATTCTCAGTTGTCTGTCCAACTTATGCCCACTCTTTGAGATATTAATCAAGGCAC
TCCCTTGATAACACTTGCATATTATTATCAAAATTATGCAATTCTTTCTAATATCAGCCCACAA.
ATACATCTCTTCCATTAAAAGTTTGACTTAATTATCTATACTACTCATTTGAAAACTAACATAG
TTAAGTTGTATTTTTAGCCATGAATTTCAGTTTCCCTAGCTCACTATACACAGAGAAGGAACTT
TTGAAATAATTGAGATGATCAAAAATATTTGCTGAAGAAATATATTTCTCCTTTTTCATTCACT
CACTAATTGAGAATGTCTTTGCACAAAACACATTGCAAAAACATTTTCAAAAAAATTCCTAATT
TCTAGAATTGATAGGAAAAACAATATGGCTACAGCATTGGAGAGAGAGAGAAAGGAGAGAGGAG
AAAGGAGAGAGAGAGAAAGGAGAGAGGAGAGAGACAGAGGAGAGAGAGAGAGGATAGAGGGGAG
AGAGAGAGAGGAGAGAGACAGAGGAGAGAGAGAGAGGATAGAGGGGAGAGAGAGGGAGAGGGAG
AGAGAGGGAGAGAGAGGGAGAGAGAGAGAGAGAGAGGGAGAGAGAGAGAGAAAGAGAGAGAGAG
GGAGAGAGAGAGAGAGAGCTCTTTAACGTGAGATATCCCCACAATGAAGCAAATCGCCCAGTTA.
TCAAAGTGAGCTATCCTTAGGAGTTGTCAGAAAATGCATCAGGATTATCAGAGAAAAGTATCAG
AAAGATTTTTTTTTCTGATACGTTGTATAAAATAAACAAACTGAAATTCAATAACATATAAGGA
ATTCTGTCTGGGCTCTGAAGACAATCTCTCTCTGCATATTGAGTTCTTCAAACATTGTAGCCTC
TTTATGGTCTCTGAGAAATAACTACCTTAAACCCATAATCTTTAATACTTCCTAAACTTTCTTA
ATAAGAGAAGCTCTATTCCTGACACTACCTCTCATTTGCAAGGTCAAATCATCATTAGTTTTGT
AGTCTATTAACTGGGTTTGCTTAGGTCAGGCATTATTATTACTAACCTTATTGTTAATATTCTA
ACCATAAGAATTAAACTATTAATGGTGAATAGAGTTTTTCACTTTAACATAGGCCTATCCCACT
GGTGGGATACGAGCCAATTCGAAAGAAAAGTCAGTCATGTGCTTTTCAGAGGATGAAAGCTTAA
GATAAAGACTAAAAGTGTTTGATGCTGGAGGTGGGAGTGGTATTATATAGGTCTCAGCCAAGAC
ATGTGATAATC
SEQ ID NO: 186 (TYRP1 Promoter Region)
CTACCAAATTCTTCATTCCCTCCTCTTGTGATGTAACTTCTAACATCCCGTCTCATGGCAGATT
ATTAGAGTGCGTAAGGAGGAAACAGAGGTAAATTTTGGTTCTGATGAAGAGGTAGAAGGGGTCA
GGAGGTTTCCATCATTGTCTGTCTTCTGCAAGGTTATCTAAAAAGTGAAGAGACATCCTTAAAG
TGAGTATTGACCAAATGTCAGTTGTCTCTTCAGACTTCCTATGGTTTAAAAATAAATGAAAAGA
AAAAAGCTCTGTTTTCTTACCTAAATTTGAGTTATATTACCAAAGGCCCCTTAGGTAATGGGGC
CTTTATACGTTCCAGAGTAAGTAGCATATGGTGTGTGCATGCATGTTTCTCTCTCTCTCTCTCT
CTCTCATTCTGTGTGTGTGTGTGTGTGTGTGTGTGTGTGTGTGTGTGTGTCAGTGGAGAGAGAG
AGAGACGAAGAAAAGGGAGGAGTGATAATCCTTCTAAACGTATACAAGTTGCTTGTGAGAAAAA
TAGCAGTAATCATAACACACCAAGTCCCTTACTGCTGGTAATGCTTTTCATGTGTCTCTCCAGA
TTGCCTAATTTTAACTTCTTAAATGTCTCATCTCCTCCTCTATACCCTGGAAAATTATAATATG
CTGATTCAATTTAAAAAAGTTATTTGAAATGTAGCCAAACTCATTTGATCTTCCTCAATTCATC
ACTGCATATGGGTTTCATCCCATTTTATTAGTAAATTTGACACTGATTATGATAAATCCATCCA
GTGCCACACATCTCCATCTGCAAATTTCAAGGTTTAATTTTAAACTTTTTAGGGTAAGAGGTTT
TTAAAATATTTTCAACCTGTAGGATTTCCAAGACCTTATTTCTAAATGTTTCAATATGACCAAT
CTTATACATGTGATTTTATATTTCTATATCAGATATAAAAAAGATTTAGATTATTTAGGCTTTC
AGTTAGTTCTTTTTATACATGAAGCTACTTTTACGAGCAAGATGAAGGGGAAGTGAGATAGTGG
CAGTTGCTGTCTTTTGGATGACAGGCCAATTTACTTCTTGCTTCTAAAGGAAGTAGGCTTTACC
CTTTAAACCTCCCCCATCATATGTTCTTGCATGTACTGTTTCCTTTCATCAGTAAGTTCTCCTG
ATCTTCTCTGTCTCTCCCTCTCTCTCTATCTCTCTCTCTGTTTGACTGTTTCTCTGACATTACC
CAAAGATAACTGTTTTTTTTTTCTGGTCTCAGAATTATTCGATGTCCTTTAGTAGTGAGGTCAG
CTTCCACAAATGCCTAATTAAAAAAAATTTTCCTTAAAATAGGCAAGAGGAGGAGGAGTTGAAT
TCCAAACACCTGGAGTCTCCTTTGATTAGCAGTATTTAATTTCTGATAGTACCCAATTTATGGC
ATTTTAGTGAAGACAATTCTAAAAGTTAACAGAGAAGTCAGGCCAATCTTTCCGATAGGATGAC
ACACTCCTTTGTAAATCTTTAAATGCTTTTCATTGCATTATTTGTGAGTAGGGACCAGCAGTGT
TCCTAAATAGGAAGTGTGTTTTTTGCCAACAGTAAGTCCCATAGATCTCTGGTTTTCCTGTTTA
GTGGAGGGTTACTAAGAGAGAGTACTTAATTTCTCACAGTTGGAAATTTTCCTGGTTTCTCTAG
ACCATGTAATTCTTCAAAATGTTATAGGGTAGACCAAGTTCTGAATTTTACCTGTGCTTTTGAT
TAGGCTTCAGTTTGTTTTACAGATTATTGTATTTCTTGTCTCCTTCTGTGCTTTTTCCACAGTG
CAGGCAGTAATCACAATATCCTTCATAGAGTGTACTACCTTGCCCTTAAGGATGTTAGAAACGC
ATGGTATCGATTCCCATTGTGCATTTAGATAATAGCAATACAAGCACAGAATATTATCTTGGCA
CCATAGAAGCTCTACAATCCCCAAGTGTCAGTAGTACATTTTCTTCAAACGTTAGCAACCTCAA
TCCCTGGCAAGAAAAAAAACAGATTATGCAATCTATAATGATCTTACTCTTCAGCAAAATTCTA
TTTTTTTGTGTTCGTTATTAGAAATTTCCACAGACATATTTTAGCAGTCATATTTCTGCCTGCC
CAGCTAGAGTAAGCTCAGTGAGGAAGGGGGTCTGTTGCTACTCTTCTTTAATGATGGAAACACT
GAGGAGTATATGAGGAATTTTACCCATTAGATGTGTGAGGCTAAGAGATCTTTGATCATGCCAC
TGCTCTCCAGCCTGGGCGACAGAGTGAGACCCTGTCCCAAAAAAAAAAAAAAATGATATGTCCA
CTCATTAAAAACTTCTGTCAGACCACTCTTTTTTATAAATTTTTAAATTTTTTAATGATCTAAC
AAGCTTTATTTTTATCATTGTTAAAAAAATCCTTCTTTTGAGTAACACAATCACAAAGTAAAGA
ATGTTATCATTGATTTAACCAGTAAAAAAACAATACTTAACAATAAAATTTTTCAAACATGGCT
CCAGGCCATCCAGATGACTTAGTTATCCTCCTTTGATGCCCTTCTCTTCTTCGAATGAATCACA
ATGAGGCAGAAAAATTTTCATGGGTGCAGAGTAGGTGTATATATTTATGGAGTATGTGAGATAT
TTTGATACAGGCATACAACATGAAATAAGCACATCATGGAGAATGGGGTATTCATACCCTCAAG
CATTTATCCTTTGAGTTACAAACAATCCAATAACACTTTTTAAGTTATTTAAAAATATACAGTT
ATGATTGACTATAGTCACCCTATTGTGCTATCAAACAGTAGGTCTTATTCATTCTTGCTAATTT
TTTTGTACCTATTGACTATCCCTACTTCCCTACTCCCCTCAGACACCCACTACCTTTCCCAGCC
TCCATTCTTCTACTCTTTATGTCCATAAGTTCAATTGTTTTGAGGTTTAGATTCCACAAATAAG
TGAGAATAAGTGATGTTTGTCTTTCTGTGCCTGGCTTATTTCACTTAACAGAATGTCTAGTTCC
ATCCATGTTGGTGCAAATGACTGGATCTCATTGTTTTACTGCATTTTGCATATGTACCATATTT
TCTTAATCCATTCATCTATTGATGAACACTTAGGTTGCTTCCAAATTTTAGCTTTGTAAACAGT
GCTGCAATAAACATAGGAGTGCAGATATCTCTTCAATATATTGATTTCCTTTCTTTTGGGTATA
TATCTAGCAGTGGGATTGCTGGATCATATAGTAGCTCAATTTTTAGTTTTTTGAGGAAACTCCA
AACTTTGCTCCATAGTGGTTGTACTAATTTACATTACTACCAACGCTGTATGAGGGTTTCCTTT
TCTCCACATCCTTGTCAACATTTCTTATTGCCTGAATTTTGGACATAAGCCATTTTAATTGGGG
TAAGAGGGTATCTCACAATAGTTTTGATTTGTACTTCTCTAATGATCAATAATGTTGAGTATCT
TTTCATAAGTCTGTTTGCCATTTGTATGTCTTCTTTTGATAAATGTCTGCTCAAATATTTTGCC
CATTTTTTTATTGGATTATTAATTTTTTTCTATAGAATTTTTTGAGCTTCTCATATATTCTGGA
TATTAATCCCTTGTCAAATGGGTAGTTTACAAATATCTTATCCCATTCTGTGGGTTGTCTCTTC
ACTTCATTGATTGTACTCTTTGCTGTGCAGAAGATTTTTAACTAGATGTGATCCCATTTGTTCA
TTTTTGCTTTGGCTACCTGTGCTTGTGTGGTATTGCTCAAGAAATCTTTGTCAAGACCAATGTC
CTCAAGATTTTCCTCAATGTTTCTTCATTGTAGTTTCATAGTTTGAGGTCTCAGATTTAAATAT
TTAATCAAATCATTTTTTATTTGATTTTTGTATATGGAACGAGATAGGGACCTAGTTTCATTCT
TTTGCATATGGGTATGTAGTTTGCCAGCACCATTTATTAAAAAGACTGTCTTTTCCCCAGTGTA
TGTTCTTGGTATCTTGGTGAAAAATGTCTTTACTGTAGGTGTGTGATTTTGTTTTTGGGTTCTC
CTGTTCCATTGCTCTATGTGTCTGTTTTTAGGCCAGTACCATGCTGTTTTTGCTACTATCACTC
CGTAGTATAATTTGAAGTAAAGTAATATGATTCCTCCAGTTTTGTTATTTTTGCTTAGGATAAC
TTTGCCTCTTCAAGGTTTGGTGTTCCATATAAATTTTAGGATTTTCTTTCTGTTTCTGTGAAGA
ATGTCTTTGGTATTTTGATAGGAATTGCATTGAATCTGTAGATTGCTTTAGGTAATACGGACTT
TTTAACAATCTTGATTCTCCCAATCCATGAACATATAATATTTTTCCATTATTTGGTGTCTTCT
TCAATTTCTTTCATCAATGTTTTGTAGTTATCATTATAGAGACCTTTCATTCATTGGTTAATTC
CTAGGTATTTAATTTTATGTGTGGCTATTGTAAATGGGATTACTTTTTTATTTCTTTTTCACAT
TTTTCACTGTTGGTATATGGAAACTTTGCTGATTTTGGTATGCTGATTTTGTATCCTGCAACTT
TACTGAATTTGTTTGTCAGCTCTAATAGTTTTTTAGAGGAGTCTTTAGGTTTTATTCAAATATA
AGATCTTATCATCAGCAAACAAGGATAATTTGACTTCTTCATTTCCAATTTGGATGCTCTTTAT
ACCTTCTCTTGCCTGGTTGCTCTAGCAAGGTAATATGTTGAATAACAGTTGAATAACAGAATAA
AAAAAAATCTCTGCAAAGTAAACAAATCTCACTAGTTTATCTGACTTGTATTCCAAATTAGTGC
TTCTGGCCTTTTCTTAAAACTTTAAGCATCACAAGGAAATCAGTTGGAAGGGAATCATGTGCTG
ATCAAGTCCTTAAAGGGCAGAAATATTCACTGAAGTGAAAAGGATTAGTAAAGGGTGGAAAAAA
AGACCAGCCCCCCGCCTAGTTTGGGTGAGCAGATTTGGGATTAATTATCAGGCAGCAATCCACA
TGCACTT
SEQ ID NO: 187 (FLT1 Promoter Region)
GCATTGCTGAATGAAAGAATACGTTTCCTGGATCATGCACCATGCTACGAGCTGTTTCCTTCGA
CTGCTCAGCCAGGTCACTGTTGGACTAAGCTGGTAGGGTTTTAAGTAAAGCAGGGATATTGATG
AGCGTGCATGTGCGTGTGTACATCTGACTAAAAACATTCCAGTGTCAAATTAAGATTATTTTGG
AATTATTTACAATGCAGAAACTGGAATGTCTAGCAAGAGGATAATAGTTTGCGGCTATTAGCTA
CAACAGTTATGAAGGCTCTATAGAAACATGGAAAGATAAAAGCCGTCTGTATGGAAAAGGCTCG
AATGAAATACACCACAGTGATCACAGTGGCTCTGCTGAGATGATGGAATTATGACTCATCCTCC
AGACCTTCCCTTCTTTTAATCTTTCTAAAATGTTATATTTAATGACTATGAGTGTATTAAAAGT
AATAAAACAGGAAAAAGAGTAAGGAGAAGAGTCAATGGCTGGCAGTTAAGTGCTGAGATACAAC
TGACCTTAGGCAAGATAGTAGTGATACTGATTAGAGGAGGTTCCTAGAGATTACTCTAATTGGC
CACTTTTCCTTTAGGCCTTATTTGCTTCTCTGATTTGCCGCTTCCCCTGCCCCATGCTTCTCTA
ACTATCCCAGTCTGGTCTTTTTGTTTTTTTTCCTTTCAACTTTTCTAGTATGCCTTCAATAAAA
TGTACCTGTCTTATTTTCTCTTGCTAGGACCATCAATTATTCCTAAATAATGACATGGTAAATG
TGAATGAGTTAATATGCAGCCTCCTTTGGTGAACATACAGCTAGAGATAATGACACAAAGGAAT
AATTTGAGTCCTTCCTTGGTTTGCTCAACCTCCACAGATCTCTACTTTCATCCATCCATTCATT
CATTCATTCATTCACTCACTCAGCTTTGTCCTTGTCAGTGATTTTGATGATGACCCAGAAGGCA
CGTTTACCGTATTTGTGGATCATTTGAAGCGGGAGCTATAGAAAGTCCACAGGATGGCAGAATC
AGAGACCAGAAAGACTTCGACATGCTGGAAAGACAGACTAAAACAAACAAAAGGCTTAAACAAC
ACGCTCAGGGCCTGCAGCCTGAGTCAACCCAAGCGGCGGCTGCACAAGGGCAGCCAGATAGCAG
CTCTCATGAGAAAGCCTTGGGGCTTTAACCAAGAATAAGTTCTGGAAGAGTCCAAGGTATGAAG
GCGCTGACAGAAAAGGCTCCTTTGATCTTAGGTCCCAGGAACAGAAGCAAGTTCTCTCCGTAGT
AGCCCCTGGGAGTAGCAGGAGGAGAATTCCAAGTAAAGCCAATGGCGATCTTATAAACACCAGC
AGCGCCCACCCATGCCCGGGAGAAGGTAGTCAGGAGCAGACGGGAGCATGAGTAGCAGGAACAG
GAGGAGCCAGGGAGCCCAGGCCGGCGGCCCCAGAGGTGCCACCAGGGCAGTGCTGCCCTGCTCT
CGGTGGCACTGGCCTTATCTCCAGGAGGGGGCTGTCCCCTTTGCTTCCTTCTCCACGCTGCTCT
TCCCACCTGAACGATTTTCCTTCCTCGCTCCCACTGGGCCTTCCCACAGGCTTTTCTCTCTGCC
TGAAAGAAAAACTACCTCTAGGAGTAGGGAAATGATAATTGTGCTGCATTTTCAATCCTAAGCG
TGTCACCTGCAGGGACACTGATATGCTGGATGGAATTTCTGGAGCGTGTCAGCTGAAGAGTGAC
CAAAGGGATGTGGATGTTGAGGCTGGAGACAAGATGCCCATGGCACAGGAGGTGTTTTTAAGTG
TTCAATAAATTTTCATGTGGAGATATTGGAGCATTGAATAGGAATTCCACGAATTGACTCGGTA
GATGGGTTGAATGGCTTTTAAACCCAGTACAAGATGTTTAAGTTCTTTGAAAATGGGGGCATTT
CAGCCACAGTGGTAGCTTCCCATAGCAAGACTTTTGAGCCAATGGGGTCATCAGCATCTGATAA
GTAAACTCATACATGTGCCTGCCTGGTGAACTCCGCAGAACTTCTTAGCAGAGATTTGGGAGCG
TGTGCTTTTTAGACCAAGGAGCCTGAACTGTCCAGAGGATATGTACTGTGAAGCTTTTTAAAAT
ATAACACAGAAGGGTTTTATGAGATCATTTGGTTCTTGTATTCAACCATACATATTTGCATTAT
GCAGAGTATGCCCATAGAGTGTATTTTTCCTTTTGTATCAAGCTACCTAACCCAAGTGGAAAAG
AAATTCCTGACTGATCCTCAGTGACTGTGGCTGATCAGCTCCATGCTTGGTCTTTGGAGGAAGG
TGAGAGCCTGGGCTCCCTCCTGTCTCTCACACTGGTGCTCCAACACTCTTATTCAGTCTCGAAT
ATTAAAGCTGATGGATTTAGCAACAGTGTGCTTCTGAATAGGGGTGAGAAAGAAACCTCAAGAG
AATGTTTGTAGCTGATAACAAGAGAGAGTGTCTGGTTTTGCTAACCAAACAGGAGATGAAATGT
TCTTTTGTATGTGAGCAGGAAGCAAGCAGATTGCCTACTTCAATAGCTTTTTATACCCACCAGG
CGTGTTGAATTCCATCTTCTCCATCAGTATTATCTATAAAACCTCTTTCATTAGTGATTTAGTT
AGGAGAGTGCTATAGCCCATTCTCATTCAAAGGTATTATCTCTTTTGGGGAGAGGATTGGGAGC
TATACATTTATTCCCCATATCTAAGCTAAAATTTGTAGCATTTTTGAGTTCTTTGTGAGAACGC
TAATGCAACTAACAAAGAAGTCATAGTCAATAAATTGCTGACTCTAATTAAAAGCAACTTTTCA
AAAGAGTAGTTGGGAAATGTGGAAGCCCACCCTTACTGCAGCATAGTTTTAATATAAATCTTAG
ATTCGACTGTGACTCTACTAACCTTAAATAATTGAGCAACATGTTAATATCATTGATTAGCATC
TTAGATTATAAAGATTGTTTCAGCTGTCCATCAAATACCTGTTCTGGCTTTACAGACACAATGT
AGATTCATTGCTGGCAGCAGGTTTGGAAAGTGTTGTCTCTTTATGAGTTAAAAATATCAAAATG
GCATTTGAAAAAATGTATTAGCAAATTAAACCTTTGTACATGGTACTATGGACCACTTTCCCCC
AGAGTCAAATGTGTGTCTCTCCTGAGAAGCCCTCCTTGGGTTTTCCCTGCCAATAAATGTGCCT
TTACTTCTCTGAGCTGATGGAGGACTAAACACCCCTCCAGCACTGGAATGCCGGTCCTGCAGCC
ACTTCAAGTGTCCACCTTCTTATCACAGTCCAAGCCCTTTAGAAGGCAAAGATGATGGCTCTCC
ATCAGTGTTTTCAGCCCTAACCCAATGCCTGGCCCCTGTTGAATGCTGCAGAAGGACAATGCTG
ACAGCTTCTCCTTGAAAGAGGGTGTGGGAGTGACAAGCAGCTCTCAGGTGAGACCAACAGACAC
TGTGGCTCCGCACGTATGTCCCAGGCTGTGTTCTGGGCTGGGGGACCAGCAAGGAGAAGGATCA
GCTGTCCGCTTCCAGCTGTTCTGAGGGATGATGCTGTGGGTTTTAGAGGAGGCAGGGGTGCGGG
AGGCAGAGAAAGGCGCCAGGAAGAGTGGTGAGGAGAACTGGAAAAACAGAAAGCGCTGGTGAAG
CCAGCAGGCAAGCAATGACTCCGGCAGTCTTCCTTGCCTCTGAAGCAGAAGGGATCCAGGGACA
GTTAGTGGCTCAGGTCCTGCGGCTCCAGGTGCCCAGACGTGCATTTCCTGTGTGGAGTGGCCGG
GGCCTCTCCTTGGTGTGCAGCCCAGAAATGTGGCAACTTTGGGTTACCCAACCTTCCTAGGCGG
GGAGGTAGTCCAGTCCTTCAGGAAGAGTCTCTGGCTCCGTTCAAGAGCCATCACAGTCCCTTGT
ATTACATCCCTCTGACGGGTTCCAATAGGACTATTTTTCAAATCTGCGGTATTTACAGAGACAA
GACTGGGCTGCTCCGTGCAGCCAGGACGACTTCAGCCTTTGAGGTAATGGAGACATAATTGAGG
AACAACGTGGAATTAGTGTCATAGCAAATGATCTAGGGCCTCAAGTTAATTTCAGCCGGTTGTG
GTCAGAGTCACTCATCTTGAGTAGCAAGCTGCCACCAGAAAGATTTCTTTTTCGAGCATTTAGG
GAATAAAGTTCAAGTGCCCTGCGCTTCCAAGTTGCAGGAGCAGTTTCACGCCTCAGCTTTTTAA
AGGTATCATAATGTTATTCCTTGTTTTGCTTCTAGGAAGCAGAAGACTGAGGAAATGACTTGGG
CGGGTGCATCAATGCGGCCGAAAAAGACACGGACACGCTCCCCTGGGACCTGAGCTGGTTCGCA
GTCTTCCCAAAGGTGCCAAGCAAGCGTCAGTTCCCCTCAGGCGCTCCAGGTTCAGTGCCTTGTG
CCGAGGGTCTCCGGTGCCTTCCTAGACTTCTCGGGACAGTCTGAAGGGGTCAGGAGCGGCGGGA
CAGCGCGGGAAGAGCAGGCAAGGGGAGACAGCCGGACTGCGCCTCAGTCCTCCGTGCCAAGAAC
ACCGTCGCGGAGGCGCGGCCAGCTTCCCTTGGATCGGACTTTCCGCCCCTAGGGCCAGGCGGCG
GAGCTTCAGCCTTGTCCCTTCCCCAGTTTCGGGGGGCCCCCAGAGCTGAGTAAGCCGGGTGGAG
GGAGTCTGCAAGGATTTCCTGAGCGCGATGGGCAGGAGGAGGGGCAAGGGCAAGAGGGCGCGGA
GCAAAGACCCTGAACCTGCCGGGGCCGCGCTCCCGGGCCCGCGTCGCCAGCACCTCCCCACGCG
CGCTCGGCCCCGGGCCACCCGCCCTCGTCGGCCCCCGCCCCTCTCCGTAGCCGCAGGGAAGCGA
GCCTGGGAGGAAGAAGAGGGTAGGTGGGGAGGCGGATGAGGGGTGGGGGACCCCTTGACGTCAC
CAGAAGGAGGTGCCGGGGTAGGAAGTGGGCTGGGGAAAGGTTATAAATCGCCCCCGCCCTCGGC
TGCTCTTC
SEQ ID NO: 188 (VWF Promoter Region)
CTTTCTTTTCCTTACGGTCTTTGTGTGTTTATATTTTAGGACTGTCTCTTGAACATTGCTGGAT
TGTGTGCCTTTTTAAAGAAATCCAATTTGATAATCTCTACCTTTTTATTATTGATTATTGATCT
GTTTGAATTTTTTGTACTGTTGCATTTACATTTTATACTATTTGTCTTGCTTTTTCCATTTTTC
TCCTAGATCAATCTAGTTTTTAAAAATTTCTTGTTTTTTTTCGATAGGTTTGGAAAACATACAT
TCAATTCTATTCTTTCAGTAGCCTCCCTTAAAATCTTACTATGCAGTATAGCCTTAACTAAGTC
TAAAGTGATCATGACCTGTTTCTTCCTTCTGAGCCATGCAAGGACCTTAATACACTTTCACTCC
TGTCTTACATGTCATTGCTATCTGGTATTTTGGTTCTAATTTTTAATAACTCTCCAACTTATCA
TTACCATTGTTATTGTTTTATATAATGACTGCCTGCTCATATTTACCCCCAGATTTACTATTTT
ATTTATTTACCTTTTCTTGGCATCCTGTTTCTAGGCTGAATTTTCTTCTTCCTGGCAGGGCGAG
GTGGCTCACACCTGTAATCCCAGCACTTTGAGAGGCCGAAGCAGGAAGACTGCTTGAGCCCAGG
AGCTCTAGGAGTTTGAGACCAGCCTAGGTAACATGGCAAAACCCTGTCTCTACAAAAAAATACA
AAAATTAGCCAGGTATGGTGGTACATGCCCATAGTCCCAGCTACTAGGGAGGCTGAGGTGGGAG
GATCGCTTGAGCCCAGAAGGTTTAGGCTGCAGTGAGTTGTGACTGTGTCACTGCGCTCCACCCT
GGGTGACAGAGACCCTGTCTCAGAAACAATAACAAAAAATGTTTTTTCTCCCTGAAATTCCTTC
TTTAGTAGTTTCTTCAGAAAGGACTGTTAATGTTAAGCCCACTCTGTTTTTCCAAAAAAAAAAA
AAAAAAAAAAGTCTATGTTTCCTCAACTCTGAACAATAATTTACAGGTAGACCAGATGTTACAG
TCCCTCTGCCACGGCCCTCTAACCCCATCTCTCCGTTTCTTCTAGACTACCAGTGTCTGCTCCT
CCGTGCCTGAGGGCCAGAAGTGTTGAGGGCTGAATGCCTCCTGAAGCAGCCTTGACAATAACTG
ACAGGAAGGAGTAAGTGTAGAAACTCTCCTGCTCCCTGACCCAAGAGTGGGAAGATTCAGAGGC
ATGGATCTTACATTTTCCCAGACTTCCCTCCAGAAGGAAGCTTTCTTGTCCACTTGGTGGCTGG
CATAATAAACATCTTTTATTGACTGCTTTCCCTGCCCCGTATCACATTCCCACTTCCCTGCCGA
TGCTTCCTGTCCTCCCAAATCAATTATCTACACTAAAATACTCAGGGTCTGCTTCTGGGACTTC
AAACTAAGACACTGCATATAGAATTCTCCACAAATGGACTTCTTCTCTCAGCACTTCTCTTTGG
ATCTTCTAATTCTATCATGTTTCTTAATTCTGCCTCCATTTTTTTTTTTTTTTTTTTTTTTTTT
TGAGATGGAGTCTCACTCTGTCACCCAGGCCAAAGTGCAGTGGTGTGATCTTGGCTCACTGCAA
CCTCTGCCTCCTGGGTTCAAGCAATCCTCCTGCCCCAGCCTCTTGAATAGCTGGAATTACAGGC
ATGCACCACCATGCAAGGCTAATAGTTGTATTTTTAGTAGAGATGGGATTTCAACACGTTGGCC
AGGCTGGTCTCGAACTCCTGACCACAAGTGATCTGCCTTACTTGGGCTCCCAAAGTGCTGGGAT
TACAGGCATAAGCCACTGTGCCTGGCTGCTCTTCCATTTTTTCATCTCTATATTTGAGTGATTT
CTTTAGAACCATTTTTCAGCTTTTTTTTTCCCAAAAATATCTGGTGTTTTTGAATTTTTCAATT
AATTTACTATATACATTTCTTAAAGTTCTTTTTTTTTTTTTTTGGGACAGTATCTCACTCTATT
GGCTAGGCTGGAGTGCGCGATTATGGCTCACTGCAACCTCAACCTCCTGGGCTCAAGGGGTCCT
CCCTCCTACCTCAGCCTCCTGAGTAGCTGTGACCACAGGCATGCACCGCCAGGCCCAGCTAATT
TTTTATTTTTATTTTTTGTAGAGATGGGGTCTTGCTGTGTTGTGTAGGCTAGTCTCAAATTACT
GAGCACAGACAATCCTCTTGCCTCTGCCTCCCAAAGTGTTAGGATTACAGGTGTGAGCCTCCAC
GTCTGGCCTTAAAGTTCTTTTTCAAGTTTTCCTCCTGTTCTTTCTTTTTTTTTTTTTTTTAACG
GACTCTCACTCGGTCACCAGGCTGGAGTGCTGTGGCATGATCTTGGCTCACTGCAGCCTCCGCT
TCCCGGGTTCAAGCGATTCTCCTGCCTCAGCCTCCAGAGTAGCTGGGACTACAGGTGCGTGCCA
CCATGCCCAGCTGATTTTTGTATTTTTAGTAGAGATGGGGTTTCACCATATTGGCCAGGATGGT
CTTGATCTGTTGACCTTGTGATCCACCCACCGTGAGCCACCGTGCCCTGCCTTGAAAAAAGTTT
ATTCTTTGTAAAGACAGGGTCTTGCTGTGTTGCCCAGGCTGGTCTTGAACTCCTGGTCTCAAGC
GATTATCCCATCTCAGCCTCCCAAAGTGCCAGGATTATAGACATGAACCACCGTGCCTAGCCTC
TATTATAATTTTAAACACTCATTTTTTTCTTTTTATCTTTCTCTGATGATTTTATTATCTGAAA
TTCTTAGGGATTTAATTCTTGCTCATGGTAGATTGTTTTCTCAAGTGTTGAAAAATTTGTGATT
TTGAATTCATCGTCAAGAGAGCTTTATTTGCATGAGTGCAAAGGATGAAAATTCTAGACTGGGC
GTGGTGGCTCACGCCTGTAATCCCAGCACTTTGGGAGACCGAGGTGGGCAGATCACGAGGTCAG
GAGTTTGAGACCAGCCTGGCTAACATAGTGGAACCCCATCTCTACTAAAAATACAAAAAATTAG
CTGGGTGTAGTGGTGTGTGCATGTAATCCCAGCTACTTGGGAGGCTGAGGCAGGAGAATTGCTT
GAAGCCGGGAGGCAGAGGTTGCAGTGAGCCATGATTGCATCACTGCACTCCAGCCCAGCGGACA
GTGCGAGACTCCATCTCAAAAAAAAAAAAAGAAAGAAAAGAATATTCTAAAAAAAGACTTAATT
CCCCCCGCCACCCCACCCCAAAACAAGTGGAGACAGGCAAACTTCCTTATCTTCTAGGTTGGGG
GATGGATTTTTTTCCTGGTCCACTGTTTGGAAGATGTTTCCCTTCAAACTTTCAGCTTTTGCAG
GGATCTCCGTTCTAGTTCTCCCTCTGGGTCAGGCCCGTAGCTGCACTGCCCATTCTTGTAATGT
GCGGCCTCCAGTCTGGAGGGTTCCCAGACTGGCCTACGCTAGGCCACCCATGGGCCTACCCTGC
CTCATGCTCATTTAGGCTCCTCTTCCTCATTGACCCTTTAAGATATTCCTTACTTTCCTCCCAG
ATCAACTGTGGATTTAAAGAACATTTGTTGTATTTAGCACAGCATTTAAAGATATTTTGTAATG
AAAGGGTTTTCAGATTAGTTATTTAGTTTTTTTAAATAAGAGCTGGAAGTGGAAATCCCGATGG
CCTTTTCTTTTCTTTTCTTTTTTTTCTTGAGACGGAGTCTTGCTCTGTCACCCAGGCTGGAGTG
CAGTGGCTCGATCTCAGCTCACTGCAAGCTCCGCCTCCCGGGTTCACGCCATTCTCCTGCCTCA
GCCTCCCGAGTAGCTGGGACTACAGGCGCCCGCCACCACGCCTGGCTAATTTTTTGTGTTTTTA
GTAGAGACGGGGTTTCACTATGTTAGCCAGGATGGTCTTGATCCCTTGACCTCGTGATCCGCCC
ACCTCGGCCTCCCAAAGTGCTGGGATTACAGGCGTGAACCACCGTGCCCGGCCCCCCAATGGCC
TTTTCTACTGTCTCATGCTGATTCTGCCTCTGGTGCCATTTTTCTTCCTTGGGAGTGTGCATCT
TCCTCTCCTGGGGCGGTAAAGGGAGTAGCAGAGTGCGAGGTATGTGGGAAGGGAGGAGGTTGGA
ACCTAGTGGTTTCTCAAAGTCTTAGGGCAGGAGGTATCATGGAGAAGCAGTGAGGAGGTCTTCC
ATACCCAGACATGCCTCAGGGTGCTTGTCTCAGTGCCTGGAATCCCAGCACGAGAGTCATCTTC
CCCCCACCGCTGCCCATTGCATCAGTTACTTATTTTAGTAGGAATTAGTTTAGCAGATGGTGTT
GAGAATTAGGCTTTTGGGAATGGGAGGCTGGGAAGAAGAATTGTGTGTGTGTGTGTGTGTGTGT
GTGTGTGTGTGTGTGTGTAAGATCAGGGTACCAGAAGTGGGTGGAAATGTCCTTGAGAATTAGA
ATTATTAGAATGTAGCAACAGTAGAAGTATTAGACTCAAACCATCACTCCCCACCTTCACCATT
TTACAAAGGCTTAGGCTTGTGGCCAAGACCTTCATCTTTAGCCGATCCATTCAACCCTGGCCAG
GATCCAAATGGACTGTTTTTGTCAGGGCCAGGACCGGATCCTTCATACCTGGGGTGCATAGGAA
GTGTTAGTACTCCCCTTCCTCCAAACACAGCAGCAAAATTGGCTCAGGTTGAGGTGTTTTTCTC
AACTTCCCTGGAGTCCAGCCCTGGAAGCTGGATCAGGAAGCTGTGTTGTTCTACTGTGATTCCC
CCTGGCCTGTATCAGCTTGCCCTGAAACAACCAGCATTCCTGGTTATCCCACACAGGTGGGGCA
CTCTAGGAAGACCAGGGATCAAGTGTGGGGGTGTAGGGATAGGGGGTGTTTGGGGAGGGCAAGG
CAGTTAATTAAGGCAGCTGCCAGGAGGTCTCCCTCCAAACTCTACAAAGCTTTATCAGCTTGGA
GGTACTTCTAATACCATTTCCTTTCATTGTTTCCTTTTGGTAATTAAAAGGAGGCCAATCCCCT
GTTGTGGC
SEQ ID NO: 189 (MGP Promoter Region)
AAACCAGAGCAAGTGTCAGATGACAGCAAACATCGGCGTTCACAGACTGGATGAGACTTGGTGA
TTCCTGAAGGAAGCTGAAAGGTTTTGGAAAGTACCATATTTGAGTTGGAGCTTGTTTGGGCAGA
TGTTTAGTGTGCAGTGTACTGGAGAGAGAAACAGGGGACTTAGGATCCAATATTAAAACTATCC
CTCTCTCCATTTTTATAAGTCATTTAACTCACTGGGCTTAAGTGTATGTAGACCTTTAATTTGT
ATTGCACTATTTTAAAGGATTTTGAGACAATTATCATTTTAAATGTGTATATCTTAATTTTGTG
TAATTTTGTCTCTAGCTATGAAATTTGAAATTCAAGGAACCCTGTAAGACGCAATCACAAAATT
ATTTAAGCAGGTGTCAGAACCTGGAGGGATCTCACATGGTTGTATCATTATTAAAAACACACGA
TTTGACATCAAACAGTCCTGCGTTTTAATCCTGTCTTTGCCATTTAGTTGTTGTGTGATCTTGG
ATAAACTATTTTACCTCTTTGAGCTTCCATTTTCTTATATATGAGATGTGGGTCATTATACTTA
GCTTATAGGATTATTAAGAGAAGTAAATAAAAAATAAAGAGTGACTATCAGAGCATCTAATATA
TAGCAAGCATTTGACATATGATAATAGCGCTTGGATAGATGTATAATTTTTATTTGATATTTGC
TATCTTATTTAAAAATTGAAGCTTTTCTTAAGAAGGGTGGAGGAAATAATCGATAGGGGAGGAA
AAGAAAAATCCCAGCCATTCCCAGCCAAAAGCTCTCCACCTCTCAGACTTCTCCACCTTTATTT
TGTTTAATAATCAGGCACCTCCAACTTTCCCTGGAAGTTTCAGCCCATGAGTCCCCAGATGCTC
CTCCCATGCCTGAGTTTGACAAACATTTATTCTTACTTAGTTCAACTAAGACAAATTAAGGAAA
GGAAAGAAGGATTCTATCTGTGCCTAGGAAGTTATGTCAGTTAAAGAATCTGACACACAGAATC
CTCTCAAGGGGCTTCCCTTAAAAGAAAAGAACTAAGAGCATGGCACTGTCCCACGACATTGGCT
GGGCTTCTGTCTCCTAGTTCATTGTTCTTTGATGTCCTAGAGAGTGATTTCTGTTTTTGTCTTC
AGGGCTGCTTCTTCCTAACTCTTCTTTCATTTGCGTTTTTCCTTTAATGGCAAGGGTAAGGACT
TTAGTGCTGCCAGTTTAAAAAAAATTATAAAATCAAAATTCAGTAAATAAAGCTGAATTTCATG
GAACAGCAAAAAAGAAAAAGAAATCTGACTGGAGCCAGGAAAATTCCTCAAAGAGACAAAATGC
CTAGCCATCTGAAAAAGAGTTAGTTAAAGGATAAGCAAGGACAGAAAGAGAAGGAGGTAGAATT
GATAGGGAAATATATTACACTTTCTACCTCTATGCCATTATTTTAGTCACAGAAATACCATGTG
AAAGGGATAACTCTTTTGGTATATTCTCTGGCAAATATACTTTTCTGAGAAGCTTCTCCCCACA
GTAATTCTCAGAAGCTCTGCCAATGTTCCTCTCCTGCATTCCCTCCCTAAATTCAGAATTATCT
GAGCTGGTCACTAGCACCTTTCTTGATGCTTTGGAAACAGAGATGCTCCTGAGTGCCCATAAGA
ATGGTCAGCCCAGTGAGAAAGCTCATCACTTGGTCTCCTTTAAGGCCAGTTGGCTGCCTAACAA
TTTTTTAAATAAGAGGAGCCACTATTAAATTTTTGTTCAAAGAGCACACTTGATGCATGAGGAC
AGGGCCCATATCTGTATTTTTCTCTACTGTATTTCCAGCCTAGAGTTGACAAACAGTAGATGCT
CAGTACATTTGTTGGCTAGATAGATAACTTGATGGATGGCTGGCTGGCTGGCTGGCTGGATGGA
TGGATGGATGGATGGGAGAATTATGAAATCATGAAGCTCCTTCTGGCCCTGACAGGCATGGTCA
TTCTTCTCTTTTCTGCCTGAGAGTAGGTGGAATAGGAGATCTGTATTACTCCATGGCTTCTCTT
GCTTCAGTTCCTACGTTGCCAACCTCACATGAGGAGAATCCTACACATGTTTAAAAACTGGCAA
TCATATCACTGTCTCATATTTCTGTTATCACTTCTGGGAGTTTCTTCAAATATTCTCTCCTCTG
AATAACACTTCTTTTTTGTTAAGGGAAAATGTCTATATAAGTGTCTTTCATAATTATCTAAAAT
CTAATTAGAATTTAGAGTTTCATGTGGTCTCGTCTTGACAAGATATCCCAATTAAGAAAATGCA
AACTAGCTGGCAAAATTAATTTGTTCAAATTTCAATATTTTCTGAAAATTTTCAGACAGTATTC
TGCAATCTCAAACAATGCTATTCCTAACCAAAGCAACTTTTATTTCTCTGTTCCCATGTCTCGC
TTTTAATATGTCTCACCTTCTACAACTGCCTCCGTTTTTCTCTGTCACTCAGTCTCTACCTAAA
ACTCACCCAGCAAACCAAATTGGTAAGGCTCTTCTCATTTCCCCTTCTCCGTTTTTTTTTTTTT
CCTACTTCCATTCTTTTCTTCTGTCTTCTCTCAGATGAGTCAATCTTGGTCCTTTCTAATGCAA
AGCTCCCATCCCTGCTTCATGCGTTAGTCCAAGTCCTCATCATAAAAACATATGACTGGAGTTG
GCATTCACAAAGTTGTCTTTGAAATGGGGAGTAAGGTGACAGAGGAGAAAAAGAAGAGCTCTGG
ATTCTCAGACATGTTAATAATTTTTACATATCATATATAAATGGGATTTTGCAGAGAAGAACCA
GAAATAGATGGGAGAGCAATGGACAGGAAAGGCAGATGAGGGACCGAAGAGACACAGCTCCCAA
AAGAAAGTTAGCCTTACAAAAACCAAGACGATAAAGAGAAATGCTTAAGTTTAGGGAATCCAGT
GGAAGCAGTGATTTAAGGTGAACAAAAGGTGAACCTTAAGTTGAAATGAGAAGTGTAGGATTTT
CAAGTTTAGTTTCTGGGAGTGTAAAAATAAAAAAACAATTGTGATGTCAGAGGTTGAAAGATTA
TAGTTGTCATTTGAACTTGGGGATAAAGGAGACATCTATGACTTGGCTGGAAAAGACAGAGCTA
ATGTACATTGCAAAGCACATATTTATAGCAGGAAAATGGGAAGATTTCTCTTTAATTCTGGAGA
TGGAGTGGGGATGGGGAGAGTAGACTACTCATTTTAAGGGTGAAACATTGGAATTCAACTTGTT
TGATGTTATATTAATTGGTGGTTAATTACTAAGCTAAGTACGTATAAAACTTTTATCTATGGCT
AGCTTGTCCCCCCAAAGTCATGCAATATAGTGAACTGGCTTTCGCACTTTAAATTATTCATTGA
TCATGTAATGATTCAGATGATTCATCTTCCAAGATGGACACTGAAACTAACACTCATAGTAGGT
TGTGGTTTAAAGAGTGGAACAACCGCCAGTCTCATTAGTGGAAATTGTGATGGTTGAATTTATC
AAGGATGAACATACACGGTCTTCTTTCTGAGATTTTCTTTAAGATTTTCGCACAGATAATCTAT
TTCTTAGGTTTTGGAGAGAAAACTTGAATTTTATTGATCCCTCAGAACTCAATCTTTCAGATTT
CAAAGGAGCTATTTCTTTTAATGGGGACTCTGTTAATATTTATAAAAGCTCTTCACAGGATGGA
GGGTGGGAGGGAAACTCCATCCCAACAAGACAAAAAGAATGAAGCATGAGGCTCCACCTAGTTC
ATCACTGCTCCTTGAAATACATCAGTATTGAAAGACACATCCACCCCACCCCCAACCCAGCCCT
ATTGCTGTTCCAGCTCAAGAGTCAGAGGTCCCGAAGCTGTAGCTCTTCTACAATCTGCTGCTCT
GTGACTTCAAGTCTGTTGTCTGCAAAGAAAACTATTGGGTTCCCAAGCAAGAGAGGCACATCTG
GTAGGACAGATTTTGTGATTGCAAAAGAAGGGGGAAAAAAAGAAAGAAAGAAAAGACCTCTCTA
TACAAGATAACCAGAGGCATCAAACTGAAATCCTCCTGTGGAAAATAAGCTAGTACTTCTGGGC
CTGATGGTGTAGTGAAAACCTGTGCTTGAGGATACATTACAGTGAAAGAGCAAAGTGAATAGTA
AGTAGCTATTACTTACCTCCTTAGGGAGGTGTGTTGTTTGTCTGTACATCCCCCACAGCACCTA
GCACAGTACCTTGCATCTCACCTGCCACTCACTAAAAAGTCTATCAAGTTAGTTAATTATCGAG
ACAACGCCCTCAGAAATGAGAGAACAGTACCCTCTTATCCTTGCTGCACTTTCCAGCACTGATA
CGCTGCCTAAAAGAGGACTAGGGCACAGGTTTGAATTAATGTCACAAAACTGGATGGGCAAGTT
ACAACGGTGTTGATTAAGGAAACAGAACTCATGGTGCACCGGATATCTCCATCCTGATGAACCC
TTGGAAAAATGCCAAAGATGCATATCCCCAGGCAAATGCCTGATTAGTCTGGGATTGATAGATT
GGTCTAGGATTCAGCCCTACTGGGAAGATGTCTAAATTATAATCAGTGTAGAAAGCGAAGTTCT
CCTAGAAGAAGAGGCAAAGGTTAAAAAGAAGAAAAGAAAAGAAAGTGAAGTCCTTTCTCCCCCA
AAACCTCTCATCAATCAATCAGGGTAACAAACAGAACACTAGGGCTCTGTCTGTGGACCAAACC
CAAAAGCCCTGCGGTCAGGGCCAGGAGGGTAGATCATGTGTTTGTGGCAACTTCCTCTGTGGGC
TTTTGCCCAGGTCTGTCCCCAAGCATACGATGGCCAAAACTTCTGCACCAGAGCAGCATCCTGT
GTAACACAGTCAGGTCCAGCAGTTAGGGAAAACTGCCCACTCAGAGTAGATAATATCTGGAAGG
AATGACTGTTTGGGAAAAGTTCCAATGCTAGTTCAGTGCCAACCCTTCCCCACCTTCTCCAGCT
CTCTCCCACTGGTTCCTCCCCTCTCAACTGCTCTGGTTCTTATAAAAACCTCACAGCCTTCCAC
TAACATCCC
SEQ ID NO: 190 (RGS5 Promoter Region)
AATCTTTCTTTTTTTTTGGTATGTAGGAAGATTAAGATTTATATTTCTGTTCTAATGGTTACCT
TTGTACATACACTTTTCTTAGTGTCTTTCATCTCCTGTTTTCTTATTTAACCTGTTATCTGGTT
TATCCAATTTTATTGGTATCCTTTAACTGCTTCTGCAACAATCAATGATCTAATTCTACTTTTC
TTCTTTCTCCCATTTTTAGTTTCATTATTTTTATAAAGAATTCTTGCACACCAATATTAAAAAG
CCAGATAGTAAAGTTGTTTAAAAAATGGAAATATACAACTAATGGAAGAAAAATTACAGTGGCC
AATGTGTATATGAATAGAAGCTCAAATTCCCTAGTAATTAGAAAGGCACAAATGAAAATGACTT
GGTATTTTTTGCCTGTCACTGGAAAAAATTAAAGCCTGGCAAGTGTTTCATATTTGGGAAAATG
AGGCTCTCATTTATTTCTGTTAGGAGTCTAAATTGGTCTGACTGTGTGATAATTTTGTACTGTT
ACACCTCTCAGCTGTCCCACTTTATGAACTACATTATAGATAAACTGCTCCGGGGGCATATATA
GGAATATTAATTTAAGTACTTTTGCCAGAAGCAAACAATTAGAAATGACCTAAATATTTAACAA
TGGGTCTATAAAGAAATTCAATTATATTTGCATATGATATAATTCTTTCCAGCAATTAAAAAGC
ATAAACTGGAGGTATAGACTTTACACTTCCTATGTTAGTACCGATGCTTTTTAATCCTGAACAA
TTTCATTTCCATTTATATGATCAGAGGGTTTTCTCTTTGAAATAGCAAAAATTATACCACTTAT
CTCATGCTTTTGTTGTGAGAATTAAATGTGTAAATATATGAAGTGTTTTAAAAAGTGATTAATA
CATAGAAATTATTCAGTAGTGTTAGTTGTCATTATAATCTAAAAACAAAAACAAAAAATCATAA
TCTTAAATAACAAAAGATGCAGATTATATTTATATAAATCAAAACACATACCAATAATTACTAT
ATACTTTTTATGAATTCATATTGTCTTAGTTTGTGCTGCTATAACAAAATACCTGAGACTGGGT
GCTTTATAAACAAAAGAAATTTATTTCTGGCAGTTTTGCAAGCTGAGAAGTTCAAGATCAAGGT
GCCAGCAGGTTCGGTGTCTGATGAGAGCTGCTCTCTGCTTCTCCAAGATGGTGCCTTGTTGCTG
TCTCCTAACATGGCAGAAAAGCAGAAGAAAATGAACCCCCTCCCTCAAGCCCTTTTATAAGGGC
TCTAATCTCATCCATGAGAGCTTTGACCTCATGGCTTAATCACCTCCTAAAGGCCCCACCTCTT
AATATTAACACATTGGTGATTATTAAGTTTTAACACATGAATTTTGAGGGAAACGTTCAGATCA
TAGTACATATGTATATGAGGACATTAAAAAATGACCTGGAATACCATACACGAAAATCTTAACA
GGTACTAAGTGTGATGAAAGGGGAAAAATAATGGACATGAGAGTCCTCTTTTTTTCATTAAAAA
AATCAAAAAGCAACATAAACTATATACTTTTAATGAATCCATATTGTCTTAGTTTGTGCTGTTA
TAACAAAATACCTGAGACTGGGTACTTTATAAGCAATGGAAATTGATTTCTCACAGCCCTGGAA
GCTGGGAAGTTCAAGATCAAGGTGCCAGCAGGTTCAGCAGGACTATATTTAATATAAAGTCCAG
GGACTTTAGTTTTATCCATTGCCTCTTTATTTCTTTAAATAAAAAAAGGCAGGGAAGCAATATC
ACAACCTATTGACAGTATATATTTCTGAGTGGTAGAATTATAAATTGAGCTTGCTTATGGTTTA
AAACATTTAGCAGAAAGACAGAAATCAAGCAACATGAAGGTGAAGACCTTGCCTCTGTTTACTC
CATGTGCTCCTAGCTGGCTTTAGGGTGAGACTCTTACTGTGAGTAGCGAGATTAGTGAATAGAC
GTCTGTTCACCATGGATCACATAGTCCAAGAGGTCCCCAAATTAGAAGTGATAAAATGCATCTC
AATAAATATATTGCTAATAATAGTTAAAATAAAGACACTTACACATGATTTAAGGAAATGAATT
AGAGATGAATCAGAGCAGTTGTGTGGGCAGGAGATCCTTTTCATCTGATGAAAATGTTCAATGT
CTCTCACTGCTTCCACTTGCACAGCTAACTCCTTCCTTCTGTAGAACTGTAATATCTAGTGCTT
TGGAGCCCAAACCACTGTGGCATCCTCCAGGGCAGATGGATATCTCTTTGAAGCACCCCCCTGA
ATTCCCTCCCCTGTTTCATTAGGTACCACTCCTACTCACCTCTTTTCACATTCCATGAAGAAGT
GGTTGAAATCACTCAGAATATGATGGCTTGTTTCTCTTTTTCTTTGCTATTACAGATTTATACC
TTTTTCATATATTTACTGTCATTTCTGAGGGATTCTCAGGAGGGAAAGGAGATTTTAAAATAGT
TCAATTTATTTTTTCAGACTTCAAATTTATTTTACGTAGTAGGCTCTCTCCTTTTTTTTTTTTT
TTTTTTTTTTTACTGTGGTTAAAAAACACGTAACACAAACTATTCTCTTCACAGATTTTTAAGT
GTACGGTACAGTATAGCTAACTATGGGCACAATGTTGTATAGCAGATCCCTAGAAATTTTTCAT
TTTGCATAACTGAAACTTTATACCTATTGAAACTTTATTGAATAGGAACTCCTCCATTTCTCCC
ATTTCTAACCTCTAGCAATCACTATTTTACTTTCTGCCTCTAGAGCACACTCTTTTACATACTT
TCTGGCTATCCCATCCTTACAAAGTTTTAAAAACAAATAAGTGTTGGATTTAACATTATTTCTA
TTGAATTTTACATTTTGGGTTCTAGTCCATCTTTTAGTAAATTGAGATAATTTTTATTCTTGAA
TAGTCCATCACATGAACTCCTTTGGGACAGTCAGAAACATGCTTTGATTTTTAGCCCTCGCCAA
GACATCACCCTCCATTGCTCTACGTTAAACTTGGGGTTTTTCTTTTCATATTATTATCTCCTCA
AGGCCCCTTGGTATCAAATAATGATTTAGACTCTTGTCCCAAGGAGTCTCTTCTACTTGTTTTA
TATTCCCTTTATTAGATTTAATAGTCAACAACAGTGTCCACATGTCTCAAAAATGATAATTAAT
TGATTTGCTGATGTTTACTCAGTCAATAGATGGCGGAACTTGGACAGTTTGTTTAACTCATTCA
GTATTTACAGATGTCTATGTGCCTGACATTGTGCTTAGTGTTTGGAAGGCTGAGGCAGGAGGAT
TGCTTGAAGCAAAGAGTTCAAGACCAGCCTGGACACCAAAACTAGACCCTGTGTATTCAAAAAA
TTTAAAAACTAGCTAGGCATGGTAGCAAGAGTCTTTAGTCCCAGTTACTTAGGAGGCTAAGGTG
GGAGGATCGCTTGTGCCCAGGAGTTTGATGATGCAGTGAGCTATAATCATGCCACTTCACTCCA
GTCTCTGGTCTGGGTAACAGAGCTAGACTCTGTCTCAAAAACGTGTGTGTCTGTGTGTGTGTGT
GTGTGTGTGTGTGTGTGTGTGTGGTTTAGAGCCACAGCTTTCAAACTTTTTTGATTATGATCCA
TAGTAAGAAATATAGTTTACCTGATGACCCTGTGCACACTTACATATTGATAATTAAAGTCAAA
ATTTCAGAAAACAATACTTTTTACATCCGATGCACTTGGATATTTGTCTATTCTATTTATTACA
TTAAAAACATCAGGTCATAATGCACTGCATTGACTTTACAAGCCAGTAACAGGGGTTGATCAAC
CATTGCCTAAAAAGGGTATATAAAGAGGGAGTCATTTAAAACAACTAATTTCAGATAACAGAGA
AAATTATGAGAGTACTGTTCAATTCCAAATTCAGAGCTTTTCTTACGGATTATTCAGTGCCACT
TTTTTTAAAGCTTGGACTCATGCCTACCTTTTAGGCATTTATTTACAGATTAACTTATCAGCAC
CTTGGGCCCCGACAAGATAAGACACAATTCAGGTAAATTTTGGTAAATATTCCTTGAGTGATAG
GAAGCAGGAAAAGTTTGAAGGAGAAGGAAAGACAAGCCTAGGCAAGGATGGCTATGGTGGGGAG
GTACAAAGAATTACAAAGCATTAGTCATCCAAGCAAAGTATAATCAGAAGACCACTGACAGAAT
TAACAGAGTTCAGAGATAATATCATTATACTCAATCCCTACTAAGATTTTGGTGTTTGGTCTCC
TAGATCACTTTTTTAGTACCTTCACAGACATATGGATTCATAAAGAATATATAGTAATTATTCA
TATTTGTTTTGATTTATATAAACAGTAATCTTGTCTCTCAGCTCCAGAAAACCTTGCCTGGTGC
TTCCTGAAGGCTCACAACATCCCAGCCCGGCCTCCATCACTTACTTATAATGTGTTTTGGGGTC
TTGAGGGCAGGGAGCTTTCTCTTTCTTCTCTGAATTCCTAGCATCCAGCTGAGTGCCTGGCACT
TGGTAAGTATTCACATACACATTCCTTGAATTCCCTAGATTTCAGTTTCCTTCACTGTGAAACA
AAGTGGGCTAAACGATCTCCAGGAGCCCTTACAGTCCTAAGAATCTATGACTTTGACCGATCTG
AAGTGGTCCAAGACACTTTGCTTCACAGGAAGCTCACATCTTCCTTGTGATTGGCCTGAGATTA
GGAGTTCAAACAACCAATGACCTGTGCACAGTTTAGGGCCTAACCCTGCCCTGGCCGGTTAGCA
AGAGAGTGTTGTGATAAGAGCAGATGAAAGCCTTATGCCACAGTAGTGCCTGTAGCAGAGAAAG
GATTCCCTCCTCCACCTCAGGGCCCACTGCTGCTGCTCTCCAGCCTGCAGTTTCCTCTAAGGCT
CTGGATTGGCTGGAAGAGCAACAGAGGGCTGGGAAAGAGCTTCTATATATACCTCAGGAGGAAA
GGCATCCC
SEQ ID NO: 191 (SFFV Promoter Region)
TGAAAGACCCCACCTGTAGGTTTGGCAAGaTAGCTGCAGTAACGCCATTTTGCAAGGCATGGAA
AAATACCAAACCAAGAATAGAGAAGTTCAGATCAAGGGCGGGTACATGAAAATAGCTAACGTTG
GGCCAAACAGGATATCTGCGGTGAGCAGTTTCGGCCCCGGCCCGGGGCCAAGAACAGATGGTCA
CCGCAGTTTCGGCCCCGGCCCGAGGCCAAGAACAGATGGTCCCCAGATATGGCCCAACCCTCAG
CAGTTTCTTAAGACCCATCAGATGTTTCCAGGCTCCCCCAAGGACCTGAAATGACCCTGCGCCT
TATTTGAATTAACCAATCAGCCTGCTTCTCGCTTCTGTTCGCGCGCTTCTGCTTCCCGAGCTCT
ATAAAAGAGCTCACAACCCCTCACTCGGCGCGCCAGTCCTCCGATTGACTGAGTCGCCC
SEQ ID NO: 218 (CFHT-18-bp spacer-IRES (551-bp)-45-bp spacer-Aflib)
GTACCACTGTGCATATGCGTTACTGGCCGAAGCCGCTTGGAATAAGGCCGGTGTGCGTTTGTCT
ATATGTTATTTTCCACCATATTGCCGTCTTTTGGCAATGTGAGGGCCAGGAAACCTGGCCCTGT
CTTCTTGACGAGCATTCCTAGGGGTCTTTCCCCTCTCGCCAAAGGAATGCAAGGTCTGTTGAAT
GTCGTGAAGGAAGCAGTTCCTCTGGAAGCTTCTTGAAGACAAACAACGTCTGTAGCGACCCTTT
GCAGGCAGCGGAACCCCCCACCTGGCGACAGGTGCCTCTGCGGCCAAAAGCCACGTGTATAAGA
TACACCTGCAAAGGCGGCACAACCCCAGTGCCACGTTGTGAGTTGGATAGTTGTGGAAAGAGTC
AAATGGCTCCCCTCAAGCGTATTCAACAAGGGGCTGAAGGATGCCCAGAAGGTACCCCATTGTA
TGGGATCTGATCTGGGGCCTCGGTGCACATGCTTTTCATGTGTTTAGTCGAGGTTAAAAAACGT
CTAGGCCCCCCGAACCACGGGGACGTGGTTTTCCTTTGAAAAACACGATGATAATAGAATTCGA
TATCAAGCTTATCGATACCGTCGACTAGAGCTCCACC
SEQ ID NO: 219 pCTM398
GCGCGCTCGCTCGCTCACTGAGGCCGCCCGGGCAAAGCCCGGGCGTCGGGCGACCTTTGGTCGC
CCGGCCTCAGTGAGCGAGCGAGCGCGCAGAGAGGGAGTGGCCAACTCCATCACTAGGGGTTCCT
TGTAGTTAATGATTAACCTCTTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGAGTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGTCGAGGTGAGCCCCACGTTCTGCTTCACTCTCCCCATCTCCCCCCCCTCCCCACCCCCAATT
TTGTATTTATTTATTTTTTAATTATTTTGTGCAGCGATGGGGGCGGGGGGGGGGGGGGGGCGCG
CGCCAGGCGGGGCGGGGGGGGGCGAGGGGCGGGGCGGGGCGAGGCGGAGAGGTGCGGCGGCAGC
CAATCAGAGCGGCGCGCTCCGAAAGTTTCCTTTTATGGCGAGGCGGCGGCGGCGGCGGCCCTAT
AAAAAGCGAAGCGCGCGGCGGGCGGGAGTCGCTGCGCGCTGCCTTCGCCCCGTGCCCCGCTCCG
CCGCCGCCTCGCGCCGCCCGCCCCGGCTCTGACTGACCGCGTTACTCCCACAGGTGAGCGGGCG
GGACGGCCCTTCTCCTCCGGGCTGTAATTAGCGCTTGGTTTAATGACGGCTTGTTTCTTTTCTG
TGGCTGCGTGAAAGCCTTGAGGGGCTCCGGGAGCTAGAGCCTCTGCTAACCATGTTCATGCCTT
CTTCTTTTTCCTACAGCTCCTGGGCAACGTGCTGGTTATTGTGCTGTCTCATCATTTTGGCAAA
GAATTCCTCGAAGATCTAGGCCTGCAGGCGGCCGCCACCATGAGACTGCTGGCCAAGATCATCT
GCCTGATGCTGTGGGCCATCTGCGTGGCCGAGGACTGCAACGAGCTGCCCCCCAGAAGAAACAC
CGAGATCCTGACCGGCTCTTGGAGCGACCAGACCTACCCTGAGGGCACCCAGGCCATCTACAAG
TGCAGACCCGGCTACCGGTCCCTGGGCAACATCATCATGGTGTGCAGAAAGGGCGAGTGGGTGG
CCCTGAACCCCCTGAGAAAGTGCCAGAAGAGGCCCTGCGGACACCCCGGCGATACCCCTTTTGG
CACCTTCACACTGACCGGCGGCAACGTGTTCGAGTACGGCGTGAAGGCCGTGTACACCTGTAAC
GAGGGCTACCAGCTGCTGGGCGAGATCAACTACAGAGAGTGCGACACCGACGGCTGGACCAACG
ATATCCCCATCTGCGAGGTCGTGAAGTGCCTGCCTGTGACCGCCCCAGAGAACGGCAAGATCGT
GTCCAGCGCCATGGAACCCGACAGAGAGTACCACTTCGGCCAGGCCGTCAGATTCGTGTGCAAC
AGCGGCTACAAGATCGAGGGCGACGAGGAAATGCACTGCAGCGACGACGGCTTCTGGTCCAAAG
AAAAGCCTAAGTGCGTGGAAATCAGCTGCAAGAGCCCCGACGTGATCAACGGCAGCCCCATCAG
CCAGAAGATCATCTACAAAGAGAACGAGCGGTTCCAGTACAAGTGTAACATGGGCTACGAGTAC
AGCGAGCGGGGCGACGCCGTGTGTACAGAATCTGGATGGCGGCCTCTGCCCAGCTGCGAGGAAA.
AGAGCTGCGACAACCCCTACATCCCCAACGGCGACTACAGCCCCCTGCGGATCAAGCACAGAAC
CGGCGACGAGATCACCTACCAGTGCCGGAACGGCTTCTACCCCGCCACCAGAGGCAATACCGCC
AAGTGTACCAGCACCGGCTGGATCCCTGCCCCCAGATGTACCCTGAAGCCCTGCGACTACCCTG
ACATCAAGCACGGCGGCCTGTACCACGAGAACATGCGGAGGCCCTACTTCCCTGTGGCCGTGGG
CAAGTACTACAGCTACTACTGCGACGAGCACTTCGAGACACCCAGCGGCAGCTACTGGGACCAC
ATCCACTGTACCCAGGACGGCTGGTCCCCTGCCGTGCCCTGCCTGAGGAAGTGCTACTTCCCCT
ACCTGGAAAACGGCTACAACCAGAACTACGGCCGGAAGTTCGTGCAGGGCAAGAGCATCGATGT
GGCCTGCCACCCTGGATACGCCCTGCCTAAGGCCCAGACCACCGTGACCTGCATGGAAAATGGA
TGGTCCCCCACCCCCCGGTGCATCAGAGTGTCCTTCACCCTGTGAGGTACCACTGTGCATATGC
GTTACTGGCCGAAGCCGCTTGGAATAAGGCCGGTGTGCGTTTGTCTATATGTTATTTTCCACCA
TATTGCCGTCTTTTGGCAATGTGAGGGCCAGGAAACCTGGCCCTGTCTTCTTGACGAGCATTCC
TAGGGGTCTTTCCCCTCTCGCCAAAGGAATGCAAGGTCTGTTGAATGTCGTGAAGGAAGCAGTT
CCTCTGGAAGCTTCTTGAAGACAAACAACGTCTGTAGCGACCCTTTGCAGGCAGCGGAACCCCC
CACCTGGCGACAGGTGCCTCTGCGGCCAAAAGCCACGTGTATAAGATACACCTGCAAAGGCGGC
ACAACCCCAGTGCCACGTTGTGAGTTGGATAGTTGTGGAAAGAGTCAAATGGCTCCCCTCAAGC
GTATTCAACAAGGGGCTGAAGGATGCCCAGAAGGTACCCCATTGTATGGGATCTGATCTGGGGC
CTCGGTGCACATGCTTTTCATGTGTTTAGTCGAGGTTAAAAAACGTCTAGGCCCCCCGAACCAC
GGGGACGTGGTTTTCCTTTGAAAAACACGATGATAATAGAATTCGATATCAAGCTTATCGATAC
CGTCGACTAGAGCTCCACCATGGCCTCTCATAGACTGCTGCTGCTGTGTCTGGCCGGCCTGGTG
TTTGTGTCTGAGGCCTCTGATACCGGCAGACCCTTCGTGGAAATGTACAGCGAGATCCCCGAGA
TCATCCACATGACCGAGGGCAGAGAGCTGGTCATCCCCTGCAGAGTGACAAGCCCCAACATCAC
CGTGACTCTGAAGAAGTTCCCTCTGGACACACTGATCCCCGACGGCAAGAGAATCATCTGGGAC
AGCCGGAAGGGCTTCATCATCAGCAACGCCACCTACAAAGAGATCGGCCTGCTGACCTGTGAAG
CCACCGTGAATGGCCACCTGTACAAGACCAACTACCTGACACACAGACAGACCAACACCATCAT
CGACGTGGTGCTGAGCCCTAGCCACGGCATTGAACTGTCTGTGGGCGAGAAGCTGGTGCTGAAC
TGTACCGCCAGAACCGAGCTGAACGTGGGCATCGACTTCAACTGGGAGTACCCCAGCAGCAAGC
ACCAGCACAAGAAACTGGTCAACCGGGACCTGAAAACCCAGAGCGGCAGCGAGATGAAGAAATT
CCTGAGCACCCTGACCATCGACGGCGTGACCAGATCTGACCAGGGCCTGTACACATGTGCCGCC
AGCTCTGGCCTGATGACCAAGAAAAACAGCACCTTCGTGCGGGTGCACGAGAAGGACAAGACCC
ACACCTGTCCTCCATGTCCTGCTCCAGAACTGCTCGGCGGACCTTCCGTGTTCCTGTTTCCTCC
AAAGCCTAAGGACACCCTGATGATCAGCAGAACCCCTGAAGTGACCTGCGTGGTGGTGGATGTG
TCCCACGAGGATCCCGAAGTGAAGTTCAATTGGTACGTGGACGGCGTGGAAGTGCACAACGCCA
AGACCAAGCCTAGAGAGGAACAGTACAATAGCACCTACAGAGTGGTGTCCGTGCTGACCGTGCT
GCACCAGGATTGGCTGAACGGCAAAGAGTACAAGTGCAAGGTGTCCAACAAGGCCCTGCCTGCT
CCTATCGAGAAAACCATCTCCAAGGCCAAGGGCCAGCCTAGGGAACCCCAGGTTTACACACTGC
CTCCAAGCAGGGACGAGCTGACAAAGAACCAGGTGTCCCTGACCTGCCTGGTCAAGGGCTTCTA
CCCTTCCGATATCGCCGTGGAATGGGAGAGCAATGGCCAGCCTGAGAACAACTACAAGACAACC
CCTCCTGTGCTGGACAGCGACGGCTCATTCTTCCTGTACAGCAAGCTGACAGTGGACAAGAGCA.
GATGGCAGCAGGGCAACGTGTTCAGCTGCAGCGTGATGCACGAGGCCCTGCACAACCACTACAC
CCAGAAGTCCCTGAGCCTGTCTCCTGGCTAAGAGCTCGCTGATCAGCCTCGACTGTGCCTTCTA
GTTGCCAGCCATCTGTTGTTTGCCCCTCCCCCGTGCCTTCCTTGACCCTGGAAGGTGCCACTCC
CACTGTCCTTTCCTAATAAAATGAGGAAATTGCATCGCATTGTCTGAGTAGGTGTCATTCTATT
CTGGGGGGTGGGGGGGGCAGGACAGCAAGGGGGAGGATTGGGAAGACAATAGCAGGCATGCTC
AGGTTAATCATTAACTACAAGGAACCCCTAGTGATGGAGTTGGCCACTCCCTCTCTGCGCGCTC
GCTCGCTCACTGAGGCCGGGCGACCAAAGGTCGCCCGACGCCCGGGCTTTGCCCGGGGGGCCTC
AGTGAGCGAGCGAGCGCGCACTGTCATTAGCAACTCCTTGTCCTTCGATCTCGTCAACAACAGC
TTGCAGTTCAAATACAAGACCCAGAAGGCGACTATTCTGGAAGCGAGCTTGAAGAGTTAACCTG
CAGAGAGCCCCCGCAGTGTCGACAATTAATCATCGGCATAGTATATCGGCATAGTATAATACGA
CAAGGTGAGGAAGTAAAAAATGAGCCATATCCAACGGGAAACGTCGAGGCCGCGATTAAATTCC
AACATGGATGCTGATTTATATGGGTATAAATGGGCTCGCGATAATGTCGGGCAATCAGGTGCGA
CAATCTATCGCTTGTATGGGAAGCCCGATGCGCCAGAGTTGTTTCTGAAACATGGCAAAGGTAG
CGTTGCCAATGATGTTACAGATGAGATGGTCAGACTAAACTGGCTGACGGAATTTATGCCACTT
CCGACCATCAAGCATTTTATCCGTACTCCTGATGATGCATGGTTACTCACCACTGCGATCCCCG
GAAAAACAGCGTTCCAGGTATTAGAAGAATATCCTGATTCAGGTGAAAATATTGTTGATGCGCT
GGCAGTGTTCCTGCGCCGGTTGCACTCGATTCCTGTTTGTAATTGTCCTTTTAACAGCGATCGC
GTATTTCGCCTCGCTCAGGCGCAATCACGAATGAATAACGGTTTGGTTGATGCGAGTGATTTTG
ATGACGAGCGTAATGGCTGGCCTGTTGAACAAGTCTGGAAAGAAATGCATAAACTTTTGCCATT
CTCACCGGATTCAGTCGTCACTCATGGTGATTTCTCACTTGATAACCTTATTTTTGACGAGGGG
AAATTAATAGGTTGTATTGATGTTGGACGAGTCGGAATCGCAGACCGATACCAGGATCTTGCCA
TCCTATGGAACTGCCTCGGTGAGTTTTCTCCTTCATTACAGAAACGGCTTTTTCAAAAATATGG
TATTGATAATCCTGATATGAATAAATTGCAGTTTCATTTGATGCTCGATGAGTTTTTCTAACTC
ATGACCAAAATCCCTTAACGTGAGTTACGCGCGCGTCGTTCCACTGAGCGTCAGACCCCGTAGA
AAAGATCAAAGGATCTTCTTGAGATCCTTTTTTTCTGCGCGTAATCTGCTGCTTGCAAACAAAA
AAACCACCGCTACCAGCGGTGGTTTGTTTGCCGGATCAAGAGCTACCAACTCTTTTTCCGAAGG
TAACTGGCTTCAGCAGAGCGCAGATACCAAATACTGTTCTTCTAGTGTAGCCGTAGTTAGCCCA
CCACTTCAAGAACTCTGTAGCACCGCCTACATACCTCGCTCTGCTAATCCTGTTACCAGTGGCT
GCTGCCAGTGGCGATAAGTCGTGTCTTACCGGGTTGGACTCAAGACGATAGTTACCGGATAAGG
CGCAGCGGTCGGGCTGAACGGGGGGTTCGTGCACACAGCCCAGCTTGGAGCGAACGACCTACAC
CGAACTGAGATACCTACAGCGTGAGCTATGAGAAAGCGCCACGCTTCCCGAAGGGAGAAAGGCG
GACAGGTATCCGGTAAGCGGCAGGGTCGGAACAGGAGAGCGCACGAGGGAGCTTCCAGGGGGAA
ACGCCTGGTATCTTTATAGTCCTGTCGGGTTTCGCCACCTCTGACTTGAGCGTCGATTTTTGTG
ATGCTCGTCAGGGGGGCGGAGCCTATGGAAAAACGCCAGCAACGCGGCCTTTTTACGGTTCCTG
GCCTTTTGCTGGCCTTTTGCTCACATGTTCTTTCCTGCGTTATCCCCTGATTCTGTGGATAACC
GTATTACCGCCTTTGAGTGAGCTGATACCGCTCAAGGCTGACTGCAGGGCGAGAAGATTGCGAG
CTGTGCGGCTGAGTTGACGTATCTGTGCTGGATGATTACTCATAACGGCACCGCTATCAAACGT
GCCACGTTCATGTCCTACA
SEQ ID NO: 220 pCTM418
GCGCGCTCGCTCGCTCACTGAGGCCGCCCGGGCAAAGCCCGGGCGTCGGGCGACCTTTGGTCGC
CCGGCCTCAGTGAGCGAGCGAGCGCGCAGAGAGGGAGTGGCCAACTCCATCACTAGGGGTTCCT
TGTAGTTAATGATTAACCTCTTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGAGTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGTCGAGGTGAGCCCCACGTTCTGCTTCACTCTCCCCATCTCCCCCCCCTCCCCACCCCCAATT
TTGTATTTATTTATTTTTTAATTATTTTGTGCAGCGATGGGGGGGGGGGGGGGGGGGGGGGGCG
CGCCAGGCGGGGGGGGGGGGGGCGAGGGGGGGGGGGGGGCGAGGCGGAGAGGTGCGGCGGCAGC
CAATCAGAGCGGCGCGCTCCGAAAGTTTCCTTTTATGGCGAGGCGGCGGCGGCGGCGGCCCTAT
AAAAAGCGAAGCGCGCGGCGGGCGGGAGTCGCTGCGACGCTGCCTTCGCCCCGTGCCCCGCTCC
GCCGCCGCCTCGCGCCGCCCGCCCCGGCTCTGACTGACCGCGTTACTCCCACAGGTGAGCGGGC
GGGACGGCCCTTCTCCTCCGGGCTGTAATTAGCGCTTGGTTTAATGACGGCTTGTTTCTTTTCT
GTGGCTGCGTGAAAGCCTTGAGGGGCTCCGGGAGCTAGAGCCTCTGCTAACCATGTTCATGCCT
TCTTCTTTTTCCTACAGCTCCTGGGCAACGTGCTGGTTATTGTGCTGTCTCATCATTTTGGCAA
AGAATTCCTCGAAGATCTAGGCCTGCAGGCCACCATGGGCAAGATTTCTAGCCTGCCTACACAG
CTGTTCAAGTGCTGCTTCTGCGACTTCCTGAAGTCCGATACCGGCAGACCCTTCGTGGAAATGT
ACAGCGAGATCCCCGAGATCATCCACATGACCGAGGGCAGAGAGCTGGTCATCCCCTGCAGAGT
GACAAGCCCCAACATCACCGTGACTCTGAAGAAGTTCCCTCTGGACACACTGATCCCCGACGGC
AAGAGAATCATCTGGGACAGCCGGAAGGGCTTCATCATCAGCAACGCCACCTACAAAGAGATCG
GCCTGCTGACCTGTGAAGCCACCGTGAATGGCCACCTGTACAAGACCAACTACCTGACACACAG
ACAGACCAACACCATCATCGACGTGGTGCTGAGCCCTAGCCACGGCATTGAACTGTCTGTGGGC
GAGAAGCTGGTGCTGAACTGTACCGCCAGAACCGAGCTGAACGTGGGCATCGACTTCAACTGGG
AGTACCCCAGCAGCAAGCACCAGCACAAGAAACTGGTCAACCGGGACCTGAAAACCCAGAGCGG
CAGCGAGATGAAGAAATTCCTGAGCACCCTGACCATCGACGGCGTGACCAGATCTGACCAGGGC
CTGTACACATGTGCCGCCAGCTCTGGCCTGATGACCAAGAAAAACAGCACCTTCGTGCGGGTGC
ACGAGAAGGACAAGACCCACACCTGTCCTCCATGTCCTGCTCCAGAACTGCTCGGCGGACCTTC
CGTGTTCCTGTTTCCTCCAAAGCCTAAGGACACCCTGATGATCAGCAGAACCCCTGAAGTGACC
TGCGTGGTGGTGGATGTGTCCCACGAGGATCCCGAAGTGAAGTTCAATTGGTACGTGGACGGCG
TGGAAGTGCACAACGCCAAGACCAAGCCTAGAGAGGAACAGTACAATAGCACCTACAGAGTGGT
GTCCGTGCTGACCGTGCTGCACCAGGATTGGCTGAACGGCAAAGAGTACAAGTGCAAGGTGTCC
AACAAGGCCCTGCCTGCTCCTATCGAGAAAACCATCTCCAAGGCCAAGGGCCAGCCTAGGGAAC
CCCAGGTTTACACACTGCCTCCAAGCAGGGACGAGCTGACAAAGAACCAGGTGTCCCTGACCTG
CCTGGTCAAGGGCTTCTACCCTTCCGATATCGCCGTGGAATGGGAGAGCAATGGCCAGCCTGAG
AACAACTACAAGACAACCCCTCCTGTGCTGGACAGCGACGGCTCATTCTTCCTGTACAGCAAGC
TGACAGTGGACAAGAGCAGATGGCAGCAGGGCAACGTGTTCAGCTGCAGCGTGATGCACGAGGC
CCTGCACAACCACTACACCCAGAAGTCCCTGAGCCTGTCTCCTGGCTAAGAGGGCCCGGAAACC
TGGCCCTGTCTTCTTGACGAGCATTCCTAGGGGTCTTTCCCCTCTCGCCAAAGGAATGCAAGGT
CTGTTGAATGTCGTGAAGGAAGCAGTTCCTCTGGAAGCTTCTTGAAGACAAACAACGTCTGTAG
CGACCCTTTGCAGGCAGCGGAACCCCCCACCTGGCGACAGGTGCCTCTGCGGCCAAAAGCCACG
TGTATAAGATACACCTGCAAAGGCGGCACAACCCCAGTGCCACGTTGTGAGTTGGATAGTTGTG
GAAAGAGTCAAATGGCTCCCCTCAAGCGTATTCAACAAGGGGCTGAAGGATGCCCAGAAGGTAC
CCCATTGTATGGGATCTGATCTGGGGCCTCGGTGCACATGCTTTTCATGTGTTTAGTCGAGGTT
AAAAAACGTCTAGGCCCCCCGAACCACGGGGACGTGGTTTTCCTTTGAAAAACACGATGATAAT
AGAATTCGATATCAAGCTTATCGATACCGTCGACTAGAGCTCGCCACCATGAGACTGCTGGCCA
AGATCATCTGCCTGATGCTGTGGGCCATCTGCGTGGCCGAGGACTGCAACGAGCTGCCCCCCAG
AAGAAACACCGAGATCCTGACCGGCTCTTGGAGCGACCAGACCTACCCTGAGGGCACCCAGGCC
ATCTACAAGTGCAGACCCGGCTACCGGTCCCTGGGCAACATCATCATGGTGTGCAGAAAGGGCG
AGTGGGTGGCCCTGAACCCCCTGAGAAAGTGCCAGAAGAGGCCCTGCGGACACCCCGGCGATAC
CCCTTTTGGCACCTTCACACTGACCGGCGGCAACGTGTTCGAGTACGGCGTGAAGGCCGTGTAC
ACCTGTAACGAGGGCTACCAGCTGCTGGGCGAGATCAACTACAGAGAGTGCGACACCGACGGCT
GGACCAACGATATCCCCATCTGCGAGGTCGTGAAGTGCCTGCCTGTGACCGCCCCAGAGAACGG
CAAGATCGTGTCCAGCGCCATGGAACCCGACAGAGAGTACCACTTCGGCCAGGCCGTCAGATTC
GTGTGCAACAGCGGCTACAAGATCGAGGGCGACGAGGAAATGCACTGCAGCGACGACGGCTTCT
GGTCCAAAGAAAAGCCTAAGTGCGTGGAAATCAGCTGCAAGAGCCCCGACGTGATCAACGGCAG
CCCCATCAGCCAGAAGATCATCTACAAAGAGAACGAGCGGTTCCAGTACAAGTGTAACATGGGC
TACGAGTACAGCGAGCGGGGCGACGCCGTGTGTACAGAATCTGGATGGCGGCCTCTGCCCAGCT
GCGAGGAAAAGAGCTGCGACAACCCCTACATCCCCAACGGCGACTACAGCCCCCTGCGGATCAA
GCACAGAACCGGCGACGAGATCACCTACCAGTGCCGGAACGGCTTCTACCCCGCCACCAGAGGC
AATACCGCCAAGTGTACCAGCACCGGCTGGATCCCTGCCCCCAGATGTACCCTGAAGCCCTGCG
ACTACCCTGACATCAAGCACGGCGGCCTGTACCACGAGAACATGCGGAGGCCCTACTTCCCTGT
GGCCGTGGGCAAGTACTACAGCTACTACTGCGACGAGCACTTCGAGACACCCAGCGGCAGCTAC
TGGGACCACATCCACTGTACCCAGGACGGCTGGTCCCCTGCCGTGCCCTGCCTGAGGAAGTGCT
ACTTCCCCTACCTGGAAAACGGCTACAACCAGAACTACGGCCGGAAGTTCGTGCAGGGCAAGAG
CATCGATGTGGCCTGCCACCCTGGATACGCCCTGCCTAAGGCCCAGACCACCGTGACCTGCATG
GAAAATGGATGGTCCCCCACCCCCCGGTGCATCAGAGTGTCCTTCACCCTGTGAGAGCTCGCTG
ATCAGCCTCGACTGTGCCTTCTAGTTGCCAGCCATCTGTTGTTTGCCCCTCCCCCGTGCCTTCC
TTGACCCTGGAAGGTGCCACTCCCACTGTCCTTTCCTAATAAAATGAGGAAATTGCATCGCATT
GTCTGAGTAGGTGTCATTCTATTCTGGGGGGTGGGGGGGGCAGGACAGCAAGGGGGAGGATTG
GGAAGACAATAGCAGGCATGCTCAGGTTAATCATTAACTACAAGGAACCCCTAGTGATGGAGTT
GGCCACTCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGCACTGTCATTAGCAACTCCTTGT
CCTTCGATCTCGTCAACAACAGCTTGCAGTTCAAATACAAGACCCAGAAGGCGACTATTCTGGA
AGCGAGCTTGAAGAGTTAACCTGCAGAGAGCCCCCGCAGTGTCGACAATTAATCATCGGCATAG
TATATCGGCATAGTATAATACGACAAGGTGAGGAAGTAAAAAATGAGCCATATCCAACGGGAAA
CGTCGAGGCCGCGATTAAATTCCAACATGGATGCTGATTTATATGGGTATAAATGGGCTCGCGA
TAATGTCGGGCAATCAGGTGCGACAATCTATCGCTTGTATGGGAAGCCCGATGCGCCAGAGTTG
TTTCTGAAACATGGCAAAGGTAGCGTTGCCAATGATGTTACAGATGAGATGGTCAGACTAAACT
GGCTGACGGAATTTATGCCACTTCCGACCATCAAGCATTTTATCCGTACTCCTGATGATGCATG
GTTACTCACCACTGCGATCCCCGGAAAAACAGCGTTCCAGGTATTAGAAGAATATCCTGATTCA
GGTGAAAATATTGTTGATGCGCTGGCAGTGTTCCTGCGCCGGTTGCACTCGATTCCTGTTTGTA
ATTGTCCTTTTAACAGCGATCGCGTATTTCGCCTCGCTCAGGCGCAATCACGAATGAATAACGG
TTTGGTTGATGCGAGTGATTTTGATGACGAGCGTAATGGCTGGCCTGTTGAACAAGTCTGGAAA
GAAATGCATAAACTTTTGCCATTCTCACCGGATTCAGTCGTCACTCATGGTGATTTCTCACTTG
ATAACCTTATTTTTGACGAGGGGAAATTAATAGGTTGTATTGATGTTGGACGAGTCGGAATCGC
AGACCGATACCAGGATCTTGCCATCCTATGGAACTGCCTCGGTGAGTTTTCTCCTTCATTACAG
AAACGGCTTTTTCAAAAATATGGTATTGATAATCCTGATATGAATAAATTGCAGTTTCATTTGA
TGCTCGATGAGTTTTTCTAACTCATGACCAAAATCCCTTAACGTGAGTTACGCGCGCGTCGTTC
CACTGAGCGTCAGACCCCGTAGAAAAGATCAAAGGATCTTCTTGAGATCCTTTTTTTCTGCGCG
TAATCTGCTGCTTGCAAACAAAAAAACCACCGCTACCAGCGGTGGTTTGTTTGCCGGATCAAGA
GCTACCAACTCTTTTTCCGAAGGTAACTGGCTTCAGCAGAGCGCAGATACCAAATACTGTTCTT
CTAGTGTAGCCGTAGTTAGCCCACCACTTCAAGAACTCTGTAGCACCGCCTACATACCTCGCTC
TGCTAATCCTGTTACCAGTGGCTGCTGCCAGTGGCGATAAGTCGTGTCTTACCGGGTTGGACTC
AAGACGATAGTTACCGGATAAGGCGCAGCGGTCGGGCTGAACGGGGGGTTCGTGCACACAGCCC
AGCTTGGAGCGAACGACCTACACCGAACTGAGATACCTACAGCGTGAGCTATGAGAAAGCGCCA
CGCTTCCCGAAGGGAGAAAGGCGGACAGGTATCCGGTAAGCGGCAGGGTCGGAACAGGAGAGCG
CACGAGGGAGCTTCCAGGGGGAAACGCCTGGTATCTTTATAGTCCTGTCGGGTTTCGCCACCTC
TGACTTGAGCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTTTCCTGCGTT
ATCCCCTGATTCTGTGGATAACCGTATTACCGCCTTTGAGTGAGCTGATACCGCTCAAGGCTGA
CTGCAGGGCGAGAAGATTGCGAGCTGTGCGGCTGAGTTGACGTATCTGTGCTGGATGATTACTC
ATAACGGCACCGCTATCAAACGTGCCACGTTCATGTCCTACA
SEQ ID NO: 221 pCTM419
GCGCGCTCGCTCGCTCACTGAGGCCGCCCGGGCAAAGCCCGGGCGTCGGGCGACCTTTGGTCGC
CCGGCCTCAGTGAGCGAGCGAGCGCGCAGAGAGGGAGTGGCCAACTCCATCACTAGGGGTTCCT
TGTAGTTAATGATTAACCTCTTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGAGTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGTCGAGGTGAGCCCCACGTTCTGCTTCACTCTCCCCATCTCCCCCCCCTCCCCACCCCCAATT
TTGTATTTATTTATTTTTTAATTATTTTGTGCAGCGATGGGGGCGGGGGGGGGGGGGGGGCGCG
CGCCAGGCGGGGGGGGGCGGGGCGAGGGGGGGGGGGGGGCGAGGCGGAGAGGTGCGGCGGCAGC
CAATCAGAGCGGCGCGCTCCGAAAGTTTCCTTTTATGGCGAGGCGGCGGCGGCGGCGGCCCTAT
AAAAAGCGAAGCGCGCGGGGGGCGGGAGTCGCTGCGACGCTGCCTTCGCCCCGTGCCCCGCTCC
GCCGCCGCCTCGCGCCGCCCGCCCCGGCTCTGACTGACCGCGTTACTCCCACAGGTGAGCGGGC
GGGACGGCCCTTCTCCTCCGGGCTGTAATTAGCGCTTGGTTTAATGACGGCTTGTTTCTTTTCT
GTGGCTGCGTGAAAGCCTTGAGGGGCTCCGGGAGCTAGAGCCTCTGCTAACCATGTTCATGCCT
TCTTCTTTTTCCTACAGCTCCTGGGCAACGTGCTGGTTATTGTGCTGTCTCATCATTTTGGCAA
AGAATTCCTCGAAGATCTAGGCCTGCAGGCCACCATGGAAACAGATACACTGCTGCTGTGGGTG
CTGCTCTTGTGGGTGCCAGGATCTACAGGCGATTCTGATACCGGCAGACCCTTCGTGGAAATGT
ACAGCGAGATCCCCGAGATCATCCACATGACCGAGGGCAGAGAGCTGGTCATCCCCTGCAGAGT
GACAAGCCCCAACATCACCGTGACTCTGAAGAAGTTCCCTCTGGACACACTGATCCCCGACGGC
AAGAGAATCATCTGGGACAGCCGGAAGGGCTTCATCATCAGCAACGCCACCTACAAAGAGATCG
GCCTGCTGACCTGTGAAGCCACCGTGAATGGCCACCTGTACAAGACCAACTACCTGACACACAG
ACAGACCAACACCATCATCGACGTGGTGCTGAGCCCTAGCCACGGCATTGAACTGTCTGTGGGC
GAGAAGCTGGTGCTGAACTGTACCGCCAGAACCGAGCTGAACGTGGGCATCGACTTCAACTGGG
AGTACCCCAGCAGCAAGCACCAGCACAAGAAACTGGTCAACCGGGACCTGAAAACCCAGAGCGG
CAGCGAGATGAAGAAATTCCTGAGCACCCTGACCATCGACGGCGTGACCAGATCTGACCAGGGC
CTGTACACATGTGCCGCCAGCTCTGGCCTGATGACCAAGAAAAACAGCACCTTCGTGCGGGTGC
ACGAGAAGGACAAGACCCACACCTGTCCTCCATGTCCTGCTCCAGAACTGCTCGGCGGACCTTC
CGTGTTCCTGTTTCCTCCAAAGCCTAAGGACACCCTGATGATCAGCAGAACCCCTGAAGTGACC
TGCGTGGTGGTGGATGTGTCCCACGAGGATCCCGAAGTGAAGTTCAATTGGTACGTGGACGGCG
TGGAAGTGCACAACGCCAAGACCAAGCCTAGAGAGGAACAGTACAATAGCACCTACAGAGTGGT
GTCCGTGCTGACCGTGCTGCACCAGGATTGGCTGAACGGCAAAGAGTACAAGTGCAAGGTGTCC
AACAAGGCCCTGCCTGCTCCTATCGAGAAAACCATCTCCAAGGCCAAGGGCCAGCCTAGGGAAC
CCCAGGTTTACACACTGCCTCCAAGCAGGGACGAGCTGACAAAGAACCAGGTGTCCCTGACCTG
CCTGGTCAAGGGCTTCTACCCTTCCGATATCGCCGTGGAATGGGAGAGCAATGGCCAGCCTGAG
AACAACTACAAGACAACCCCTCCTGTGCTGGACAGCGACGGCTCATTCTTCCTGTACAGCAAGC
TGACAGTGGACAAGAGCAGATGGCAGCAGGGCAACGTGTTCAGCTGCAGCGTGATGCACGAGGC
CCTGCACAACCACTACACCCAGAAGTCCCTGAGCCTGTCTCCTGGCTAAGAGGGCCCGGAAACC
TGGCCCTGTCTTCTTGACGAGCATTCCTAGGGGTCTTTCCCCTCTCGCCAAAGGAATGCAAGGT
CTGTTGAATGTCGTGAAGGAAGCAGTTCCTCTGGAAGCTTCTTGAAGACAAACAACGTCTGTAG
CGACCCTTTGCAGGCAGCGGAACCCCCCACCTGGCGACAGGTGCCTCTGCGGCCAAAAGCCACG
TGTATAAGATACACCTGCAAAGGCGGCACAACCCCAGTGCCACGTTGTGAGTTGGATAGTTGTG
GAAAGAGTCAAATGGCTCCCCTCAAGCGTATTCAACAAGGGGCTGAAGGATGCCCAGAAGGTAC
CCCATTGTATGGGATCTGATCTGGGGCCTCGGTGCACATGCTTTTCATGTGTTTAGTCGAGGTT
AAAAAACGTCTAGGCCCCCCGAACCACGGGGACGTGGTTTTCCTTTGAAAAACACGATGATAAT
AGAATTCGATATCAAGCTTATCGATACCGTCGACTAGAGCTCGCCACCATGAGACTGCTGGCCA
AGATCATCTGCCTGATGCTGTGGGCCATCTGCGTGGCCGAGGACTGCAACGAGCTGCCCCCCAG
AAGAAACACCGAGATCCTGACCGGCTCTTGGAGCGACCAGACCTACCCTGAGGGCACCCAGGCC
ATCTACAAGTGCAGACCCGGCTACCGGTCCCTGGGCAACATCATCATGGTGTGCAGAAAGGGCG
AGTGGGTGGCCCTGAACCCCCTGAGAAAGTGCCAGAAGAGGCCCTGCGGACACCCCGGCGATAC
CCCTTTTGGCACCTTCACACTGACCGGCGGCAACGTGTTCGAGTACGGCGTGAAGGCCGTGTAC
ACCTGTAACGAGGGCTACCAGCTGCTGGGCGAGATCAACTACAGAGAGTGCGACACCGACGGCT
GGACCAACGATATCCCCATCTGCGAGGTCGTGAAGTGCCTGCCTGTGACCGCCCCAGAGAACGG
CAAGATCGTGTCCAGCGCCATGGAACCCGACAGAGAGTACCACTTCGGCCAGGCCGTCAGATTC
GTGTGCAACAGCGGCTACAAGATCGAGGGCGACGAGGAAATGCACTGCAGCGACGACGGCTTCT
GGTCCAAAGAAAAGCCTAAGTGCGTGGAAATCAGCTGCAAGAGCCCCGACGTGATCAACGGCAG
CCCCATCAGCCAGAAGATCATCTACAAAGAGAACGAGCGGTTCCAGTACAAGTGTAACATGGGC
TACGAGTACAGCGAGCGGGGCGACGCCGTGTGTACAGAATCTGGATGGCGGCCTCTGCCCAGCT
GCGAGGAAAAGAGCTGCGACAACCCCTACATCCCCAACGGCGACTACAGCCCCCTGCGGATCAA
GCACAGAACCGGCGACGAGATCACCTACCAGTGCCGGAACGGCTTCTACCCCGCCACCAGAGGC
AATACCGCCAAGTGTACCAGCACCGGCTGGATCCCTGCCCCCAGATGTACCCTGAAGCCCTGCG
ACTACCCTGACATCAAGCACGGCGGCCTGTACCACGAGAACATGCGGAGGCCCTACTTCCCTGT
GGCCGTGGGCAAGTACTACAGCTACTACTGCGACGAGCACTTCGAGACACCCAGCGGCAGCTAC
TGGGACCACATCCACTGTACCCAGGACGGCTGGTCCCCTGCCGTGCCCTGCCTGAGGAAGTGCT
ACTTCCCCTACCTGGAAAACGGCTACAACCAGAACTACGGCCGGAAGTTCGTGCAGGGCAAGAG
CATCGATGTGGCCTGCCACCCTGGATACGCCCTGCCTAAGGCCCAGACCACCGTGACCTGCATG
GAAAATGGATGGTCCCCCACCCCCCGGTGCATCAGAGTGTCCTTCACCCTGTGAGAGCTCGCTG
ATCAGCCTCGACTGTGCCTTCTAGTTGCCAGCCATCTGTTGTTTGCCCCTCCCCCGTGCCTTCC
TTGACCCTGGAAGGTGCCACTCCCACTGTCCTTTCCTAATAAAATGAGGAAATTGCATCGCATT
GTCTGAGTAGGTGTCATTCTATTCTGGGGGGTGGGGTGGGGCAGGACAGCAAGGGGGAGGATTG
GGAAGACAATAGCAGGCATGCTCAGGTTAATCATTAACTACAAGGAACCCCTAGTGATGGAGTT
GGCCACTCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGCACTGTCATTAGCAACTCCTTGT
CCTTCGATCTCGTCAACAACAGCTTGCAGTTCAAATACAAGACCCAGAAGGCGACTATTCTGGA
AGCGAGCTTGAAGAGTTAACCTGCAGAGAGCCCCCGCAGTGTCGACAATTAATCATCGGCATAG
TATATCGGCATAGTATAATACGACAAGGTGAGGAAGTAAAAAATGAGCCATATCCAACGGGAAA
CGTCGAGGCCGCGATTAAATTCCAACATGGATGCTGATTTATATGGGTATAAATGGGCTCGCGA
TAATGTCGGGCAATCAGGTGCGACAATCTATCGCTTGTATGGGAAGCCCGATGCGCCAGAGTTG
TTTCTGAAACATGGCAAAGGTAGCGTTGCCAATGATGTTACAGATGAGATGGTCAGACTAAACT
GGCTGACGGAATTTATGCCACTTCCGACCATCAAGCATTTTATCCGTACTCCTGATGATGCATG
GTTACTCACCACTGCGATCCCCGGAAAAACAGCGTTCCAGGTATTAGAAGAATATCCTGATTCA
GGTGAAAATATTGTTGATGCGCTGGCAGTGTTCCTGCGCCGGTTGCACTCGATTCCTGTTTGTA
ATTGTCCTTTTAACAGCGATCGCGTATTTCGCCTCGCTCAGGCGCAATCACGAATGAATAACGG
TTTGGTTGATGCGAGTGATTTTGATGACGAGCGTAATGGCTGGCCTGTTGAACAAGTCTGGAAA
GAAATGCATAAACTTTTGCCATTCTCACCGGATTCAGTCGTCACTCATGGTGATTTCTCACTTG
ATAACCTTATTTTTGACGAGGGGAAATTAATAGGTTGTATTGATGTTGGACGAGTCGGAATCGC
AGACCGATACCAGGATCTTGCCATCCTATGGAACTGCCTCGGTGAGTTTTCTCCTTCATTACAG
AAACGGCTTTTTCAAAAATATGGTATTGATAATCCTGATATGAATAAATTGCAGTTTCATTTGA
TGCTCGATGAGTTTTTCTAACTCATGACCAAAATCCCTTAACGTGAGTTACGCGCGCGTCGTTC
CACTGAGCGTCAGACCCCGTAGAAAAGATCAAAGGATCTTCTTGAGATCCTTTTTTTCTGCGCG
TAATCTGCTGCTTGCAAACAAAAAAACCACCGCTACCAGCGGTGGTTTGTTTGCCGGATCAAGA
GCTACCAACTCTTTTTCCGAAGGTAACTGGCTTCAGCAGAGCGCAGATACCAAATACTGTTCTT
CTAGTGTAGCCGTAGTTAGCCCACCACTTCAAGAACTCTGTAGCACCGCCTACATACCTCGCTC
TGCTAATCCTGTTACCAGTGGCTGCTGCCAGTGGCGATAAGTCGTGTCTTACCGGGTTGGACTC
AAGACGATAGTTACCGGATAAGGCGCAGCGGTCGGGCTGAACGGGGGGTTCGTGCACACAGCCC
AGCTTGGAGCGAACGACCTACACCGAACTGAGATACCTACAGCGTGAGCTATGAGAAAGCGCCA.
CGCTTCCCGAAGGGAGAAAGGCGGACAGGTATCCGGTAAGCGGCAGGGTCGGAACAGGAGAGCG
CACGAGGGAGCTTCCAGGGGGAAACGCCTGGTATCTTTATAGTCCTGTCGGGTTTCGCCACCTC
TGACTTGAGCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTTTCCTGCGTT
ATCCCCTGATTCTGTGGATAACCGTATTACCGCCTTTGAGTGAGCTGATACCGCTCAAGGCTGA
CTGCAGGGCGAGAAGATTGCGAGCTGTGCGGCTGAGTTGACGTATCTGTGCTGGATGATTACTC
ATAACGGCACCGCTATCAAACGTGCCACGTTCATGTCCTACA
SEQ ID NO: 222 pCTM428
GCGCGCTCGCTCGCTCACTGAGGCCGCCCGGGCAAAGCCCGGGCGTCGGGCGACCTTTGGTCGC
CCGGCCTCAGTGAGCGAGCGAGCGCGCAGAGAGGGAGTGGCCAACTCCATCACTAGGGGTTCCT
TGTAGTTAATGATTAACCTCTTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGAGTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGTCGAGGTGAGCCCCACGTTCTGCTTCACTCTCCCCATCTCCCCCCCCTCCCCACCCCCAATT
TTGTATTTATTTATTTTTTAATTATTTTGTGCAGCGATGGGGGCGGGGGGGGGGGGGGGGCGCG
CGCCAGGCGGGGCGGGGGGGGGCGAGGGGCGGGGGGGGGCGAGGCGGAGAGGTGCGGCGGCAGC
CAATCAGAGCGGCGCGCTCCGAAAGTTTCCTTTTATGGCGAGGGGGGGGCGGCGGCGGCCCTAT
AAAAAGCGAAGCGCGCGGCGGGGGGAGTCGCTGCGACGCTGCCTTCGCCCCGTGCCCCGCTCC
GCCGCCGCCTCGCGCCGCCCGCCCCGGCTCTGACTGACCGCGTTACTCCCACAGGTGAGCGGGC
GGGACGGCCCTTCTCCTCCGGGCTGTAATTAGCGCTTGGTTTAATGACGGCTTGTTTCTTTTCT
GTGGCTGCGTGAAAGCCTTGAGGGGCTCCGGGAGCTAGAGCCTCTGCTAACCATGTTCATGCCT
TCTTCTTTTTCCTACAGCTCCTGGGCAACGTGCTGGTTATTGTGCTGTCTCATCATTTTGGCAA
AGAATTCCTCGAAGATCTAGGCCTGCAGGCCACCATGGGCAAGATTTCTAGCCTGCCTACACAG
CTGTTCAAGTGCTGCTTCTGCGACTTCCTGAAGTCCGATACCGGCAGACCCTTCGTGGAAATGT
ACAGCGAGATCCCCGAGATCATCCACATGACCGAGGGCAGAGAGCTGGTCATCCCCTGCAGAGT
GACAAGCCCCAACATCACCGTGACTCTGAAGAAGTTCCCTCTGGACACACTGATCCCCGACGGC
AAGAGAATCATCTGGGACAGCCGGAAGGGCTTCATCATCAGCAACGCCACCTACAAAGAGATCG
GCCTGCTGACCTGTGAAGCCACCGTGAATGGCCACCTGTACAAGACCAACTACCTGACACACAG
ACAGACCAACACCATCATCGACGTGGTGCTGAGCCCTAGCCACGGCATTGAACTGTCTGTGGGC
GAGAAGCTGGTGCTGAACTGTACCGCCAGAACCGAGCTGAACGTGGGCATCGACTTCAACTGGG
AGTACCCCAGCAGCAAGCACCAGCACAAGAAACTGGTCAACCGGGACCTGAAAACCCAGAGCGG
CAGCGAGATGAAGAAATTCCTGAGCACCCTGACCATCGACGGCGTGACCAGATCTGACCAGGGC
CTGTACACATGTGCCGCCAGCTCTGGCCTGATGACCAAGAAAAACAGCACCTTCGTGCGGGTGC
ACGAGAAGGACAAGACCCACACCTGTCCTCCATGTCCTGCTCCAGAACTGCTCGGCGGACCTTC
CGTGTTCCTGTTTCCTCCAAAGCCTAAGGACACCCTGATGATCAGCAGAACCCCTGAAGTGACC
TGCGTGGTGGTGGATGTGTCCCACGAGGATCCCGAAGTGAAGTTCAATTGGTACGTGGACGGCG
TGGAAGTGCACAACGCCAAGACCAAGCCTAGAGAGGAACAGTACAATAGCACCTACAGAGTGGT
GTCCGTGCTGACCGTGCTGCACCAGGATTGGCTGAACGGCAAAGAGTACAAGTGCAAGGTGTCC
AACAAGGCCCTGCCTGCTCCTATCGAGAAAACCATCTCCAAGGCCAAGGGCCAGCCTAGGGAAC
CCCAGGTTTACACACTGCCTCCAAGCAGGGACGAGCTGACAAAGAACCAGGTGTCCCTGACCTG
CCTGGTCAAGGGCTTCTACCCTTCCGATATCGCCGTGGAATGGGAGAGCAATGGCCAGCCTGAG
AACAACTACAAGACAACCCCTCCTGTGCTGGACAGCGACGGCTCATTCTTCCTGTACAGCAAGC
TGACAGTGGACAAGAGCAGATGGCAGCAGGGCAACGTGTTCAGCTGCAGCGTGATGCACGAGGC
CCTGCACAACCACTACACCCAGAAGTCCCTGAGCCTGTCTCCTGGCGGCGGCAGGGGTCGACGC
GGCGGCATGAGACTGCTGGCCAAGATCATCTGCCTGATGCTGTGGGCCATCTGCGTGGCCGAGG
ACTGCAACGAGCTGCCCCCCAGAAGAAACACCGAGATCCTGACCGGCTCTTGGAGCGACCAGAC
CTACCCTGAGGGCACCCAGGCCATCTACAAGTGCAGACCCGGCTACCGGTCCCTGGGCAACATC
ATCATGGTGTGCAGAAAGGGCGAGTGGGTGGCCCTGAACCCCCTGAGAAAGTGCCAGAAGAGGC
CCTGCGGACACCCCGGCGATACCCCTTTTGGCACCTTCACACTGACCGGCGGCAACGTGTTCGA
GTACGGCGTGAAGGCCGTGTACACCTGTAACGAGGGCTACCAGCTGCTGGGCGAGATCAACTAC
AGAGAGTGCGACACCGACGGCTGGACCAACGATATCCCCATCTGCGAGGTCGTGAAGTGCCTGC
CTGTGACCGCCCCAGAGAACGGCAAGATCGTGTCCAGCGCCATGGAACCCGACAGAGAGTACCA
CTTCGGCCAGGCCGTCAGATTCGTGTGCAACAGCGGCTACAAGATCGAGGGCGACGAGGAAATG
CACTGCAGCGACGACGGCTTCTGGTCCAAAGAAAAGCCTAAGTGCGTGGAAATCAGCTGCAAGA
GCCCCGACGTGATCAACGGCAGCCCCATCAGCCAGAAGATCATCTACAAAGAGAACGAGCGGTT
CCAGTACAAGTGTAACATGGGCTACGAGTACAGCGAGCGGGGCGACGCCGTGTGTACAGAATCT
GGATGGCGGCCTCTGCCCAGCTGCGAGGAAAAGAGCTGCGACAACCCCTACATCCCCAACGGCG
ACTACAGCCCCCTGCGGATCAAGCACAGAACCGGCGACGAGATCACCTACCAGTGCCGGAACGG
CTTCTACCCCGCCACCAGAGGCAATACCGCCAAGTGTACCAGCACCGGCTGGATCCCTGCCCCC
AGATGTACCCTGAAGCCCTGCGACTACCCTGACATCAAGCACGGCGGCCTGTACCACGAGAACA
TGCGGAGGCCCTACTTCCCTGTGGCCGTGGGCAAGTACTACAGCTACTACTGCGACGAGCACTT
CGAGACACCCAGCGGCAGCTACTGGGACCACATCCACTGTACCCAGGACGGCTGGTCCCCTGCC
GTGCCCTGCCTGAGGAAGTGCTACTTCCCCTACCTGGAAAACGGCTACAACCAGAACTACGGCC
GGAAGTTCGTGCAGGGCAAGAGCATCGATGTGGCCTGCCACCCTGGATACGCCCTGCCTAAGGC
CCAGACCACCGTGACCTGCATGGAAAATGGATGGTCCCCCACCCCCCGGTGCATCAGAGTGTCC
TTCACCCTGTGAGAGCTCGCTGATCAGCCTCGACTGTGCCTTCTAGTTGCCAGCCATCTGTTGT
TTGCCCCTCCCCCGTGCCTTCCTTGACCCTGGAAGGTGCCACTCCCACTGTCCTTTCCTAATAA
AATGAGGAAATTGCATCGCATTGTCTGAGTAGGTGTCATTCTATTCTGGGGGGTGGGGTGGGGC
AGGACAGCAAGGGGGAGGATTGGGAAGACAATAGCAGGCATGCTCAGGTTAATCATTAACTACA
AGGAACCCCTAGTGATGGAGTTGGCCACTCCCTCTCTGCGCGCTCGCTCGCTCACTGAGGCCGG
GCGACCAAAGGTCGCCCGACGCCCGGGCTTTGCCCGGGCGGCCTCAGTGAGCGAGCGAGCGCGC
ACTGTCATTAGCAACTCCTTGTCCTTCGATCTCGTCAACAACAGCTTGCAGTTCAAATACAAGA
CCCAGAAGGCGACTATTCTGGAAGCGAGCTTGAAGAGTTAACCTGCAGAGAGCCCCCGCAGTGT
CGACAATTAATCATCGGCATAGTATATCGGCATAGTATAATACGACAAGGTGAGGAAGTAAAAA
ATGAGCCATATCCAACGGGAAACGTCGAGGCCGCGATTAAATTCCAACATGGATGCTGATTTAT
ATGGGTATAAATGGGCTCGCGATAATGTCGGGCAATCAGGTGCGACAATCTATCGCTTGTATGG
GAAGCCCGATGCGCCAGAGTTGTTTCTGAAACATGGCAAAGGTAGCGTTGCCAATGATGTTACA
GATGAGATGGTCAGACTAAACTGGCTGACGGAATTTATGCCACTTCCGACCATCAAGCATTTTA
TCCGTACTCCTGATGATGCATGGTTACTCACCACTGCGATCCCCGGAAAAACAGCGTTCCAGGT
ATTAGAAGAATATCCTGATTCAGGTGAAAATATTGTTGATGCGCTGGCAGTGTTCCTGCGCCGG
TTGCACTCGATTCCTGTTTGTAATTGTCCTTTTAACAGCGATCGCGTATTTCGCCTCGCTCAGG
CGCAATCACGAATGAATAACGGTTTGGTTGATGCGAGTGATTTTGATGACGAGCGTAATGGCTG
GCCTGTTGAACAAGTCTGGAAAGAAATGCATAAACTTTTGCCATTCTCACCGGATTCAGTCGTC
ACTCATGGTGATTTCTCACTTGATAACCTTATTTTTGACGAGGGGAAATTAATAGGTTGTATTG
ATGTTGGACGAGTCGGAATCGCAGACCGATACCAGGATCTTGCCATCCTATGGAACTGCCTCGG
TGAGTTTTCTCCTTCATTACAGAAACGGCTTTTTCAAAAATATGGTATTGATAATCCTGATATG
AATAAATTGCAGTTTCATTTGATGCTCGATGAGTTTTTCTAACTCATGACCAAAATCCCTTAAC
GTGAGTTACGCGCGCGTCGTTCCACTGAGCGTCAGACCCCGTAGAAAAGATCAAAGGATCTTCT
TGAGATCCTTTTTTTCTGCGCGTAATCTGCTGCTTGCAAACAAAAAAACCACCGCTACCAGCGG
TGGTTTGTTTGCCGGATCAAGAGCTACCAACTCTTTTTCCGAAGGTAACTGGCTTCAGCAGAGC
GCAGATACCAAATACTGTTCTTCTAGTGTAGCCGTAGTTAGCCCACCACTTCAAGAACTCTGTA
GCACCGCCTACATACCTCGCTCTGCTAATCCTGTTACCAGTGGCTGCTGCCAGTGGCGATAAGT
CGTGTCTTACCGGGTTGGACTCAAGACGATAGTTACCGGATAAGGCGCAGCGGTCGGGCTGAAC
GGGGGGTTCGTGCACACAGCCCAGCTTGGAGCGAACGACCTACACCGAACTGAGATACCTACAG
CGTGAGCTATGAGAAAGCGCCACGCTTCCCGAAGGGAGAAAGGCGGACAGGTATCCGGTAAGCG
GCAGGGTCGGAACAGGAGAGCGCACGAGGGAGCTTCCAGGGGGAAACGCCTGGTATCTTTATAG
TCCTGTCGGGTTTCGCCACCTCTGACTTGAGCGTCGATTTTTGTGATGCTCGTCAGGGGGGCGG
AGCCTATGGAAAAACGCCAGCAACGCGGCCTTTTTACGGTTCCTGGCCTTTTGCTGGCCTTTTG
CTCACATGTTCTTTCCTGCGTTATCCCCTGATTCTGTGGATAACCGTATTACCGCCTTTGAGTG
AGCTGATACCGCTCAAGGCTGACTGCAGGGCGAGAAGATTGCGAGCTGTGCGGCTGAGTTGACG
TATCTGTGCTGGATGATTACTCATAACGGCACCGCTATCAAACGTGCCACGTTCATGTCCTACA
SEQ ID NO: 223 pCTM429
GCGCGCTCGCTCGCTCACTGAGGCCGCCCGGGCAAAGCCCGGGCGTCGGGCGACCTTTGGTCGC
CCGGCCTCAGTGAGCGAGCGAGCGCGCAGAGAGGGAGTGGCCAACTCCATCACTAGGGGTTCCT
TGTAGTTAATGATTAACCTCTTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGAGTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGTCGAGGTGAGCCCCACGTTCTGCTTCACTCTCCCCATCTCCCCCCCCTCCCCACCCCCAATT
TTGTATTTATTTATTTTTTAATTATTTTGTGCAGCGATGGGGGGGGGGGGGGGGGGGGGGGGCG
CGCCAGGCGGGGGGGGGGGGGGCGAGGGGCGGGGGGGGGCGAGGCGGAGAGGTGCGGCGGCAGC
CAATCAGAGCGGCGCGCTCCGAAAGTTTCCTTTTATGGCGAGGGGGGGGCGGCGGCGGCCCTAT
AAAAAGCGAAGCGCGCGGCGGGCGGGAGTCGCTGCGCGCTGCCTTCGCCCCGTGCCCCGCTCCG
CCGCCGCCTCGCGCCGCCCGCCCCGGCTCTGACTGACCGCGTTACTCCCACAGGTGAGCGGGCG
GGACGGCCCTTCTCCTCCGGGCTGTAATTAGCGCTTGGTTTAATGACGGCTTGTTTCTTTTCTG
TGGCTGCGTGAAAGCCTTGAGGGGCTCCGGGAGCTAGAGCCTCTGCTAACCATGTTCATGCCTT
CTTCTTTTTCCTACAGCTCCTGGGCAACGTGCTGGTTATTGTGCTGTCTCATCATTTTGGCAAA
GAATTCCTCGAAGATCTAGGCCTGCAGGCGGCCGCCACCATGAGACTGCTGGCCAAGATCATCT
GCCTGATGCTGTGGGCCATCTGCGTGGCCGAGGACTGCAACGAGCTGCCCCCCAGAAGAAACAC
CGAGATCCTGACCGGCTCTTGGAGCGACCAGACCTACCCTGAGGGCACCCAGGCCATCTACAAG
TGCAGACCCGGCTACCGGTCCCTGGGCAACATCATCATGGTGTGCAGAAAGGGCGAGTGGGTGG
CCCTGAACCCCCTGAGAAAGTGCCAGAAGAGGCCCTGCGGACACCCCGGCGATACCCCTTTTGG
CACCTTCACACTGACCGGCGGCAACGTGTTCGAGTACGGCGTGAAGGCCGTGTACACCTGTAAC
GAGGGCTACCAGCTGCTGGGCGAGATCAACTACAGAGAGTGCGACACCGACGGCTGGACCAACG
ATATCCCCATCTGCGAGGTCGTGAAGTGCCTGCCTGTGACCGCCCCAGAGAACGGCAAGATCGT
GTCCAGCGCCATGGAACCCGACAGAGAGTACCACTTCGGCCAGGCCGTCAGATTCGTGTGCAAC
AGCGGCTACAAGATCGAGGGCGACGAGGAAATGCACTGCAGCGACGACGGCTTCTGGTCCAAAG
AAAAGCCTAAGTGCGTGGAAATCAGCTGCAAGAGCCCCGACGTGATCAACGGCAGCCCCATCAG
CCAGAAGATCATCTACAAAGAGAACGAGCGGTTCCAGTACAAGTGTAACATGGGCTACGAGTAC
AGCGAGCGGGGCGACGCCGTGTGTACAGAATCTGGATGGCGGCCTCTGCCCAGCTGCGAGGAAA
AGAGCTGCGACAACCCCTACATCCCCAACGGCGACTACAGCCCCCTGCGGATCAAGCACAGAAC
CGGCGACGAGATCACCTACCAGTGCCGGAACGGCTTCTACCCCGCCACCAGAGGCAATACCGCC
AAGTGTACCAGCACCGGCTGGATCCCTGCCCCCAGATGTACCCTGAAGCCCTGCGACTACCCTG
ACATCAAGCACGGCGGCCTGTACCACGAGAACATGCGGAGGCCCTACTTCCCTGTGGCCGTGGG
CAAGTACTACAGCTACTACTGCGACGAGCACTTCGAGACACCCAGCGGCAGCTACTGGGACCAC
ATCCACTGTACCCAGGACGGCTGGTCCCCTGCCGTGCCCTGCCTGAGGAAGTGCTACTTCCCCT
ACCTGGAAAACGGCTACAACCAGAACTACGGCCGGAAGTTCGTGCAGGGCAAGAGCATCGATGT
GGCCTGCCACCCTGGATACGCCCTGCCTAAGGCCCAGACCACCGTGACCTGCATGGAAAATGGA
TGGTCCCCCACCCCCCGGTGCATCAGAGTGTCCTTCACCCTGGGCGGCAGGGGTCGACGCGGCG
GCATGGCCTCTCATAGACTGCTGCTGCTGTGTCTGGCCGGCCTGGTGTTTGTGTCTGAGGCCTC
TGATACCGGCAGACCCTTCGTGGAAATGTACAGCGAGATCCCCGAGATCATCCACATGACCGAG
GGCAGAGAGCTGGTCATCCCCTGCAGAGTGACAAGCCCCAACATCACCGTGACTCTGAAGAAGT
TCCCTCTGGACACACTGATCCCCGACGGCAAGAGAATCATCTGGGACAGCCGGAAGGGCTTCAT
CATCAGCAACGCCACCTACAAAGAGATCGGCCTGCTGACCTGTGAAGCCACCGTGAATGGCCAC
CTGTACAAGACCAACTACCTGACACACAGACAGACCAACACCATCATCGACGTGGTGCTGAGCC
CTAGCCACGGCATTGAACTGTCTGTGGGCGAGAAGCTGGTGCTGAACTGTACCGCCAGAACCGA
GCTGAACGTGGGCATCGACTTCAACTGGGAGTACCCCAGCAGCAAGCACCAGCACAAGAAACTG
GTCAACCGGGACCTGAAAACCCAGAGCGGCAGCGAGATGAAGAAATTCCTGAGCACCCTGACCA
TCGACGGCGTGACCAGATCTGACCAGGGCCTGTACACATGTGCCGCCAGCTCTGGCCTGATGAC
CAAGAAAAACAGCACCTTCGTGCGGGTGCACGAGAAGGACAAGACCCACACCTGTCCTCCATGT
CCTGCTCCAGAACTGCTCGGCGGACCTTCCGTGTTCCTGTTTCCTCCAAAGCCTAAGGACACCC
TGATGATCAGCAGAACCCCTGAAGTGACCTGCGTGGTGGTGGATGTGTCCCACGAGGATCCCGA.
AGTGAAGTTCAATTGGTACGTGGACGGCGTGGAAGTGCACAACGCCAAGACCAAGCCTAGAGAG
GAACAGTACAATAGCACCTACAGAGTGGTGTCCGTGCTGACCGTGCTGCACCAGGATTGGCTGA
ACGGCAAAGAGTACAAGTGCAAGGTGTCCAACAAGGCCCTGCCTGCTCCTATCGAGAAAACCAT
CTCCAAGGCCAAGGGCCAGCCTAGGGAACCCCAGGTTTACACACTGCCTCCAAGCAGGGACGAG
CTGACAAAGAACCAGGTGTCCCTGACCTGCCTGGTCAAGGGCTTCTACCCTTCCGATATCGCCG
TGGAATGGGAGAGCAATGGCCAGCCTGAGAACAACTACAAGACAACCCCTCCTGTGCTGGACAG
CGACGGCTCATTCTTCCTGTACAGCAAGCTGACAGTGGACAAGAGCAGATGGCAGCAGGGCAAC
GTGTTCAGCTGCAGCGTGATGCACGAGGCCCTGCACAACCACTACACCCAGAAGTCCCTGAGCC
TGTCTCCTGGCTAAGAGCTCGCTGATCAGCCTCGACTGTGCCTTCTAGTTGCCAGCCATCTGTT
GTTTGCCCCTCCCCCGTGCCTTCCTTGACCCTGGAAGGTGCCACTCCCACTGTCCTTTCCTAAT
AAAATGAGGAAATTGCATCGCATTGTCTGAGTAGGTGTCATTCTATTCTGGGGGGTGGGGTGGG
GCAGGACAGCAAGGGGGAGGATTGGGAAGACAATAGCAGGCATGCTCAGGTTAATCATTAACTA
CAAGGAACCCCTAGTGATGGAGTTGGCCACTCCCTCTCTGCGCGCTCGCTCGCTCACTGAGGCC
GGGCGACCAAAGGTCGCCCGACGCCCGGGCTTTGCCCGGGCGGCCTCAGTGAGCGAGCGAGCGC
GCACTGTCATTAGCAACTCCTTGTCCTTCGATCTCGTCAACAACAGCTTGCAGTTCAAATACAA
GACCCAGAAGGCGACTATTCTGGAAGCGAGCTTGAAGAGTTAACCTGCAGAGAGCCCCCGCAGT
GTCGACAATTAATCATCGGCATAGTATATCGGCATAGTATAATACGACAAGGTGAGGAAGTAAA
AAATGAGCCATATCCAACGGGAAACGTCGAGGCCGCGATTAAATTCCAACATGGATGCTGATTT
ATATGGGTATAAATGGGCTCGCGATAATGTCGGGCAATCAGGTGCGACAATCTATCGCTTGTAT
GGGAAGCCCGATGCGCCAGAGTTGTTTCTGAAACATGGCAAAGGTAGCGTTGCCAATGATGTTA
CAGATGAGATGGTCAGACTAAACTGGCTGACGGAATTTATGCCACTTCCGACCATCAAGCATTT
TATCCGTACTCCTGATGATGCATGGTTACTCACCACTGCGATCCCCGGAAAAACAGCGTTCCAG
GTATTAGAAGAATATCCTGATTCAGGTGAAAATATTGTTGATGCGCTGGCAGTGTTCCTGCGCC
GGTTGCACTCGATTCCTGTTTGTAATTGTCCTTTTAACAGCGATCGCGTATTTCGCCTCGCTCA
GGCGCAATCACGAATGAATAACGGTTTGGTTGATGCGAGTGATTTTGATGACGAGCGTAATGGC
TGGCCTGTTGAACAAGTCTGGAAAGAAATGCATAAACTTTTGCCATTCTCACCGGATTCAGTCG
TCACTCATGGTGATTTCTCACTTGATAACCTTATTTTTGACGAGGGGAAATTAATAGGTTGTAT
TGATGTTGGACGAGTCGGAATCGCAGACCGATACCAGGATCTTGCCATCCTATGGAACTGCCTC
GGTGAGTTTTCTCCTTCATTACAGAAACGGCTTTTTCAAAAATATGGTATTGATAATCCTGATA
TGAATAAATTGCAGTTTCATTTGATGCTCGATGAGTTTTTCTAACTCATGACCAAAATCCCTTA
ACGTGAGTTACGCGCGCGTCGTTCCACTGAGCGTCAGACCCCGTAGAAAAGATCAAAGGATCTT
CTTGAGATCCTTTTTTTCTGCGCGTAATCTGCTGCTTGCAAACAAAAAAACCACCGCTACCAGC
GGTGGTTTGTTTGCCGGATCAAGAGCTACCAACTCTTTTTCCGAAGGTAACTGGCTTCAGCAGA
GCGCAGATACCAAATACTGTTCTTCTAGTGTAGCCGTAGTTAGCCCACCACTTCAAGAACTCTG
TAGCACCGCCTACATACCTCGCTCTGCTAATCCTGTTACCAGTGGCTGCTGCCAGTGGCGATAA
GTCGTGTCTTACCGGGTTGGACTCAAGACGATAGTTACCGGATAAGGCGCAGCGGTCGGGCTGA
ACGGGGGGTTCGTGCACACAGCCCAGCTTGGAGCGAACGACCTACACCGAACTGAGATACCTAC
AGCGTGAGCTATGAGAAAGCGCCACGCTTCCCGAAGGGAGAAAGGCGGACAGGTATCCGGTAAG
CGGCAGGGTCGGAACAGGAGAGCGCACGAGGGAGCTTCCAGGGGGAAACGCCTGGTATCTTTAT
AGTCCTGTCGGGTTTCGCCACCTCTGACTTGAGCGTCGATTTTTGTGATGCTCGTCAGGGGGGC
GGAGCCTATGGAAAAACGCCAGCAACGCGGCCTTTTTACGGTTCCTGGCCTTTTGCTGGCCTTT
TGCTCACATGTTCTTTCCTGCGTTATCCCCTGATTCTGTGGATAACCGTATTACCGCCTTTGAG
TGAGCTGATACCGCTCAAGGCTGACTGCAGGGCGAGAAGATTGCGAGCTGTGCGGCTGAGTTGA
CGTATCTGTGCTGGATGATTACTCATAACGGCACCGCTATCAAACGTGCCACGTTCATGTCCTA
CA
SEQ ID NO: 226 pCTM417
GCGCGCTCGCTCGCTCACTGAGGCCGCCCGGGCAAAGCCCGGGCGTCGGGCGACCTTTGGTCGC
CCGGCCTCAGTGAGCGAGCGAGCGCGCAGAGAGGGAGTGGCCAACTCCATCACTAGGGGTTCCT
TGTAGTTAATGATTAACCTCTTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGAGTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGTCGAGGTGAGCCCCACGTTCTGCTTCACTCTCCCCATCTCCCCCCCCTCCCCACCCCCAATT
TTGTATTTATTTATTTTTTAATTATTTTGTGCAGCGATGGGGGGGGGGGGGGGGGGGGGGGGCG
CGCCAGGCGGGGGGGGGCGGGGCGAGGGGCGGGGGGGGGCGAGGCGGAGAGGTGCGGCGGCAGC
CAATCAGAGCGGCGCGCTCCGAAAGTTTCCTTTTATGGCGAGGGGGGGGCGGCGGCGGCCCTAT
AAAAAGCGAAGCGCGCGGCGGGCGGGAGTCGCTGCGACGCTGCCTTCGCCCCGTGCCCCGCTCC
GCCGCCGCCTCGCGCCGCCCGCCCCGGCTCTGACTGACCGCGTTACTCCCACAGGTGAGCGGGC
GGGACGGCCCTTCTCCTCCGGGCTGTAATTAGCGCTTGGTTTAATGACGGCTTGTTTCTTTTCT
GTGGCTGCGTGAAAGCCTTGAGGGGCTCCGGGAGCTAGAGCCTCTGCTAACCATGTTCATGCCT
TCTTCTTTTTCCTACAGCTCCTGGGCAACGTGCTGGTTATTGTGCTGTCTCATCATTTTGGCAA
AGAATTCCTCGAAGATCTAGGCCTGCAGGCCACCATGGCCTCTCATAGACTGCTGCTGCTGTGT
CTGGCCGGCCTGGTGTTTGTGTCTGAGGCCTCTGATACCGGCAGACCCTTCGTGGAAATGTACA
GCGAGATCCCCGAGATCATCCACATGACCGAGGGCAGAGAGCTGGTCATCCCCTGCAGAGTGAC
AAGCCCCAACATCACCGTGACTCTGAAGAAGTTCCCTCTGGACACACTGATCCCCGACGGCAAG
AGAATCATCTGGGACAGCCGGAAGGGCTTCATCATCAGCAACGCCACCTACAAAGAGATCGGCC
TGCTGACCTGTGAAGCCACCGTGAATGGCCACCTGTACAAGACCAACTACCTGACACACAGACA
GACCAACACCATCATCGACGTGGTGCTGAGCCCTAGCCACGGCATTGAACTGTCTGTGGGCGAG
AAGCTGGTGCTGAACTGTACCGCCAGAACCGAGCTGAACGTGGGCATCGACTTCAACTGGGAGT
ACCCCAGCAGCAAGCACCAGCACAAGAAACTGGTCAACCGGGACCTGAAAACCCAGAGCGGCAG
CGAGATGAAGAAATTCCTGAGCACCCTGACCATCGACGGCGTGACCAGATCTGACCAGGGCCTG
TACACATGTGCCGCCAGCTCTGGCCTGATGACCAAGAAAAACAGCACCTTCGTGCGGGTGCACG
AGAAGGACAAGACCCACACCTGTCCTCCATGTCCTGCTCCAGAACTGCTCGGCGGACCTTCCGT
GTTCCTGTTTCCTCCAAAGCCTAAGGACACCCTGATGATCAGCAGAACCCCTGAAGTGACCTGC
GTGGTGGTGGATGTGTCCCACGAGGATCCCGAAGTGAAGTTCAATTGGTACGTGGACGGCGTGG
AAGTGCACAACGCCAAGACCAAGCCTAGAGAGGAACAGTACAATAGCACCTACAGAGTGGTGTC
CGTGCTGACCGTGCTGCACCAGGATTGGCTGAACGGCAAAGAGTACAAGTGCAAGGTGTCCAAC
AAGGCCCTGCCTGCTCCTATCGAGAAAACCATCTCCAAGGCCAAGGGCCAGCCTAGGGAACCCC
AGGTTTACACACTGCCTCCAAGCAGGGACGAGCTGACAAAGAACCAGGTGTCCCTGACCTGCCT
GGTCAAGGGCTTCTACCCTTCCGATATCGCCGTGGAATGGGAGAGCAATGGCCAGCCTGAGAAC
AACTACAAGACAACCCCTCCTGTGCTGGACAGCGACGGCTCATTCTTCCTGTACAGCAAGCTGA
CAGTGGACAAGAGCAGATGGCAGCAGGGCAACGTGTTCAGCTGCAGCGTGATGCACGAGGCCCT
GCACAACCACTACACCCAGAAGTCCCTGAGCCTGTCTCCTGGCTAAGAGGGCCCGGAAACCTGG
CCCTGTCTTCTTGACGAGCATTCCTAGGGGTCTTTCCCCTCTCGCCAAAGGAATGCAAGGTCTG
TTGAATGTCGTGAAGGAAGCAGTTCCTCTGGAAGCTTCTTGAAGACAAACAACGTCTGTAGCGA
CCCTTTGCAGGCAGCGGAACCCCCCACCTGGCGACAGGTGCCTCTGCGGCCAAAAGCCACGTGT
ATAAGATACACCTGCAAAGGCGGCACAACCCCAGTGCCACGTTGTGAGTTGGATAGTTGTGGAA
AGAGTCAAATGGCTCCCCTCAAGCGTATTCAACAAGGGGCTGAAGGATGCCCAGAAGGTACCCC
ATTGTATGGGATCTGATCTGGGGCCTCGGTGCACATGCTTTTCATGTGTTTAGTCGAGGTTAAA
AAACGTCTAGGCCCCCCGAACCACGGGGACGTGGTTTTCCTTTGAAAAACACGATGATAATAGA
ATTCGATATCAAGCTTATCGATACCGTCGACTAGAGCTCGCCACCATGAGACTGCTGGCCAAGA
TCATCTGCCTGATGCTGTGGGCCATCTGCGTGGCCGAGGACTGCAACGAGCTGCCCCCCAGAAG
AAACACCGAGATCCTGACCGGCTCTTGGAGCGACCAGACCTACCCTGAGGGCACCCAGGCCATC
TACAAGTGCAGACCCGGCTACCGGTCCCTGGGCAACATCATCATGGTGTGCAGAAAGGGCGAGT
GGGTGGCCCTGAACCCCCTGAGAAAGTGCCAGAAGAGGCCCTGCGGACACCCCGGCGATACCCC
TTTTGGCACCTTCACACTGACCGGCGGCAACGTGTTCGAGTACGGCGTGAAGGCCGTGTACACC
TGTAACGAGGGCTACCAGCTGCTGGGCGAGATCAACTACAGAGAGTGCGACACCGACGGCTGGA
CCAACGATATCCCCATCTGCGAGGTCGTGAAGTGCCTGCCTGTGACCGCCCCAGAGAACGGCAA
GATCGTGTCCAGCGCCATGGAACCCGACAGAGAGTACCACTTCGGCCAGGCCGTCAGATTCGTG
TGCAACAGCGGCTACAAGATCGAGGGCGACGAGGAAATGCACTGCAGCGACGACGGCTTCTGGT
CCAAAGAAAAGCCTAAGTGCGTGGAAATCAGCTGCAAGAGCCCCGACGTGATCAACGGCAGCCC
CATCAGCCAGAAGATCATCTACAAAGAGAACGAGCGGTTCCAGTACAAGTGTAACATGGGCTAC
GAGTACAGCGAGCGGGGCGACGCCGTGTGTACAGAATCTGGATGGCGGCCTCTGCCCAGCTGCG
AGGAAAAGAGCTGCGACAACCCCTACATCCCCAACGGCGACTACAGCCCCCTGCGGATCAAGCA
CAGAACCGGCGACGAGATCACCTACCAGTGCCGGAACGGCTTCTACCCCGCCACCAGAGGCAAT
ACCGCCAAGTGTACCAGCACCGGCTGGATCCCTGCCCCCAGATGTACCCTGAAGCCCTGCGACT
ACCCTGACATCAAGCACGGCGGCCTGTACCACGAGAACATGCGGAGGCCCTACTTCCCTGTGGC
CGTGGGCAAGTACTACAGCTACTACTGCGACGAGCACTTCGAGACACCCAGCGGCAGCTACTGG
GACCACATCCACTGTACCCAGGACGGCTGGTCCCCTGCCGTGCCCTGCCTGAGGAAGTGCTACT
TCCCCTACCTGGAAAACGGCTACAACCAGAACTACGGCCGGAAGTTCGTGCAGGGCAAGAGCAT
CGATGTGGCCTGCCACCCTGGATACGCCCTGCCTAAGGCCCAGACCACCGTGACCTGCATGGAA
AATGGATGGTCCCCCACCCCCCGGTGCATCAGAGTGTCCTTCACCCTGTGAGAGCTCGCTGATC
AGCCTCGACTGTGCCTTCTAGTTGCCAGCCATCTGTTGTTTGCCCCTCCCCCGTGCCTTCCTTG
ACCCTGGAAGGTGCCACTCCCACTGTCCTTTCCTAATAAAATGAGGAAATTGCATCGCATTGTC
TGAGTAGGTGTCATTCTATTCTGGGGGGTGGGGTGGGGCAGGACAGCAAGGGGGAGGATTGGGA
AGACAATAGCAGGCATGCTCAGGTTAATCATTAACTACAAGGAACCCCTAGTGATGGAGTTGGC
CACTCCCTCTCTGCGCGCTCGCTCGCTCACTGAGGCCGGGCGACCAAAGGTCGCCCGACGCCCG
GGCTTTGCCCGGGCGGCCTCAGTGAGCGAGCGAGCGCGCACTGTCATTAGCAACTCCTTGTCCT
TCGATCTCGTCAACAACAGCTTGCAGTTCAAATACAAGACCCAGAAGGCGACTATTCTGGAAGC
GAGCTTGAAGAGTTAACCTGCAGAGAGCCCCCGCAGTGTCGACAATTAATCATCGGCATAGTAT
ATCGGCATAGTATAATACGACAAGGTGAGGAAGTAAAAAATGAGCCATATCCAACGGGAAACGT
CGAGGCCGCGATTAAATTCCAACATGGATGCTGATTTATATGGGTATAAATGGGCTCGCGATAA
TGTCGGGCAATCAGGTGCGACAATCTATCGCTTGTATGGGAAGCCCGATGCGCCAGAGTTGTTT
CTGAAACATGGCAAAGGTAGCGTTGCCAATGATGTTACAGATGAGATGGTCAGACTAAACTGGC
TGACGGAATTTATGCCACTTCCGACCATCAAGCATTTTATCCGTACTCCTGATGATGCATGGTT
ACTCACCACTGCGATCCCCGGAAAAACAGCGTTCCAGGTATTAGAAGAATATCCTGATTCAGGT
GAAAATATTGTTGATGCGCTGGCAGTGTTCCTGCGCCGGTTGCACTCGATTCCTGTTTGTAATT
GTCCTTTTAACAGCGATCGCGTATTTCGCCTCGCTCAGGCGCAATCACGAATGAATAACGGTTT
GGTTGATGCGAGTGATTTTGATGACGAGCGTAATGGCTGGCCTGTTGAACAAGTCTGGAAAGAA.
ATGCATAAACTTTTGCCATTCTCACCGGATTCAGTCGTCACTCATGGTGATTTCTCACTTGATA
ACCTTATTTTTGACGAGGGGAAATTAATAGGTTGTATTGATGTTGGACGAGTCGGAATCGCAGA
CCGATACCAGGATCTTGCCATCCTATGGAACTGCCTCGGTGAGTTTTCTCCTTCATTACAGAAA
CGGCTTTTTCAAAAATATGGTATTGATAATCCTGATATGAATAAATTGCAGTTTCATTTGATGC
TCGATGAGTTTTTCTAACTCATGACCAAAATCCCTTAACGTGAGTTACGCGCGCGTCGTTCCAC
TGAGCGTCAGACCCCGTAGAAAAGATCAAAGGATCTTCTTGAGATCCTTTTTTTCTGCGCGTAA
TCTGCTGCTTGCAAACAAAAAAACCACCGCTACCAGCGGTGGTTTGTTTGCCGGATCAAGAGCT
ACCAACTCTTTTTCCGAAGGTAACTGGCTTCAGCAGAGCGCAGATACCAAATACTGTTCTTCTA
GTGTAGCCGTAGTTAGCCCACCACTTCAAGAACTCTGTAGCACCGCCTACATACCTCGCTCTGC
TAATCCTGTTACCAGTGGCTGCTGCCAGTGGCGATAAGTCGTGTCTTACCGGGTTGGACTCAAG
ACGATAGTTACCGGATAAGGCGCAGCGGTCGGGCTGAACGGGGGGTTCGTGCACACAGCCCAGC
TTGGAGCGAACGACCTACACCGAACTGAGATACCTACAGCGTGAGCTATGAGAAAGCGCCACGC
TTCCCGAAGGGAGAAAGGCGGACAGGTATCCGGTAAGCGGCAGGGTCGGAACAGGAGAGCGCAC
GAGGGAGCTTCCAGGGGGAAACGCCTGGTATCTTTATAGTCCTGTCGGGTTTCGCCACCTCTGA
CTTGAGCGTCGATTTTTGTGATGCTCGTCAGGGGGGCGGAGCCTATGGAAAAACGCCAGCAACG
CGGCCTTTTTACGGTTCCTGGCCTTTTGCTGGCCTTTTGCTCACATGTTCTTTCCTGCGTTATC
CCCTGATTCTGTGGATAACCGTATTACCGCCTTTGAGTGAGCTGATACCGCTCAAGGCTGACTG
CAGGGCGAGAAGATTGCGAGCTGTGCGGCTGAGTTGACGTATCTGTGCTGGATGATTACTCATA
ACGGCACCGCTATCAAACGTGCCACGTTCATGTCCTACA
SEQ ID NO: 227 pCTM369
GCGCGCTCGCTCGCTCACTGAGGCCGCCCGGGCAAAGCCCGGGCGTCGGGCGACCTTTGGTCGC
CCGGCCTCAGTGAGCGAGCGAGCGCGCAGAGAGGGAGTGGCCAACTCCATCACTAGGGGTTCCT
TGTAGTTAATGATTAACCTCTTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGAGTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGTCGAGGTGAGCCCCACGTTCTGCTTCACTCTCCCCATCTCCCCCCCCTCCCCACCCCCAATT
TTGTATTTATTTATTTTTTAATTATTTTGTGCAGCGATGGGGGGGGGGGGGGGGGGGGGGGGCG
CGCCAGGCGGGGCGGGGCGGGGCGAGGGGCGGGGCGGGGCGAGGCGGAGAGGTGCGGCGGCAGC
CAATCAGAGCGGCGCGCTCCGAAAGTTTCCTTTTATGGCGAGGGGGGGGCGGCGGCGGCCCTAT
AAAAAGCGAAGCGCGCGGCGGGCGGGAGTCGCTGCGACGCTGCCTTCGCCCCGTGCCCCGCTCC
GCCGCCGCCTCGCGCCGCCCGCCCCGGCTCTGACTGACCGCGTTACTCCCACAGGTGAGCGGGC
GGGACGGCCCTTCTCCTCCGGGCTGTAATTAGCGCTTGGTTTAATGACGGCTTGTTTCTTTTCT
GTGGCTGCGTGAAAGCCTTGAGGGGCTCCGGGAGCTAGAGCCTCTGCTAACCATGTTCATGCCT
TCTTCTTTTTCCTACAGCTCCTGGGCAACGTGCTGGTTATTGTGCTGTCTCATCATTTTGGCAA
AGAATTCCTCGAAGATCTAGGCCTGCAGGCGGCCGCCACCATGAGACTGCTGGCCAAGATCATC
TGCCTGATGCTGTGGGCCATCTGCGTGGCCGAGGACTGCAACGAGCTGCCCCCCAGAAGAAACA
CCGAGATCCTGACCGGCTCTTGGAGCGACCAGACCTACCCTGAGGGCACCCAGGCCATCTACAA
GTGCAGACCCGGCTACCGGTCCCTGGGCAACATCATCATGGTGTGCAGAAAGGGCGAGTGGGTG
GCCCTGAACCCCCTGAGAAAGTGCCAGAAGAGGCCCTGCGGACACCCCGGCGATACCCCTTTTG
GCACCTTCACACTGACCGGCGGCAACGTGTTCGAGTACGGCGTGAAGGCCGTGTACACCTGTAA
CGAGGGCTACCAGCTGCTGGGCGAGATCAACTACAGAGAGTGCGACACCGACGGCTGGACCAAC
GATATCCCCATCTGCGAGGTCGTGAAGTGCCTGCCTGTGACCGCCCCAGAGAACGGCAAGATCG
TGTCCAGCGCCATGGAACCCGACAGAGAGTACCACTTCGGCCAGGCCGTCAGATTCGTGTGCAA
CAGCGGCTACAAGATCGAGGGCGACGAGGAAATGCACTGCAGCGACGACGGCTTCTGGTCCAAA
GAAAAGCCTAAGTGCGTGGAAATCAGCTGCAAGAGCCCCGACGTGATCAACGGCAGCCCCATCA
GCCAGAAGATCATCTACAAAGAGAACGAGCGGTTCCAGTACAAGTGTAACATGGGCTACGAGTA
CAGCGAGCGGGGCGACGCCGTGTGTACAGAATCTGGATGGCGGCCTCTGCCCAGCTGCGAGGAA
AAGAGCTGCGACAACCCCTACATCCCCAACGGCGACTACAGCCCCCTGCGGATCAAGCACAGAA
CCGGCGACGAGATCACCTACCAGTGCCGGAACGGCTTCTACCCCGCCACCAGAGGCAATACCGC
CAAGTGTACCAGCACCGGCTGGATCCCTGCCCCCAGATGTACCCTGAAGCCCTGCGACTACCCT
GACATCAAGCACGGCGGCCTGTACCACGAGAACATGCGGAGGCCCTACTTCCCTGTGGCCGTGG
GCAAGTACTACAGCTACTACTGCGACGAGCACTTCGAGACACCCAGCGGCAGCTACTGGGACCA
CATCCACTGTACCCAGGACGGCTGGTCCCCTGCCGTGCCCTGCCTGAGGAAGTGCTACTTCCCC
TACCTGGAAAACGGCTACAACCAGAACTACGGCCGGAAGTTCGTGCAGGGCAAGAGCATCGATG
TGGCCTGCCACCCTGGATACGCCCTGCCTAAGGCCCAGACCACCGTGACCTGCATGGAAAATGG
ATGGTCCCCCACCCCCCGGTGCATCAGAGTGTCCTTCACCCTGTGAGGTACCACTGTGCATATG
CGTTACTGGCCGAAGCCGCTTGGAATAAGGCCGGTGTGCGTTTGTCTATATGTTATTTTCCACC
ATATTGCCGTCTTTTGGCAATGTGAGGGCCCGGAAACCTGGCCCTGTCTTCTTGACGAGCATTC
CTAGGGGTCTTTCCCCTCTCGCCAAAGGAATGCAAGGTCTGTTGAATGTCGTGAAGGAAGCAGT
TCCTCTGGAAGCTTCTTGAAGACAAACAACGTCTGTAGCGACCCTTTGCAGGCAGCGGAACCCC
CCACCTGGCGACAGGTGCCTCTGCGGCCAAAAGCCACGTGTATAAGATACACCTGCAAAGGCGG
CACAACCCCAGTGCCACGTTGTGAGTTGGATAGTTGTGGAAAGAGTCAAATGGCTCCCCTCAAG
CGTATTCAACAAGGGGCTGAAGGATGCCCAGAAGGTACCCCATTGTATGGGATCTGATCTGGGG
CCTCGGTGCACATGCTTTTCATGTGTTTAGTCGAGGTTAAAAAACGTCTAGGCCCCCCGAACCA
CGGGGACGTGGTTTTCCTTTGAAAAACACGATGATAATAGAATTCGATATCAAGCTTATCGATA
CCGTCGACTAGAGCTCCACCATGAAGCTGCTGCATGTGTTTCTGCTGTTCCTCTGCTTCCACCT
GAGGTTCTGCAAAGTGACCTACACCAGCCAAGAGGACCTGGTGGAAAAGAAGTGCCTGGCCAAG
AAGTACACCCACCTGAGCTGCGACAAGGTGTTCTGCCAGCCTTGGCAGAGATGCATCGAGGGCA
CCTGTGTGTGCAAGCTGCCCTATCAGTGCCCCAAGAATGGCACAGCCGTGTGCGCCACCAACAG
AAGAAGCTTCCCTACCTACTGCCAGCAGAAAAGCCTGGAATGTCTGCACCCCGGCACCAAGTTT
CTGAACAACGGCACCTGTACCGCCGAGGGCAAGTTTAGCGTGTCCCTGAAGCACGGCAACACCG
ACTCTGAGGGCATCGTGGAAGTGAAGCTGGTGGACCAGGACAAGACCATGTTCATCTGCAAGAG
CAGCTGGTCCATGCGCGAGGCCAATGTGGCTTGTCTGGATCTGGGATTCCAGCAGGGCGCCGAC
ACACAGAGAAGATTCAAGCTGAGCGACCTGAGCATCAACAGCACCGAGTGCCTGCATGTGCACT
GTAGAGGCCTGGAAACAAGCCTGGCCGAGTGCACCTTCACCAAGAGAAGGACCATGGGCTACCA
GGACTTCGCCGACGTCGTGTGCTACACCCAGAAAGCCGACTCTCCCATGGACGATTTCTTCCAG
TGCGTGAACGGCAAGTACATCAGCCAGATGAAGGCCTGCGACGGCATCAACGATTGCGGCGATC
AGAGCGACGAGCTGTGCTGCAAAGCCTGTCAAGGCAAGGGCTTCCACTGCAAGTCCGGCGTGTG
TATCCCTAGCCAGTACCAGTGCAATGGCGAGGTGGACTGTATCACCGGCGAGGATGAAGTGGGC
TGTGCCGGATTTGCCAGCGTGGCCCAAGAGGAAACCGAGATCCTGACCGCCGATATGGACGCCG
AGCGGCGGAGAATCAAAAGCCTGCTGCCTAAGCTGTCCTGCGGCGTGAAGAACCGGATGCACAT
CCGGCGCAAGAGAATCGTCGGAGGCAAAAGAGCACAGCTGGGCGATCTGCCTTGGCAAGTGGCC
ATCAAGGATGCCTCCGGCATCACTTGTGGCGGCATCTACATCGGCGGCTGCTGGATTCTGACAG
CCGCTCATTGTCTGCGGGCCAGCAAGACCCACCGGTATCAGATCTGGACCACCGTGGTGGACTG
GATTCACCCCGACCTGAAGCGGATCGTGATCGAGTACGTGGACCGGATCATCTTCCACGAGAAC
TACAACGCCGGCACCTACCAGAACGATATCGCCCTGATCGAGATGAAGAAGGACGGGAACAAGA
AGGACTGCGAGCTGCCTAGATCTATCCCCGCCTGTGTTCCTTGGAGCCCCTACCTGTTCCAGCC
TAACGATACCTGCATCGTGTCCGGCTGGGGCAGAGAGAAGGATAACGAGAGGGTGTTCAGCCTG
CAGTGGGGCGAAGTGAAACTGATCAGCAACTGCAGCAAGTTCTACGGCAACCGGTTCTACGAGA
AAGAAATGGAATGCGCCGGCACATACGACGGCTCCATCGATGCCTGTAAAGGCGATTCTGGCGG
CCCTCTCGTGTGCATGGATGCCAACAATGTGACCTACGTGTGGGGCGTCGTGTCCTGGGGAGAG
AATTGTGGCAAGCCTGAGTTCCCCGGCGTGTACACCAAGGTGGCCAACTACTTCGACTGGATCA
GCTACCACGTGGGCAGACCCTTTATCAGCCAGTACAACGTCTGAGAGCTCGCTGATCAGCCTCG
ACTGTGCCTTCTAGTTGCCAGCCATCTGTTGTTTGCCCCTCCCCCGTGCCTTCCTTGACCCTGG
AAGGTGCCACTCCCACTGTCCTTTCCTAATAAAATGAGGAAATTGCATCGCATTGTCTGAGTAG
GTGTCATTCTATTCTGGGGGGTGGGGTGGGGCAGGACAGCAAGGGGGAGGATTGGGAAGACAAT
AGCAGGCATGCTCAGGTTAATCATTAACTACAAGGAACCCCTAGTGATGGAGTTGGCCACTCCC
TCTCTGCGCGCTCGCTCGCTCACTGAGGCCGGGCGACCAAAGGTCGCCCGACGCCCGGGCTTTG
CCCGGGCGGCCTCAGTGAGCGAGCGAGCGCGCACTGTCATTAGCAACTCCTTGTCCTTCGATCT
CGTCAACAACAGCTTGCAGTTCAAATACAAGACCCAGAAGGCGACTATTCTGGAAGCGAGCTTG
AAGAGTTAACCTGCAGAGAGCCCCCGCAGTGTCGACAATTAATCATCGGCATAGTATATCGGCA
TAGTATAATACGACAAGGTGAGGAAGTAAAAAATGAGCCATATCCAACGGGAAACGTCGAGGCC
GCGATTAAATTCCAACATGGATGCTGATTTATATGGGTATAAATGGGCTCGCGATAATGTCGGG
CAATCAGGTGCGACAATCTATCGCTTGTATGGGAAGCCCGATGCGCCAGAGTTGTTTCTGAAAC
ATGGCAAAGGTAGCGTTGCCAATGATGTTACAGATGAGATGGTCAGACTAAACTGGCTGACGGA
ATTTATGCCACTTCCGACCATCAAGCATTTTATCCGTACTCCTGATGATGCATGGTTACTCACC
ACTGCGATCCCCGGAAAAACAGCGTTCCAGGTATTAGAAGAATATCCTGATTCAGGTGAAAATA
TTGTTGATGCGCTGGCAGTGTTCCTGCGCCGGTTGCACTCGATTCCTGTTTGTAATTGTCCTTT
TAACAGCGATCGCGTATTTCGCCTCGCTCAGGCGCAATCACGAATGAATAACGGTTTGGTTGAT
GCGAGTGATTTTGATGACGAGCGTAATGGCTGGCCTGTTGAACAAGTCTGGAAAGAAATGCATA
AACTTTTGCCATTCTCACCGGATTCAGTCGTCACTCATGGTGATTTCTCACTTGATAACCTTAT
TTTTGACGAGGGGAAATTAATAGGTTGTATTGATGTTGGACGAGTCGGAATCGCAGACCGATAC
CAGGATCTTGCCATCCTATGGAACTGCCTCGGTGAGTTTTCTCCTTCATTACAGAAACGGCTTT
TTCAAAAATATGGTATTGATAATCCTGATATGAATAAATTGCAGTTTCATTTGATGCTCGATGA
GTTTTTCTAACTCATGACCAAAATCCCTTAACGTGAGTTACGCGCGCGTCGTTCCACTGAGCGT
CAGACCCCGTAGAAAAGATCAAAGGATCTTCTTGAGATCCTTTTTTTCTGCGCGTAATCTGCTG
CTTGCAAACAAAAAAACCACCGCTACCAGCGGTGGTTTGTTTGCCGGATCAAGAGCTACCAACT
CTTTTTCCGAAGGTAACTGGCTTCAGCAGAGCGCAGATACCAAATACTGTTCTTCTAGTGTAGC
CGTAGTTAGCCCACCACTTCAAGAACTCTGTAGCACCGCCTACATACCTCGCTCTGCTAATCCT
GTTACCAGTGGCTGCTGCCAGTGGCGATAAGTCGTGTCTTACCGGGTTGGACTCAAGACGATAG
TTACCGGATAAGGCGCAGCGGTCGGGCTGAACGGGGGGTTCGTGCACACAGCCCAGCTTGGAGC
GAACGACCTACACCGAACTGAGATACCTACAGCGTGAGCTATGAGAAAGCGCCACGCTTCCCGA
AGGGAGAAAGGCGGACAGGTATCCGGTAAGCGGCAGGGTCGGAACAGGAGAGCGCACGAGGGAG
CTTCCAGGGGGAAACGCCTGGTATCTTTATAGTCCTGTCGGGTTTCGCCACCTCTGACTTGAGC
GTCGATTTTTGTGATGCTCGTCAGGGGGGCGGAGCCTATGGAAAAACGCCAGCAACGCGGCCTT
TTTACGGTTCCTGGCCTTTTGCTGGCCTTTTGCTCACATGTTCTTTCCTGCGTTATCCCCTGAT
TCTGTGGATAACCGTATTACCGCCTTTGAGTGAGCTGATACCGCTCAAGGCTGACTGCAGGGCG
AGAAGATTGCGAGCTGTGCGGCTGAGTTGACGTATCTGTGCTGGATGATTACTCATAACGGCAC
CGCTATCAAACGTGCCACGTTCATGTCCTACA
SEQ ID NO: 228 FHR-2 dimerization domain (SCR1-2)
EAMFCDFPKINHGILYDEEKYKPFSQVPTGEVFYYSCEYNFVSPSKSFWTRITCAEEGWSPTPK
CLRLCFFPFVENGHSESSGQTHLEGDTVQIICNTGYRLQNNENNISCVERGWSTPPKCRS
SEQ ID NO: 229 FHR-2 SCR1-Non-codon Optimized
GAAGCAATGTTCTGTGATTTTCCAAAAATAAACCATGGAATTCTATATGATGAAGAAAAATATA
AGCCATTTTCCCAAGTTCCTACAGGGGAAGTTTTCTATTACTCCTGTGAATATAATTTTGTGTC
TCCTTCAAAATCCTTTTGGACTCGCATAACGTGCGCAGAAGAAGGATGGTCACCAACACCAAAG
TGTCTC
SEQ ID NO: 230 FHR-2 SCR1-GeneArt Codon Optimized
GAGGCCATGTTCTGCGACTTCCCCAAGATCAACCACGGCATCCTGTACGACGAGGAAAAGTACA
AGCCATTCAGCCAGGTGCCAACCGGCGAGGTGTTCTACTACAGCTGCGAGTACAACTTCGTGTC
CCCTAGCAAGAGCTTCTGGACCCGGATCACCTGTGCCGAAGAAGGCTGGTCCCCTACACCTAAG
TGTCTG
SEQ ID NO: 231 FHR-2 SCR2-Non-codon Optimized
AGACTGTGTTTCTTTCCTTTTGTGGAAAATGGTCATTCTGAATCTTCAGGACAAACACATCTGG
AAGGTGATACTGTACAAATTATTTGCAACACAGGATACAGACTTCAAAACAATGAGAACAACAT
TTCATGTGTAGAACGGGGCTGGTCCACTCCTCCCAAATGCAGGTCC
SEQ ID NO: 232 FHR-2 SCR2-GeneArt Codon Optimized
CGGCTGTGCTTCTTCCCCTTCGTGGAAAATGGCCACAGCGAGAGCAGCGGCCAGACACATCTTG
AGGGCGATACCGTGCAGATCATCTGTAACACCGGCTACCGGCTGCAGAACAACGAGAACAACAT
CAGCTGCGTGGAACGCGGCTGGTCTACCCCTCCTAAGTGTAGATCT
SEQ ID NO: 233 FHR-5 dimerization domain (SCR1-2)
EGTLCDFPKIHHGFLYDEEDYNPFSQVPTGEVFYYSCEYNFVSPSKSFWTRITCTEEGWSPTPK
CLRMCSFPFVKNGHSESSGLIHLEGDTVQIICNTGYSLQNNEKNISCVERGWSTPPICSF
SEQ ID NO: 234 FHR-5 SCR1-Non-codon Optimized
GAAGGAACACTTTGTGATTTTCCAAAAATACACCATGGATTTCTGTATGATGAAGAAGATTATA
ACCCTTTTTCCCAAGTTCCTACAGGGGAAGTTTTCTATTACTCCTGTGAATATAATTTTGTGTC
TCCTTCAAAATCCTTTTGGACTCGCATAACATGCACAGAAGAAGGATGGTCACCAACACCGAAG
TGTCTC
SEQ ID NO: 235 FHR-5 SCR1-GeneArt Codon Optimized
GAGGGCACCCTGTGCGACTTTCCAAAGATCCACCACGGCTTTCTGTACGACGAAGAGGACTACA
ACCCATTCAGCCAGGTGCCAACCGGCGAGGTGTTCTACTACAGCTGCGAGTACAACTTCGTGTC
CCCTAGCAAGAGCTTCTGGACCCGGATCACCTGTACCGAGGAAGGCTGGTCCCCTACACCTAAG
TGTCTG
SEQ ID NO: 236 FHR-5 SCR2-Non-codon Optimized
AGAATGTGTTCCTTTCCTTTTGTGAAAAATGGTCATTCTGAATCTTCAGGACTAATACATCTGG
AAGGTGATACTGTACAAATTATTTGCAACACAGGATACAGCCTTCAAAACAATGAGAAAAACAT
TTCGTGTGTAGAACGGGGCTGGTCCACTCCTCCCATATGCAGCTTC
SEQ ID NO: 237 FHR-5 SCR2-GeneArt Codon Optimized
CGGATGTGCAGCTTCCCATTCGTGAAGAACGGCCACAGCGAGAGCAGCGGACTGATTCACCTGG
AAGGCGACACCGTGCAGATCATCTGTAACACCGGCTACAGCCTGCAGAACAACGAGAAGAACAT
CAGCTGCGTGGAACGCGGCTGGTCTACCCCTCCTATCTGCAGCTTT
SEQ ID NO: 238 BEST1-EP-454 Enhancer Promoter
CGTTACATAACTTACGGTAAATGGCCCGCCTGGCTGACCGCCCAACGACCCCCGCCCATTGACG
TCAATAATGACGTATGTTCCCATAGTAACGCCAATAGGGACTTTCCATTGACGTCAATGGGTGG
AGTATTTACGGTAAACTGCCCACTTGGCAGTACATCAAGTGTATCATATGCCAAGTACGCCCCC
TATTGACGTCAATGACGGTAAATGGCCCGCCTGGCATTATGCCCAGTACATGACCTTATGGGAC
TTTCCTACTTGGCAGTACATCTACGTATTAGTCATCGCTATTACCATGCTCGAGCTAGGGTGAT
GAAATTCCCAAGCAACACCATCCTTTTCAAGTGACGGCGGCTCAGCACTCACGTGGGCAGTGCC
AGCCTCTAAGAGTGGGCAGGGGCACTGGCCACAGAGTCCCAGGGAGTCCCACCAGCCTAGTCGC
CAGACC
SEQ ID NO: 239 BEST1-V3 Promoter-144 b
CTAGGGTGATGAAATTCCCAAGCAACACCATCCTTTTCAAGTGACGGCGGCTCAGCACTCACGT
GGGCAGTGCCAGCCTCTAAGAGTGGGCAGGGGCACTGGCCACAGAGTCCCAGGGAGTCCCACCA
GCCTAGTCGCCAGACC
SEQ ID NO: 240 HTRAI protein sequence
MQIPRAALLPLLLLLLAAPASAQLSRAGRSAPLAAGCPDRCEPARCPPQPEHCEGGRARDACGC
CEVCGAPEGAACGLQEGPCGEGLQCVVPFGVPASATVRRRAQAGLCVCASSEPVCGSDANTYAN
LCQLRAASRRSERLHRPPVIVLQRGACGQGQEDPNSLRHKYNFIADVVEKIAPAVVHIELFRKL
PFSKREVPVASGSGFIVSEDGLIVTNAHVVTNKHRVKVELKNGATYEAKIKDVDEKADIALIKI
DHQGKLPVLLLGRSSELRPGEFVVAIGSPFSLQNTVTTGIVSTTQRGGKELGLRNSDMDYIQTD
AIINYGNSGGPLVNLDGEVIGINTLKVTAGISFAIPSDKIKKFLTESHDRQAKGKAITKKKYIG
IRMMSLTSSKAKELKDRHRDFPDVISGAYIIEVIPDTPAEAGGLKENDVIISINGQSVVSANDV
SDVIKRESTINMVVRRGNEDIMITVIPEEIDP
SEQ ID NO: 241 Control sequence, Section 10.4.1.3
GCACTACCAGAGCTAACTCAGATAGTACT
SEQ ID NO: 242 SCR7 protein sequence
KCYFPYLENGYNQNYGRKFVQGKSIDVACHPGYALPKAQTTVTCMENGWSPTPRCIRV
SEQ ID NO: 243 C7 shRNA sequence:
GAAGGTCCTTCAGCATTTCTCTGTGGCTCCAAGAGAGCCACAGAGAAATGCTGAAGGACCTTC
SEQ ID NO: 244 C7 shRNA sequence:
CAGAATGCAATGGCTGTACCAAGACTCAGCAAGACTGAGTCTTGGTACAGCCATTGCATTCTG
SEQ ID NO: 245 C7 shRNA sequence:
TCTGTCCATCACCTCCTGCCTTGAAAGATCAAGAATCTTTCAAGGCAGGAGGTGATGGACAGA
SEQ ID NO: 246 mCFHT.1 cDNA sequence
ATGAGACTGCTGGCCAAGATCATCTGCCTGATGCTGTGGGCCATCTGCGTGGCCGAGGACTGCA
ACGAGCTGCCCCCCAGAAGAAACACCGAGATCCTGACCGGCTCTTGGAGCGACCAGACCTACCC
TGAGGGCACCCAGGCCATCTACAAGTGCAGACCCGGCTACCGGTCCCTGGGCAACATCATCATG
GTGTGCAGAAAGGGCGAGTGGGTGGCCCTGAACCCCCTGAGAAAGTGCCAGAAGAGGCCCTGCG
GACACCCCGGCGATACCCCTTTTGGCACCTTCACACTGACCGGCGGCAACGTGTTCGAGTACGG
CGTGAAGGCCGTGTACACCTGTAACGAGGGCTACCAGCTGCTGGGCGAGATCAACTACAGAGAG
TGCGACACCGACGGCTGGACCAACGATATCCCCATCTGCGAGGTCGTGAAGTGCCTGCCTGTGA
CCGCCCCAGAGAACGGCAAGATCGTGTCCAGCGCCATGGAACCCGACAGAGAGTACCACTTCGG
CCAGGCCGTCAGATTCGTGTGCAACAGCGGCTACAAGATCGAGGGCGACGAGGAAATGCACTGC
AGCGACGACGGCTTCTGGTCCAAAGAAAAGCCTAAGTGCGTGGAAATCAGCTGCAAGAGCCCCG
ACGTGATCAACGGCAGCCCCATCAGCCAGAAGATCATCTACAAAGAGAACGAGCGGTTCCAGTA
CAAGTGTAACATGGGCTACGAGTACAGCGAGCGGGGCGACGCCGTGTGTACAGAATCTGGATGG
CGGCCTCTGCCCAGCTGCGAGGAAAAGAGCTGCGACAACCCCTACATCCCCAACGGCGACTACA
GCCCCCTGCGGATCAAGCACAGAACCGGCGACGAGATCACCTACCAGTGCCGGAACGGCTTCTA
CCCCGCCACCAGAGGCAATACCGCCAAGTGTACCAGCACCGGCTGGATCCCTGCCCCCAGATGT
ACCCTGAAGCCCTGCGACTACCCTGACATCAAGCACGGCGGCCTGTACCACGAGAACATGCGGA
GGCCCTACTTCCCTGTGGCCGTGGGCAAGTACTACAGCTACTACTGCGACGAGCACTTCGAGAC
ACCCAGCGGCAGCTACTGGGACCACATCCACTGTACCCAGGACGGCTGGTCCCCTGCCGTGCCC
TGCCTGAGGAAGTGCTACTTCCCCTACCTGGAAAACGGCTACAACCAGAACTACGGCCGGAAGT
TCGTGCAGGGCAAGAGCATCGATGTGGCCTGCCACCCTGGATACGCCCTGCCTAAGGCCCAGAC
CACCGTGACCTGCATGGAAAATGGATGGTCCCCCACCCCCCGGTGCATCAGAGTGAGGAAGTGC
TATTTCCCTTATTTGGAAAACGGATATAATCAAAATTACGGTCGGAAATTTGTCCAAGGTAAGT
CAATAGATGTCGCGTGTCATCCTGGCTATGCTCTTCCGAAAGCACAGACAACGGTTACGTGTAT
GGAGAACGGTTGGTCTCCAACCCCTCGATGTATTAGGGTGTCCTTCACCCTGTGA
SEQ ID NO: 247 mCFHT.1 protein sequence
MRLLAKIICLMLWAICVAEDCNELPPRRNTEILTGSWSDQTYPEGTQAIYKCRPGYRSLGNIIM
VCRKGEWVALNPLRKCQKRPCGHPGDTPFGTFTLTGGNVFEYGVKAVYTCNEGYQLLGEINYRE
CDTDGWTNDIPICEVVKCLPVTAPENGKIVSSAMEPDREYHFGQAVRFVCNSGYKIEGDEEMHC
SDDGFWSKEKPKCVEISCKSPDVINGSPISQKIIYKENERFQYKCNMGYEYSERGDAVCTESGW
RPLPSCEEKSCDNPYIPNGDYSPLRIKHRTGDEITYQCRNGFYPATRGNTAKCTSTGWIPAPRC
TLKPCDYPDIKHGGLYHENMRRPYFPVAVGKYYSYYCDEHFETPSGSYWDHIHCTQDGWSPAVP
CIRKCYFRYLENGYNQNYGRKEVQGKSIDVACHPGYALPKAQTTVTCMENGWSPTPRCIRVRKC
YFPYLENGYNQNYGRKFVQGKSIDVACHPGYALPKAQTTVTCMENGWSPTPRCIRVSFTL*
SEQ ID NO: 248 CFHT.2 cDNA sequence
ATGAGACTGCTGGCCAAGATCATCTGCCTGATGCTGTGGGCCATCTGCGTGGCCGAGGACTGCA
ACGAGCTGCCCCCCAGAAGAAACACCGAGATCCTGACCGGCTCTTGGAGCGACCAGACCTACCC
TGAGGGCACCCAGGCCATCTACAAGTGCAGACCCGGCTACCGGTCCCTGGGCAACATCATCATG
GTGTGCAGAAAGGGCGAGTGGGTGGCCCTGAACCCCCTGAGAAAGTGCCAGAAGAGGCCCTGCG
GACACCCCGGCGATACCCCTTTTGGCACCTTCACACTGACCGGCGGCAACGTGTTCGAGTACGG
CGTGAAGGCCGTGTACACCTGTAACGAGGGCTACCAGCTGCTGGGCGAGATCAACTACAGAGAG
TGCGACACCGACGGCTGGACCAACGATATCCCCATCTGCGAGGTCGTGAAGTGCCTGCCTGTGA
CCGCCCCAGAGAACGGCAAGATCGTGTCCAGCGCCATGGAACCCGACAGAGAGTACCACTTCGG
CCAGGCCGTCAGATTCGTGTGCAACAGCGGCTACAAGATCGAGGGCGACGAGGAAATGCACTGC
AGCGACGACGGCTTCTGGTCCAAAGAAAAGCCTAAGTGCGTGGAAATCAGCTGCAAGAGCCCCG
ACGTGATCAACGGCAGCCCCATCAGCCAGAAGATCATCTACAAAGAGAACGAGCGGTTCCAGTA
CAAGTGTAACATGGGCTACGAGTACAGCGAGCGGGGCGACGCCGTGTGTACAGAATCTGGATGG
CGGCCTCTGCCCAGCTGCGAGGAAAAGAGCTGCGACAACCCCTACATCCCCAACGGCGACTACA
GCCCCCTGCGGATCAAGCACAGAACCGGCGACGAGATCACCTACCAGTGCCGGAACGGCTTCTA
CCCCGCCACCAGAGGCAATACCGCCAAGTGTACCAGCACCGGCTGGATCCCTGCCCCCAGATGT
ACCCTGAAGCCCTGCGACTACCCTGACATCAAGCACGGCGGCCTGTACCACGAGAACATGCGGA
GGCCCTACTTCCCTGTGGCCGTGGGCAAGTACTACAGCTACTACTGCGACGAGCACTTCGAGAC
ACCCAGCGGCAGCTACTGGGACCACATCCACTGTACCCAGGACGGCTGGTCCCCTGCCGTGCCC
TGCCTGAGGAAGTGCTACTTCCCCTACCTGGAAAACGGCTACAACCAGAACTACGGCCGGAAGT
TCGTGCAGGGCAAGAGCATCGATGTGGCCTGCCACCCTGGATACGCCCTGCCTAAGGCCCAGAC
CACCGTGACCTGCATGGAAAATGGATGGTCCCCCACCCCCCGGTGCATCAGAGTGAGGAAGTGC
TATTTCCCTTATTTGGAAAACGGATATAATCAAAATTACGGTCGGAAATTTGTCCAAGGTAAGT
CAATAGATGTCGCGTGTCATCCTGGCTATGCTCTTCCGAAAGCACAGACAACGGTTACGTGTAT
GGAGAACGGTTGGTCTCCAACCCCTCGATGTATTAGGGTGCGGAAATGCTACTTCCCATACCTG
GAAAACGGATATAACCAGAACTACGGCAGAAAGTTCGTGCAGGGCAAGTCTATCGACGTGGCTT
GTCACCCCGGCTACGCCCTGCCTAAGGCCCAGACCACCGTCACATGCATGGAGAATGGCTGGAG
CCCTACCCCTAGATGCATCAGAGTGTCCTTCACCCTGTGA
SEQ ID NO: 249 CFHT.2 protein sequence
MRLLAKIICLMLWAICVAEDCNELPPRRNTEILTGSWSDQTYPEGTQAIYKCRPGYRSLGNIIM
VCRKGEWVALNPLRKCQKRPCGHPGDTPFGTFTLTGGNVFEYGVKAVYTCNEGYQLLGEINYRE
CDTDGWINDIPICEVVKCLPVTAPENGKIVSSAMEPDREYHFGQAVRFVCNSGYKIEGDEEMHC
SDDGFWSKEKPKCVEISCKSPDVINGSPISQKIIYKENERFQYKCNMGYEYSERGDAVCTESGW
RPLPSCEEKSCDNPYIPNGDYSPLRIKHRTGDEITYQCRNGFYPATRGNTAKCTSTGWIPAPRC
TLKPCDYPDIKHGGLYHENMRRPYFPVAVGKYYSYYCDEHFETPSGSYWDHIHCTQDGWSPAVP
CLRKCYEPYLENGYNQNYGRKFVQGKSIDVACHPGYALPKAQTTVTCMENGWSPTPRCIRVRKC
YEPYLENGYNQNYGRKFVQGKSIDVACHPGYALPKAQTTVTCMENGWSPTPRCIRVRKCYFPYL
ENGYNQNYGRKFVQGKSIDVACHPGYALPKAQTTVTCMENGWSPTPRCIRVSFTL*
SEQ ID NO: 250 dCFHT.0 cDNA sequence
ATGAGACTGCTGGCCAAGATCATCTGCCTGATGCTGTGGGCCATCTGCGTGGCCGAAGCAACAT
TTTGTGATTTTCCAAAAATAAACCATGGAATTCTATATGATGAAGAAAAATATAAGCCATTTTC
CCAGGTTCCTACAGGGGAAGTTTTCTATTACTCCTGTGAATATAATTTTGTGTCTCCTTCAAAA
TCATTTTGGACTCGCATAACATGCACAGAAGAAGGATGGTCACCAACACCAAAGTGTCTCAGAC
TGTGTTTCTTTCCTTTTGTGGAAAATGGTCATTCTGAATCTTCAGGACAAACACATCTGGAAGG
TGATACTGTGCAAATTATTTGCAACACAGGATACAGACTTCAAAACAATGAGAACAACATTTCA.
TGTGTAGAACGGGGCTGGTCCACCCCTCCCAAATGCAGGTCCGGGGGGGGCGGCTCCGGCGGCG
GCGGCTCCGAGGACTGCAACGAGCTGCCCCCCAGAAGAAACACCGAGATCCTGACCGGCTCTTG
GAGCGACCAGACCTACCCTGAGGGCACCCAGGCCATCTACAAGTGCAGACCCGGCTACCGGTCC
CTGGGCAACATCATCATGGTGTGCAGAAAGGGCGAGTGGGTGGCCCTGAACCCCCTGAGAAAGT
GCCAGAAGAGGCCCTGCGGACACCCCGGCGATACCCCTTTTGGCACCTTCACACTGACCGGCGG
CAACGTGTTCGAGTACGGCGTGAAGGCCGTGTACACCTGTAACGAGGGCTACCAGCTGCTGGGC
GAGATCAACTACAGAGAGTGCGACACCGACGGCTGGACCAACGATATCCCCATCTGCGAGGTCG
TGAAGTGCCTGCCTGTGACCGCCCCAGAGAACGGCAAGATCGTGTCCAGCGCCATGGAACCCGA.
CAGAGAGTACCACTTCGGCCAGGCCGTCAGATTCGTGTGCAACAGCGGCTACAAGATCGAGGGC
GACGAGGAAATGCACTGCAGCGACGACGGCTTCTGGTCCAAAGAAAAGCCTAAGTGCGTGGAAA
TCAGCTGCAAGAGCCCCGACGTGATCAACGGCAGCCCCATCAGCCAGAAGATCATCTACAAAGA
GAACGAGCGGTTCCAGTACAAGTGTAACATGGGCTACGAGTACAGCGAGCGGGGCGACGCCGTG
TGTACAGAATCTGGATGGCGGCCTCTGCCCAGCTGCGAGGAAAAGAGCTGCGACAACCCCTACA
TCCCCAACGGCGACTACAGCCCCCTGCGGATCAAGCACAGAACCGGCGACGAGATCACCTACCA
GTGCCGGAACGGCTTCTACCCCGCCACCAGAGGCAATACCGCCAAGTGTACCAGCACCGGCTGG
ATCCCTGCCCCCAGATGTACCCTGAAGCCCTGCGACTACCCTGACATCAAGCACGGCGGCCTGT
ACCACGAGAACATGCGGAGGCCCTACTTCCCTGTGGCCGTGGGCAAGTACTACAGCTACTACTG
CGACGAGCACTTCGAGACACCCAGCGGCAGCTACTGGGACCACATCCACTGTACCCAGGACGGC
TGGTCCCCTGCCGTGCCCTGCCTGAGGAAGTGCTACTTCCCCTACCTGGAAAACGGCTACAACC
AGAACTACGGCCGGAAGTTCGTGCAGGGCAAGAGCATCGATGTGGCCTGCCACCCTGGATACGC
CCTGCCTAAGGCCCAGACCACCGTGACCTGCATGGAAAATGGATGGTCCCCCACCCCCCGGTGC
ATCAGAGTGTCCTTCACCCTGTGA
SEQ ID NO: 251 dCFHT.0 protein sequence
MRLLAKIICLMLWAICVAEATFCDFPKINHGILYDEEKYKPFSQVPTGEVFYYSCEYNFVSPSK
SFWTRITCTEEGWSPTPKCLRLCFFPFVENGHSESSGQTHLEGDTVQIICNTGYRLQNNENNIS
CVERGWSTPPKCRSGGGGGGGGSEDCNELPPRRNTEILTGSWSDQTYPEGTQAIYKCRPGYRS
LGNIIMVCRKGEWVALNPLRKCQKRPCGHPGDTPFGTFTLTGGNVFEYGVKAVYTCNEGYQLLG
EINYRECDTDGWINDIPICEVVKCLPVTAPENGKIVSSAMEPDREYHFGQAVRFVCNSGYKIEG
DEEMHCSDDGEWSKEKPKCVEISCKSPDVINGSPISQKIIYKENERFQYKCNMGYEYSERGDAV
CTESGWRPLPSCEEKSCDNPYIPNGDYSPLRIKHRTGDEITYQCRNGFYPATRGNTAKCTSTGW
IPAPRCTLKPCDYPDIKHGGLYHENMRRPYFPVAVGKYYSYYCDEHFETPSGSYWDHIHCTQDG
WSPAVPCLRKCYFPYLENGYNQNYGRKFVQGKSIDVACHPGYALPKAQTTVTCMENGWSPTPRC
IRVSFTL*
SEQ ID NO: 252 Wild-type Human SCR7 cDNA sequence
AGAAAATGTTATTTTCCTTATTTGGAAAATGGATATAATCAAAATTATGGAAGAAAGTTTGTAC
AGGGTAAATCTATAGACGTTGCCTGCCATCCTGGCTACGCTCTTCCAAAAGCGCAGACCACAGT
TACATGTATGGAGAATGGCTGGTCTCCTACTCCCAGATGCATCCGT
SEQ ID NO: 253 protective CFHT protein sequence lacking the
signal polypeptide The isoleucine corresponding to postion 62
and Y corresponding to position 402 of por of the sequence
containing the signal peptide is underlined
EDCNELPPRRNTEILTGSWSDQTYPEGTQAIYKCRPGYRSLGNIIMVCRKGEWVALNPLRKCQK
RPCGHPGDTPFGTFTLTGGNVFEYGVKAVYTCNEGYQLLGEINYRECDTDGWINDIPICEVVKC
LPVTAPENGKIVSSAMEPDREYHFGQAVRFVCNSGYKIEGDEEMHCSDDGFWSKEKPKCVEISC
KSPDVINGSPISQKIIYKENERFQYKCNMGYEYSERGDAVCTESGWRPLPSCEEKSCDNPYIPN
GDYSPLRIKHRTGDEITYQCRNGFYPATRGNTAKCTSTGWIPAPRCTLKPCDYPDIKHGGLYHE
NMRRPYFPVAVGKYYSYYCDEHFETPSGSYWDHIHCTQDGWSPAVPCLRKCYFPYLENGYNQNY
GRKFVQGKSIDVACHPGYALPKAQTTVTCMENGWSPTPRCIRVSFTL

1. A gene therapy vector for treatment of conditions associated with complement dysfunction comprising a cargo encoding two, three, or more than three activities selected from:
a) one or more of an optimized Truncated Complement Factor H (oCFHT) protein (optionally comprising SEQ ID NO: 247, 251, or 249);
b) an inhibitory RNA that targets C7 (optionally hpRNA_B comprising SEQ ID NO:59) CFB, CFD, CFP, CFHR-1, CFHR-2, CFHR-3, CFHR-4, CFHR-5, C3, C4A, C4B, C5, C6, C8A, C8B, C8G, or C9;
c) one or more complement proteins selected from CFHT, CFH, and CFI;
d) one or more binding proteins that specifically binds CFB, CFD, CFP, CFHR-1, CFHR-2, CFHR-3, CFHR-4, CFHR-5, C3, C4A, C4B, C5, C6, C7, C8A, C8B, C8G, or C9;
e) HTRA1 protein or a transcriptional activator protein that increases expression of HTRA1;
f) a binding protein that specifically binds ApoE2 or VEGFA;
g) an inhibitory RNA that targets ApoE2 or VEGFA.

2. The gene therapy vector of claim 1 encoding two said activities.

3. The gene therapy vector of claim 1 encoding three said activities or four said activities.

4. The gene therapy vector of claim 1 encoding four said activities.

5. The gene therapy vector of claim 1 comprising a cargo encoding an oCFHT complement protein.

6. The gene therapy vector of claim 5 wherein the oCFHT is mCFHT (optionally mCFHT.1), dCFHT, or mdCFHT).

7. The gene therapy vector of claim 6 wherein the oCFHT is mCFHT.

8. The gene therapy vector of claim 7 encoding a fusion protein comprising a protective CFHT domain with 90% identity to SEQ ID NO:253 and 1, 2, 3 additional SCR7 domains.

9. The gene therapy vector of claim 8 wherein the additional SCR7 domains are C-terminal to SCR6.

10. The gene therapy vector of claim 7, wherein the ocFHT is mCFHT.1, optionally comprising SEQ ID NO:247.

11. The gene therapy vector of claim 6 wherein the oCFHT is dCFHT, optionally comprising SEQ ID NO:251.

12. The gene therapy vector of claim 11 encoding a fusion protein comprising a protective CFHT domain with 90% identity to SEQ ID NO:253 and an SCR1-2 dimerization domain comprising SEQ ID NO. 1.

13. The gene therapy vector of claim 12 wherein the SCR1-2 dimerization domain is at the amino terminus of the fusion protein.

14. The gene therapy vector of claim 7 wherein the oCFHT is mdCFHT.

15. The gene therapy vector of claim 14 encoding a fusion protein comprising a CFHT domain with 90% identity to SEQ ID NO:253, and 1, 2 or 3 additional SCR7 domains C-terminal to SCR6, and a SCR1-2 dimerization domain at the amino terminus of the fusion protein.

16. The gene therapy vector of claim 1 comprising a cargo that encodes a complement protein selected from CFHT, CFH, and CFI.

17. The gene therapy vector of claim 16 that encodes CFHT.

18. The gene therapy vector of claim 1 or 16 comprising a cargo encoding a complement protein CFI.

19. The gene therapy vector of claim 1-18 comprising a cargo encoding an inhibitory RNA that targets C7, CFB, CFD, CFP, CFHR-1, CFHR-2, CFHR-3, CFHR-4, CFHR-5, C3, C4A, C4B, C5, C6, C8A, C8B, C8G, or C9.

20. The gene therapy vector of claim 19 wherein the inhibitory RNA is hpRNA, siRNA, or miRNA.

21. The gene therapy vector of claim 20 wherein the inhibitory RNA is hpRNA.

22. The gene therapy vector of claim 19 wherein the inhibitory RNA targets C7.

23. The gene therapy vector of claim 1-21 that encodes a binding protein that specifically binds CFB, CFD, CFP, CFHR-1, CFHR-2, CFHR-3, CFHR-4, CFHR-5, C3, C4A, C4B, C5, C6, C7, C8A, C8B, C8G, or C9.

24. The gene therapy vector of claim 23 wherein the binding protein is a nanobody (single domain antibody) or single chain antibody (ScFv).

25. The gene therapy vector of claim 24 wherein the binding protein binds two or more of CFHR-1, CFHR-2, CFHR-3, CFHR-4, CFHR-5 and does not bind CFH or CFHT.

26. The gene therapy vector of claim 25 wherein the binding protein is cross-reactive and binds two CFHR proteins.

27. The gene therapy vector of claim 25 wherein the binding protein is bifunctional.

28. The gene therapy vector of claim 25 or 26 wherein the protein is binds CFHR1 and CFHR-4.

29. The gene therapy vector of claim 1 or 5 that encodes a binding protein that specifically binds VEGFA.

30. The gene therapy vector of claim 29 wherein the binding protein is aflibercept.

31. The gene therapy vector of claim 1 or 5 encoding a binding protein that binds HTRA1 or an expression activator that increases expression of HTRA1.

32. The gene therapy vector of claim 31 wherein the expression activator is a CRISPRa system.

33. The gene therapy vector of claim 1-32 encoding an inhibitory RNA that targets ApoE2 or VEGFA or a binding protein that targets ApoE2.

34. The gene therapy vector of claim 1-33 wherein the cargo is less that 5 KB in length, optionally less than 4.7 kb in length.

35. The gene therapy vector of claim 1-33 wherein the vector is a viral vector.

36. The gene therapy vector of claim 35 wherein the vector is AAV or LV.

37. The gene therapy vector of claim 36 wherein the vector is AAV2 comprising AAV2 ITRs or is AAV8 comprising AAV8 ITRs.

38. The gene therapy vector of claim 37 wherein the vector is AAV and comprises an AAV2 or AAV8 capsid.

39. The gene therapy vector of claim 1-34 wherein the vector is a non-viral vector.

40. The gene therapy vector of claim 1-39 comprising a cargo that encodes at least 2 activities, optionally three activities, selected from:
a complement protein listed in claim 1(a) or 1(c),
a binding protein listed in claim 1(d), wherein said binding protein is optionally a nanobody, and
an inhibitory RNA listed in claim 1(b).

41. The gene therapy vector of claim 40 wherein the complement protein is oCFHT or CFHT; the inhibitory RNA is an hpRNA; and the binding protein is a nanobody.

42. The gene therapy of claim 40 or 41 comprising a cargo encoding two complement proteins listed in claim 1(a) or two binding proteins listed in claim 1(d) or two inhibitory RNA listed in claim 1(b).

43. The gene therapy vector of claim 41 that comprises a cargo encoding two proteins, wherein expression of the two proteins is under the control of one promoter.

44. The gene therapy vector of claim 43 wherein the promoter is CBA, smCBA, VMD2, or SFFV.

45. The gene therapy vector of claim 43 or 44 wherein the cargo encodes a multicistronic RNA.

46. The gene therapy vector of claim 43 wherein the promoter is a bidirectional.

47. The gene therapy vector of claim 43 wherein the cargo comprises a sequence encoding an internal ribosome entry site (IRES), a ribosome skipping element (RSE) and/or a furin cleavage site.

48. The gene therapy vector of claim 44 in which each of two proteins is operably linked to a different promoter.

49. The gene therapy vector of claim 44-48 wherein one protein is a complement protein and the other protein is a binding protein.

50. The gene therapy vector of claim 1-49 that comprises a cargo encoding at least one inhibitory RNA, optionally an inhibitory RNA that targets C7.

51. The gene therapy vector of claim 50 in which the inhibitory RNA is operably linked to a Pol Ill promoter, optionally selected from a U6 promoter, an H1 promoter, or a 7SK promoter.

52. A gene therapy vector for treatment of conditions associated with complement dysfunction comprising a polynucleotide that comprises elements in the following order, not necessarily contiguously:
i) A-C-E-K-J
ii) A-J-E-C
iii) A-C-E-D
iv) A-C-E-D
v) A-C-E-D-B-H
vi) A-C-D-B
vii) A-C-E-D-F-B-H
wherein (A) means a Pol II promoter, optionally selected from CBA, smCBA, VMD2, or SFFV promoter,
wherein (B) means a Pol III promoter, optionally selected from a U6, H1, or 7SK promoter,
wherein (C) means a nucleotide sequence encoding a first protein activity, optionally CFHT or oCFHT,
wherein (D) a nucleotide sequence encoding a second protein activity, optionally a binding protein,
wherein (E) means an IRES, mIRES, T2A, or cleavage element, such as a furan cleavage element
wherein (F) means a poly-A signal, optionally a bGH poly-A signal, wherein the poly-A signal is optionally downstream of the nucleotide sequences encoding the (i) and (ii),
wherein (G) means nucleotide sequence encoding a first inhibitory RNA, optionally a first hpRNA,
wherein (H) means nucleotide sequence encoding a second inhibitory RNA, optionally a hpRNA with a specificity different from the first hpRNA,
wherein (1) means a WORE regulatory element,
wherein (J) means a nucleotide sequence encoding aflibercept,
wherein (K) means a signal peptide, optionally TTR, IGF-1 and
wherein (L) means a nucleotide sequence encoding a 5′ or 3′ LTR or ITR.

53. A cell transduced with the vector of any one of claims 1-50.

54. A cell comprising the cargo portion of a vector of any of claims 1-50.

55. A pharmaceutical composition comprising the vector of any of claims 1-50 and a pharmaceutically acceptable carrier.

56. A method for treatment of a condition associated with complement dysfunction in a patient comprising administering a gene therapy vector of any of claims 1-50.

57. The method of claim 56 wherein the condition is an ocular disorder.

58. The method of claim 57 wherein the ocular disorder is age-related macular degeneration (AMD).

59. The method of claim 56-58 comprising intraocularly administering an effective dose of a gene therapy vector of on one of claims 1-50.

60. The method of claim 59 wherein the vector or cell is administered by subretinal injection, direct retinal injection, suprachoroidal injection or intravitreal injection.

61. A gene therapy vector for treatment of conditions associated with complement dysfunction comprising a cargo encoding an optimized CFHT (oCFHT) selected from mCFHT, dCFHT, or mdCFHT wherein
a) mCFHT is a fusion protein comprising a CFHT domain with 90% identity to naturally occurring protective CFHT (SEQ ID NO:253) and 1, 2, 3 additional SCR7 domains, optionally comprising SEQ ID NO:242, wherein the additional SCR7 domains are C-terminal to SCR6;
b) dCFHT is a fusion protein comprising a CFHT domain with 90% identity to naturally occurring protective CFHT (SEQ ID NO:253) and an SCR1-2 dimerization domain, optionally comprising SEQ ID NO:1, at the amino terminus of the fusion protein; and
c) mdCFHT is a fusion protein comprising a CFHT domain with 90% identity to naturally occurring protective CFHT (SEQ ID NO:253); 1, 2 or 3 additional SCR7 domains, optionally comprising SEQ ID NO:242, C-terminal to SCR6; and a SCR1-2 dimerization domain, optionally comprising SEQ ID NO:1, at the amino terminus of the fusion protein.


SEQUENCE LISTING (253 sequences)

SEQ ID NO: 1 - protein, 124 aa, synthetic construct
  REGION 1..124: note = Description of Sequence: FHR-1 dimerization motif (SCR1-2)
EATFCDFPKINHGILYDEEKYKPFSQVPTGEVFYYSCEYNFVSPSKSFWTRITCTEEGWSPTPKCLRLCFFPFVENGHSESSGQTHLEGDTVQIICNTGYRLQNNENNISCVERGWSTPPKCRS

SEQ ID NO: 2 - DNA, 177 bp, synthetic construct
  misc_feature 1..177: note = Description of Sequence: Human codon optimized Wild-Type SCR7 gene
  source 1..177: mol_type = other DNA
aggaagtgctacttcccctacctggaaaacggctacaaccagaactacggccggaagttcgtgcagggcaagagcatcgatgtggcctgccaccctggatacgccctgcctaaggcccagaccaccgtgacctgcatggaaaatggatggtcccccaccccccggtgcatcagagtg

SEQ ID NO: 3 - DNA, 177 bp, synthetic construct
  misc_feature 1..177: note = Description of Sequence: IDT Codon Optimized CFHT SCR7.1 cDNA
  source 1..177: mol_type = other DNA
aggaagtgctatttcccttatttggaaaacggatataatcaaaattacggtcggaaatttgtccaaggtaagtcaatagatgtcgcgtgtcatcctggctatgctcttccgaaagcacagacaacggttacgtgtatggagaacggttggtctccaacccctcgatgtattagggtg

SEQ ID NO: 4 - DNA, 177 bp, synthetic construct; identical to SEQ ID NO: 2
  misc_feature 1..177: note = Description of Sequence: GeneScript Codon Optimized CFHT SCR7.2 cDNA
  source 1..177: mol_type = other DNA

SEQ ID NO: 5 - DNA, 177 bp, synthetic construct
  misc_feature 1..177: note = Description of Sequence: NovoPro Codon Optimized CFHT SCR7.3 cDNA
  source 1..177: mol_type = other DNA
aggaaatgctattttccctatttggaaaacggctacaaccagaattatggaaggaagttcgtccagggaaaatcaatcgacgtggcatgccaccctgggtacgccttgccaaaagcccagacaaccgtgacatgcatggagaacggatggtctccaacacccagatgtatccgggtc

SEQ ID NO: 6 - DNA, 1353 bp, synthetic construct
  misc_feature 1..1353: note = Description of Sequence: Aflibercept-TTR signal peptide
  source 1..1353: mol_type = other DNA
atggcctctcatagactgctgctgctgtgtctggccggcctggtgtttgtgtctgaggcctctgataccggcagacccttcgtggaaatgtacagcgagatccccgagatcatccacatgaccgagggcagagagctggtcatcccctgcagagtgacaagccccaacatcaccgtgactctgaagaagttccctctggacacactgatccccgacggcaagagaatcatctgggacagccggaagggcttcatcatcagcaacgccacctacaaagagatcggcctgctgacctgtgaagccaccgtgaatggccacctgtacaagaccaactacctgacacacagacagaccaacaccatcatcgacgtggtgctgagccctagccacggcattgaactgtctgtgggcgagaagctggtgctgaactgtaccgccagaaccgagctgaacgtgggcatcgacttcaactgggagtaccccagcagcaagcaccagcacaagaaactggtcaaccgggacctgaaaacccagagcggcagcgagatgaagaaattcctgagcaccctgaccatcgacggcgtgaccagatctgaccagggcctgtacacatgtgccgccagctctggcctgatgaccaagaaaaacagcaccttcgtgcgggtgcacgagaaggacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaatagcacctacagagtggtgtccgtgctgaccgtgctgcaccaggattggctgaacggcaaagagtacaagtgcaaggtgtccaacaaggccctgcctgctcctatcgagaaaaccatctccaaggccaagggccagcctagggaaccccaggtttacacactgcctccaagcagggacgagctgacaaagaaccaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgcagcgtgatgcacgaggccctgcacaaccactacacccagaagtccctgagcctgtctcctggc

SEQ ID NO: 7 - DNA, 1359 bp, synthetic construct
  misc_feature 1..1359: note = Description of Sequence: Aflibercept 1359-bp with IGF1 Signal Peptide
  source 1..1359: mol_type = other DNA
atgggcaagatttctagcctgcctacacagctgttcaagtgctgcttctgcgacttcctgaagtccgataccggcagacccttcgtggaaatgtacagcgagatccccgagatcatccacatgaccgagggcagagagctggtcatcccctgcagagtgacaagccccaacatcaccgtgactctgaagaagttccctctggacacactgatccccgacggcaagagaatcatctgggacagccggaagggcttcatcatcagcaacgccacctacaaagagatcggcctgctgacctgtgaagccaccgtgaatggccacctgtacaagaccaactacctgacacacagacagaccaacaccatcatcgacgtggtgctgagccctagccacggcattgaactgtctgtgggcgagaagctggtgctgaactgtaccgccagaaccgagctgaacgtgggcatcgacttcaactgggagtaccccagcagcaagcaccagcacaagaaactggtcaaccgggacctgaaaacccagagcggcagcgagatgaagaaattcctgagcaccctgaccatcgacggcgtgaccagatctgaccagggcctgtacacatgtgccgccagctctggcctgatgaccaagaaaaacagcaccttcgtgcgggtgcacgagaaggacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaatagcacctacagagtggtgtccgtgctgaccgtgctgcaccaggattggctgaacggcaaagagtacaagtgcaaggtgtccaacaaggccctgcctgctcctatcgagaaaaccatctccaaggccaagggccagcctagggaaccccaggtttacacactgcctccaagcagggacgagctgacaaagaaccaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgcagcgtgatgcacgaggccctgcacaaccactacacccagaagtccctgagcctgtctcctggctaa

SEQ ID NO: 8 - DNA, 1359 bp, synthetic construct
  misc_feature 1..1359: note = Description of Sequence: Aflibercept 1359-bp with IgK Signal Peptide
  source 1..1359: mol_type = other DNA
atggaaacagatacactgctgctgtgggtgctgctcttgtgggtgccaggatctacaggcgattctgataccggcagacccttcgtggaaatgtacagcgagatccccgagatcatccacatgaccgagggcagagagctggtcatcccctgcagagtgacaagccccaacatcaccgtgactctgaagaagttccctctggacacactgatccccgacggcaagagaatcatctgggacagccggaagggcttcatcatcagcaacgccacctacaaagagatcggcctgctgacctgtgaagccaccgtgaatggccacctgtacaagaccaactacctgacacacagacagaccaacaccatcatcgacgtggtgctgagccctagccacggcattgaactgtctgtgggcgagaagctggtgctgaactgtaccgccagaaccgagctgaacgtgggcatcgacttcaactgggagtaccccagcagcaagcaccagcacaagaaactggtcaaccgggacctgaaaacccagagcggcagcgagatgaagaaattcctgagcaccctgaccatcgacggcgtgaccagatctgaccagggcctgtacacatgtgccgccagctctggcctgatgaccaagaaaaacagcaccttcgtgcgggtgcacgagaaggacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaatagcacctacagagtggtgtccgtgctgaccgtgctgcaccaggattggctgaacggcaaagagtacaagtgcaaggtgtccaacaaggccctgcctgctcctatcgagaaaaccatctccaaggccaagggccagcctagggaaccccaggtttacacactgcctccaagcagggacgagctgacaaagaaccaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgcagcgtgatgcacgaggccctgcacaaccactacacccagaagtccctgagcctgtctcctggctaa

SEQ ID NO: 9 - DNA, 2700 bp, synthetic construct
  misc_feature 1..2700: note = Description of Sequence: CFHT cDNA
  source 1..2700: mol_type = other DNA
atgagactgctggccaagatcatctgcctgatgctgtgggccatctgcgtggccgaggactgcaacgagctgccccccagaagaaacaccgagatcctgaccggctcttggagcgaccagacctaccctgagggcacccaggccatctacaagtgcagacccggctaccggtccctgggcaacatcatcatggtgtgcagaaagggcgagtgggtggccctgaaccccctgagaaagtgccagaagaggccctgcggacaccccggcgataccccttttggcaccttcacactgaccggcggcaacgtgttcgagtacggcgtgaaggccgtgtacacctgtaacgagggctaccagctgctgggcgagatcaactacagagagtgcgacaccgacggctggaccaacgatatccccatctgcgaggtcgtgaagtgcctgcctgtgaccgccccagagaacggcaagatcgtgtccagcgccatggaacccgacagagagtaccacttcggccaggccgtcagattcgtgtgcaacagcggctacaagatcgagggcgacgaggaaatgcactgcagcgacgacggcttctggtccaaagaaaagcctaagtgcgtggaaatcagctgcaagagccccgacgtgatcaacggcagccccatcagccagaagatcatctacaaagagaacgagcggttccagtacaagtgtaacatgggctacgagtacagcgagcggggcgacgccgtgtgtacagaatctggatggcggcctctgcccagctgcgaggaaaagagctgcgacaacccctacatccccaacggcgactacagccccctgcggatcaagcacagaaccggcgacgagatcacctaccagtgccggaacggcttctaccccgccaccagaggcaataccgccaagtgtaccagcaccggctggatccctgcccccagatgtaccctgaagccctgcgactaccctgacatcaagcacggcggcctgtaccacgagaacatgcggaggccctacttccctgtggccgtgggcaagtactacagctactactgcgacgagcacttcgagacacccagcggcagctactgggaccacatccactgtacccaggacggctggtcccctgccgtgccctgcctgaggaagtgctacttcccctacctggaaaacggctacaaccagaactacggccggaagttcgtgcagggcaagagcatcgatgtggcctgccaccctggatacgccctgcctaaggcccagaccaccgtgacctgcatggaaaatggatggtcccccaccccccggtgcatcagagtgtccttcaccctgtgaatgagactgctggccaagatcatctgcctgatgctgtgggccatctgcgtggccgaggactgcaacgagctgccccccagaagaaacaccgagatcctgaccggctcttggagcgaccagacctaccctgagggcacccaggccatctacaagtgcagacccggctaccggtccctgggcaacatcatcatggtgtgcagaaagggcgagtgggtggccctgaaccccctgagaaagtgccagaagaggccctgcggacaccccggcgataccccttttggcaccttcacactgaccggcggcaacgtgttcgagtacggcgtgaaggccgtgtacacctgtaacgagggctaccagctgctgggcgagatcaactacagagagtgcgacaccgacggctggaccaacgatatccccatctgcgaggtcgtgaagtgcctgcctgtgaccgccccagagaacggcaagatcgtgtccagcgccatggaacccgacagagagtaccacttcggccaggccgtcagattcgtgtgcaacagcggctacaagatcgagggcgacgaggaaatgcactgcagcgacgacggcttctggtccaaagaaaagcctaagtgcgtggaaatcagctgcaagagccccgacgtgatcaacggcagccccatcagccagaagatcatctacaaagagaacgagcggttccagtacaagtgtaacatgggctacgagtacagcgagcggggcgacgccgtgtgtacagaatctggatggcggcctctgcccagctgcgaggaaaagagctgcgacaacccctacatccccaacggcgactacagccccctgcggatcaagcacagaaccggcgacgagatcacctaccagtgccggaacggcttctaccccgccaccagaggcaataccgccaagtgtaccagcaccggctggatccctgcccccagatgtaccctgaagccctgcgactaccctgacatcaagcacggcggcctgtaccacgagaacatgcggaggccctacttccctgtggccgtgggcaagtactacagctactactgcgacgagcacttcgagacacccagcggcagctactgggaccacatccactgtacccaggacggctggtcccctgccgtgccctgcctgaggaagtgctacttcccctacctggaaaacggctacaaccagaactacggccggaagttcgtgcagggcaagagcatcgatgtggcctgccaccctggatacgccctgcctaaggcccagaccaccgtgacctgcatggaaaatggatggtcccccaccccccggtgcatcagagtgtccttcaccctgtga

SEQ ID NO: 10 - DNA, 880 bp, synthetic construct
  misc_feature 1..880: note = Description of Sequence: smCBA promoter 881-bp
  source 1..880: mol_type = other DNA
ccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggca

SEQ ID NO: 11 - DNA, 886 bp, synthetic construct
  misc_feature 1..886: note = Description of Sequence: smCBA promoter 886-bp
  source 1..886: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattc

SEQ ID NO: 12 - DNA, 1654 bp, synthetic construct
  misc_feature 1..1654: note = Description of Sequence: CBA/CAG promoter 1654-bp
  source 1..1654: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 13 - DNA, 796 bp, synthetic construct
  misc_feature 1..796: note = Description of Sequence: VMD2 -796-bp (n. 1-624) + SV40 splice donor/acceptor (n. 625-796)
  source 1..796: mol_type = other DNA
caattctgtcattttactagggtgatgaaattcccaagcaacaccatccttttcagataagggcactgaggctgagagaggagctgaaacctacccggcgtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggaccaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagaccggggatcctctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttac

SEQ ID NO: 14 - DNA, 673 bp, synthetic construct
  misc_feature 1..673: note = Description of Sequence: RPE65 promoter 750-bp
  source 1..673: mol_type = other DNA
atactctcagagtgccaaacatataccaatggacaagaaggtgaggcagagagcagacaggcattagtgacaagcaaagatatgcagaatttcattctcagcaaatcaaaagtcctcaacctggttggaagaatattggcactgaatggtatcaataaggttgctagagagggttagaggtgcacaatgtgcttccataacattttatacttctccaatcttagcactaatcaaacatggttgaatactttgtttactataactcttacagagttataagatctgtgaagacagggacagggacaatacccatctctgtctggttcataggtggtatgtaatagatatttttaaaaataagtgagttaatgaatgagggtgagaatgaaggcacagaggtattagggggaggtgggccccagagaatggtgccaaggtccagtggggtgactgggatcagctcaggcctgacgctggccactcccacctagctcctttctttctaatctgttctcattctccttgggaaggattgaggtctctggaaaacagccaaacaactgttatgggaacagcaagcccaaataaagccaagcatcagggggatctgagagctgaaagcaacttctgttccccctccctcagctgaaggggtggggaagggctcccaaag

SEQ ID NO: 15 - DNA, 314 bp, synthetic construct
  misc_feature 1..314: note = Description of Sequence: U6 promoter 313-bp
  source 1..314: mol_type = other DNA
tatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtt

SEQ ID NO: 16 - DNA, 443 bp, synthetic construct
  misc_feature 1..443: note = Description of Sequence: SFFV promoter 443-bp
  source 1..443: mol_type = other DNA
tgaaagaccccacctgtaggtttggcaagatagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgattgactgagtcgccc

SEQ ID NO: 17 - DNA, 1350 bp, synthetic construct
  misc_feature 1..1350: note = Description of Sequence: CFHT 1350-bp
  source 1..1350: mol_type = other DNA
atgagactgctggccaagatcatctgcctgatgctgtgggccatctgcgtggccgaggactgcaacgagctgccccccagaagaaacaccgagatcctgaccggctcttggagcgaccagacctaccctgagggcacccaggccatctacaagtgcagacccggctaccggtccctgggcaacatcatcatggtgtgcagaaagggcgagtgggtggccctgaaccccctgagaaagtgccagaagaggccctgcggacaccccggcgataccccttttggcaccttcacactgaccggcggcaacgtgttcgagtacggcgtgaaggccgtgtacacctgtaacgagggctaccagctgctgggcgagatcaactacagagagtgcgacaccgacggctggaccaacgatatccccatctgcgaggtcgtgaagtgcctgcctgtgaccgccccagagaacggcaagatcgtgtccagcgccatggaacccgacagagagtaccacttcggccaggccgtcagattcgtgtgcaacagcggctacaagatcgagggcgacgaggaaatgcactgcagcgacgacggcttctggtccaaagaaaagcctaagtgcgtggaaatcagctgcaagagccccgacgtgatcaacggcagccccatcagccagaagatcatctacaaagagaacgagcggttccagtacaagtgtaacatgggctacgagtacagcgagcggggcgacgccgtgtgtacagaatctggatggcggcctctgcccagctgcgaggaaaagagctgcgacaacccctacatccccaacggcgactacagccccctgcggatcaagcacagaaccggcgacgagatcacctaccagtgccggaacggcttctaccccgccaccagaggcaataccgccaagtgtaccagcaccggctggatccctgcccccagatgtaccctgaagccctgcgactaccctgacatcaagcacggcggcctgtaccacgagaacatgcggaggccctacttccctgtggccgtgggcaagtactacagctactactgcgacgagcacttcgagacacccagcggcagctactgggaccacatccactgtacccaggacggctggtcccctgccgtgccctgcctgaggaagtgctacttcccctacctggaaaacggctacaaccagaactacggccggaagttcgtgcagggcaagagcatcgatgtggcctgccaccctggatacgccctgcctaaggcccagaccaccgtgacctgcatggaaaatggatggtcccccaccccccggtgcatcagagtgtccttcaccctgtga

SEQ ID NO: 18 - DNA, 719 bp, synthetic construct
  misc_feature 1..719: note = Description of Sequence: sctmCBA Enhancer Promoter -719 bp
  source 1..719: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagc

SEQ ID NO: 19 - DNA, 63 bp, synthetic construct
  misc_feature 1..63: note = Description of Sequence: IGF-1 signal peptide
  source 1..63: mol_type = other DNA
atgggcaagatttctagcctgcctacacagctgttcaagtgctgcttctgcgacttcctgaag

SEQ ID NO: 20 - DNA, 63 bp, synthetic construct
  misc_feature 1..63: note = Description of Sequence: IgK signal peptide
  source 1..63: mol_type = other DNA
atggaaacagatacactgctgctgtgggtgctgctcttgtgggtgccaggatctacaggcgat

SEQ ID NO: 21 - DNA, 60 bp, synthetic construct
  misc_feature 1..60: note = Description of Sequence: TTR Signal peptide
  source 1..60: mol_type = other DNA
atggcctctcatagactgctgctgctgtgtctggccggcctggtgtttgtgtctgaggcc

SEQ ID NO: 22 - DNA, 549 bp, synthetic construct
  misc_feature 1..549: note = Description of Sequence: IRES 549-bp
  source 1..549: mol_type = other DNA
cgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcccctcaagcgtattcaacaaggggctgaaggatgcccagaaggtaccccattgtatgggatctgatctggggcctcggtgcacatgcttttcatgtgtttagtcgaggttaaaaaacgtctaggccccccgaaccacggggacgtggttttcctttgaaaaacacgatgataa

SEQ ID NO: 23 - DNA, 462 bp, synthetic construct
  misc_feature 1..462: note = Description of Sequence: Minimal IRES 462-bp
  source 1..462: mol_type = other DNA
gagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcccctcaagcgtattcaacaaggggctgaaggatgcccagaaggtaccccattgtatgggatctgatctggggcctcggtgcacatgcttttcatgtgtttagtcgaggttaaaaaacgtctaggccccccgaaccacggggacgtggttttcctttgaaaaacacgatgataa

SEQ ID NO: 24 - DNA, 613 bp, synthetic construct
  misc_feature 1..613: note = Description of Sequence: 18-bp spacer - IRES (551-bp) - 45-bp spacer -
  source 1..613: mol_type = other DNA
gtaccactgtgcatatgcgttactggccgaagccgcttggaataaggccggtgtgcgtttgtctatatgttattttccaccatattgccgtcttttggcaatgtgagggccaggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcccctcaagcgtattcaacaaggggctgaaggatgcccagaaggtaccccattgtatgggatctgatctggggcctcggtgcacatgcttttcatgtgtttagtcgaggttaaaaaacgtctaggccccccgaaccacggggacgtggttttcctttgaaaaacacgatgataatagaattcgatatcaagcttatcgataccgtcgactagagctccacc

SEQ ID NO: 25 - DNA, 589 bp, synthetic construct
  misc_feature 1..589: note = Description of Sequence: WPRE 589-bp
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 26 - DNA, 247 bp, synthetic construct
  misc_feature 1..247: note = Description of Sequence: WPRE3 247-bp
  source 1..247: mol_type = other DNA
ataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggt

SEQ ID NO: 27 - DNA, 122 bp, synthetic construct
  misc_feature 1..122: note = Description of Sequence: SV40 Poly A 122-bp
  source 1..122: mol_type = other DNA
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 28 - DNA, 49 bp, synthetic construct
  misc_feature 1..49: note = Description of Sequence: HSV TK 49-bp
  source 1..49: mol_type = other DNA
cggcaataaaaagacagaataaaacgcacgggtgttgggtcgtttgttc

SEQ ID NO: 29 - DNA, 185 bp, synthetic construct
  misc_feature 1..185: note = Description of Sequence: bGH 185-bp
  source 1..185: mol_type = other DNA
ttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggc

SEQ ID NO: 30 - DNA, 54 bp, synthetic construct
  misc_feature 1..54: note = Description of Sequence: Encodes SCPS T2A peptide 54-bp
  source 1..54: mol_type = other DNA
gagggcaggggaagtcttctaacatgcggggacgtggaggaaaatcccggcccc

SEQ ID NO: 31 - protein, 17 aa, synthetic construct
  REGION 1..17: note = Description of Sequence: SCPS T2A
EGRGSLLTCGDVEENPG

SEQ ID NO: 32 - DNA, 60 bp, synthetic construct
  misc_feature 1..60: note = Description of Sequence: Encodes SCPS E2A 60-bp
  source 1..60: mol_type = other DNA
cagtgtactaattatgctctcttgaaattggctggagatgttgagagcaaccctggacct

SEQ ID NO: 33 - protein, 19 aa, synthetic construct
  REGION 1..19: note = Description of Sequence: SCPS E2A
QCTNYALLKLAGDVESNPG

SEQ ID NO: 34 - DNA, 57 bp, synthetic construct
  misc_feature 1..57: note = Description of Sequence: Encodes SCPS P2A peptide 57-bp
  source 1..57: mol_type = other DNA
gccaccaacttcagcctgctgaagcaggccggcgacgtggaggagaaccccggcccc

SEQ ID NO: 35 - protein, 18 aa, synthetic construct
  REGION 1..18: note = Description of Sequence: SCPS P2A
ATNFSLLKQAGDVEENPG

SEQ ID NO: 36 - DNA, 66 bp, synthetic construct
  misc_feature 1..66: note = Description of Sequence: Encodes SCPS F2A peptide 66-bp
  source 1..66: mol_type = other DNA
gtgaaacagactttgaattttgaccttctcaagttggcgggagacgtggagtccaaccctggacct

SEQ ID NO: 37 - protein, 21 aa, synthetic construct
  REGION 1..21: note = Description of Sequence: SCPS F2A
VKQTLNFDLLKLAGDVESNPG

SEQ ID NO: 38 - DNA, 24 bp, synthetic construct
  misc_feature 1..24: note = Description of Sequence: Encodes SCPS Furin 2A Cleavage Peptide 24-bp
  source 1..24: mol_type = other DNA
ggcggcaggggtcgacgcggcggc

SEQ ID NO: 39 - protein, 6 aa, synthetic construct
  REGION 1..6: note = Description of Sequence: SCPS Furin
GGRGRR

SEQ ID NO: 40 - protein, 8 aa, synthetic construct
  REGION 1..8: note = Description of Sequence: SCPS Furin
GGRGRRGG

SEQ ID NO: 41 - DNA, 12 bp, synthetic construct
  misc_feature 1..12: note = Description of Sequence: SPACER
  source 1..12: mol_type = other DNA
taagtgacctag

SEQ ID NO: 42 - skipped

SEQ ID NO: 43 - DNA, 24 bp, synthetic construct
  misc_feature 1..24: note = Description of Sequence: SPACER
  source 1..24: mol_type = other DNA
taagtgacctagaaattcaggcac

SEQ ID NO: 44-46 - skipped (3 entries)

SEQ ID NO: 47 - DNA, 511 bp, synthetic construct
  misc_feature 1..511: note = Description of Sequence: mIRES and flanking
  source 1..511: mol_type = other DNA
gagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcccctcaagcgtattcaacaaggggctgaaggatgcccagaaggtaccccattgtatgggatctgatctggggcctcggtgcacatgcttttcatgtgtttagtcgaggttaaaaaacgtctaggccccccgaaccacggggacgtggttttcctttgaaaaacacgatgataatagaattcgatatcaagcttatcgataccgtcgactagagctcgccacc

SEQ ID NO: 48 - DNA, 128 bp, synthetic construct
  misc_feature 1..128: note = Description of Sequence: ITR_AAV2_R_short 128-bp
  source 1..128: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 49 - DNA, 181 bp, synthetic construct
  misc_feature 1..181: note = Description of Sequence: Lentiviral 5' LTR (Truncated) 181-bp
  source 1..181: mol_type = other DNA
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 50 - DNA, 234 bp, synthetic construct
  misc_feature 1..234: note = Description of Sequence: Lentiviral 3' LTR (Delta-U3) 234-bp
  source 1..234: mol_type = other DNA
tggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 51 - DNA, 624 bp, synthetic construct
  misc_feature 1..624: note = Description of Sequence: VMD2 promoter
  source 1..624: mol_type = other DNA
caattctgtcattttactagggtgatgaaattcccaagcaacaccatccttttcagataagggcactgaggctgagagaggagctgaaacctacccggcgtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggaccaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagacc

SEQ ID NO: 52 - DNA, 198 bp, synthetic construct
  misc_feature 1..198: note = Description of Sequence: FHR-1 SCR1 - Non-codon Optimized
  source 1..198: mol_type = other DNA
gaagcaacattttgtgattttccaaaaataaaccatggaattctatatgatgaagaaaaatataagccattttcccaggttcctacaggggaagttttctattactcctgtgaatataattttgtgtctccttcaaaatcattttggactcgcataacatgcacagaagaaggatggtcaccaacaccaaagtgtctc

SEQ ID NO: 53 - DNA, 198 bp, synthetic construct
  misc_feature 1..198: note = Description of Sequence: FHR-1 SCR1 - GeneArt Codon Optimized
  source 1..198: mol_type = other DNA
gaggccaccttctgcgacttccccaagatcaaccacggcatcctgtacgacgaggaaaagtacaagccattcagccaggtgccaaccggcgaggtgttctactacagctgcgagtacaacttcgtgtcccctagcaagagcttctggacccggatcacctgtaccgaggaaggctggtcccctacacctaagtgcctg

SEQ ID NO: 54 - DNA, 174 bp, synthetic construct
  misc_feature 1..174: note = Description of Sequence: FHR-1 SCR2 - Non-codon Optimized
  source 1..174: mol_type = other DNA
agactgtgtttctttccttttgtggaaaatggtcattctgaatcttcaggacaaacacatctggaaggtgatactgtgcaaattatttgcaacacaggatacagacttcaaaacaatgagaacaacatttcatgtgtagaacggggctggtccacccctcccaaatgcaggtcc

SEQ ID NO: 55 - DNA, 174 bp, synthetic construct
  misc_feature 1..174: note = Description of Sequence: FHR-1 SCR2 - GeneArt Codon Optimized
  source 1..174: mol_type = other DNA
cggctgtgcttcttccccttcgtggaaaatggccacagcgagagcagcggccagacacatcttgagggcgataccgtgcagatcatctgtaacaccggctaccggctgcagaacaacgagaacaacatcagctgcgtggaacgcggctggtctacccctcctaagtgcagaagc

SEQ ID NO: 56 - protein, 4 aa, synthetic construct
  REGION 1..4: note = Description of Sequence: Linker amino acid sequence
GGGS

SEQ ID NO: 57 - protein, 5 aa, synthetic construct
  REGION 1..5: note = Description of Sequence: Linker amino acid sequence
GGGGS

SEQ ID NO: 58 - DNA, 29 bp, synthetic construct
  misc_feature 1..29: note = Description of Sequence: C7 Hairpin A
  source 1..29: mol_type = other DNA
gagccacagagaaatgctgaaggaccttc

SEQ ID NO: 59 - DNA, 29 bp, synthetic construct
  misc_feature 1..29: note = Description of Sequence: C7 Hairpin B
  source 1..29: mol_type = other DNA
ctgagtcttggtacagccattgcattctg

SEQ ID NO: 60 - DNA, 29 bp, synthetic construct
  misc_feature 1..29: note = Description of Sequence: C7 Hairpin C
  source 1..29: mol_type = other DNA
aaccaatgatttgctgatgcactgacctg

SEQ ID NO: 61 - DNA, 29 bp, synthetic construct
  misc_feature 1..29: note = Description of Sequence: C7 Hairpin D
  source 1..29: mol_type = other DNA
atctttcaaggcaggaggtgatggacaga

SEQ ID NO: 62 - DNA, 19 bp, synthetic construct
  misc_feature 1..19: note = Description of Sequence: C3 Hairpin A
  source 1..19: mol_type = other DNA
aattgttggagttgcccac

SEQ ID NO: 63 - DNA, 19 bp, synthetic construct
  misc_feature 1..19: note = Description of Sequence: C3 Hairpin B
  source 1..19: mol_type = other DNA
atctttagcctcctgcagc

SEQ ID NO: 64 - DNA, 19 bp, synthetic construct
  misc_feature 1..19: note = Description of Sequence: C3 Hairpin C
  source 1..19: mol_type = other DNA
ttcgaacaacagagtaggg

SEQ ID NO: 65 - DNA, 19 bp, synthetic construct
  misc_feature 1..19: note = Description of Sequence: C3 Hairpin D
  source 1..19: mol_type = other DNA
ttatggtgaccttgaggtc

SEQ ID NO: 66 - DNA, 19 bp, synthetic construct
  misc_feature 1..19: note = Description of Sequence: C3 Hairpin E
  source 1..19: mol_type = other DNA
agatggtcttgtctgtctg

SEQ ID NO: 67 - DNA, 21 bp, synthetic construct
  misc_feature 1..21: note = Description of Sequence: C3 Hairpin F
  source 1..21: mol_type = other DNA
ttcctccaggttgtaataggc

SEQ ID NO: 68 - DNA, 21 bp, synthetic construct
  misc_feature 1..21: note = Description of Sequence: C3 Hairpin G
  source 1..21: mol_type = other DNA
tttcttggtgtatcacgggcg

SEQ ID NO: 69 - DNA, 21 bp, synthetic construct
  misc_feature 1..21: note = Description of Sequence: C3 Hairpin H
  source 1..21: mol_type = other DNA
atgatgagggtgttcctatcg

SEQ ID NO: 70 - DNA, 21 bp, synthetic construct
  misc_feature 1..21: note = Description of Sequence: C3 Hairpin I
  source 1..21: mol_type = other DNA
ttcattctgattccttccggc

SEQ ID NO: 71 - DNA, 21 bp, synthetic construct
  misc_feature 1..21: note = Description of Sequence: C3 Hairpin J
  source 1..21: mol_type = other DNA
ttgttcttcttattcagcacg

SEQ ID NO: 72 - DNA, 21 bp, synthetic construct
  misc_feature 1..21: note = Description of Sequence: C4A Hairpin A
  source 1..21: mol_type = other DNA
attctttcccaggttccaagc

SEQ ID NO: 73 - DNA, 21 bp, synthetic construct
  misc_feature 1..21: note = Description of Sequence: C4A Hairpin B
  source 1..21: mol_type = other DNA
ttacgagatgggtttctcagg

SEQ ID NO: 74 - DNA, 21 bp, synthetic construct
  misc_feature 1..21: note = Description of Sequence: C4A Hairpin C
  source 1..21: mol_type = other DNA
ttgacatccttggtgaagtgc

SEQ ID NO: 75 - DNA, 21 bp, synthetic construct
  misc_feature 1..21: note = Description of Sequence: C4A Hairpin D
  source 1..21: mol_type = other DNA
taaggtgaagaagctggatgc

SEQ ID NO: 76 - DNA, 21 bp, synthetic construct
  misc_feature 1..21: note = Description of Sequence: C4A Hairpin E
  source 1..21: mol_type = other DNA
ttaccatcctcatctaggagc

SEQ ID NO: 77 - DNA, 21 bp, synthetic construct
  misc_feature 1..21: note = Description of Sequence: C4B Hairpin A
  source 1..21: mol_type = other DNA
ttatccaggtagttatagagg

SEQ ID NO: 78 - DNA, 21 bp, synthetic construct
  misc_feature 1..21: note = Description of Sequence: C4B Hairpin B
  source 1..21: mol_type = other DNA
tttgcttccatcgtgtactcg

SEQ ID NO: 79 - DNA, 21 bp, synthetic construct
  misc_feature 1..21: note = Description of Sequence: C4B Hairpin C
  source 1..21: mol_type = other DNA
aagtgtccaatggatctgagg

SEQ ID NO: 80 - DNA, 21 bp, synthetic construct
  misc_feature 1..21: note = Description of Sequence: C4B Hairpin D
  source 1..21: mol_type = other DNA
ttcacctcaaagttgggaagg

SEQ ID NO: 81 - DNA, 21 bp, synthetic construct
  misc_feature 1..21: note = Description of Sequence: C4B Hairpin E
  source 1..21: mol_type = other DNA
tttctggataaggtccactgg

SEQ ID NO: 82 - DNA, 19 bp, synthetic construct
  misc_feature 1..19: note = Description of Sequence: C5 Hairpin A
  source 1..19: mol_type = other DNA
tattaacgcaggctccatc

SEQ ID NO: 83 - DNA, 19 bp, synthetic construct
  misc_feature 1..19: note = Description of Sequence: C5 Hairpin B
  source 1..19: mol_type = other DNA
ttatgagagatattagcac

SEQ ID NO: 84 - DNA, 19 bp, synthetic construct
  misc_feature 1..19: note = Description of Sequence: C5 Hairpin C
  source 1..19: mol_type = other DNA
aacacagaactgcatccca

SEQ ID NO: 85 - DNA, 19 bp, synthetic construct
  misc_feature 1..19: note = Description of Sequence: C5 Hairpin D
  source 1..19: mol_type = other DNA
attccaatcacagtaaagg

SEQ ID NO: 86 - DNA, 19 bp, synthetic construct
  misc_feature 1..19: note = Description of Sequence: C5 Hairpin E
  source 1..19: mol_type = other DNA
aattaagtactgtcttcct

SEQ ID NO: 87 - DNA, 19 bp, synthetic construct
  misc_feature 1..19: note = Description of Sequence: C5 Hairpin F
  source 1..19: mol_type = other DNA
attagctaaaaatgcttga

SEQ ID NO: 88 - DNA, 21 bp, synthetic construct
  misc_feature 1..21: note = Description of Sequence: C5 Hairpin G
  source 1..21: mol_type = other DNA
taagtgcaaactgtatgcagc

SEQ ID NO: 89 - DNA, 21 bp, synthetic construct
  misc_feature 1..21: note = Description of Sequence: C5 Hairpin H
  source 1..21: mol_type = other DNA
ttcaaagagttcaaatatccg

SEQ ID NO: 90 - DNA, 21 bp, synthetic construct
  misc_feature 1..21: note = Description of Sequence: C5 Hairpin I
  source 1..21: mol_type = other DNA
aaccaaattcagtttgtaggg

SEQ ID NO: 91 - DNA, 21 bp, synthetic construct
  misc_feature 1..21: note = Description of Sequence: C5 Hairpin J
  source 1..21: mol_type = other DNA
ttatgagagatattagcacgg

SEQ ID NO: 92 - DNA, 21 bp, synthetic construct
  misc_feature 1..21: note = Description of Sequence: C5 Hairpin K
  source 1..21: mol_type = other DNA
atataagctgttctctcgggc

SEQ ID NO: 93 - DNA, 19 bp, synthetic construct
  misc_feature 1..19: note = Description of Sequence: C6 Hairpin A
  source 1..19: mol_type = other DNA
ttatttaaacatttctcag

SEQ ID NO: 94 - DNA, 19 bp, synthetic construct
  misc_feature 1..19: note = Description of Sequence: C6 Hairpin B
  source 1..19: mol_type = other DNA
ttgtgagaggcttgaatgg

SEQ ID NO: 95 - DNA, 19 bp, synthetic construct
  misc_feature 1..19: note = Description of Sequence: C6 Hairpin C
  source 1..19: mol_type = other DNA
atagttgcttttcattccg

SEQ ID NO: 96 - DNA, 19 bp, synthetic construct
  misc_feature 1..19: note = Description of Sequence: C6 Hairpin D
  source 1..19: mol_type = other DNA
agtcttgtcctttcaagac

SEQ ID NO: 97 - DNA, 19 bp, synthetic construct
  misc_feature 1..19: note = Description of Sequence: C6 Hairpin E
  source 1..19: mol_type = other DNA
tctctgacagcttgttggt

SEQ ID NO: 98 - DNA, 19 bp, synthetic construct
  misc_feature 1..19: note = Description of Sequence: C6 Hairpin F
  source 1..19: mol_type = other DNA
actcttgcaaagctttcct

SEQ ID NO: 99 - DNA, 21 bp, synthetic construct
  misc_feature 1..21: note = Description of Sequence: C6 Hairpin G
  source 1..21: mol_type = other DNA
tatcaaggacttctcctctgg

SEQ ID NO: 100 - DNA, 21 bp, synthetic construct
  misc_feature 1..21: note = Description of Sequence: C6 Hairpin H
  source 1..21: mol_type = other DNA
tagaggcagatggttaagtgc

SEQ ID NO: 101 - DNA, 21 bp, synthetic construct
  misc_feature 1..21: note = Description of Sequence: C6 Hairpin I
  source 1..21: mol_type = other DNA
tttcaaagccagtaaggcatg

SEQ ID NO: 102 - DNA, 21 bp, synthetic construct
  misc_feature 1..21: note = Description of Sequence: C6 Hairpin J
  source 1..21: mol_type = other DNA
attgcattctaactttctggc

SEQ ID NO: 103 - DNA, 21 bp, synthetic construct
  misc_feature 1..21: note = Description of Sequence: C6 Hairpin K
  source 1..21: mol_type = other DNA
ataggcagacacatttggaag

SEQ ID NO: 104 - DNA, 19 bp, synthetic construct
  misc_feature 1..19: note = Description of Sequence: C7 Hairpin E
  source 1..19: mol_type = other DNA
actggtcgattaatcttcg

SEQ ID NO: 105 - DNA, 19 bp, synthetic construct
  misc_feature 1..19: note = Description of Sequence: C7 Hairpin F
  source 1..19: mol_type = other DNA
tcacttccacacaaatgct

SEQ ID NO: 106 - DNA, 21 bp, synthetic construct
  misc_feature 1..21: note = Description of Sequence: C7 Hairpin G
  source 1..21: mol_type = other DNA
aaatccatctttcaaggcagg

SEQ ID NO: 107 - DNA, 21 bp, synthetic construct
  misc_feature 1..21: note = Description of Sequence: C7 Hairpin H
  source 1..21: mol_type = other DNA
aaaccaatgatttgctgatgc

SEQ ID NO: 108 - DNA, 21 bp, synthetic construct
  misc_feature 1..21: note = Description of Sequence: C7 Hairpin I
  source 1..21: mol_type = other DNA
tactggtcgattaatcttcgg

SEQ ID NO: 109 - DNA, 21 bp, synthetic construct
  misc_feature 1..21: note = Description of Sequence: C7 Hairpin J
  source 1..21: mol_type = other DNA
tttctgtcgtttctccaacgc

SEQ ID NO: 110 - DNA, 21 bp, synthetic construct
  misc_feature 1..21: note = Description of Sequence: C7 Hairpin K
  source 1..21: mol_type = other DNA
tttgcttctctcatcttgagc

SEQ ID NO: 111 - DNA, 19 bp, synthetic construct
  misc_feature 1..19: note = Description of Sequence: C8A Hairpin A
  source 1..19: mol_type = other DNA
taccctaccaattaataat

SEQ ID NO: 112 - DNA, 19 bp, synthetic construct
  misc_feature 1..19: note = Description of Sequence: C8A Hairpin B
  source 1..19: mol_type = other DNA
agttaacagtcaacatcca

SEQ ID NO: 113 - DNA, 19 bp, synthetic construct
  misc_feature 1..19: note = Description of Sequence: C8A Hairpin C
  source 1..19: mol_type = other DNA
taacagtcaacatccagtt

SEQ ID NO: 114 - DNA, 19 bp, synthetic construct
  misc_feature 1..19: note = Description of Sequence: C8A Hairpin D
  source 1..19: mol_type = other DNA
ttgctttgtcaatcaccag

SEQ ID NO: 115 - DNA, 19 bp, synthetic construct
  misc_feature 1..19: note = Description of Sequence: C8A Hairpin E
  source 1..19: mol_type = other DNA
tacacaagttgtagaactg

SEQ ID NO: 116 - DNA, 21 bp, synthetic construct
  misc_feature 1..21: note = Description of Sequence: C8A Hairpin F
  source 1..21: mol_type = other DNA
atgaagtgtcttgtgagtggg

SEQ ID NO: 117 - DNA, 21 bp, synthetic construct
  misc_feature 1..21: note = Description of Sequence: C8A Hairpin G
  source 1..21: mol_type = other DNA
ttacacaagttgtagaactgg

SEQ ID NO: 118 - DNA, 21 bp, synthetic construct
  misc_feature 1..21: note = Description of Sequence: C8A Hairpin H
  source 1..21: mol_type = other DNA
aatggtctcctgataaacctc

SEQ ID NO: 119 - DNA, 21 bp, synthetic construct
  misc_feature 1..21: note = Description of Sequence: C8A Hairpin I
  source 1..21: mol_type = other DNA
tttgctttgtcaatcaccagg

SEQ ID NO: 120 - DNA, 21 bp, synthetic construct
  misc_feature 1..21: note = Description of Sequence: C8A Hairpin J
  source 1..21: mol_type = other DNA
atacatgccataattgtactg

SEQ ID NO: 121 - DNA, 19 bp, synthetic construct
  misc_feature 1..19: note = Description of Sequence: C8B Hairpin A
  source 1..19: mol_type = other DNA
tagctttccacattgtagg

SEQ ID NO: 122 - DNA, 19 bp, synthetic construct
  misc_feature 1..19: note = Description of Sequence: C8B Hairpin B
  source 1..19: mol_type = other DNA
taaatccttctacagtttg

SEQ ID NO: 123 - DNA, 19 bp, synthetic construct
  misc_feature 1..19: note = Description of Sequence: C8B Hairpin C
  source 1..19: mol_type = other DNA
ttctctgtgacattgcgtt

SEQ ID NO: 124 - DNA, 19 bp, synthetic construct
  misc_feature 1..19: note = Description of Sequence: C8B Hairpin D
  source 1..19: mol_type = other DNA
tacacaccttcctgtctgt

SEQ ID NO: 125 - DNA, 21 bp, synthetic construct
  misc_feature 1..21: note = Description of Sequence: C8B Hairpin E
  source 1..21: mol_type = other DNA
ttgtcttacgtcttccagagc

SEQ ID NO: 126 - DNA, 21 bp, synthetic construct
  misc_feature 1..21: note = Description of Sequence: C8B Hairpin F
  source 1..21: mol_type = other DNA
tagtatgagagaatcgtttgg

SEQ ID NO: 127 - DNA, 21 bp, synthetic construct
  misc_feature 1..21: note = Description of Sequence: C8B Hairpin G
  source 1..21: mol_type = other DNA
atttcattcagaatacctctg

SEQ ID NO: 128 - DNA, 21 bp, synthetic construct
  misc_feature 1..21: note = Description of Sequence: C8B Hairpin H
  source 1..21: mol_type = other DNA
tagacagctcacaatcaatgg

SEQ ID NO: 129 - DNA, 21 bp, synthetic construct
  misc_feature 1..21: note = Description of Sequence: C8B Hairpin I
  source 1..21: mol_type = other DNA
tttctctgtgacattgcgttc

SEQ ID NO: 130 - DNA, 19 bp, synthetic construct
  misc_feature 1..19: note = Description of Sequence: C8G Hairpin A
  source 1..19: mol_type = other DNA
cggaaggtactgacagcca

SEQ ID NO: 131 - DNA, 19 bp, synthetic construct
  misc_feature 1..19: note = Description of Sequence: C8G Hairpin B
  source 1..19: mol_type = other DNA
tggggaagtagaagatctg

SEQ ID NO: 132 - DNA, 21 bp, synthetic construct
  misc_feature 1..21: note = Description of Sequence: C8G Hairpin C
  source 1..21: mol_type = other DNA
aaactctggtagtcggtctca

SEQ ID NO: 133 - DNA, 21 bp, synthetic construct
  misc_feature 1..21: note = Description of Sequence: C8G Hairpin D
  source 1..21: mol_type = other DNA
atcccatccagctttcggaag

SEQ ID NO: 134 - DNA, 21 bp, synthetic construct
  misc_feature 1..21: note = Description of Sequence: C8G Hairpin E
  source 1..21: mol_type = other DNA
aaacccactcaggaccgagtc

SEQ ID NO: 135 - DNA, 21 bp, synthetic construct
  misc_feature 1..21: note = Description of Sequence: C8G Hairpin F
  source 1..21: mol_type = other DNA
tacaggacagcgaaactctgg

SEQ ID NO: 136 - DNA, 21 bp, synthetic construct
  misc_feature 1..21: note = Description of Sequence: C8G Hairpin G
  source 1..21: mol_type = other DNA
ttcgtccaggacgtggaactg

SEQ ID NO: 137 - DNA, 19 bp, synthetic construct
  misc_feature 1..19: note = Description of Sequence: C9 Hairpin A
  source 1..19: mol_type = other DNA
atgttgatcccatagcctg

SEQ ID NO: 138 - DNA, 19 bp, synthetic construct
  misc_feature 1..19: note = Description of Sequence: C9 Hairpin B
  source 1..19: mol_type = other DNA
tgttcttcgtaatgttcgg

SEQ ID NO: 139 - DNA, 19 bp, synthetic construct
  misc_feature 1..19: note = Description of Sequence: C9 Hairpin C
  source 1..19: mol_type = other DNA
tatgagtgaaacaacatca

SEQ ID NO: 140 - DNA, 19 bp, synthetic construct
  misc_feature 1..19: note = Description of Sequence: C9 Hairpin D
  source 1..19: mol_type = other DNA
gaaacaacatcatctatga

SEQ ID NO: 141 - DNA, 19 bp, synthetic construct
  misc_feature 1..19: note = Description of Sequence: C9 Hairpin E
  source 1..19: mol_type = other DNA
agttcaacacctttccgct

SEQ ID NO: 142 - DNA, 19 bp, synthetic construct
  misc_feature 1..19: note = Description of Sequence: C9 Hairpin F
  source 1..19: mol_type = other DNA
aaaatttgatgtcttctct

SEQ ID NO: 143 - DNA, 19 bp, synthetic construct
  misc_feature 1..19: note = Description of Sequence: C9 Hairpin G
  source 1..19: mol_type = other DNA
tgaacgaaacatttgtctg

SEQ ID NO: 144 - DNA, 21 bp, synthetic construct
  misc_feature 1..21: note = Description of Sequence: C9 Hairpin H
  source 1..21: mol_type = other DNA
attatagacctaagagagaag

SEQ ID NO: 145 - DNA, 21 bp, synthetic construct
  misc_feature 1..21: note = Description of Sequence: C9 Hairpin I
  source 1..21: mol_type = other DNA
ttgtttactgatttcacaggc

SEQ ID NO: 146 - DNA, 21 bp, synthetic construct
  misc_feature 1..21: note = Description of Sequence: C9 Hairpin J
  source 1..21: mol_type = other DNA
aatgttgatcccatagcctgc

SEQ ID NO: 147 - DNA, 21 bp, synthetic construct
  misc_feature 1..21: note = Description of Sequence: C9 Hairpin K
  source 1..21: mol_type = other DNA
tttgttcttcgtaatgttcgg

SEQ ID NO: 148 - DNA, 21 bp, synthetic construct
  misc_feature 1..21: note = Description of Sequence: C9 Hairpin L
  source 1..21: mol_type = other DNA
ttagctctgggtcataactgg

SEQ ID NO: 149 - DNA, 19 bp, synthetic construct
  misc_feature 1..19: note = Description of Sequence: CFB Hairpin A
  source 1..19: mol_type = other DNA
tattcttgagcttgatcag

SEQ ID NO: 150 - DNA, 19 bp, synthetic construct
  misc_feature 1..19: note = Description of Sequence: CFB Hairpin B
  source 1..19: mol_type = other DNA
tggtcttgagtcttcaggg

SEQ ID NO: 151 - DNA, 19 bp, synthetic construct
  misc_feature 1..19: note = Description of Sequence: CFB Hairpin C
  source 1..19: mol_type = other DNA
tgatgtagacctccttccg

SEQ ID NO: 152 - DNA, 19 bp, synthetic construct
  misc_feature 1..19: note = Description of Sequence: CFB Hairpin D
  source 1..19: mol_type = other DNA
tgaatgaaacgacttctct

SEQ ID NO: 153 - DNA, 19 bp, synthetic construct
  misc_feature 1..19: note = Description of Sequence: CFB Hairpin E
  source 1..19: mol_type = other DNA
attcttgagcttgatcagg

SEQ ID NO: 154 - DNA, 19 bp, synthetic construct
  misc_feature 1..19: note = Description of Sequence: CFB Hairpin F
  source 1..19: mol_type = other DNA
tgagctgcttcgtgaccca

SEQ ID NO: 155 - DNA, 19 bp, synthetic construct
  misc_feature 1..19: note = Description of Sequence: CFB Hairpin G
  source 1..19: mol_type = other DNA
aggactacttctatctcca

SEQ ID NO: 156 - DNA, 21 bp, synthetic construct
  misc_feature 1..21: note = Description of Sequence: CFB Hairpin H
  source 1..21: mol_type = other DNA
tttggctcctgtgaagttgct

SEQ ID NO: 157 - DNA, 21 bp, synthetic construct
  misc_feature 1..21: note = Description of Sequence: CFB Hairpin I
  source 1..21: mol_type = other DNA
tacgctgaccttgattgagtg

SEQ ID NO: 158 - DNA, 21 bp, synthetic construct
  misc_feature 1..21: note = Description of Sequence: CFB Hairpin J
  source 1..21: mol_type = other DNA
ttgaagggcgaatgactgaga

SEQ ID NO: 159 - DNA, 21 bp, synthetic construct
  misc_feature 1..21: note = Description of Sequence: CFB Hairpin K
  source 1..21: mol_type = other DNA
ttgatgtgaaagtctcgggcg

SEQ ID NO: 160 - DNA, 21 bp, synthetic construct
  misc_feature 1..21: note = Description of Sequence: CFB Hairpin L
  source 1..21: mol_type = other DNA
atatccttgactttgaacaca

SEQ ID NO: 161 - DNA, 19 bp, synthetic construct
  misc_feature 1..19: note = Description of Sequence: CFD Hairpin A
  source 1..19: mol_type = other DNA
tgctcgggactttgttgct

SEQ ID NO: 162 - DNA, 19 bp, synthetic construct
  misc_feature 1..19: note = Description of Sequence: CFD Hairpin B
  source 1..19: mol_type = other DNA
agatcccgggcttcttgcg

SEQ ID NO: 163 - DNA, 19 bp, synthetic construct
  misc_feature 1..19: note = Description of Sequence: CFD Hairpin C
  source 1..19: mol_type = other DNA
ttgttgcttgggtgaccct

SEQ ID NO: 164 - DNA, 19 bp, synthetic construct
  misc_feature 1..19: note = Description of Sequence: CFD Hairpin D
  source 1..19: mol_type = other DNA
cacccttgcagctgtcccg

SEQ ID NO: 165 - DNA, 21 bp, synthetic construct
  misc_feature 1..21: note = Description of Sequence: CFD Hairpin E
  source 1..21: mol_type = other DNA
atttgtagagatggagtcgcg

SEQ ID NO: 166 - DNA, 21 bp, synthetic construct
  misc_feature 1..21: note = Description of Sequence: CFD Hairpin F
  source 1..21: mol_type = other DNA
ttcttgcggttgccgcaaacg

SEQ ID NO: 167 - DNA, 21 bp, synthetic construct
  misc_feature 1..21: note = Description of Sequence: CFD Hairpin G
  source 1..21: mol_type = other DNA
aacctgcaccttcccgtcggc

SEQ ID NO: 168 - DNA, 21 bp, synthetic construct
  misc_feature 1..21: note = Description of Sequence: CFD Hairpin H
  source 1..21: mol_type = other DNA
tccgcgcacatcaagcgctcg

SEQ ID NO: 169 - DNA, 19 bp, synthetic construct
  misc_feature 1..19: note = Description of Sequence: VEGFA Hairpin A
  source 1..19: mol_type = other DNA
tataccgggatttcttgcg

SEQ ID NO: 170 - DNA, 19 bp, synthetic construct
  misc_feature 1..19: note = Description of Sequence: VEGFA Hairpin B
  source 1..19: mol_type = other DNA
ttctgtatcgatcgttctg

SEQ ID NO: 171 - DNA, 19 bp, synthetic construct
  misc_feature 1..19: note = Description of Sequence: VEGFA Hairpin C
  source 1..19: mol_type = other DNA
tctgtatcgatcgttctgt

SEQ ID NO: 172 - DNA, 21 bp, synthetic construct
  misc_feature 1..21: note = Description of Sequence: VEGFA Hairpin D
  source 1..21: mol_type = other DNA
acttcgtgatgattctgccct

SEQ ID NO: 173 - DNA, 21 bp, synthetic construct
  misc_feature 1..21: note = Description of Sequence: VEGFA Hairpin E
  source 1..21: mol_type = other DNA
ttataccgggatttcttgcgc

SEQ ID NO: 174 - DNA, 21 bp, synthetic construct
  misc_feature 1..21: note = Description of Sequence: VEGFA Hairpin F
  source 1..21: mol_type = other DNA
ttggtgaggtttgatccgcat

SEQ ID NO: 175 - DNA, 21 bp, synthetic construct
  misc_feature 1..21: note = Description of Sequence: VEGFA Hairpin G
  source 1..21: mol_type = other DNA
tcacatctgcaagtacgttcg

SEQ ID NO: 176 - DNA, 21 bp, synthetic construct
  misc_feature 1..21: note = Description of Sequence: VEGFA Hairpin H
  source 1..21: mol_type = other DNA
ttgcaggaacatttacacgtc

SEQ ID NO: 177 - DNA, 19 bp, synthetic construct
  misc_feature 1..19: note = Description of Sequence: CFP Hairpin A
  source 1..19: mol_type = other DNA
aggattaggtccacagggg

SEQ ID NO: 178 - DNA, 19 bp, synthetic construct
  misc_feature 1..19: note = Description of Sequence: CFP Hairpin B
  source 1..19: mol_type = other DNA
agacatggtcgtttctcct

SEQ ID NO: 179 - DNA, 19 bp, synthetic construct
  misc_feature 1..19: note = Description of Sequence: CFP Hairpin C
  source 1..19: mol_type = other DNA
tcgtgtctccttaggttcg

SEQ ID NO: 180 - DNA, 21 bp, synthetic construct
  misc_feature 1..21: note = Description of Sequence: CFP Hairpin D
  source 1..21: mol_type = other DNA
tacgtttctggtaggcaaagg

SEQ ID NO: 181 - DNA, 21 bp, synthetic construct
  misc_feature 1..21: note = Description of Sequence: CFP Hairpin E
  source 1..21: mol_type = other DNA
atggacttcatgttccgtcgg

SEQ ID NO: 182 - DNA, 21 bp, synthetic construct
  misc_feature 1..21: note = Description of Sequence: CFP Hairpin F
  source 1..21: mol_type = other DNA
ttgccggaggattcttcatac

SEQ ID NO: 183 - DNA, 21 bp, synthetic construct
  misc_feature 1..21: note = Description of Sequence: CFP Hairpin G
  source 1..21: mol_type = other DNA
atcgatgtccgtcaaacttgc

SEQ ID NO: 184 - DNA, 21 bp, synthetic construct
  misc_feature 1..21: note = Description of Sequence: CFP Hairpin H
  source 1..21: mol_type = other DNA
ttcgtgtctccttaggttcgt

SEQ ID NO: 185 - DNA, 5003 bp, synthetic construct
  misc_feature 1..5003: note = Description of Sequence: (TYR Promoter Region)
  source 1..5003: mol_type = other DNA
cactaaatacacaaaactttagaagtcttcactacagttaaaaatatatcatgtttagaggttgttaggtagccacattattattctgaaaattgaaaaatgaaggacaagaatcaaacatttaccccgcttttcttgtacagggtaactaaatagttcatgaagaaagttattctttacaaaaaaaaggggataaccattcttgcaacctttaataaaataccaaatgtaggatatacttatcagtgaaaataaaaccatataaaagtttgctagagaattatgtaattgaaaggacagtctgacatcacttcaagctgctgataaattttaaaaacaaatactgagaaaaccagataatatggtcctctagtatatcaaaataagtattacttaacaatgcctgtgaagtaattgttaagaaaagcaataacaaaattcaacttgtatataataaaatctctagatctaactccctgattgtagaaaatgcaagatattattattgaaaatgtataaaatgatagaaaatacaggataatgagcatgttaaattcatagagataaaagttgttaaccccgaaatgtgagaaatccttagggcaaaaactcagtttcctctaaaaataaattgcattttttttttttttttttttttttttgagacggagtctcgctctgtcgtccaggctggagtgcagtggcgggatctcggctcactgcaagctccgcctcccgggttcacgccattctcctgcctcagcctcccaagtagctgggactacaggcgcccgccactacgcccggctaattttttgtatttttagtagagacggggtttcaccgttttagccgggatggtctcgatctcctgacctcgtgatccgcccgcctcggcctcccaaagtgctgggattacaggcgtgagccaccgcgcccggccgcatttttttttaaaaaaggaaacttataacttaatagaaattgataaaatagatcaaccaaaatgcagtgtatatacctgtttgaatcttgacaaaaatacttttgagataattaaagtaaattaaccataaatttgatacctgatattaaagaattattgttctttttgtttgagatgtgtttgtgtaagatacgtggttttgtttttatgttcttaattgttagcaatatataccgaaattagaggtagaatatgctatctgagttttgttttaaaacattccagaatttttaaactagtagaaacaaaacagaaaaatgctggatgttgaaacttggaggtaagtaaatgctagtttattatattattttttactgatttgagcatatttctttccacagtaaatttttttctttaaatggtagctactgatactgagataattatattagttgtatttaataaatggtagctaccattataattgaaaggagggctgagaaacaaagacagaaaatggattcaaactactaccaaataaaagagcaaataccagtgatctgagtgaggaaaatgaggaaaattgaccaggttaacacaagtgagtggctgaataatctatacaaataccagaagtagaggaccagtgattgtggctagttgatactcaaactcaacttatctaaaattgagcctatattcaattcccataaacatttatcctctattgtttcttattttagttaatggcacaagccaaaaacctagcacatagccctaaatcttctttcccttcacttcttactgctcttgaatttatcacctatgtatctctcaaattcattaacttatgcccatctcagagatcattactctagtctaagcaatgtcatgtttgcctggtttacatcaataatatcctaacagtaatctccacatttaatctagctcccaccccacatctgttctcacctcagtagcttgagtgaaaagttttaaattcatgtatcgttacccaaaattttgctacaaagcctttatggactatgagctggtccatacacacaccttcattcttatatcacgcctctaccatattgggggttgcagccaccttaactgtccttaaatcccttgaatataccaacctacttccactttggagactttgcacatgctattctctctgcctggaaagctcacactcctactgttctctctgtcatcaaacagacttctagtgatccctcaagtgtcacttgaaatgtcacttctcccataaaattatccctgactatcccttctcttgtgagactagaaaggcccttctgtcatgttccctaatttctctctttaattttctcttatagcaacctaaaaagtaatttaatccagaaaaaaagttatttaaagtaatgtagttaaaattattatttaaaatcactaaaattaaaatagtattttaagaggcaatatccttcctgatgatagagatttttgttgtgcttaccatcacattcccatctctccccatgtctgcaagtaaatacttgcagagtatttactgaacccctaattctactttatagagcattgcagtaatttcatgtcaggtcatacaataatctcccaaaataggataggaccctttcaaatcccacaactgaagatacctaaagaatgtacatctagagtcaggaagaaagcactctaaatgggatttaatatttaggaacccaagactggcaataagactgaagactacaacacgtgtaggccagaggagacagtggcctatactggggacaaataaagaggtctgtcctatttaagaaaatcaaccctgtaaaggaaattaataggactaagtacattttagtaattcctctaagcaggctctaaagattatgaaaaatagacgggacagcagacacaaaagcccttaaagagcatgatgaagactttctaagttatttcactggaagcctgatagtggggcaagtgtaaggcaaaattcttaattaaattgaacatgataagttgaattctgtcttcgagaacatagaaaagaattatgaaatgccacatgtggttacaagtaatgcagacccaaggctccccagggacaagaagtcttgtgttaatctctttgtggctctgaaagaaagagagagagaaaagattaagcctccttgtggagatcatgtgatgacttcctgattccagccagaggcagcatttccatggaaacttctcttcctcttcacccacacactgctccatgtacctgcaaagcctgttctgtctcaaaaaagttgtttggatgagccgtgacttttttttttcttaaataatgagacaaactccagaaaaagagaaaaaagcagagcagtctgacattccggcatcatcgaaatagtgatggcttttcctagaatgcttcagctaaggacccaaatactaatgatctcctcaaagctttcactttcttttactttttcattaatttcagtggacccccaaactttaagtatggaagaggacaaagaaggaagcttcagaggggcaactttgatttgactactctttttgtcactcttcagctcacaaaagagctcactttagttcaaaacacaaagtctttaagcccctccatagattggtcccaggtttaattttctatgatgtgtggaggcctcagtttaatgctccaacttgatagatgaaacacagttcccacctctacacatttcccctgtctcaggagttgtatatattctcagttgtctgtccaacttatgcccactctttgagatattaatcaaggcactcccttgataacacttgcatattattatcaaaattatgcaattctttctaatatcagcccacaaatacatctcttccattaaaagtttgacttaattatctatactactcatttgaaaactaacatagttaagttgtatttttagccatgaatttcagtttccctagctcactatacacagagaaggaacttttgaaataattgagatgatcaaaaatatttgctgaagaaatatatttctcctttttcattcactcactaattgagaatgtctttgcacaaaacacattgcaaaaacattttcaaaaaaattcctaatttctagaattgataggaaaaacaatatggctacagcattggagagagagagaaaggagagaggagaaaggagagagagagaaaggagagaggagagagacagaggagagagagagaggatagaggggagagagagagaggagagagacagaggagagagagagaggatagaggggagagagagggagagggagagagagggagagagagggagagagagagagagagagggagagagagagagaaagagagagagagggagagagagagagagagctctttaacgtgagatatccccacaatgaagcaaatcgcccagttatcaaagtgagctatccttaggagttgtcagaaaatgcatcaggattatcagagaaaagtatcagaaagatttttttttctgatacgttgtataaaataaacaaactgaaattcaataacatataaggaattctgtctgggctctgaagacaatctctctctgcatattgagttcttcaaacattgtagcctctttatggtctctgagaaataactaccttaaacccataatctttaatacttcctaaactttcttaataagagaagctctattcctgacactacctctcatttgcaaggtcaaatcatcattagttttgtagtctattaactgggtttgcttaggtcaggcattattattactaaccttattgttaatattctaaccataagaattaaactattaatggtgaatagagtttttcactttaacataggcctatcccactggtgggatacgagccaattcgaaagaaaagtcagtcatgtgcttttcagaggatgaaagcttaagataaagactaaaagtgtttgatgctggaggtgggagtggtattatataggtctcagccaagacatgtgataatc

SEQ ID NO: 186 - DNA, 4999 bp, synthetic construct
  misc_feature 1..4999: note = Description of Sequence: (TYRP1 Promoter Region)
  source 1..4999: mol_type = other DNA
ctaccaaattcttcattccctcctcttgtgatgtaacttctaacatcccgtctcatggcagattattagagtgcgtaaggaggaaacagaggtaaattttggttctgatgaagaggtagaaggggtcaggaggtttccatcattgtctgtcttctgcaaggttatctaaaaagtgaagagacatccttaaagtgagtattgaccaaatgtcagttgtctcttcagacttcctatggtttaaaaataaatgaaaagaaaaaagctctgttttcttacctaaatttgagttatattaccaaaggccccttaggtaatggggcctttatacgttccagagtaagtagcatatggtgtgtgcatgcatgtttctctctctctctctctctctcattctgtgtgtgtgtgtgtgtgtgtgtgtgtgtgtgtgtgtgtgtcagtggagagagagagagacgaagaaaagggaggagtgataatccttctaaacgtatacaagttgcttgtgagaaaaatagcagtaatcataacacaccaagtcccttactgctggtaatgcttttcatgtgtctctccagattgcctaattttaacttcttaaatgtctcatctcctcctctataccctggaaaattataatatgctgattcaatttaaaaaagttatttgaaatgtagccaaactcatttgatcttcctcaattcatcactgcatatgggtttcatcccattttattagtaaatttgacactgattatgataaatccatccagtgccacacatctccatctgcaaatttcaaggtttaattttaaactttttagggtaagaggtttttaaaatattttcaacctgtaggatttccaagaccttatttctaaatgtttcaatatgaccaatcttatacatgtgattttatatttctatatcagatataaaaaagatttagattatttaggctttcagttagttctttttatacatgaagctacttttacgagcaagatgaaggggaagtgagatagtggcagttgctgtcttttggatgacaggccaatttacttcttgcttctaaaggaagtaggctttaccctttaaacctcccccatcatatgttcttgcatgtactgtttcctttcatcagtaagttctcctgatcttctctgtctctccctctctctctatctctctctctgtttgactgtttctctgacattacccaaagataactgttttttttttctggtctcagaattattcgatgtcctttagtagtgaggtcagcttccacaaatgcctaattaaaaaaaattttccttaaaataggcaagaggaggaggagttgaattccaaacacctggagtctcctttgattagcagtatttaatttctgatagtacccaatttatggcattttagtgaagacaattctaaaagttaacagagaagtcaggccaatctttccgataggatgacacactcctttgtaaatctttaaatgcttttcattgcattatttgtgagtagggaccagcagtgttcctaaataggaagtgtgttttttgccaacagtaagtcccatagatctctggttttcctgtttagtggagggttactaagagagagtacttaatttctcacagttggaaattttcctggtttctctagaccatgtaattcttcaaaatgttatagggtagaccaagttctgaattttacctgtgcttttgattaggcttcagtttgttttacagattattgtatttcttgtctccttctgtgctttttccacagtgcaggcagtaatcacaatatccttcatagagtgtactaccttgcccttaaggatgttagaaacgcatggtatcgattcccattgtgcatttagataatagcaatacaagcacagaatattatcttggcaccatagaagctctacaatccccaagtgtcagtagtacattttcttcaaacgttagcaacctcaatccctggcaagaaaaaaaacagattatgcaatctataatgatcttactcttcagcaaaattctatttttttgtgttcgttattagaaatttccacagacatattttagcagtcatatttctgcctgcccagctagagtaagctcagtgaggaagggggtctgttgctactcttctttaatgatggaaacactgaggagtatatgaggaattttacccattagatgtgtgaggctaagagatctttgatcatgccactgctctccagcctgggcgacagagtgagaccctgtcccaaaaaaaaaaaaaaatgatatgtccactcattaaaaacttctgtcagaccactcttttttataaatttttaaattttttaatgatctaacaagctttatttttatcattgttaaaaaaatccttcttttgagtaacacaatcacaaagtaaagaatgttatcattgatttaaccagtaaaaaaacaatacttaacaataaaatttttcaaacatggctccaggccatccagatgacttagttatcctcctttgatgcccttctcttcttcgaatgaatcacaatgaggcagaaaaattttcatgggtgcagagtaggtgtatatatttatggagtatgtgagatattttgatacaggcatacaacatgaaataagcacatcatggagaatggggtattcataccctcaagcatttatcctttgagttacaaacaatccaataacactttttaagttatttaaaaatatacagttatgattgactatagtcaccctattgtgctatcaaacagtaggtcttattcattcttgctaatttttttgtacctattgactatccctacttccctactcccctcagacacccactacctttcccagcctccattcttctactctttatgtccataagttcaattgttttgaggtttagattccacaaataagtgagaataagtgatgtttgtctttctgtgcctggcttatttcacttaacagaatgtctagttccatccatgttggtgcaaatgactggatctcattgttttactgcattttgcatatgtaccatattttcttaatccattcatctattgatgaacacttaggttgcttccaaattttagctttgtaaacagtgctgcaataaacataggagtgcagatatctcttcaatatattgatttcctttcttttgggtatatatctagcagtgggattgctggatcatatagtagctcaatttttagttttttgaggaaactccaaactttgctccatagtggttgtactaatttacattactaccaacgctgtatgagggtttccttttctccacatccttgtcaacatttcttattgcctgaattttggacataagccattttaattggggtaagagggtatctcacaatagttttgatttgtacttctctaatgatcaataatgttgagtatcttttcataagtctgtttgccatttgtatgtcttcttttgataaatgtctgctcaaatattttgcccatttttttattggattattaatttttttctatagaattttttgagcttctcatatattctggatattaatcccttgtcaaatgggtagtttacaaatatcttatcccattctgtgggttgtctcttcacttcattgattgtactctttgctgtgcagaagatttttaactagatgtgatcccatttgttcatttttgctttggctacctgtgcttgtgtggtattgctcaagaaatctttgtcaagaccaatgtcctcaagattttcctcaatgtttcttcattgtagtttcatagtttgaggtctcagatttaaatatttaatcaaatcattttttatttgatttttgtatatggaacgagatagggacctagtttcattcttttgcatatgggtatgtagtttgccagcaccatttattaaaaagactgtcttttccccagtgtatgttcttggtatcttggtgaaaaatgtctttactgtaggtgtgtgattttgtttttgggttctcctgttccattgctctatgtgtctgtttttaggccagtaccatgctgtttttgctactatcactccgtagtataatttgaagtaaagtaatatgattcctccagttttgttatttttgcttaggataactttgcctcttcaaggtttggtgttccatataaattttaggattttctttctgtttctgtgaagaatgtctttggtattttgataggaattgcattgaatctgtagattgctttaggtaatacggactttttaacaatcttgattctcccaatccatgaacatataatatttttccattatttggtgtcttcttcaatttctttcatcaatgttttgtagttatcattatagagacctttcattcattggttaattcctaggtatttaattttatgtgtggctattgtaaatgggattacttttttatttctttttcacatttttcactgttggtatatggaaactttgctgattttggtatgctgattttgtatcctgcaactttactgaatttgtttgtcagctctaatagttttttagaggagtctttaggttttattcaaatataagatcttatcatcagcaaacaaggataatttgacttcttcatttccaatttggatgctctttataccttctcttgcctggttgctctagcaaggtaatatgttgaataacagttgaataacagaataaaaaaaaatctctgcaaagtaaacaaatctcactagtttatctgacttgtattccaaattagtgcttctggccttttcttaaaactttaagcatcacaaggaaatcagttggaagggaatcatgtgctgatcaagtccttaaagggcagaaatattcactgaagtgaaaaggattagtaaagggtggaaaaaaagaccagccccccgcctagtttgggtgagcagatttgggattaattatcaggcagcaatccacatgcactt

SEQ ID NO: 187 - DNA, 5000 bp, synthetic construct
  misc_feature 1..5000: note = Description of Sequence: (FLT1 Promoter Region)
  source 1..5000: mol_type = other DNA
gcattgctgaatgaaagaatacgtttcctggatcatgcaccatgctacgagctgtttccttcgactgctcagccaggtcactgttggactaagctggtagggttttaagtaaagcagggatattgatgagcgtgcatgtgcgtgtgtacatctgactaaaaacattccagtgtcaaattaagattattttggaattatttacaatgcagaaactggaatgtctagcaagaggataatagtttgcggctattagctacaacagttatgaaggctctatagaaacatggaaagataaaagccgtctgtatggaaaaggctcgaatgaaatacaccacagtgatcacagtggctctgctgagatgatggaattatgactcatcctccagaccttcccttcttttaatctttctaaaatgttatatttaatgactatgagtgtattaaaagtaataaaacaggaaaaagagtaaggagaagagtcaatggctggcagttaagtgctgagatacaactgaccttaggcaagatagtagtgatactgattagaggaggttcctagagattactctaattggccacttttcctttaggccttatttgcttctctgatttgccgcttcccctgccccatgcttctctaactatcccagtctggtctttttgttttttttcctttcaacttttctagtatgccttcaataaaatgtacctgtcttattttctcttgctaggaccatcaattattcctaaataatgacatggtaaatgtgaatgagttaatatgcagcctcctttggtgaacatacagctagagataatgacacaaaggaataatttgagtccttccttggtttgctcaacctccacagatctctactttcatccatccattcattcattcattcattcactcactcagctttgtccttgtcagtgattttgatgatgacccagaaggcacgtttaccgtatttgtggatcatttgaagcgggagctatagaaagtccacaggatggcagaatcagagaccagaaagacttcgacatgctggaaagacagactaaaacaaacaaaaggcttaaacaacacgctcagggcctgcagcctgagtcaacccaagcggcggctgcacaagggcagccagatagcagctctcatgagaaagccttggggctttaaccaagaataagttctggaagagtccaaggtatgaaggcgctgacagaaaaggctcctttgatcttaggtcccaggaacagaagcaagttctctccgtagtagcccctgggagtagcaggaggagaattccaagtaaagccaatggcgatcttataaacaccagcagcgcccacccatgcccgggagaaggtagtcaggagcagacgggagcatgagtagcaggaacaggaggagccagggagcccaggccggcggccccagaggtgccaccagggcagtgctgccctgctctcggtggcactggccttatctccaggagggggctgtcccctttgcttccttctccacgctgctcttcccacctgaacgattttccttcctcgctcccactgggccttcccacaggcttttctctctgcctgaaagaaaaactacctctaggagtagggaaatgataattgtgctgcattttcaatcctaagcgtgtcacctgcagggacactgatatgctggatggaatttctggagcgtgtcagctgaagagtgaccaaagggatgtggatgttgaggctggagacaagatgcccatggcacaggaggtgtttttaagtgttcaataaattttcatgtggagatattggagcattgaataggaattccacgaattgactcggtagatgggttgaatggcttttaaacccagtacaagatgtttaagttctttgaaaatgggggcatttcagccacagtggtagcttcccatagcaagacttttgagccaatggggtcatcagcatctgataagtaaactcatacatgtgcctgcctggtgaactccgcagaacttcttagcagagatttgggagcgtgtgctttttagaccaaggagcctgaactgtccagaggatatgtactgtgaagctttttaaaatataacacagaagggttttatgagatcatttggttcttgtattcaaccatacatatttgcattatgcagagtatgcccatagagtgtatttttccttttgtatcaagctacctaacccaagtggaaaagaaattcctgactgatcctcagtgactgtggctgatcagctccatgcttggtctttggaggaaggtgagagcctgggctccctcctgtctctcacactggtgctccaacactcttattcagtctcgaatattaaagctgatggatttagcaacagtgtgcttctgaataggggtgagaaagaaacctcaagagaatgtttgtagctgataacaagagagagtgtctggttttgctaaccaaacaggagatgaaatgttcttttgtatgtgagcaggaagcaagcagattgcctacttcaatagctttttatacccaccaggcgtgttgaattccatcttctccatcagtattatctataaaacctctttcattagtgatttagttaggagagtgctatagcccattctcattcaaaggtattatctcttttggggagaggattgggagctatacatttattccccatatctaagctaaaatttgtagcatttttgagttctttgtgagaacgctaatgcaactaacaaagaagtcatagtcaataaattgctgactctaattaaaagcaacttttcaaaagagtagttgggaaatgtggaagcccacccttactgcagcatagttttaatataaatcttagattcgactgtgactctactaaccttaaataattgagcaacatgttaatatcattgattagcatcttagattataaagattgtttcagctgtccatcaaatacctgttctggctttacagacacaatgtagattcattgctggcagcaggtttggaaagtgttgtctctttatgagttaaaaatatcaaaatggcatttgaaaaaatgtattagcaaattaaacctttgtacatggtactatggaccactttcccccagagtcaaatgtgtgtctctcctgagaagccctccttgggttttccctgccaataaatgtgcctttacttctctgagctgatggaggactaaacacccctccagcactggaatgccggtcctgcagccacttcaagtgtccaccttcttatcacagtccaagccctttagaaggcaaagatgatggctctccatcagtgttttcagccctaacccaatgcctggcccctgttgaatgctgcagaaggacaatgctgacagcttctccttgaaagagggtgtgggagtgacaagcagctctcaggtgagaccaacagacactgtggctccgcacgtatgtcccaggctgtgttctgggctgggggaccagcaaggagaaggatcagctgtccgcttccagctgttctgagggatgatgctgtgggttttagaggaggcaggggtgcgggaggcagagaaaggcgccaggaagagtggtgaggagaactggaaaaacagaaagcgctggtgaagccagcaggcaagcaatgactccggcagtcttccttgcctctgaagcagaagggatccagggacagttagtggctcaggtcctgcggctccaggtgcccagacgtgcatttcctgtgtggagtggccggggcctctccttggtgtgcagcccagaaatgtggcaactttgggttacccaaccttcctaggcggggaggtagtccagtccttcaggaagagtctctggctccgttcaagagccatcacagtcccttgtattacatccctctgacgggttccaataggactatttttcaaatctgcggtatttacagagacaagactgggctgctccgtgcagccaggacgacttcagcctttgaggtaatggagacataattgaggaacaacgtggaattagtgtcatagcaaatgatctagggcctcaagttaatttcagccggttgtggtcagagtcactcatcttgagtagcaagctgccaccagaaagatttctttttcgagcatttagggaataaagttcaagtgccctgcgcttccaagttgcaggagcagtttcacgcctcagctttttaaaggtatcataatgttattccttgttttgcttctaggaagcagaagactgaggaaatgacttgggcgggtgcatcaatgcggccgaaaaagacacggacacgctcccctgggacctgagctggttcgcagtcttcccaaaggtgccaagcaagcgtcagttcccctcaggcgctccaggttcagtgccttgtgccgagggtctccggtgccttcctagacttctcgggacagtctgaaggggtcaggagcggcgggacagcgcgggaagagcaggcaaggggagacagccggactgcgcctcagtcctccgtgccaagaacaccgtcgcggaggcgcggccagcttcccttggatcggactttccgcccctagggccaggcggcggagcttcagccttgtcccttccccagtttcgggcggcccccagagctgagtaagccgggtggagggagtctgcaaggatttcctgagcgcgatgggcaggaggaggggcaagggcaagagggcgcggagcaaagaccctgaacctgccggggccgcgctcccgggcccgcgtcgccagcacctccccacgcgcgctcggccccgggccacccgccctcgtcggcccccgcccctctccgtagccgcagggaagcgagcctgggaggaagaagagggtaggtggggaggcggatgaggggtgggggaccccttgacgtcaccagaaggaggtgccggggtaggaagtgggctggggaaaggttataaatcgcccccgccctcggctgctcttc

SEQ ID NO: 188 - DNA, 5000 bp, synthetic construct
  misc_feature 1..5000: note = Description of Sequence: (VWF Promoter Region)
  source 1..5000: mol_type = other DNA
ctttcttttccttacggtctttgtgtgtttatattttaggactgtctcttgaacattgctggattgtgtgcctttttaaagaaatccaatttgataatctctacctttttattattgattattgatctgtttgaattttttgtactgttgcatttacattttatactatttgtcttgctttttccatttttctcctagatcaatctagtttttaaaaatttcttgttttttttcgataggtttggaaaacatacattcaattctattctttcagtagcctcccttaaaatcttactatgcagtatagccttaactaagtctaaagtgatcatgacctgtttcttccttctgagccatgcaaggaccttaatacactttcactcctgtcttacatgtcattgctatctggtattttggttctaatttttaataactctccaacttatcattaccattgttattgttttatataatgactgcctgctcatatttacccccagatttactattttatttatttaccttttcttggcatcctgtttctaggctgaattttcttcttcctggcagggcgaggtggctcacacctgtaatcccagcactttgagaggccgaagcaggaagactgcttgagcccaggagctctaggagtttgagaccagcctaggtaacatggcaaaaccctgtctctacaaaaaaatacaaaaattagccaggtatggtggtacatgcccatagtcccagctactagggaggctgaggtgggaggatcgcttgagcccagaaggtttaggctgcagtgagttgtgactgtgtcactgcgctccaccctgggtgacagagaccctgtctcagaaacaataacaaaaaatgttttttctccctgaaattccttctttagtagtttcttcagaaaggactgttaatgttaagcccactctgtttttccaaaaaaaaaaaaaaaaaaaaagtctatgtttcctcaactctgaacaataatttacaggtagaccagatgttacagtccctctgccacggccctctaaccccatctctccgtttcttctagactaccagtgtctgctcctccgtgcctgagggccagaagtgttgagggctgaatgcctcctgaagcagccttgacaataactgacaggaaggagtaagtgtagaaactctcctgctccctgacccaagagtgggaagattcagaggcatggatcttacattttcccagacttccctccagaaggaagctttcttgtccacttggtggctggcataataaacatcttttattgactgctttccctgccccgtatcacattcccacttccctgccgatgcttcctgtcctcccaaatcaattatctacactaaaatactcagggtctgcttctgggacttcaaactaagacactgcatatagaattctccacaaatggacttcttctctcagcacttctctttggatcttctaattctatcatgtttcttaattctgcctccatttttttttttttttttttttttttttgagatggagtctcactctgtcacccaggccaaagtgcagtggtgtgatcttggctcactgcaacctctgcctcctgggttcaagcaatcctcctgccccagcctcttgaatagctggaattacaggcatgcaccaccatgcaaggctaatagttgtatttttagtagagatgggatttcaacacgttggccaggctggtctcgaactcctgaccacaagtgatctgccttacttgggctcccaaagtgctgggattacaggcataagccactgtgcctggctgctcttccattttttcatctctatatttgagtgatttctttagaaccatttttcagctttttttttcccaaaaatatctggtgtttttgaatttttcaattaatttactatatacatttcttaaagttctttttttttttttttgggacagtatctcactctattggctaggctggagtgcgcgattatggctcactgcaacctcaacctcctgggctcaaggggtcctccctcctacctcagcctcctgagtagctgtgaccacaggcatgcaccgccaggcccagctaattttttatttttattttttgtagagatggggtcttgctgtgttgtgtaggctagtctcaaattactgagcacagacaatcctcttgcctctgcctcccaaagtgttaggattacaggtgtgagcctccacgtctggccttaaagttctttttcaagttttcctcctgttctttcttttttttttttttttaacggactctcactcggtcaccaggctggagtgctgtggcatgatcttggctcactgcagcctccgcttcccgggttcaagcgattctcctgcctcagcctccagagtagctgggactacaggtgcgtgccaccatgcccagctgatttttgtatttttagtagagatggggtttcaccatattggccaggatggtcttgatctgttgaccttgtgatccacccaccgtgagccaccgtgccctgccttgaaaaaagtttattctttgtaaagacagggtcttgctgtgttgcccaggctggtcttgaactcctggtctcaagcgattatcccatctcagcctcccaaagtgccaggattatagacatgaaccaccgtgcctagcctctattataattttaaacactcatttttttctttttatctttctctgatgattttattatctgaaattcttagggatttaattcttgctcatggtagattgttttctcaagtgttgaaaaatttgtgattttgaattcatcgtcaagagagctttatttgcatgagtgcaaaggatgaaaattctagactgggcgtggtggctcacgcctgtaatcccagcactttgggagaccgaggtgggcagatcacgaggtcaggagtttgagaccagcctggctaacatagtggaaccccatctctactaaaaatacaaaaaattagctgggtgtagtggtgtgtgcatgtaatcccagctacttgggaggctgaggcaggagaattgcttgaagccgggaggcagaggttgcagtgagccatgattgcatcactgcactccagcccagcggacagtgcgagactccatctcaaaaaaaaaaaaagaaagaaaagaatattctaaaaaaagacttaattccccccgccaccccaccccaaaacaagtggagacaggcaaacttccttatcttctaggttgggggatggatttttttcctggtccactgtttggaagatgtttcccttcaaactttcagcttttgcagggatctccgttctagttctccctctgggtcaggcccgtagctgcactgcccattcttgtaatgtgcggcctccagtctggagggttcccagactggcctacgctaggccacccatgggcctaccctgcctcatgctcatttaggctcctcttcctcattgaccctttaagatattccttactttcctcccagatcaactgtggatttaaagaacatttgttgtatttagcacagcatttaaagatattttgtaatgaaagggttttcagattagttatttagtttttttaaataagagctggaagtggaaatcccgatggccttttcttttcttttcttttttttcttgagacggagtcttgctctgtcacccaggctggagtgcagtggctcgatctcagctcactgcaagctccgcctcccgggttcacgccattctcctgcctcagcctcccgagtagctgggactacaggcgcccgccaccacgcctggctaattttttgtgtttttagtagagacggggtttcactatgttagccaggatggtcttgatcccttgacctcgtgatccgcccacctcggcctcccaaagtgctgggattacaggcgtgaaccaccgtgcccggccccccaatggccttttctactgtctcatgctgattctgcctctggtgccatttttcttccttgggagtgtgcatcttcctctcctggggcggtaaagggagtagcagagtgcgaggtatgtgggaagggaggaggttggaacctagtggtttctcaaagtcttagggcaggaggtatcatggagaagcagtgaggaggtcttccatacccagacatgcctcagggtgcttgtctcagtgcctggaatcccagcacgagagtcatcttccccccaccgctgcccattgcatcagttacttattttagtaggaattagtttagcagatggtgttgagaattaggcttttgggaatgggaggctgggaagaagaattgtgtgtgtgtgtgtgtgtgtgtgtgtgtgtgtgtgtgtgtaagatcagggtaccagaagtgggtggaaatgtccttgagaattagaattattagaatgtagcaacagtagaagtattagactcaaaccatcactccccaccttcaccattttacaaaggcttaggcttgtggccaagaccttcatctttagccgatccattcaaccctggccaggatccaaatggactgtttttgtcagggccaggaccggatccttcatacctggggtgcataggaagtgttagtactccccttcctccaaacacagcagcaaaattggctcaggttgaggtgtttttctcaacttccctggagtccagccctggaagctggatcaggaagctgtgttgttctactgtgattccccctggcctgtatcagcttgccctgaaacaaccagcattcctggttatcccacacaggtggggcactctaggaagaccagggatcaagtgtgggggtgtagggatagggggtgtttggggagggcaaggcagttaattaaggcagctgccaggaggtctccctccaaactctacaaagctttatcagcttggaggtacttctaataccatttcctttcattgtttccttttggtaattaaaaggaggccaatcccctgttgtggc

SEQ ID NO: 189 - DNA, 5065 bp, synthetic construct
  misc_feature 1..5065: note = Description of Sequence: (MGP Promoter Region)
  source 1..5065: mol_type = other DNA
aaaccagagcaagtgtcagatgacagcaaacatcggcgttcacagactggatgagacttggtgattcctgaaggaagctgaaaggttttggaaagtaccatatttgagttggagcttgtttgggcagatgtttagtgtgcagtgtactggagagagaaacaggggacttaggatccaatattaaaactatccctctctccatttttataagtcatttaactcactgggcttaagtgtatgtagacctttaatttgtattgcactattttaaaggattttgagacaattatcattttaaatgtgtatatcttaattttgtgtaattttgtctctagctatgaaatttgaaattcaaggaaccctgtaagacgcaatcacaaaattatttaagcaggtgtcagaacctggagggatctcacatggttgtatcattattaaaaacacacgatttgacatcaaacagtcctgcgttttaatcctgtctttgccatttagttgttgtgtgatcttggataaactattttacctctttgagcttccattttcttatatatgagatgtgggtcattatacttagcttataggattattaagagaagtaaataaaaaataaagagtgactatcagagcatctaatatatagcaagcatttgacatatgataatagcgcttggatagatgtataatttttatttgatatttgctatcttatttaaaaattgaagcttttcttaagaagggtggaggaaataatcgataggggaggaaaagaaaaatcccagccattcccagccaaaagctctccacctctcagacttctccacctttattttgtttaataatcaggcacctccaactttccctggaagtttcagcccatgagtccccagatgctcctcccatgcctgagtttgacaaacatttattcttacttagttcaactaagacaaattaaggaaaggaaagaaggattctatctgtgcctaggaagttatgtcagttaaagaatctgacacacagaatcctctcaaggggcttcccttaaaagaaaagaactaagagcatggcactgtcccacgacattggctgggcttctgtctcctagttcattgttctttgatgtcctagagagtgatttctgtttttgtcttcagggctgcttcttcctaactcttctttcatttgcgtttttcctttaatggcaagggtaaggactttagtgctgccagtttaaaaaaaattataaaatcaaaattcagtaaataaagctgaatttcatggaacagcaaaaaagaaaaagaaatctgactggagccaggaaaattcctcaaagagacaaaatgcctagccatctgaaaaagagttagttaaaggataagcaaggacagaaagagaaggaggtagaattgatagggaaatatattacactttctacctctatgccattattttagtcacagaaataccatgtgaaagggataactcttttggtatattctctggcaaatatacttttctgagaagcttctccccacagtaattctcagaagctctgccaatgttcctctcctgcattccctccctaaattcagaattatctgagctggtcactagcacctttcttgatgctttggaaacagagatgctcctgagtgcccataagaatggtcagcccagtgagaaagctcatcacttggtctcctttaaggccagttggctgcctaacaattttttaaataagaggagccactattaaatttttgttcaaagagcacacttgatgcatgaggacagggcccatatctgtatttttctctactgtatttccagcctagagttgacaaacagtagatgctcagtacatttgttggctagatagataacttgatggatggctggctggctggctggctggatggatggatggatggatgggagaattatgaaatcatgaagctccttctggccctgacaggcatggtcattcttctcttttctgcctgagagtaggtggaataggagatctgtattactccatggcttctcttgcttcagttcctacgttgccaacctcacatgaggagaatcctacacatgtttaaaaactggcaatcatatcactgtctcatatttctgttatcacttctgggagtttcttcaaatattctctcctctgaataacacttcttttttgttaagggaaaatgtctatataagtgtctttcataattatctaaaatctaattagaatttagagtttcatgtggtctcgtcttgacaagatatcccaattaagaaaatgcaaactagctggcaaaattaatttgttcaaatttcaatattttctgaaaattttcagacagtattctgcaatctcaaacaatgctattcctaaccaaagcaacttttatttctctgttcccatgtctcgcttttaatatgtctcaccttctacaactgcctccgtttttctctgtcactcagtctctacctaaaactcacccagcaaaccaaattggtaaggctcttctcatttccccttctccgtttttttttttttcctacttccattcttttcttctgtcttctctcagatgagtcaatcttggtcctttctaatgcaaagctcccatccctgcttcatgcgttagtccaagtcctcatcataaaaacatatgactggagttggcattcacaaagttgtctttgaaatggggagtaaggtgacagaggagaaaaagaagagctctggattctcagacatgttaataatttttacatatcatatataaatgggattttgcagagaagaaccagaaatagatgggagagcaatggacaggaaaggcagatgagggaccgaagagacacagctcccaaaagaaagttagccttacaaaaaccaagacgataaagagaaatgcttaagtttagggaatccagtggaagcagtgatttaaggtgaacaaaaggtgaaccttaagttgaaatgagaagtgtaggattttcaagtttagtttctgggagtgtaaaaataaaaaaacaattgtgatgtcagaggttgaaagattatagttgtcatttgaacttggggataaaggagacatctatgacttggctggaaaagacagagctaatgtacattgcaaagcacatatttatagcaggaaaatgggaagatttctctttaattctggagatggagtggggatggggagagtagactactcattttaagggtgaaacattggaattcaacttgtttgatgttatattaattggtggttaattactaagctaagtacgtataaaacttttatctatggctagcttgtccccccaaagtcatgcaatatagtgaactggctttcgcactttaaattattcattgatcatgtaatgattcagatgattcatcttccaagatggacactgaaactaacactcatagtaggttgtggtttaaagagtggaacaaccgccagtctcattagtggaaattgtgatggttgaatttatcaaggatgaacatacacggtcttctttctgagattttctttaagattttcgcacagataatctatttcttaggttttggagagaaaacttgaattttattgatccctcagaactcaatctttcagatttcaaaggagctatttcttttaatggggactctgttaatatttataaaagctcttcacaggatggagggtgggagggaaactccatcccaacaagacaaaaagaatgaagcatgaggctccacctagttcatcactgctccttgaaatacatcagtattgaaagacacatccaccccacccccaacccagccctattgctgttccagctcaagagtcagaggtcccgaagctgtagctcttctacaatctgctgctctgtgacttcaagtctgttgtctgcaaagaaaactattgggttcccaagcaagagaggcacatctggtaggacagattttgtgattgcaaaagaagggggaaaaaaagaaagaaagaaaagacctctctatacaagataaccagaggcatcaaactgaaatcctcctgtggaaaataagctagtacttctgggcctgatggtgtagtgaaaacctgtgcttgaggatacattacagtgaaagagcaaagtgaatagtaagtagctattacttacctccttagggaggtgtgttgtttgtctgtacatcccccacagcacctagcacagtaccttgcatctcacctgccactcactaaaaagtctatcaagttagttaattatcgagacaacgccctcagaaatgagagaacagtaccctcttatccttgctgcactttccagcactgatacgctgcctaaaagaggactagggcacaggtttgaattaatgtcacaaaactggatgggcaagttacaacggtgttgattaaggaaacagaactcatggtgcaccggatatctccatcctgatgaacccttggaaaaatgccaaagatgcatatccccaggcaaatgcctgattagtctgggattgatagattggtctaggattcagccctactgggaagatgtctaaattataatcagtgtagaaagcgaagttctcctagaagaagaggcaaaggttaaaaagaagaaaagaaaagaaagtgaagtcctttctcccccaaaacctctcatcaatcaatcagggtaacaaacagaacactagggctctgtctgtggaccaaacccaaaagccctgcggtcagggccaggagggtagatcatgtgtttgtggcaacttcctctgtgggcttttgcccaggtctgtccccaagcatacgatggccaaaacttctgcaccagagcagcatcctgtgtaacacagtcaggtccagcagttagggaaaactgcccactcagagtagataatatctggaaggaatgactgtttgggaaaagttccaatgctagttcagtgccaacccttccccaccttctccagctctctcccactggttcctcccctctcaactgctctggttcttataaaaacctcacagccttccactaacatccc

SEQ ID NO: 190 - DNA, 5000 bp, synthetic construct
  misc_feature 1..5000: note = Description of Sequence: RGS5 Promoter Region)
  source 1..5000: mol_type = other DNA
aatctttctttttttttggtatgtaggaagattaagatttatatttctgttctaatggttacctttgtacatacacttttcttagtgtctttcatctcctgttttcttatttaacctgttatctggtttatccaattttattggtatcctttaactgcttctgcaacaatcaatgatctaattctacttttcttctttctcccatttttagtttcattatttttataaagaattcttgcacaccaatattaaaaagccagatagtaaagttgtttaaaaaatggaaatatacaactaatggaagaaaaattacagtggccaatgtgtatatgaatagaagctcaaattccctagtaattagaaaggcacaaatgaaaatgacttggtattttttgcctgtcactggaaaaaattaaagcctggcaagtgtttcatatttgggaaaatgaggctctcatttatttctgttaggagtctaaattggtctgactgtgtgataattttgtactgttacacctctcagctgtcccactttatgaactacattatagataaactgctccgggggcatatataggaatattaatttaagtacttttgccagaagcaaacaattagaaatgacctaaatatttaacaatgggtctataaagaaattcaattatatttgcatatgatataattctttccagcaattaaaaagcataaactggaggtatagactttacacttcctatgttagtaccgatgctttttaatcctgaacaatttcatttccatttatatgatcagagggttttctctttgaaatagcaaaaattataccacttatctcatgcttttgttgtgagaattaaatgtgtaaatatatgaagtgttttaaaaagtgattaatacatagaaattattcagtagtgttagttgtcattataatctaaaaacaaaaacaaaaaatcataatcttaaataacaaaagatgcagattatatttatataaatcaaaacacataccaataattactatatactttttatgaattcatattgtcttagtttgtgctgctataacaaaatacctgagactgggtgctttataaacaaaagaaatttatttctggcagttttgcaagctgagaagttcaagatcaaggtgccagcaggttcggtgtctgatgagagctgctctctgcttctccaagatggtgccttgttgctgtctcctaacatggcagaaaagcagaagaaaatgaaccccctccctcaagcccttttataagggctctaatctcatccatgagagctttgacctcatggcttaatcacctcctaaaggccccacctcttaatattaacacattggtgattattaagttttaacacatgaattttgagggaaacgttcagatcatagtacatatgtatatgaggacattaaaaaatgacctggaataccatacacgaaaatcttaacaggtactaagtgtgatgaaaggggaaaaataatggacatgagagtcctctttttttcattaaaaaaatcaaaaagcaacataaactatatacttttaatgaatccatattgtcttagtttgtgctgttataacaaaatacctgagactgggtactttataagcaatggaaattgatttctcacagccctggaagctgggaagttcaagatcaaggtgccagcaggttcagcaggactatatttaatataaagtccagggactttagttttatccattgcctctttatttctttaaataaaaaaaggcagggaagcaatatcacaacctattgacagtatatatttctgagtggtagaattataaattgagcttgcttatggtttaaaacatttagcagaaagacagaaatcaagcaacatgaaggtgaagaccttgcctctgtttactccatgtgctcctagctggctttagggtgagactcttactgtgagtagcgagattagtgaatagacgtctgttcaccatggatcacatagtccaagaggtccccaaattagaagtgataaaatgcatctcaataaatatattgctaataatagttaaaataaagacacttacacatgatttaaggaaatgaattagagatgaatcagagcagttgtgtgggcaggagatccttttcatctgatgaaaatgttcaatgtctctcactgcttccacttgcacagctaactccttccttctgtagaactgtaatatctagtgctttggagcccaaaccactgtggcatcctccagggcagatggatatctctttgaagcacccccctgaattccctcccctgtttcattaggtaccactcctactcacctcttttcacattccatgaagaagtggttgaaatcactcagaatatgatggcttgtttctctttttctttgctattacagatttatacctttttcatatatttactgtcatttctgagggattctcaggagggaaaggagattttaaaatagttcaatttattttttcagacttcaaatttattttacgtagtaggctctctccttttttttttttttttttttttttactgtggttaaaaaacacgtaacacaaactattctcttcacagatttttaagtgtacggtacagtatagctaactatgggcacaatgttgtatagcagatccctagaaatttttcattttgcataactgaaactttatacctattgaaactttattgaataggaactcctccatttctcccatttctaacctctagcaatcactattttactttctgcctctagagcacactcttttacatactttctggctatcccatccttacaaagttttaaaaacaaataagtgttggatttaacattatttctattgaattttacattttgggttctagtccatcttttagtaaattgagataatttttattcttgaatagtccatcacatgaactcctttgggacagtcagaaacatgctttgatttttagccctcgccaagacatcaccctccattgctctacgttaaacttggggtttttcttttcatattattatctcctcaaggccccttggtatcaaataatgatttagactcttgtcccaaggagtctcttctacttgttttatattccctttattagatttaatagtcaacaacagtgtccacatgtctcaaaaatgataattaattgatttgctgatgtttactcagtcaatagatggcggaacttggacagtttgtttaactcattcagtatttacagatgtctatgtgcctgacattgtgcttagtgtttggaaggctgaggcaggaggattgcttgaagcaaagagttcaagaccagcctggacaccaaaactagaccctgtgtattcaaaaaatttaaaaactagctaggcatggtagcaagagtctttagtcccagttacttaggaggctaaggtgggaggatcgcttgtgcccaggagtttgatgatgcagtgagctataatcatgccacttcactccagtctctggtctgggtaacagagctagactctgtctcaaaaacgtgtgtgtctgtgtgtgtgtgtgtgtgtgtgtgtgtgtgtgtgtggtttagagccacagctttcaaacttttttgattatgatccatagtaagaaatatagtttacctgatgaccctgtgcacacttacatattgataattaaagtcaaaatttcagaaaacaatactttttacatccgatgcacttggatatttgtctattctatttattacattaaaaacatcaggtcataatgcactgcattgactttacaagccagtaacaggggttgatcaaccattgcctaaaaagggtatataaagagggagtcatttaaaacaactaatttcagataacagagaaaattatgagagtactgttcaattccaaattcagagcttttcttacggattattcagtgccactttttttaaagcttggactcatgcctaccttttaggcatttatttacagattaacttatcagcaccttgggccccgacaagataagacacaattcaggtaaattttggtaaatattccttgagtgataggaagcaggaaaagtttgaaggagaaggaaagacaagcctaggcaaggatggctatggtggggaggtacaaagaattacaaagcattagtcatccaagcaaagtataatcagaagaccactgacagaattaacagagttcagagataatatcattatactcaatccctactaagattttggtgtttggtctcctagatcacttttttagtaccttcacagacatatggattcataaagaatatatagtaattattcatatttgttttgatttatataaacagtaatcttgtctctcagctccagaaaaccttgcctggtgcttcctgaaggctcacaacatcccagcccggcctccatcacttacttataatgtgttttggggtcttgagggcagggagctttctctttcttctctgaattcctagcatccagctgagtgcctggcacttggtaagtattcacatacacattccttgaattccctagatttcagtttccttcactgtgaaacaaagtgggctaaacgatctccaggagcccttacagtcctaagaatctatgactttgaccgatctgaagtggtccaagacactttgcttcacaggaagctcacatcttccttgtgattggcctgagattaggagttcaaacaaccaatgacctgtgcacagtttagggcctaaccctgccctggccggttagcaagagagtgttgtgataagagcagatgaaagccttatgccacagtagtgcctgtagcagagaaaggattccctcctccacctcagggcccactgctgctgctctccagcctgcagtttcctctaaggctctggattggctggaagagcaacagagggctgggaaagagcttctatatatacctcaggaggaaaggcatccc

SEQ ID NO: 191 - DNA, 443 bp, synthetic construct; identical to SEQ ID NO: 16
  misc_feature 1..443: note = Description of Sequence: (SFFV Promoter Region)
  source 1..443: mol_type = other DNA

SEQ ID NO: 192-217 - skipped (26 entries)

SEQ ID NO: 218 - DNA, 613 bp, synthetic construct; identical to SEQ ID NO: 24
  misc_feature 1..613: note = Description of Sequence: (CFHT - 18-bp spacer - IRES (551-bp) - 45-bp spacer - Aflib)
  source 1..613: mol_type = other DNA

SEQ ID NO: 219 - DNA, 6739 bp, synthetic construct
  misc_feature 1..6739: note = Description of Sequence: pCTM398
  source 1..6739: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacctcttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatctaggcctgcaggcggccgccaccatgagactgctggccaagatcatctgcctgatgctgtgggccatctgcgtggccgaggactgcaacgagctgccccccagaagaaacaccgagatcctgaccggctcttggagcgaccagacctaccctgagggcacccaggccatctacaagtgcagacccggctaccggtccctgggcaacatcatcatggtgtgcagaaagggcgagtgggtggccctgaaccccctgagaaagtgccagaagaggccctgcggacaccccggcgataccccttttggcaccttcacactgaccggcggcaacgtgttcgagtacggcgtgaaggccgtgtacacctgtaacgagggctaccagctgctgggcgagatcaactacagagagtgcgacaccgacggctggaccaacgatatccccatctgcgaggtcgtgaagtgcctgcctgtgaccgccccagagaacggcaagatcgtgtccagcgccatggaacccgacagagagtaccacttcggccaggccgtcagattcgtgtgcaacagcggctacaagatcgagggcgacgaggaaatgcactgcagcgacgacggcttctggtccaaagaaaagcctaagtgcgtggaaatcagctgcaagagccccgacgtgatcaacggcagccccatcagccagaagatcatctacaaagagaacgagcggttccagtacaagtgtaacatgggctacgagtacagcgagcggggcgacgccgtgtgtacagaatctggatggcggcctctgcccagctgcgaggaaaagagctgcgacaacccctacatccccaacggcgactacagccccctgcggatcaagcacagaaccggcgacgagatcacctaccagtgccggaacggcttctaccccgccaccagaggcaataccgccaagtgtaccagcaccggctggatccctgcccccagatgtaccctgaagccctgcgactaccctgacatcaagcacggcggcctgtaccacgagaacatgcggaggccctacttccctgtggccgtgggcaagtactacagctactactgcgacgagcacttcgagacacccagcggcagctactgggaccacatccactgtacccaggacggctggtcccctgccgtgccctgcctgaggaagtgctacttcccctacctggaaaacggctacaaccagaactacggccggaagttcgtgcagggcaagagcatcgatgtggcctgccaccctggatacgccctgcctaaggcccagaccaccgtgacctgcatggaaaatggatggtcccccaccccccggtgcatcagagtgtccttcaccctgtgaggtaccactgtgcatatgcgttactggccgaagccgcttggaataaggccggtgtgcgtttgtctatatgttattttccaccatattgccgtcttttggcaatgtgagggccaggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcccctcaagcgtattcaacaaggggctgaaggatgcccagaaggtaccccattgtatgggatctgatctggggcctcggtgcacatgcttttcatgtgtttagtcgaggttaaaaaacgtctaggccccccgaaccacggggacgtggttttcctttgaaaaacacgatgataatagaattcgatatcaagcttatcgataccgtcgactagagctccaccatggcctctcatagactgctgctgctgtgtctggccggcctggtgtttgtgtctgaggcctctgataccggcagacccttcgtggaaatgtacagcgagatccccgagatcatccacatgaccgagggcagagagctggtcatcccctgcagagtgacaagccccaacatcaccgtgactctgaagaagttccctctggacacactgatccccgacggcaagagaatcatctgggacagccggaagggcttcatcatcagcaacgccacctacaaagagatcggcctgctgacctgtgaagccaccgtgaatggccacctgtacaagaccaactacctgacacacagacagaccaacaccatcatcgacgtggtgctgagccctagccacggcattgaactgtctgtgggcgagaagctggtgctgaactgtaccgccagaaccgagctgaacgtgggcatcgacttcaactgggagtaccccagcagcaagcaccagcacaagaaactggtcaaccgggacctgaaaacccagagcggcagcgagatgaagaaattcctgagcaccctgaccatcgacggcgtgaccagatctgaccagggcctgtacacatgtgccgccagctctggcctgatgaccaagaaaaacagcaccttcgtgcgggtgcacgagaaggacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaatagcacctacagagtggtgtccgtgctgaccgtgctgcaccaggattggctgaacggcaaagagtacaagtgcaaggtgtccaacaaggccctgcctgctcctatcgagaaaaccatctccaaggccaagggccagcctagggaaccccaggtttacacactgcctccaagcagggacgagctgacaaagaaccaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgcagcgtgatgcacgaggccctgcacaaccactacacccagaagtccctgagcctgtctcctggctaagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctcaggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcactgtcattagcaactccttgtccttcgatctcgtcaacaacagcttgcagttcaaatacaagacccagaaggcgactattctggaagcgagcttgaagagttaacctgcagagagcccccgcagtgtcgacaattaatcatcggcatagtatatcggcatagtataatacgacaaggtgaggaagtaaaaaatgagccatatccaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcaaggctgactgcagggcgagaagattgcgagctgtgcggctgagttgacgtatctgtgctggatgattactcataacggcaccgctatcaaacgtgccacgttcatgtcctaca

SEQ ID NO: 220 - DNA, 6634 bp, synthetic construct
  misc_feature 1..6634: note = Description of Sequence: pCTM418
  source 1..6634: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacctcttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatctaggcctgcaggccaccatgggcaagatttctagcctgcctacacagctgttcaagtgctgcttctgcgacttcctgaagtccgataccggcagacccttcgtggaaatgtacagcgagatccccgagatcatccacatgaccgagggcagagagctggtcatcccctgcagagtgacaagccccaacatcaccgtgactctgaagaagttccctctggacacactgatccccgacggcaagagaatcatctgggacagccggaagggcttcatcatcagcaacgccacctacaaagagatcggcctgctgacctgtgaagccaccgtgaatggccacctgtacaagaccaactacctgacacacagacagaccaacaccatcatcgacgtggtgctgagccctagccacggcattgaactgtctgtgggcgagaagctggtgctgaactgtaccgccagaaccgagctgaacgtgggcatcgacttcaactgggagtaccccagcagcaagcaccagcacaagaaactggtcaaccgggacctgaaaacccagagcggcagcgagatgaagaaattcctgagcaccctgaccatcgacggcgtgaccagatctgaccagggcctgtacacatgtgccgccagctctggcctgatgaccaagaaaaacagcaccttcgtgcgggtgcacgagaaggacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaatagcacctacagagtggtgtccgtgctgaccgtgctgcaccaggattggctgaacggcaaagagtacaagtgcaaggtgtccaacaaggccctgcctgctcctatcgagaaaaccatctccaaggccaagggccagcctagggaaccccaggtttacacactgcctccaagcagggacgagctgacaaagaaccaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgcagcgtgatgcacgaggccctgcacaaccactacacccagaagtccctgagcctgtctcctggctaagagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcccctcaagcgtattcaacaaggggctgaaggatgcccagaaggtaccccattgtatgggatctgatctggggcctcggtgcacatgcttttcatgtgtttagtcgaggttaaaaaacgtctaggccccccgaaccacggggacgtggttttcctttgaaaaacacgatgataatagaattcgatatcaagcttatcgataccgtcgactagagctcgccaccatgagactgctggccaagatcatctgcctgatgctgtgggccatctgcgtggccgaggactgcaacgagctgccccccagaagaaacaccgagatcctgaccggctcttggagcgaccagacctaccctgagggcacccaggccatctacaagtgcagacccggctaccggtccctgggcaacatcatcatggtgtgcagaaagggcgagtgggtggccctgaaccccctgagaaagtgccagaagaggccctgcggacaccccggcgataccccttttggcaccttcacactgaccggcggcaacgtgttcgagtacggcgtgaaggccgtgtacacctgtaacgagggctaccagctgctgggcgagatcaactacagagagtgcgacaccgacggctggaccaacgatatccccatctgcgaggtcgtgaagtgcctgcctgtgaccgccccagagaacggcaagatcgtgtccagcgccatggaacccgacagagagtaccacttcggccaggccgtcagattcgtgtgcaacagcggctacaagatcgagggcgacgaggaaatgcactgcagcgacgacggcttctggtccaaagaaaagcctaagtgcgtggaaatcagctgcaagagccccgacgtgatcaacggcagccccatcagccagaagatcatctacaaagagaacgagcggttccagtacaagtgtaacatgggctacgagtacagcgagcggggcgacgccgtgtgtacagaatctggatggcggcctctgcccagctgcgaggaaaagagctgcgacaacccctacatccccaacggcgactacagccccctgcggatcaagcacagaaccggcgacgagatcacctaccagtgccggaacggcttctaccccgccaccagaggcaataccgccaagtgtaccagcaccggctggatccctgcccccagatgtaccctgaagccctgcgactaccctgacatcaagcacggcggcctgtaccacgagaacatgcggaggccctacttccctgtggccgtgggcaagtactacagctactactgcgacgagcacttcgagacacccagcggcagctactgggaccacatccactgtacccaggacggctggtcccctgccgtgccctgcctgaggaagtgctacttcccctacctggaaaacggctacaaccagaactacggccggaagttcgtgcagggcaagagcatcgatgtggcctgccaccctggatacgccctgcctaaggcccagaccaccgtgacctgcatggaaaatggatggtcccccaccccccggtgcatcagagtgtccttcaccctgtgagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctcaggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcactgtcattagcaactccttgtccttcgatctcgtcaacaacagcttgcagttcaaatacaagacccagaaggcgactattctggaagcgagcttgaagagttaacctgcagagagcccccgcagtgtcgacaattaatcatcggcatagtatatcggcatagtataatacgacaaggtgaggaagtaaaaaatgagccatatccaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcaaggctgactgcagggcgagaagattgcgagctgtgcggctgagttgacgtatctgtgctggatgattactcataacggcaccgctatcaaacgtgccacgttcatgtcctaca

SEQ ID NO: 221 - DNA, 6634 bp, synthetic construct
  misc_feature 1..6634: note = Description of Sequence: pCTM419
  source 1..6634: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacctcttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatctaggcctgcaggccaccatggaaacagatacactgctgctgtgggtgctgctcttgtgggtgccaggatctacaggcgattctgataccggcagacccttcgtggaaatgtacagcgagatccccgagatcatccacatgaccgagggcagagagctggtcatcccctgcagagtgacaagccccaacatcaccgtgactctgaagaagttccctctggacacactgatccccgacggcaagagaatcatctgggacagccggaagggcttcatcatcagcaacgccacctacaaagagatcggcctgctgacctgtgaagccaccgtgaatggccacctgtacaagaccaactacctgacacacagacagaccaacaccatcatcgacgtggtgctgagccctagccacggcattgaactgtctgtgggcgagaagctggtgctgaactgtaccgccagaaccgagctgaacgtgggcatcgacttcaactgggagtaccccagcagcaagcaccagcacaagaaactggtcaaccgggacctgaaaacccagagcggcagcgagatgaagaaattcctgagcaccctgaccatcgacggcgtgaccagatctgaccagggcctgtacacatgtgccgccagctctggcctgatgaccaagaaaaacagcaccttcgtgcgggtgcacgagaaggacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaatagcacctacagagtggtgtccgtgctgaccgtgctgcaccaggattggctgaacggcaaagagtacaagtgcaaggtgtccaacaaggccctgcctgctcctatcgagaaaaccatctccaaggccaagggccagcctagggaaccccaggtttacacactgcctccaagcagggacgagctgacaaagaaccaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgcagcgtgatgcacgaggccctgcacaaccactacacccagaagtccctgagcctgtctcctggctaagagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcccctcaagcgtattcaacaaggggctgaaggatgcccagaaggtaccccattgtatgggatctgatctggggcctcggtgcacatgcttttcatgtgtttagtcgaggttaaaaaacgtctaggccccccgaaccacggggacgtggttttcctttgaaaaacacgatgataatagaattcgatatcaagcttatcgataccgtcgactagagctcgccaccatgagactgctggccaagatcatctgcctgatgctgtgggccatctgcgtggccgaggactgcaacgagctgccccccagaagaaacaccgagatcctgaccggctcttggagcgaccagacctaccctgagggcacccaggccatctacaagtgcagacccggctaccggtccctgggcaacatcatcatggtgtgcagaaagggcgagtgggtggccctgaaccccctgagaaagtgccagaagaggccctgcggacaccccggcgataccccttttggcaccttcacactgaccggcggcaacgtgttcgagtacggcgtgaaggccgtgtacacctgtaacgagggctaccagctgctgggcgagatcaactacagagagtgcgacaccgacggctggaccaacgatatccccatctgcgaggtcgtgaagtgcctgcctgtgaccgccccagagaacggcaagatcgtgtccagcgccatggaacccgacagagagtaccacttcggccaggccgtcagattcgtgtgcaacagcggctacaagatcgagggcgacgaggaaatgcactgcagcgacgacggcttctggtccaaagaaaagcctaagtgcgtggaaatcagctgcaagagccccgacgtgatcaacggcagccccatcagccagaagatcatctacaaagagaacgagcggttccagtacaagtgtaacatgggctacgagtacagcgagcggggcgacgccgtgtgtacagaatctggatggcggcctctgcccagctgcgaggaaaagagctgcgacaacccctacatccccaacggcgactacagccccctgcggatcaagcacagaaccggcgacgagatcacctaccagtgccggaacggcttctaccccgccaccagaggcaataccgccaagtgtaccagcaccggctggatccctgcccccagatgtaccctgaagccctgcgactaccctgacatcaagcacggcggcctgtaccacgagaacatgcggaggccctacttccctgtggccgtgggcaagtactacagctactactgcgacgagcacttcgagacacccagcggcagctactgggaccacatccactgtacccaggacggctggtcccctgccgtgccctgcctgaggaagtgctacttcccctacctggaaaacggctacaaccagaactacggccggaagttcgtgcagggcaagagcatcgatgtggcctgccaccctggatacgccctgcctaaggcccagaccaccgtgacctgcatggaaaatggatggtcccccaccccccggtgcatcagagtgtccttcaccctgtgagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctcaggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcactgtcattagcaactccttgtccttcgatctcgtcaacaacagcttgcagttcaaatacaagacccagaaggcgactattctggaagcgagcttgaagagttaacctgcagagagcccccgcagtgtcgacaattaatcatcggcatagtatatcggcatagtataatacgacaaggtgaggaagtaaaaaatgagccatatccaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcaaggctgactgcagggcgagaagattgcgagctgtgcggctgagttgacgtatctgtgctggatgattactcataacggcaccgctatcaaacgtgccacgttcatgtcctaca

SEQ ID NO: 222 - DNA, 6144 bp, synthetic construct
  misc_feature 1..6144: note = Description of Sequence: pCTM428
  source 1..6144: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacctcttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatctaggcctgcaggccaccatgggcaagatttctagcctgcctacacagctgttcaagtgctgcttctgcgacttcctgaagtccgataccggcagacccttcgtggaaatgtacagcgagatccccgagatcatccacatgaccgagggcagagagctggtcatcccctgcagagtgacaagccccaacatcaccgtgactctgaagaagttccctctggacacactgatccccgacggcaagagaatcatctgggacagccggaagggcttcatcatcagcaacgccacctacaaagagatcggcctgctgacctgtgaagccaccgtgaatggccacctgtacaagaccaactacctgacacacagacagaccaacaccatcatcgacgtggtgctgagccctagccacggcattgaactgtctgtgggcgagaagctggtgctgaactgtaccgccagaaccgagctgaacgtgggcatcgacttcaactgggagtaccccagcagcaagcaccagcacaagaaactggtcaaccgggacctgaaaacccagagcggcagcgagatgaagaaattcctgagcaccctgaccatcgacggcgtgaccagatctgaccagggcctgtacacatgtgccgccagctctggcctgatgaccaagaaaaacagcaccttcgtgcgggtgcacgagaaggacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaatagcacctacagagtggtgtccgtgctgaccgtgctgcaccaggattggctgaacggcaaagagtacaagtgcaaggtgtccaacaaggccctgcctgctcctatcgagaaaaccatctccaaggccaagggccagcctagggaaccccaggtttacacactgcctccaagcagggacgagctgacaaagaaccaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgcagcgtgatgcacgaggccctgcacaaccactacacccagaagtccctgagcctgtctcctggcggcggcaggggtcgacgcggcggcatgagactgctggccaagatcatctgcctgatgctgtgggccatctgcgtggccgaggactgcaacgagctgccccccagaagaaacaccgagatcctgaccggctcttggagcgaccagacctaccctgagggcacccaggccatctacaagtgcagacccggctaccggtccctgggcaacatcatcatggtgtgcagaaagggcgagtgggtggccctgaaccccctgagaaagtgccagaagaggccctgcggacaccccggcgataccccttttggcaccttcacactgaccggcggcaacgtgttcgagtacggcgtgaaggccgtgtacacctgtaacgagggctaccagctgctgggcgagatcaactacagagagtgcgacaccgacggctggaccaacgatatccccatctgcgaggtcgtgaagtgcctgcctgtgaccgccccagagaacggcaagatcgtgtccagcgccatggaacccgacagagagtaccacttcggccaggccgtcagattcgtgtgcaacagcggctacaagatcgagggcgacgaggaaatgcactgcagcgacgacggcttctggtccaaagaaaagcctaagtgcgtggaaatcagctgcaagagccccgacgtgatcaacggcagccccatcagccagaagatcatctacaaagagaacgagcggttccagtacaagtgtaacatgggctacgagtacagcgagcggggcgacgccgtgtgtacagaatctggatggcggcctctgcccagctgcgaggaaaagagctgcgacaacccctacatccccaacggcgactacagccccctgcggatcaagcacagaaccggcgacgagatcacctaccagtgccggaacggcttctaccccgccaccagaggcaataccgccaagtgtaccagcaccggctggatccctgcccccagatgtaccctgaagccctgcgactaccctgacatcaagcacggcggcctgtaccacgagaacatgcggaggccctacttccctgtggccgtgggcaagtactacagctactactgcgacgagcacttcgagacacccagcggcagctactgggaccacatccactgtacccaggacggctggtcccctgccgtgccctgcctgaggaagtgctacttcccctacctggaaaacggctacaaccagaactacggccggaagttcgtgcagggcaagagcatcgatgtggcctgccaccctggatacgccctgcctaaggcccagaccaccgtgacctgcatggaaaatggatggtcccccaccccccggtgcatcagagtgtccttcaccctgtgagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctcaggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcactgtcattagcaactccttgtccttcgatctcgtcaacaacagcttgcagttcaaatacaagacccagaaggcgactattctggaagcgagcttgaagagttaacctgcagagagcccccgcagtgtcgacaattaatcatcggcatagtatatcggcatagtataatacgacaaggtgaggaagtaaaaaatgagccatatccaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcaaggctgactgcagggcgagaagattgcgagctgtgcggctgagttgacgtatctgtgctggatgattactcataacggcaccgctatcaaacgtgccacgttcatgtcctaca

SEQ ID NO: 223 - DNA, 6146 bp, synthetic construct
  source 1..6146: mol_type = other DNA
  misc_feature 1..6146: note = Description of Sequence: pCTM429
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacctcttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatctaggcctgcaggcggccgccaccatgagactgctggccaagatcatctgcctgatgctgtgggccatctgcgtggccgaggactgcaacgagctgccccccagaagaaacaccgagatcctgaccggctcttggagcgaccagacctaccctgagggcacccaggccatctacaagtgcagacccggctaccggtccctgggcaacatcatcatggtgtgcagaaagggcgagtgggtggccctgaaccccctgagaaagtgccagaagaggccctgcggacaccccggcgataccccttttggcaccttcacactgaccggcggcaacgtgttcgagtacggcgtgaaggccgtgtacacctgtaacgagggctaccagctgctgggcgagatcaactacagagagtgcgacaccgacggctggaccaacgatatccccatctgcgaggtcgtgaagtgcctgcctgtgaccgccccagagaacggcaagatcgtgtccagcgccatggaacccgacagagagtaccacttcggccaggccgtcagattcgtgtgcaacagcggctacaagatcgagggcgacgaggaaatgcactgcagcgacgacggcttctggtccaaagaaaagcctaagtgcgtggaaatcagctgcaagagccccgacgtgatcaacggcagccccatcagccagaagatcatctacaaagagaacgagcggttccagtacaagtgtaacatgggctacgagtacagcgagcggggcgacgccgtgtgtacagaatctggatggcggcctctgcccagctgcgaggaaaagagctgcgacaacccctacatccccaacggcgactacagccccctgcggatcaagcacagaaccggcgacgagatcacctaccagtgccggaacggcttctaccccgccaccagaggcaataccgccaagtgtaccagcaccggctggatccctgcccccagatgtaccctgaagccctgcgactaccctgacatcaagcacggcggcctgtaccacgagaacatgcggaggccctacttccctgtggccgtgggcaagtactacagctactactgcgacgagcacttcgagacacccagcggcagctactgggaccacatccactgtacccaggacggctggtcccctgccgtgccctgcctgaggaagtgctacttcccctacctggaaaacggctacaaccagaactacggccggaagttcgtgcagggcaagagcatcgatgtggcctgccaccctggatacgccctgcctaaggcccagaccaccgtgacctgcatggaaaatggatggtcccccaccccccggtgcatcagagtgtccttcaccctgggcggcaggggtcgacgcggcggcatggcctctcatagactgctgctgctgtgtctggccggcctggtgtttgtgtctgaggcctctgataccggcagacccttcgtggaaatgtacagcgagatccccgagatcatccacatgaccgagggcagagagctggtcatcccctgcagagtgacaagccccaacatcaccgtgactctgaagaagttccctctggacacactgatccccgacggcaagagaatcatctgggacagccggaagggcttcatcatcagcaacgccacctacaaagagatcggcctgctgacctgtgaagccaccgtgaatggccacctgtacaagaccaactacctgacacacagacagaccaacaccatcatcgacgtggtgctgagccctagccacggcattgaactgtctgtgggcgagaagctggtgctgaactgtaccgccagaaccgagctgaacgtgggcatcgacttcaactgggagtaccccagcagcaagcaccagcacaagaaactggtcaaccgggacctgaaaacccagagcggcagcgagatgaagaaattcctgagcaccctgaccatcgacggcgtgaccagatctgaccagggcctgtacacatgtgccgccagctctggcctgatgaccaagaaaaacagcaccttcgtgcgggtgcacgagaaggacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaatagcacctacagagtggtgtccgtgctgaccgtgctgcaccaggattggctgaacggcaaagagtacaagtgcaaggtgtccaacaaggccctgcctgctcctatcgagaaaaccatctccaaggccaagggccagcctagggaaccccaggtttacacactgcctccaagcagggacgagctgacaaagaaccaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgcagcgtgatgcacgaggccctgcacaaccactacacccagaagtccctgagcctgtctcctggctaagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctcaggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcactgtcattagcaactccttgtccttcgatctcgtcaacaacagcttgcagttcaaatacaagacccagaaggcgactattctggaagcgagcttgaagagttaacctgcagagagcccccgcagtgtcgacaattaatcatcggcatagtatatcggcatagtataatacgacaaggtgaggaagtaaaaaatgagccatatccaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcaaggctgactgcagggcgagaagattgcgagctgtgcggctgagttgacgtatctgtgctggatgattactcataacggcaccgctatcaaacgtgccacgttcatgtcctaca

SEQ ID NO: 224-225 - skipped (2 entries)

SEQ ID NO: 226 - DNA, 6631 bp, synthetic construct
  misc_feature 1..6631: note = Description of Sequence: pCTM417
  source 1..6631: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacctcttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatctaggcctgcaggccaccatggcctctcatagactgctgctgctgtgtctggccggcctggtgtttgtgtctgaggcctctgataccggcagacccttcgtggaaatgtacagcgagatccccgagatcatccacatgaccgagggcagagagctggtcatcccctgcagagtgacaagccccaacatcaccgtgactctgaagaagttccctctggacacactgatccccgacggcaagagaatcatctgggacagccggaagggcttcatcatcagcaacgccacctacaaagagatcggcctgctgacctgtgaagccaccgtgaatggccacctgtacaagaccaactacctgacacacagacagaccaacaccatcatcgacgtggtgctgagccctagccacggcattgaactgtctgtgggcgagaagctggtgctgaactgtaccgccagaaccgagctgaacgtgggcatcgacttcaactgggagtaccccagcagcaagcaccagcacaagaaactggtcaaccgggacctgaaaacccagagcggcagcgagatgaagaaattcctgagcaccctgaccatcgacggcgtgaccagatctgaccagggcctgtacacatgtgccgccagctctggcctgatgaccaagaaaaacagcaccttcgtgcgggtgcacgagaaggacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaatagcacctacagagtggtgtccgtgctgaccgtgctgcaccaggattggctgaacggcaaagagtacaagtgcaaggtgtccaacaaggccctgcctgctcctatcgagaaaaccatctccaaggccaagggccagcctagggaaccccaggtttacacactgcctccaagcagggacgagctgacaaagaaccaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgcagcgtgatgcacgaggccctgcacaaccactacacccagaagtccctgagcctgtctcctggctaagagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcccctcaagcgtattcaacaaggggctgaaggatgcccagaaggtaccccattgtatgggatctgatctggggcctcggtgcacatgcttttcatgtgtttagtcgaggttaaaaaacgtctaggccccccgaaccacggggacgtggttttcctttgaaaaacacgatgataatagaattcgatatcaagcttatcgataccgtcgactagagctcgccaccatgagactgctggccaagatcatctgcctgatgctgtgggccatctgcgtggccgaggactgcaacgagctgccccccagaagaaacaccgagatcctgaccggctcttggagcgaccagacctaccctgagggcacccaggccatctacaagtgcagacccggctaccggtccctgggcaacatcatcatggtgtgcagaaagggcgagtgggtggccctgaaccccctgagaaagtgccagaagaggccctgcggacaccccggcgataccccttttggcaccttcacactgaccggcggcaacgtgttcgagtacggcgtgaaggccgtgtacacctgtaacgagggctaccagctgctgggcgagatcaactacagagagtgcgacaccgacggctggaccaacgatatccccatctgcgaggtcgtgaagtgcctgcctgtgaccgccccagagaacggcaagatcgtgtccagcgccatggaacccgacagagagtaccacttcggccaggccgtcagattcgtgtgcaacagcggctacaagatcgagggcgacgaggaaatgcactgcagcgacgacggcttctggtccaaagaaaagcctaagtgcgtggaaatcagctgcaagagccccgacgtgatcaacggcagccccatcagccagaagatcatctacaaagagaacgagcggttccagtacaagtgtaacatgggctacgagtacagcgagcggggcgacgccgtgtgtacagaatctggatggcggcctctgcccagctgcgaggaaaagagctgcgacaacccctacatccccaacggcgactacagccccctgcggatcaagcacagaaccggcgacgagatcacctaccagtgccggaacggcttctaccccgccaccagaggcaataccgccaagtgtaccagcaccggctggatccctgcccccagatgtaccctgaagccctgcgactaccctgacatcaagcacggcggcctgtaccacgagaacatgcggaggccctacttccctgtggccgtgggcaagtactacagctactactgcgacgagcacttcgagacacccagcggcagctactgggaccacatccactgtacccaggacggctggtcccctgccgtgccctgcctgaggaagtgctacttcccctacctggaaaacggctacaaccagaactacggccggaagttcgtgcagggcaagagcatcgatgtggcctgccaccctggatacgccctgcctaaggcccagaccaccgtgacctgcatggaaaatggatggtcccccaccccccggtgcatcagagtgtccttcaccctgtgagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctcaggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcactgtcattagcaactccttgtccttcgatctcgtcaacaacagcttgcagttcaaatacaagacccagaaggcgactattctggaagcgagcttgaagagttaacctgcagagagcccccgcagtgtcgacaattaatcatcggcatagtatatcggcatagtataatacgacaaggtgaggaagtaaaaaatgagccatatccaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcaaggctgactgcagggcgagaagattgcgagctgtgcggctgagttgacgtatctgtgctggatgattactcataacggcaccgctatcaaacgtgccacgttcatgtcctaca

SEQ ID NO: 227 - DNA, 7136 bp, synthetic construct
  misc_feature 1..7136: note = Description of Sequence: pCTM369
  source 1..7136: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacctcttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatctaggcctgcaggcggccgccaccatgagactgctggccaagatcatctgcctgatgctgtgggccatctgcgtggccgaggactgcaacgagctgccccccagaagaaacaccgagatcctgaccggctcttggagcgaccagacctaccctgagggcacccaggccatctacaagtgcagacccggctaccggtccctgggcaacatcatcatggtgtgcagaaagggcgagtgggtggccctgaaccccctgagaaagtgccagaagaggccctgcggacaccccggcgataccccttttggcaccttcacactgaccggcggcaacgtgttcgagtacggcgtgaaggccgtgtacacctgtaacgagggctaccagctgctgggcgagatcaactacagagagtgcgacaccgacggctggaccaacgatatccccatctgcgaggtcgtgaagtgcctgcctgtgaccgccccagagaacggcaagatcgtgtccagcgccatggaacccgacagagagtaccacttcggccaggccgtcagattcgtgtgcaacagcggctacaagatcgagggcgacgaggaaatgcactgcagcgacgacggcttctggtccaaagaaaagcctaagtgcgtggaaatcagctgcaagagccccgacgtgatcaacggcagccccatcagccagaagatcatctacaaagagaacgagcggttccagtacaagtgtaacatgggctacgagtacagcgagcggggcgacgccgtgtgtacagaatctggatggcggcctctgcccagctgcgaggaaaagagctgcgacaacccctacatccccaacggcgactacagccccctgcggatcaagcacagaaccggcgacgagatcacctaccagtgccggaacggcttctaccccgccaccagaggcaataccgccaagtgtaccagcaccggctggatccctgcccccagatgtaccctgaagccctgcgactaccctgacatcaagcacggcggcctgtaccacgagaacatgcggaggccctacttccctgtggccgtgggcaagtactacagctactactgcgacgagcacttcgagacacccagcggcagctactgggaccacatccactgtacccaggacggctggtcccctgccgtgccctgcctgaggaagtgctacttcccctacctggaaaacggctacaaccagaactacggccggaagttcgtgcagggcaagagcatcgatgtggcctgccaccctggatacgccctgcctaaggcccagaccaccgtgacctgcatggaaaatggatggtcccccaccccccggtgcatcagagtgtccttcaccctgtgaggtaccactgtgcatatgcgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcccctcaagcgtattcaacaaggggctgaaggatgcccagaaggtaccccattgtatgggatctgatctggggcctcggtgcacatgcttttcatgtgtttagtcgaggttaaaaaacgtctaggccccccgaaccacggggacgtggttttcctttgaaaaacacgatgataatagaattcgatatcaagcttatcgataccgtcgactagagctccaccatgaagctgctgcatgtgtttctgctgttcctctgcttccacctgaggttctgcaaagtgacctacaccagccaagaggacctggtggaaaagaagtgcctggccaagaagtacacccacctgagctgcgacaaggtgttctgccagccttggcagagatgcatcgagggcacctgtgtgtgcaagctgccctatcagtgccccaagaatggcacagccgtgtgcgccaccaacagaagaagcttccctacctactgccagcagaaaagcctggaatgtctgcaccccggcaccaagtttctgaacaacggcacctgtaccgccgagggcaagtttagcgtgtccctgaagcacggcaacaccgactctgagggcatcgtggaagtgaagctggtggaccaggacaagaccatgttcatctgcaagagcagctggtccatgcgcgaggccaatgtggcttgtctggatctgggattccagcagggcgccgacacacagagaagattcaagctgagcgacctgagcatcaacagcaccgagtgcctgcatgtgcactgtagaggcctggaaacaagcctggccgagtgcaccttcaccaagagaaggaccatgggctaccaggacttcgccgacgtcgtgtgctacacccagaaagccgactctcccatggacgatttcttccagtgcgtgaacggcaagtacatcagccagatgaaggcctgcgacggcatcaacgattgcggcgatcagagcgacgagctgtgctgcaaagcctgtcaaggcaagggcttccactgcaagtccggcgtgtgtatccctagccagtaccagtgcaatggcgaggtggactgtatcaccggcgaggatgaagtgggctgtgccggatttgccagcgtggcccaagaggaaaccgagatcctgaccgccgatatggacgccgagcggcggagaatcaaaagcctgctgcctaagctgtcctgcggcgtgaagaaccggatgcacatccggcgcaagagaatcgtcggaggcaaaagagcacagctgggcgatctgccttggcaagtggccatcaaggatgcctccggcatcacttgtggcggcatctacatcggcggctgctggattctgacagccgctcattgtctgcgggccagcaagacccaccggtatcagatctggaccaccgtggtggactggattcaccccgacctgaagcggatcgtgatcgagtacgtggaccggatcatcttccacgagaactacaacgccggcacctaccagaacgatatcgccctgatcgagatgaagaaggacgggaacaagaaggactgcgagctgcctagatctatccccgcctgtgttccttggagcccctacctgttccagcctaacgatacctgcatcgtgtccggctggggcagagagaaggataacgagagggtgttcagcctgcagtggggcgaagtgaaactgatcagcaactgcagcaagttctacggcaaccggttctacgagaaagaaatggaatgcgccggcacatacgacggctccatcgatgcctgtaaaggcgattctggcggccctctcgtgtgcatggatgccaacaatgtgacctacgtgtggggcgtcgtgtcctggggagagaattgtggcaagcctgagttccccggcgtgtacaccaaggtggccaactacttcgactggatcagctaccacgtgggcagaccctttatcagccagtacaacgtctgagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctcaggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcactgtcattagcaactccttgtccttcgatctcgtcaacaacagcttgcagttcaaatacaagacccagaaggcgactattctggaagcgagcttgaagagttaacctgcagagagcccccgcagtgtcgacaattaatcatcggcatagtatatcggcatagtataatacgacaaggtgaggaagtaaaaaatgagccatatccaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcaaggctgactgcagggcgagaagattgcgagctgtgcggctgagttgacgtatctgtgctggatgattactcataacggcaccgctatcaaacgtgccacgttcatgtcctaca

SEQ ID NO: 228 - protein, 124 aa, synthetic construct
  REGION 1..124: note = Description of Sequence: FHR-2 dimerization domain (SCR1-2)
EAMFCDFPKINHGILYDEEKYKPFSQVPTGEVFYYSCEYNFVSPSKSFWTRITCAEEGWSPTPKCLRLCFFPFVENGHSESSGQTHLEGDTVQIICNTGYRLQNNENNISCVERGWSTPPKCRS

SEQ ID NO: 229 - DNA, 198 bp, synthetic construct
  misc_feature 1..198: note = Description of Sequence: FHR-2 SCR1 - Non-codon Optimized
  source 1..198: mol_type = other DNA
gaagcaatgttctgtgattttccaaaaataaaccatggaattctatatgatgaagaaaaatataagccattttcccaagttcctacaggggaagttttctattactcctgtgaatataattttgtgtctccttcaaaatccttttggactcgcataacgtgcgcagaagaaggatggtcaccaacaccaaagtgtctc

SEQ ID NO: 230 - DNA, 198 bp, synthetic construct
  misc_feature 1..198: note = Description of Sequence: FHR-2 SCR1 - GeneArt Codon Optimized
  source 1..198: mol_type = other DNA
gaggccatgttctgcgacttccccaagatcaaccacggcatcctgtacgacgaggaaaagtacaagccattcagccaggtgccaaccggcgaggtgttctactacagctgcgagtacaacttcgtgtcccctagcaagagcttctggacccggatcacctgtgccgaagaaggctggtcccctacacctaagtgtctg

SEQ ID NO: 231 - DNA, 174 bp, synthetic construct
  misc_feature 1..174: note = Description of Sequence: FHR-2 SCR2 - Non-codon Optimized
  source 1..174: mol_type = other DNA
agactgtgtttctttccttttgtggaaaatggtcattctgaatcttcaggacaaacacatctggaaggtgatactgtacaaattatttgcaacacaggatacagacttcaaaacaatgagaacaacatttcatgtgtagaacggggctggtccactcctcccaaatgcaggtcc

SEQ ID NO: 232 - DNA, 174 bp, synthetic construct
  misc_feature 1..174: note = Description of Sequence: FHR-2 SCR2 - GeneArt Codon Optimized
  source 1..174: mol_type = other DNA
cggctgtgcttcttccccttcgtggaaaatggccacagcgagagcagcggccagacacatcttgagggcgataccgtgcagatcatctgtaacaccggctaccggctgcagaacaacgagaacaacatcagctgcgtggaacgcggctggtctacccctcctaagtgtagatct

SEQ ID NO: 233 - protein, 124 aa, synthetic construct
  REGION 1..124: note = Description of Sequence: FHR-5 dimerization domain (SCR1-2)
EGTLCDFPKIHHGFLYDEEDYNPFSQVPTGEVFYYSCEYNFVSPSKSFWTRITCTEEGWSPTPKCLRMCSFPFVKNGHSESSGLIHLEGDTVQIICNTGYSLQNNEKNISCVERGWSTPPICSF

SEQ ID NO: 234 - DNA, 198 bp, synthetic construct
  misc_feature 1..198: note = Description of Sequence: FHR-5 SCR1 - Non-codon Optimized
  source 1..198: mol_type = other DNA
gaaggaacactttgtgattttccaaaaatacaccatggatttctgtatgatgaagaagattataaccctttttcccaagttcctacaggggaagttttctattactcctgtgaatataattttgtgtctccttcaaaatccttttggactcgcataacatgcacagaagaaggatggtcaccaacaccgaagtgtctc

SEQ ID NO: 235 - DNA, 198 bp, synthetic construct
  misc_feature 1..198: note = Description of Sequence: FHR-5 SCR1 - GeneArt Codon Optimized
  source 1..198: mol_type = other DNA
gagggcaccctgtgcgactttccaaagatccaccacggctttctgtacgacgaagaggactacaacccattcagccaggtgccaaccggcgaggtgttctactacagctgcgagtacaacttcgtgtcccctagcaagagcttctggacccggatcacctgtaccgaggaaggctggtcccctacacctaagtgtctg

SEQ ID NO: 236 - DNA, 174 bp, synthetic construct
  misc_feature 1..174: note = Description of Sequence: FHR-5 SCR2 - Non-codon Optimized
  source 1..174: mol_type = other DNA
agaatgtgttcctttccttttgtgaaaaatggtcattctgaatcttcaggactaatacatctggaaggtgatactgtacaaattatttgcaacacaggatacagccttcaaaacaatgagaaaaacatttcgtgtgtagaacggggctggtccactcctcccatatgcagcttc

SEQ ID NO: 237 - DNA, 174 bp, synthetic construct
  misc_feature 1..174: note = Description of Sequence: FHR-5 SCR2 - GeneArt Codon Optimized
  source 1..174: mol_type = other DNA
cggatgtgcagcttcccattcgtgaagaacggccacagcgagagcagcggactgattcacctggaaggcgacaccgtgcagatcatctgtaacaccggctacagcctgcagaacaacgagaagaacatcagctgcgtggaacgcggctggtctacccctcctatctgcagcttt

SEQ ID NO: 238 - DNA, 454 bp, synthetic construct
  misc_feature 1..454: note = Description of Sequence: BEST1-EP-454 Enhancer Promoter
  source 1..454: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctcgagctagggtgatgaaattcccaagcaacaccatccttttcaagtgacggcggctcagcactcacgtgggcagtgccagcctctaagagtgggcaggggcactggccacagagtcccagggagtcccaccagcctagtcgccagacc

SEQ ID NO: 239 - DNA, 144 bp, synthetic construct
  misc_feature 1..144: note = Description of Sequence: BEST1-V3 Promoter -144 b
  source 1..144: mol_type = other DNA
ctagggtgatgaaattcccaagcaacaccatccttttcaagtgacggcggctcagcactcacgtgggcagtgccagcctctaagagtgggcaggggcactggccacagagtcccagggagtcccaccagcctagtcgccagacc

SEQ ID NO: 240 - protein, 480 aa, synthetic construct
  REGION 1..480: note = Description of Sequence: HTRA1 protein sequence
MQIPRAALLPLLLLLLAAPASAQLSRAGRSAPLAAGCPDRCEPARCPPQPEHCEGGRARDACGCCEVCGAPEGAACGLQEGPCGEGLQCVVPFGVPASATVRRRAQAGLCVCASSEPVCGSDANTYANLCQLRAASRRSERLHRPPVIVLQRGACGQGQEDPNSLRHKYNFIADVVEKIAPAVVHIELFRKLPFSKREVPVASGSGFIVSEDGLIVTNAHVVTNKHRVKVELKNGATYEAKIKDVDEKADIALIKIDHQGKLPVLLLGRSSELRPGEFVVAIGSPFSLQNTVTTGIVSTTQRGGKELGLRNSDMDYIQTDAIINYGNSGGPLVNLDGEVIGINTLKVTAGISFAIPSDKIKKFLTESHDRQAKGKAITKKKYIGIRMMSLTSSKAKELKDRHRDFPDVISGAYIIEVIPDTPAEAGGLKENDVIISINGQSVVSANDVSDVIKRESTLNMVVRRGNEDIMITVIPEEIDP

SEQ ID NO: 241 - DNA, 29 bp, synthetic construct
  misc_feature 1..29: note = Description of Sequence: Control sequence, Section 10.4.1.3
  source 1..29: mol_type = other DNA
gcactaccagagctaactcagatagtact

SEQ ID NO: 242 - protein, 58 aa, synthetic construct
  REGION 1..58: note = SCR7 protein sequence encoded by SEQ ID NO: 2
KCYFPYLENGYNQNYGRKFVQGKSIDVACHPGYALPKAQTTVTCMENGWSPTPRCIRV

SEQ ID NO: 243 - DNA, 63 bp, synthetic construct
  misc_feature 1..63: note = Description of Sequence: C7 shRNA sequence:
  source 1..63: mol_type = other DNA
gaaggtccttcagcatttctctgtggctccaagagagccacagagaaatgctgaaggaccttc

SEQ ID NO: 244 - DNA, 63 bp, synthetic construct
  misc_feature 1..63: note = Description of Sequence: C7 shRNA sequence:
  source 1..63: mol_type = other DNA
cagaatgcaatggctgtaccaagactcagcaagactgagtcttggtacagccattgcattctg

SEQ ID NO: 245 - DNA, 63 bp, synthetic construct
  misc_feature 1..63: note = Description of Sequence: C7 shRNA sequence:
  source 1..63: mol_type = other DNA
tctgtccatcacctcctgccttgaaagatcaagaatctttcaaggcaggaggtgatggacaga

SEQ ID NO: 246 - DNA, 1527 bp, synthetic construct
  misc_feature 1..1527: note = Description of Sequence: mCFHT.1 cDNA sequence
  source 1..1527: mol_type = other DNA
atgagactgctggccaagatcatctgcctgatgctgtgggccatctgcgtggccgaggactgcaacgagctgccccccagaagaaacaccgagatcctgaccggctcttggagcgaccagacctaccctgagggcacccaggccatctacaagtgcagacccggctaccggtccctgggcaacatcatcatggtgtgcagaaagggcgagtgggtggccctgaaccccctgagaaagtgccagaagaggccctgcggacaccccggcgataccccttttggcaccttcacactgaccggcggcaacgtgttcgagtacggcgtgaaggccgtgtacacctgtaacgagggctaccagctgctgggcgagatcaactacagagagtgcgacaccgacggctggaccaacgatatccccatctgcgaggtcgtgaagtgcctgcctgtgaccgccccagagaacggcaagatcgtgtccagcgccatggaacccgacagagagtaccacttcggccaggccgtcagattcgtgtgcaacagcggctacaagatcgagggcgacgaggaaatgcactgcagcgacgacggcttctggtccaaagaaaagcctaagtgcgtggaaatcagctgcaagagccccgacgtgatcaacggcagccccatcagccagaagatcatctacaaagagaacgagcggttccagtacaagtgtaacatgggctacgagtacagcgagcggggcgacgccgtgtgtacagaatctggatggcggcctctgcccagctgcgaggaaaagagctgcgacaacccctacatccccaacggcgactacagccccctgcggatcaagcacagaaccggcgacgagatcacctaccagtgccggaacggcttctaccccgccaccagaggcaataccgccaagtgtaccagcaccggctggatccctgcccccagatgtaccctgaagccctgcgactaccctgacatcaagcacggcggcctgtaccacgagaacatgcggaggccctacttccctgtggccgtgggcaagtactacagctactactgcgacgagcacttcgagacacccagcggcagctactgggaccacatccactgtacccaggacggctggtcccctgccgtgccctgcctgaggaagtgctacttcccctacctggaaaacggctacaaccagaactacggccggaagttcgtgcagggcaagagcatcgatgtggcctgccaccctggatacgccctgcctaaggcccagaccaccgtgacctgcatggaaaatggatggtcccccaccccccggtgcatcagagtgaggaagtgctatttcccttatttggaaaacggatataatcaaaattacggtcggaaatttgtccaaggtaagtcaatagatgtcgcgtgtcatcctggctatgctcttccgaaagcacagacaacggttacgtgtatggagaacggttggtctccaacccctcgatgtattagggtgtccttcaccctgtga

SEQ ID NO: 247 - protein, 508 aa, synthetic construct
  REGION 1..508: note = Description of Sequence: mCFHT.1 protein sequence
MRLLAKIICLMLWAICVAEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVRKCYFPYLENGYNQNYGRKFVQGKSIDVACHPGYALPKAQTTVTCMENGWSPTPRCIRVSFTL

SEQ ID NO: 248 - DNA, 1704 bp, synthetic construct
  misc_feature 1..1704: note = Description of Sequence: CFHT.2 cDNA sequence
  source 1..1704: mol_type = other DNA
atgagactgctggccaagatcatctgcctgatgctgtgggccatctgcgtggccgaggactgcaacgagctgccccccagaagaaacaccgagatcctgaccggctcttggagcgaccagacctaccctgagggcacccaggccatctacaagtgcagacccggctaccggtccctgggcaacatcatcatggtgtgcagaaagggcgagtgggtggccctgaaccccctgagaaagtgccagaagaggccctgcggacaccccggcgataccccttttggcaccttcacactgaccggcggcaacgtgttcgagtacggcgtgaaggccgtgtacacctgtaacgagggctaccagctgctgggcgagatcaactacagagagtgcgacaccgacggctggaccaacgatatccccatctgcgaggtcgtgaagtgcctgcctgtgaccgccccagagaacggcaagatcgtgtccagcgccatggaacccgacagagagtaccacttcggccaggccgtcagattcgtgtgcaacagcggctacaagatcgagggcgacgaggaaatgcactgcagcgacgacggcttctggtccaaagaaaagcctaagtgcgtggaaatcagctgcaagagccccgacgtgatcaacggcagccccatcagccagaagatcatctacaaagagaacgagcggttccagtacaagtgtaacatgggctacgagtacagcgagcggggcgacgccgtgtgtacagaatctggatggcggcctctgcccagctgcgaggaaaagagctgcgacaacccctacatccccaacggcgactacagccccctgcggatcaagcacagaaccggcgacgagatcacctaccagtgccggaacggcttctaccccgccaccagaggcaataccgccaagtgtaccagcaccggctggatccctgcccccagatgtaccctgaagccctgcgactaccctgacatcaagcacggcggcctgtaccacgagaacatgcggaggccctacttccctgtggccgtgggcaagtactacagctactactgcgacgagcacttcgagacacccagcggcagctactgggaccacatccactgtacccaggacggctggtcccctgccgtgccctgcctgaggaagtgctacttcccctacctggaaaacggctacaaccagaactacggccggaagttcgtgcagggcaagagcatcgatgtggcctgccaccctggatacgccctgcctaaggcccagaccaccgtgacctgcatggaaaatggatggtcccccaccccccggtgcatcagagtgaggaagtgctatttcccttatttggaaaacggatataatcaaaattacggtcggaaatttgtccaaggtaagtcaatagatgtcgcgtgtcatcctggctatgctcttccgaaagcacagacaacggttacgtgtatggagaacggttggtctccaacccctcgatgtattagggtgcggaaatgctacttcccatacctggaaaacggatataaccagaactacggcagaaagttcgtgcagggcaagtctatcgacgtggcttgtcaccccggctacgccctgcctaaggcccagaccaccgtcacatgcatggagaatggctggagccctacccctagatgcatcagagtgtccttcaccctgtga

SEQ ID NO: 249 - protein, 567 aa, synthetic construct
  REGION 1..567: note = Description of Sequence: CFHT.2 protein sequence
MRLLAKIICLMLWAICVAEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVRKCYFPYLENGYNQNYGRKFVQGKSIDVACHPGYALPKAQTTVTCMENGWSPTPRCIRVRKCYFPYLENGYNQNYGRKFVQGKSIDVACHPGYALPKAQTTVTCMENGWSPTPRCIRVSFTL

SEQ ID NO: 250 - DNA, 1752 bp, synthetic construct
  misc_feature 1..1752: note = Description of Sequence: CFHT.0 cDNA sequence
  source 1..1752: mol_type = other DNA
atgagactgctggccaagatcatctgcctgatgctgtgggccatctgcgtggccgaagcaacattttgtgattttccaaaaataaaccatggaattctatatgatgaagaaaaatataagccattttcccaggttcctacaggggaagttttctattactcctgtgaatataattttgtgtctccttcaaaatcattttggactcgcataacatgcacagaagaaggatggtcaccaacaccaaagtgtctcagactgtgtttctttccttttgtggaaaatggtcattctgaatcttcaggacaaacacatctggaaggtgatactgtgcaaattatttgcaacacaggatacagacttcaaaacaatgagaacaacatttcatgtgtagaacggggctggtccacccctcccaaatgcaggtccggcggcggcggctccggcggcggcggctccgaggactgcaacgagctgccccccagaagaaacaccgagatcctgaccggctcttggagcgaccagacctaccctgagggcacccaggccatctacaagtgcagacccggctaccggtccctgggcaacatcatcatggtgtgcagaaagggcgagtgggtggccctgaaccccctgagaaagtgccagaagaggccctgcggacaccccggcgataccccttttggcaccttcacactgaccggcggcaacgtgttcgagtacggcgtgaaggccgtgtacacctgtaacgagggctaccagctgctgggcgagatcaactacagagagtgcgacaccgacggctggaccaacgatatccccatctgcgaggtcgtgaagtgcctgcctgtgaccgccccagagaacggcaagatcgtgtccagcgccatggaacccgacagagagtaccacttcggccaggccgtcagattcgtgtgcaacagcggctacaagatcgagggcgacgaggaaatgcactgcagcgacgacggcttctggtccaaagaaaagcctaagtgcgtggaaatcagctgcaagagccccgacgtgatcaacggcagccccatcagccagaagatcatctacaaagagaacgagcggttccagtacaagtgtaacatgggctacgagtacagcgagcggggcgacgccgtgtgtacagaatctggatggcggcctctgcccagctgcgaggaaaagagctgcgacaacccctacatccccaacggcgactacagccccctgcggatcaagcacagaaccggcgacgagatcacctaccagtgccggaacggcttctaccccgccaccagaggcaataccgccaagtgtaccagcaccggctggatccctgcccccagatgtaccctgaagccctgcgactaccctgacatcaagcacggcggcctgtaccacgagaacatgcggaggccctacttccctgtggccgtgggcaagtactacagctactactgcgacgagcacttcgagacacccagcggcagctactgggaccacatccactgtacccaggacggctggtcccctgccgtgccctgcctgaggaagtgctacttcccctacctggaaaacggctacaaccagaactacggccggaagttcgtgcagggcaagagcatcgatgtggcctgccaccctggatacgccctgcctaaggcccagaccaccgtgacctgcatggaaaatggatggtcccccaccccccggtgcatcagagtgtccttcaccctgtga

SEQ ID NO: 251 - protein, 583 aa, synthetic construct
  REGION 1..583: note = Description of Sequence: CFHT.0 protein sequence
MRLLAKIICLMLWAICVAEATFCDFPKINHGILYDEEKYKPFSQVPTGEVFYYSCEYNFVSPSKSFWTRITCTEEGWSPTPKCLRLCFFPFVENGHSESSGQTHLEGDTVQIICNTGYRLQNNENNISCVERGWSTPPKCRSGGGGSGGGGSEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVSFTL

SEQ ID NO: 252 - DNA, 1752 bp, synthetic construct; identical to SEQ ID NO: 250
  misc_feature 1..1752: note = Description of Sequence: dCFHT.0 cDNA sequence
  source 1..1752: mol_type = other DNA

SEQ ID NO: 253 - protein, 583 aa, synthetic construct; identical to SEQ ID NO: 251
  REGION 1..583: note = Description of Sequence: dCFHT.0 protein sequence